(19) United States
(12) Patent Application Publication
(10) Pub. No.: US 2025/0122224 A1
(43) Pub. Date: Apr. 17, 2025

(54) Crystalline Salt and Solvate Forms Of Murizatoclax (AMG 397)

(71) Applicant: AMGEN INC., Thousand Oaks, CA (US)

(72) Inventors: Ron C. Kelly, Thousand Oaks, CA (US); Mary Chaves, Arlington, MA (US); Jing Teng, Thousand Oaks, CA (US); Stephan Parent, Thousand Oaks, CA (US); Van Luu, Thousand Oaks, CA (US); Robert P. Farrell, Thousand Oaks, CA (US); James E. Huckle, Thousand Oaks, CA (US); Michal Achmatowicz, Simi Valley, CA (US); Tian Wu, Newbury Park, CA (US); Darren Leonard Reid, Belmont, MA (US); Lingyun Xiao, Thousand Oaks, CA (US)

(21) Appl. No.: 18/730,353

(22) PCT Filed: Feb. 3, 2023
(86) PCT No.: PCT/US2023/012252

§ 371 Date: Jul. 19, 2024

Related U.S. Application Data
(60) Provisional application No. 63/306,781, filed on Feb. 4, 2022.

(51) Int. Cl. C07D 519/00; A61K 31/553

(52) U.S. Cl. CPC C07D 519/00; A61K 31/553

(57) ABSTRACT

Disclosed herein are salt and solvate forms of (4S,7aR,9aR,10R,11E,14S,15R)-6′-chloro-10-methoxy-14,15-dimethyl-10-{[(9aR)-octahydro-2H-pyrido[1,2-a]pyrazin-2-yl]methyl}-3′,4′,7a,8,9,9a,10,13,14,15-decahydro-2′H,3H,5H-spiro[1,19-etheno-16,16-cyclobuta[i][1,4]oxazepino[3,4-f][1,2,7]thiadlazacyclohexadecine-4,1 naphthalene]-16,16,18 (7H,17H)-trione (AMG 397): (AMG 397), such as crystalline salt and solvate forms thereof. Also disclosed are methods of making the salt and solvate forms, and methods of treating diseases and disorders with the salt and solvate forms.

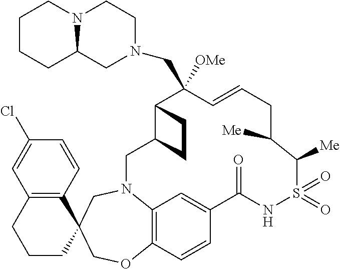

### BACKGROUND

### Technical Field

The present disclosure relates to salt and solvate forms of (4S,7aR,9aR,10R,11E,14S,15R)-6′-chloro-10-methoxy-14,15-dimethyl-10-{[(9aR)-octahydro-2H-pyrido[1,2-a]pyrazin-2-yl]methyl}-3′,4′,7a,8,9,9a,10,13,14,15-decahydro-2′H,3H,5H-spiro[1,19-etheno-16,16-cyclobuta[i][1,4]oxazepino[3,4-f][1,2,7]thiadiazacyclohexadecine-4,1′-naphthalene]-16,16,18 (7H,17H)-trione (AMG 397), such as crystalline salt and solvate forms, which functions as an inhibitor of myeloid cell leukemia 1 protein (Mcl-1).

### Description of Related Technology

The compound, (4S,7aR,9aR,10R,11E,14S,15R)-6′-chloro-10-methoxy-14,15-dimethyl-10-{[(9aR)-octahydro-2H-pyrido[1,2-a]pyrazin-2-yl]methyl}-3′,4′,7a,8,9,9a,10,13,14,15-decahydro-2′H,3H,5H-spiro[1,19-etheno-16,16-cyclobuta[i][1,4]oxazepino[3,4-f][1,2,7]thiadiazacyclohexadecine-4,1′-naphthalene]-16,16,18(7H,17H)-trione (AMG 397), is useful as an inhibitor of myeloid cell leukemia 1 (“Mcl-1):

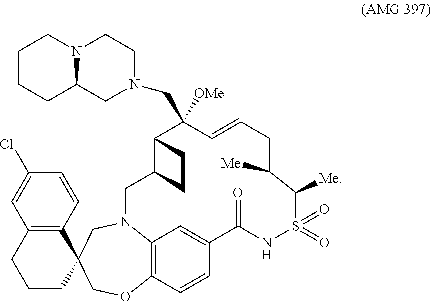

One common characteristic of human cancer is overexpression of Mcl-1. Mcl-1 overexpression prevents cancer cells from undergoing programmed cell death (apoptosis), allowing the cells to survive despite widespread genetic damage.

Mcl-1 is a member of the Bcl-2 family of proteins. The Bcl-2 family includes pro-apoptotic members (such as BAX and BAK) which, upon activation, form a homo-oligomer in the outer mitochondrial membrane that leads to pore formation and the escape of mitochondrial contents, a step in triggering apoptosis. Antiapoptotic members of the Bcl-2 family (such as Bcl-2, Bcl-XL, and Mcl-1) block the activity of BAX and BAK. Other proteins (such as BID, BIM, BIK, and BAD) exhibit additional regulatory functions. Research has shown that Mcl-1 inhibitors can be useful for the treatment of cancers. MCI-1 is overexpressed in numerous cancers.

U.S. Pat. No. 10,300,075, which is incorporated herein by reference in its entirety, discloses AMG 397 as an Mcl-1 inhibitor and provides a method for preparing it. However, alternative forms of AMG 397 with improved properties are desirable, particularly for clinical use of AMG 397.

### SUMMARY

Provided herein are salt and solvate forms of AMG 397, such as crystalline salt and solvate forms thereof, wherein AMG 397 has the structure

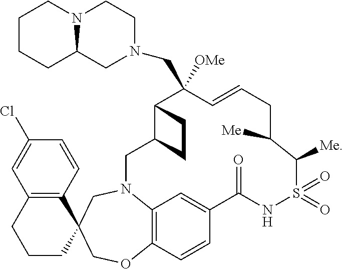

Also provided herein are crystalline forms of AMG 397 as a trifluoroethanol solvate, characterized by XRPD pattern peaks at 17.5, 19.2, 19.4, and 21.7±0.2° 2θ using Cu Kα radiation.

Also provided herein are crystalline forms of AMG 397 as a hexafluoroisopropanol solvate, characterized by XRPD pattern peaks at 11.4, 18.6, and 18.8±0.2° 2θ using Cu Kα radiation.

Also provided herein are crystalline forms of AMG 397 as a 1-propanol solvate, characterized by XRPD pattern peaks at 13.3, 15.1, and 18.5±0.2° 2θ using Cu Kα radiation.

Also provided herein are crystalline forms of AMG 397 as an isopropanol solvate, characterized by XRPD pattern peaks at 6.1, 7.1, and 10.0±0.2° 2θ using Cu Kα radiation.

Also provided herein are crystalline forms of AMG 397 as an isopropanol solvate, characterized by XRPD pattern peaks at 13.3, 15.1, and 18.6±0.2° 2θ using Cu Kα radiation.

Also provided herein are crystalline forms of AMG 397 as an acetonitrile solvate, characterized by XRPD pattern peaks at 10.2, 17.0, and 20.5±0.2° 2θ using Cu Kα radiation.

Also provided herein are crystalline forms of AMG 397 as an acetic acid solvate, characterized by solid state ¹³C NMR peaks at 13.63, 19.22, 20.40, 24.22, 25.69, 26.57, 27.75, 29.81, 30.40, 31.28, 36.57, 38.34, 40.10, 43.04, 49.51, 50.10, 51.86, 54.51, 56.28, 57.16, 57.75, 60.10, 62.16, 65.39, 77.75, 85.10, 115.39, 123.63, 125.10, 128.04, 131.27, 133.04, 133.92, 135.98, 139.80, 141.27, 143.04, 151.86, and 173.92±0.5 ppm.

Also provided herein are crystalline forms of AMG 397 as a hydrochloride salt, characterized by XRPD pattern peaks at 12.9, 16.2, and 17.9±0.2° 2θ using Cu Kα radiation.

Also provided herein are amorphous forms of AMG 397 as a sodium salt, having an XRPD pattern substantially as shown in FIG. 21.

Also provided herein are crystalline forms of AMG 397 as a potassium salt, characterized by XRPD pattern peaks at 12.8, 13.4, and 17.2±0.2° 2θ using Cu Kα radiation.

Also provided herein are crystalline forms of AMG 397 as a potassium salt (ethyl acetate solvate), characterized by XRPD pattern peaks at 2.7, 11.7, and 12.2±0.2° 2θ using Cu Kα radiation.

Also provided herein are crystalline forms of AMG 397 as a sulfate salt, characterized by XRPD pattern peaks at 9.3, 13.9, and 19.2±0.2° 2θ using Cu Kα radiation.

Also provided herein are crystalline forms of AMG 397 as a sulfate salt, characterized by XRPD pattern peaks at 11.7, 17.1, and 20.1±0.2° 2θ using Cu Kα radiation.

Also provided herein are crystalline forms of AMG 397 as a sulfate salt, characterized by XRPD pattern peaks at 12.3, 17.7, 18.4, and 20.6±0.2° 2θ using Cu Kα radiation.

Also provided herein are crystalline forms of AMG 397 as a phosphate salt, characterized by ¹³C NMR peaks at 5.8, 15.0, 18.3, 21.2, 22.2, 23.6, 27.6, 27.6, 29.3, 31.7, 31.9, 35.7, 41.3, 43.6, 49.9, 51.7, 53.3, 53.7, 55.8, 57.7, 58.8, 58.9, 59.8, 61.0, 79.6, 80.9, 115.4, 117.3, 119.1, 126.0, 127.9, 128.7, 129.4, 129.5, 130.2, 139.2, 139.8, 139.9, 150.8, and 168.8±0.5 ppm.

Also provided herein are crystalline forms of AMG 397 as a fumarate salt acetone sovate, characterized by XRPD pattern peaks at 17.6, 18.2, and 18.4±0.2° 2θ using Cu Kα radiation.

Also provided herein are crystalline forms of AMG 397 as a fumarate salt, characterized by XRPD pattern peaks at 11.9, 17.9, and 18.1±0.2° 2θ using Cu Kα radiation.

Also provided herein are crystalline forms of AMG 397 as a citrate salt, characterized by XRPD pattern peaks at 10.6, 17.6, and 18.3±0.2° 2θ using Cu Kα radiation.

Also provided herein are crystalline forms of AMG 397 as a citrate salt, characterized by XRPD pattern peaks at 17.7, 18.4, and 18.5±0.2° 2θ using Cu Kα radiation.

Also provided herein are crystalline forms of AMG 397 as a lactate salt, characterized by XRPD pattern peaks at 12.1, 17.8, and 18.3±0.2° 2θ using Cu Kα radiation.

Also provided herein are crystalline forms of AMG 397 as a succinate salt, characterized by XRPD pattern peaks at 17.6, 18.4, and 18.7±0.2° 2θ using Cu Kα radiation.

Also provided herein are crystalline forms of AMG 397 as an ammonium salt, characterized by XRPD pattern peaks at 6.2, 10.3, and 17.2±0.2° 2θ using Cu Kα radiation.

Also provided herein are crystalline forms AMG 397 as a besylate salt, characterized by XRPD pattern peaks at 17.6, 18.4, and 18.7±0.2° 2θ using Cu Kα radiation.

Also provided herein are crystalline forms of AMG 397 as a tosylate salt, characterized by XRPD pattern peaks at 18.2, 18.4, and 20.5±0.2° 2θ using Cu Kα radiation.

Also provided herein are crystalline forms of AMG 397 as a maleate salt, characterized by XRPD pattern peaks at 18.2, 18.9, and 19.9±0.2° 2θ using Cu Kα radiation.

Also provided herein are crystalline forms of AMG 397 as a maleate salt, characterized by XRPD pattern peaks at 10.6, 18.6, and 20.3±0.2° 2θ using Cu Kα radiation.

Also provided herein are crystalline forms of AMG 397 as a malonate salt, characterized by XRPD pattern peaks at 12.2, 18.8, and 20.4±0.2° 2θ using Cu Kα radiation.

Also provided herein are crystalline forms of AMG 397 as a malonate salt, characterized by XRPD pattern peaks at 10.6, 18.5, and 20.2±0.2° 2θ using Cu Kα radiation.

Also provided herein are crystalline forms of AMG 397 as a tartrate salt, characterized by XRPD pattern peaks at 18.2, 18.6, and 20.2±0.2° 2θ using Cu Kα radiation.

Also provided herein are crystalline forms of AMG 397 as a tris(hydroxymethyl)aminomethane salt acetone solvate, characterized by XRPD pattern peaks at 10.0, 16.8, and 20.0±0.2° 2θ using Cu Kα radiation.

Also provided herein are crystalline forms of AMG 397 as an iodide salt, characterized by XRPD pattern peaks at 17.0, 18.0, and 18.1±0.2° 2θ using Cu Kα radiation.

Also provided herein are pharmaceutical formulations comprising the salt and solvate forms of AMG 397, such as the crystalline salt and solvate forms thereof, as described herein and a pharmaceutically acceptable excipient.

Also provided herein are methods of treating a subject suffering from cancer, comprising administering to the subject a therapeutically effective amount of the pharmaceutical formulation comprising the salt and solvate forms of AMG 397, such as the crystalline salt and solvate forms thereof as described herein and a pharmaceutically acceptable excipient.

### BRIEF DESCRIPTION OF THE DRAWINGS

FIG. 1 depicts an X-ray powder diffraction (“XRPD”) pattern of the crystalline trifluoroethanol solvate form of AMG 397.

FIG. 2 depicts an X-ray powder diffraction (“XRPD”) pattern of the crystalline hexafluoroisopropanol solvate form of AMG 397.

FIG. 3 depicts an X-ray powder diffraction (“XRPD”) pattern of the crystalline 1-propanol solvate form of AMG 397.

FIG. 4 depicts depicts a differential scanning calorimetry (“DSC”) thermograph of the crystalline 1-propanol solvate form of AMG 397 indicating a Tm of 234° C.

FIG. 5 depicts a thermogravimetric analysis (“TGA”) trace of the crystalline 1-propanol solvate form of AMG 397 showing 5.6% weight loss form 38-190° C., prior to melt/degradation.

FIG. 6 depicts an X-ray powder diffraction (“XRPD”) pattern of the crystalline isopropanol solvate form 1 of AMG 397.

FIG. 7 depicts a differential scanning calorimetry (“DSC”) thermograph of the crystalline isopropanol solvate form 1 of AMG 397 indicating a Tm of 247° C.

FIG. 8 depicts a thermogravimetric analysis (“TGA”) trace of the crystalline isopropanol solvate form 1 of AMG 397 showing 0.5% weight loss from 39-120° C., prior to melt/degradation.

FIG. 9 depicts an X-ray powder diffraction (“XRPD”) pattern of the crystalline isopropanol solvate form 2 of AMG 397.

FIG. 10 depicts a differential scanning calorimetry (“DSC”) thermograph of the crystalline isopropanol solvate form 2 of AMG 397 indicating a Tm of 83° C. and 239° C.

FIG. 11 depicts a thermogravimetric analysis (“TGA”) trace of the crystalline isopropanol solvate form 2 of AMG 397 showing 19.0% weight loss from 37-111° C., prior to melt/degradation.

FIG. 12 depicts an X-ray powder diffraction (“XRPD”) pattern of the crystalline acetonitrile solvate form of AMG 397.

FIG. 13 depicts an X-ray powder diffraction (“XRPD”) pattern of the crystalline acetic acid solvate form of AMG 397.

FIG. 14 depicts a differential scanning calorimetry (“DSC”) thermograph of the crystalline acetic acid solvate form 2 of AMG 397 indicating a Tm of 95° C. and 155° C.

FIG. 15 depicts a thermogravimetric analysis (“TGA”) trace of the crystalline acetic acid solvate form of AMG 397 showing 3.1% weight loss to 150° C., with an additional 10.4% weight loss to 250° C. prior to melt/degradation.

FIG. 16 depicts a solid state ¹³C NMR of the crystalline acetic acid solvate form of AMG 397.

FIG. 17 depicts an X-ray powder diffraction (“XRPD”) pattern of the crystalline hydrochloride salt form 1 of AMG 397.

FIG. 18 depicts a differential scanning calorimetry (“DSC”) thermograph of the crystalline hydrochloride salt form 1 of AMG 397 indicating a Tm of 267° C.

FIG. 19 depicts a thermogravimetric analysis (“TGA”) trace of the crystalline hydrochloride salt form 1 of AMG 397 showing 9.5% weight loss from 35-275° C., with 5.3% weight loss from 35-250° C. prior to melt/degradation.

FIG. 20 depicts a moisture sorption profile (DVS) of the crystalline hydrochloride salt form 1 of AMG 397 showing weight gain of ˜0.7% by 95% relative humidity.

FIG. 21 depicts an X-ray powder diffraction (“XRPD”) pattern of the amorphous sodium salt form of AMG 397.

FIG. 22 depicts a differential scanning calorimetry (“DSC”) thermograph of the amorphous sodium salt form of AMG 397 indicating a Tm of 216° C. by reverse heat flow.

FIG. 23 depicts a thermogravimetric analysis (“TGA”) trace of the crystalline hydrochloride salt form of AMG 397 showing 4.9% weight loss to 210° C.

FIG. 24 depicts a moisture sorption profile (DVS) of the amorphous sodium salt of AMG 397 showing weight gain of ˜11.4% by 95% relative humidity and no form change after the test.

FIG. 25 depicts an X-ray powder diffraction (“XRPD”) pattern of the crystalline potassium salt form 1 of AMG 397.

FIG. 26 depicts a differential scanning calorimetry (“DSC”) thermograph of the crystalline potassium salt form 1 of AMG 397 indicating a Tm of 161 and 227° C.

FIG. 27 depicts an X-ray powder diffraction (“XRPD”) pattern of the crystalline potassium salt form 2 (ethyl acetate solvate) of AMG 397.

FIG. 28 depicts a differential scanning calorimetry (“DSC”) thermograph of the crystalline potassium salt form 2 (ethyl acetate solvate) of AMG 397 indicating a Tm of 67 and 149° C.

FIG. 29 depicts a thermogravimetric analysis (“TGA”) trace of the crystalline potassium salt form 2 (ethyl acetate solvate) of AMG 397 showing 23.8% weight loss to 200° C.

FIG. 30 depicts an overlay of the X-ray powder diffraction (“XRPD”) patterns of the crystalline potassium salt forms 1 and 2 of AMG 397.

FIG. 31 depicts an X-ray powder diffraction (“XRPD”) pattern of the crystalline sulfate salt form 1 of AMG 397.

FIG. 32 depicts a differential scanning calorimetry (“DSC”) thermograph of the crystalline sulfate salt form 1 of AMG 397 indicating a Tm of 191° C.

FIG. 33 depicts an X-ray powder diffraction (“XRPD”) pattern of the crystalline sulfate salt form 2 of AMG 397.

FIG. 34 depicts an X-ray powder diffraction (“XRPD”) pattern of the crystalline sulfate salt form 3 of AMG 397.

FIG. 35 depicts a differential scanning calorimetry (“DSC”) thermograph of the crystalline sulfate salt form 3 of AMG 397 indicating a Tm of 218° C.

FIG. 36 depicts a thermogravimetric analysis (“TGA”) trace of the crystalline sulfate salt form 3 of AMG 397 showing 5.6% weight loss to 150° C., with a further 4.8% weight loss to 250° C. prior to melt/degradation.

FIG. 37 depicts an X-ray powder diffraction (“XRPD”) pattern of the crystalline phosphate salt form 1 of AMG 397.

FIG. 38 depicts a differential scanning calorimetry (“DSC”) thermograph of the crystalline phosphate salt form 1 of AMG 397 indicating a Tm of 210° C.

FIG. 39 depicts a thermogravimetric analysis (“TGA”) trace of the crystalline phosphate salt form 1 of AMG 397 showing 2.3% weight loss to 200° C., with an additional 4.2% weight loss to 240° C.

FIG. 40 depicts a moisture sorption profile (DVS) of the crystalline phosphate salt form 1 of AMG 397 showing weight gain of ˜13% by 95% relative humidity.

FIG. 41 depicts an X-ray powder diffraction (“XRPD”) pattern of the crystalline fumarate salt form 1 of AMG 397.

FIG. 42 depicts a differential scanning calorimetry (“DSC”) thermograph of the crystalline fumarate salt form 1 of AMG 397 indicating a Tm of 232° C.

FIG. 43 depicts a thermogravimetric analysis (“TGA”) trace of the crystalline fumarate salt form 1 of AMG 397 showing 21.3% weight loss to 271° C.

FIG. 44 depicts a moisture sorption profile (DVS) of the crystalline fumarate salt form 1 of AMG 397 showing weight gain of ˜3.5% by 95% relative humidity, and weight loss at 0% relative humidity with form change.

FIG. 45 depicts an X-ray powder diffraction (“XRPD”) pattern of the crystalline fumarate salt form 2 of AMG 397.

FIG. 46 depicts a differential scanning calorimetry (“DSC”) thermograph of the crystalline fumarate salt form 2 of AMG 397 indicating a Tm of 243° C.

FIG. 47 depicts a thermogravimetric analysis (“TGA”) trace of the crystalline fumarate salt form 2 of AMG 397 showing 8% weight loss to 150° C., with an additional 9.3% weight loss between 200-275° C.

FIG. 48 depicts an overlay of the X-ray powder diffraction (“XRPD”) patterns of the crystalline fumarate salt forms 1 and 2 of AMG 397.

FIG. 49 depicts an X-ray powder diffraction (“XRPD”) pattern of the crystalline citrate salt form 1 of AMG 397.

FIG. 50 depicts a differential scanning calorimetry (“DSC”) thermograph of the crystalline citrate salt form 1 of AMG 397 indicating a Tm of 214° C.

FIG. 51 depicts a thermogravimetric analysis (“TGA”) trace of the crystalline citrate salt form 1 of AMG 397 showing 7.1% weight loss to 190° C., with an additional 16.9% weight loss to 245° C.

FIG. 52 depicts an X-ray powder diffraction (“XRPD”) pattern of the crystalline citrate salt form 2 hydrate of AMG 397.

FIG. 53 depicts a differential scanning calorimetry (“DSC”) thermograph of the crystalline citrate salt form 2 hydrate of AMG 397 indicating a Tm of 206° C.

FIG. 54 depicts a thermogravimetric analysis (“TGA”) trace of the crystalline citrate salt form 2 hydrate of AMG 397 showing 1.6% weight loss to 178° C., with an additional 13.5% weight loss to 250° C.

FIG. 55 depicts a moisture sorption profile (DVS) of the crystalline citrate salt form 2 hydrate of AMG 397 showing weight gain of ˜1.8% by 40% relative humidity, and weight loss between 40% and 0% relative humidity, indicating a monohydrate.

FIG. 56 depicts an overlay of the X-ray powder diffraction (“XRPD”) patterns of the crystalline citrate salt forms 1 and 2 hydrate of AMG 397.

FIG. 57 depicts an X-ray powder diffraction (“XRPD”) pattern of the crystalline lactate salt form 1 of AMG 397.

FIG. 58 depicts a differential scanning calorimetry (“DSC”) thermograph of the crystalline lactate salt form 1 of AMG 397 indicating a Tm of 219° C.

FIG. 59 depicts a thermogravimetric analysis (“TGA”) trace of the crystalline lactate salt form 1 of AMG 397 showing 4.7% weight loss to 150° C., with an additional 12.7% weight loss to 250° C.

FIG. 60 depicts a moisture sorption profile (DVS) of the crystalline lactate salt form 1 of AMG 397 showing weight gain of 0% by 95% relative humidity and a weight loss at 0% relative humidity with a form change.

FIG. 61 depicts an X-ray powder diffraction (“XRPD”) pattern of the crystalline succinate salt form 1 of AMG 397.

FIG. 62 depicts a differential scanning calorimetry (“DSC”) thermograph of the crystalline succinate salt form 1 of AMG 397 indicating a Tm of 210° C.

FIG. 63 depicts a thermogravimetric analysis (“TGA”) trace of the crystalline succinate salt form 1 of AMG 397 showing 1.1% weight loss to 115° C., with an additional 15.9% weight loss to 235° C.

FIG. 64 depicts a moisture sorption profile (DVS) of the crystalline succinate salt form 1 of AMG 397 showing weight gain of 5.1% by 95% relative humidity.

FIG. 65 depicts an X-ray powder diffraction (“XRPD”) pattern of the crystalline ammonium salt form 1 of AMG 397.

FIG. 66 depicts a differential scanning calorimetry (“DSC”) thermograph of the crystalline ammonium salt form 1 of AMG 397 indicating a Tm of 227° C.

FIG. 67 depicts a thermogravimetric analysis (“TGA”) trace of the crystalline ammonium salt form 1 of AMG 397 showing 5.7% weight loss to 170° C., with an additional 3.3% weight loss to 255° C.

FIG. 68 depicts an X-ray powder diffraction (“XRPD”) pattern of the crystalline besylate salt form 1 hydrate of AMG 397.

FIG. 69 depicts a differential scanning calorimetry (“DSC”) thermograph of the crystalline besylate salt form 1 hydrate of AMG 397 indicating a Tm of 57 and 234° C.

FIG. 70 depicts a thermogravimetric analysis (“TGA”) trace of the crystalline besylate salt form 1 hydrate of AMG 397 showing 4.1% weight loss to 75° C., with an additional 4.6% weight loss to 260° C.

FIG. 71 depicts a moisture sorption profile (DVS) of the crystalline besylate salt form 1 hydrate of AMG 397 showing weight gain of 8.4% by 95% relative humidity, with no form change.

FIG. 72 depicts an X-ray powder diffraction (“XRPD”) pattern of the crystalline tosylate salt form 1 of AMG 397.

FIG. 73 depicts a differential scanning calorimetry (“DSC”) thermograph of the crystalline tosylate salt form 1 of AMG 397 indicating a Tm of 40 and 226° C.

FIG. 74 depicts a thermogravimetric analysis (“TGA”) trace of the crystalline tosylate salt form 1 of AMG 397 showing 1.6% weight loss to 75° C., with an additional 3.9% weight loss to 250° C.

FIG. 75 depicts a moisture sorption profile (DVS) of the crystalline besylate salt form 1 hydrate of AMG 397 showing weight gain of 4.7% by 95% relative humidity, with no form change.

FIG. 76 depicts an X-ray powder diffraction (“XRPD”) pattern of the crystalline maleate salt form 1 of AMG 397 (family of isostructural solvates from acetone, MeCN, DCM, DMF/ACN, DMF/EtOH, and THF).

FIG. 77 depicts a differential scanning calorimetry (“DSC”) thermograph of the crystalline maleate salt form 1 of AMG 397 indicating a Tm of 222° C.

FIG. 78 depicts a thermogravimetric analysis (“TGA”) trace of the crystalline maleate salt form 1 of AMG 397 showing 11.9% weight loss to 250° C.

FIG. 79 depicts an X-ray powder diffraction (“XRPD”) pattern of the crystalline maleate salt form 2 of AMG 397.

FIG. 80 depicts a moisture sorption profile (DVS) of the crystalline maleate salt form 2 of AMG 397 showing weight gain of 7.7% by 95% relative humidity, with no form change.

FIG. 81 depicts an overlay of the X-ray powder diffraction (“XRPD”) patterns of the crystalline maleate salt forms 1 and 2 of AMG 397.

FIG. 82 depicts an X-ray powder diffraction (“XRPD”) pattern of the crystalline malonate salt form 1 of AMG 397.

FIG. 83 depicts a differential scanning calorimetry (“DSC”) thermograph of the crystalline malonate salt form 1 of AMG 397 indicating a Tm of 161° C. and 187° C.

FIG. 84 depicts a thermogravimetric analysis (“TGA”) trace of the crystalline malonate salt form 1 of AMG 397 showing 17.5% weight loss to 250° C.

FIG. 85 depicts an X-ray powder diffraction (“XRPD”) pattern of the crystalline malonate salt form 2 of AMG 397.

FIG. 86 depicts an overlay of the X-ray powder diffraction (“XRPD”) patterns of the crystalline malonate salt forms 1 and 2 of AMG 397.

FIG. 87 depicts an X-ray powder diffraction (“XRPD”) pattern of the crystalline tartrate salt form 1 of AMG 397 (family of isostructural solvates from acetone, MeCN, DCM, EtOH, MeOH and water).

FIG. 88 depicts a differential scanning calorimetry (“DSC”) thermograph of the crystalline tartrate salt form 1 of AMG 397 indicating a Tm of 227° C.

FIG. 89 depicts a thermogravimetric analysis (“TGA”) trace of the crystalline tartrate salt form 1 of AMG 397 showing 23.0% weight loss to 255° C.

FIG. 90 depicts an X-ray powder diffraction (“XRPD”) pattern of the crystalline tris(hydroxymethyl)aminomethane (tris) salt form 1 acetone solvate of AMG 397.

FIG. 91 depicts a differential scanning calorimetry (“DSC”) thermograph of the crystalline tris(hydroxymethyl)aminomethane (tris) salt form 1 acetone solvate of AMG 397 indicating a Tm of 59° C. and 134° C.

FIG. 92 depicts a thermogravimetric analysis (“TGA”) trace of the crystalline tris(hydroxymethyl)aminomethane (tris) salt form 1 acetone solvate of AMG 397 showing 7.9% weight loss to 150° C.

FIG. 93 depicts an X-ray powder diffraction (“XRPD”) pattern of the crystalline iodide salt form 1 of AMG 397.

FIG. 94 depicts a differential scanning calorimetry (“DSC”) thermograph of the crystalline iodide salt form 1 of AMG 397 indicating a Tm of 231° C.

FIG. 95 depicts a single crystal X-ray crystal structure of the crystalline DMSO solvate of AMG 397.

### DETAILED DESCRIPTION

Disclosed herein are salt and solvate forms of (4S,7aR,9aR,10R,11E,14S,15R)-6′-chloro-10-methoxy-14,15-dimethyl-10-{[(9aR)-octahydro-2H-pyrido[1,2-a]pyrazin-2-yl]methyl}-3′,4′,7a,8,9,9a,10,13,14,15-decahydro-2′H,3H,5H-spiro[1,19-etheno-16,16-cyclobuta[i][1,4]oxazepino[3,4-f][1,2,7]thiadiazacyclohexadecine-4,1′-naphthalene]-16,16,18(7H,17H)-trione (AMG 397), such as crystalline salt and solvate forms thereof:

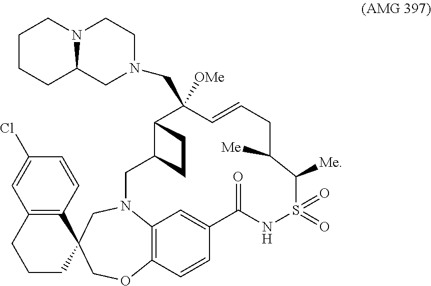

AMG 397 anhydrous form 4 is a thermodynamically stable form. The crystal forms described here have unique physical properties which can be advantageous for new formulations of AMG 397.

Also provided herein pharmaceutical formulations of salt and solvate forms of AMG 397, and methods of treating a subject suffering from cancer, comprising administering to the subject a therapeutically effective amount of a pharmaceutical formulation of a salt or solvate form as disclosed herein.

U.S. Pat. No. 10,300,075, which is incorporated by reference herein in its entirety, discloses synthetic procedures for synthesizing Mcl-1 inhibitors, such as AMG 397.

Further provided herein are crystalline salt and solvate forms of AMG 397, pharmaceutical formulations thereof, and methods of treating a subject suffering from cancer, comprising administering to the subject a therapeutically effective amount of a pharmaceutical formulation of a crystalline salt or solvate form as disclosed herein.

The compounds disclosed herein may be identified either by their chemical structure and/or chemical name herein. When the chemical structure and chemical name conflict, the chemical structure is determinative of the identity of the compound.

When ranges are used herein for physical properties, such as molecular weight, or chemical properties, such as chemical formulae, all combinations and subcombinations of ranges and specific embodiments therein are intended to be included.

As used herein, chemical structures which contain one or more stereocenters depicted with dashed and bold bonds (i.e., [symbol] and [symbol] ) are meant to indicate absolute stereochemistry of the stereocenter(s) present in the chemical structure. As used herein, bonds symbolized by a simple line do not indicate a stereo-preference. Unless otherwise indicated to the contrary, chemical structures that include one or more stereocenters which are illustrated herein without indicating absolute or relative stereochemistry encompass all possible stereoisomeric forms of the compound (e.g., diastereomers, enantiomers) and mixtures thereof. Structures with a single bold or dashed line, and at least one additional simple line, encompass a single enantiomeric series of all possible diastereomers.

The term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

“Treatment” or “treating” means any treatment of a disease in a patient, including: a) preventing the disease, that is, causing the clinical symptoms of the disease not to develop; b) inhibiting the disease; c) slowing or arresting the development of clinical symptoms; and/or d) relieving the disease, that is, causing the regression of clinical symptoms. Treatment of diseases and disorders herein is intended to also include the prophylactic administration of a pharmaceutical formulation described herein to a subject (i.e., an animal, preferably a mammal, most preferably a human) believed to be in need of treatment, such as, for example, cancer.

“Salts” are ionic compounds formed by the treatment of AMG 397 with an acid or base. Any salt that is consistent with the overall stability and utility of the compounds of AMG 397 may be provided using conventional methods. Suitable salts include, without limitation, salts of acidic or basic groups that can be present in the compounds provided herein. Under certain acidic conditions, the compound can form a wide variety of salts with various inorganic and organic acids. Acids that can be used to prepare pharmaceutically acceptable salts of such basic compounds are those that form salts comprising pharmacologically acceptable anions including, but not limited to, acetate, benzenesulfonate, benzoate, bicarbonate, bitartrate, bromide, calcium edetate, camsylate, carbonate, chloride, bromide, iodide, citrate, dihydrochloride, edetate, edisylate, estolate, esylate, fumarate, gluceptate, gluconate, glutamate, glycollylarsanilate, hexylresorcinate, hydrabamine, hydroxynaphthoate, isethionate, lactate, lactobionate, malate, maleate, mandelate, mesylate (methylenesulfonate), methylsulfate, muscate, napsylate, nitrate, panthothenate, phosphate/diphosphate, polygalacturonate, salicylate, stearate, succinate, sulfate, tannate, tartrate, teoclate, triethiodide, and pamoate. Under certain basic conditions, the compound can form base salts with various pharmacologically acceptable cations. Non-limiting examples of such salts include alkali metal or alkaline earth metal salts and, particularly, calcium, magnesium, sodium, lithium, zinc, potassium and iron salts, as well as tetraalkylammonium salts. General information regarding pharmaceutically acceptable salts may be found in Stahl PH, and Wermuth CG, eds., Handbook of Pharmaceutical Salts: Properties, Selection and Use, 2002, Wiley-VCH/VHCA Weinheim/Zürich.

The term “therapeutically effective amount” means an amount effective, when administered to a human or non-human patient, to treat a disease, e.g., a therapeutically effective amount may be an amount sufficient to treat a disease or disorder responsive to myosin activation. The therapeutically effective amount may be ascertained experimentally, for example by assaying blood concentration of the chemical entity, or theoretically, by calculating bioavailability.

The term “solvate” refers to the chemical entity formed by the interaction of a solvate and a compound. Crystalline solvates of AMG 397 used in formulations herein are specifically contemplated. Solvents that can form crystalline solvate forms of AMG 397 include without limitation, ethanol. In some cases, a solvate has 0.5 to 2 solvent molecules per AMG 397 molecule.

### Salt and Solvate Forms

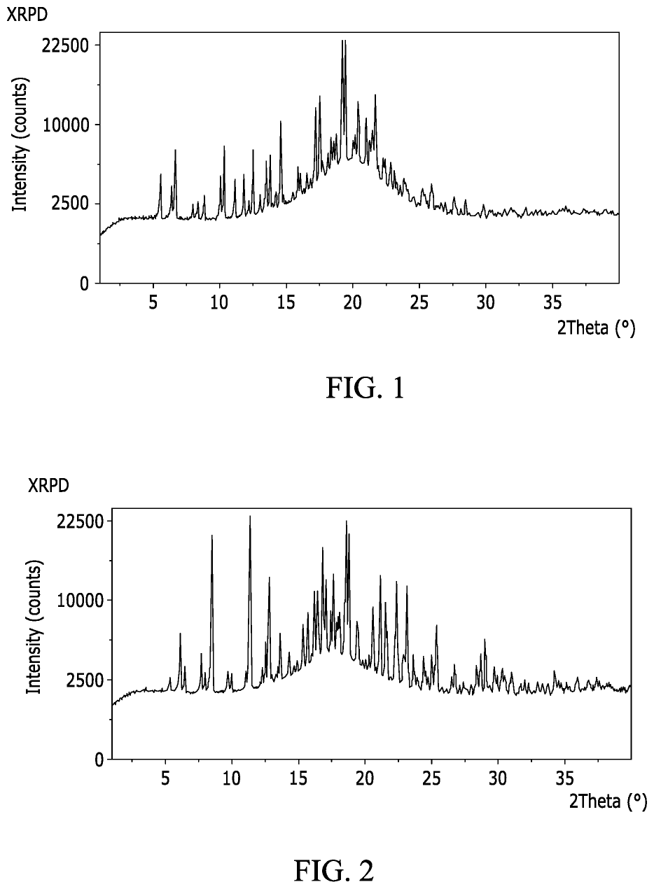

Trifluoroethanol Solvate Form: The crystalline trifluoroethanol solvate form of AMG 397 can be characterized by an X-ray powder diffraction pattern, obtained as set forth in the Examples, having peaks at 17.5, 19.2, 19.4, and 21.7±0.2° 2θ using Cu Kα radiation, optionally further characterized by additional peaks at 14.6, 17.2, 18.4, 18.5, 18.8, 20.0, 20.2, 20.4, 21.0, 21.2, and 21.5±0.2° 2θ using Cu Kα radiation, and/or additional peaks at 6.7, 10.3, 12.5, 13.5, 13.8, 17.7, 17.8, 18.1, 21.9, 22.3, 22.4, and 22.9±0.2° 2θ using Cu Kα radiation. In some embodiments, the crystalline trifluoroethanol solvate form has an X-ray powder diffraction pattern substantially as shown in FIG. 1, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

Hexafluoroisopropanol solvate: The crystalline hexafluoroisopropanol solvate can be characterized by an X-ray powder diffraction pattern, obtained as set forth in the Examples, having peaks at 11.4, 18.6, and 18.8±0.2° 2θ using Cu Kα radiation, optionally further characterized by additional peaks at 8.5, 12.8, 17.1, 17.6, 21.1, 22.4, and 23.1±0.2° 2θ using Cu Kα radiation, and/or additional peaks at 6.1, 13.6, 15.3, 15.7, 16.2, 16.4, 16.5, 17.4, 17.8, 18.0, 18.1, 19.4, 20.6, 21.5, 21.7, 22.2, and 25.4±0.2° 2θ using Cu Kα radiation. In some embodiments, hydrate form 1 has an X-ray powder diffraction pattern substantially as shown in FIG. 2, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

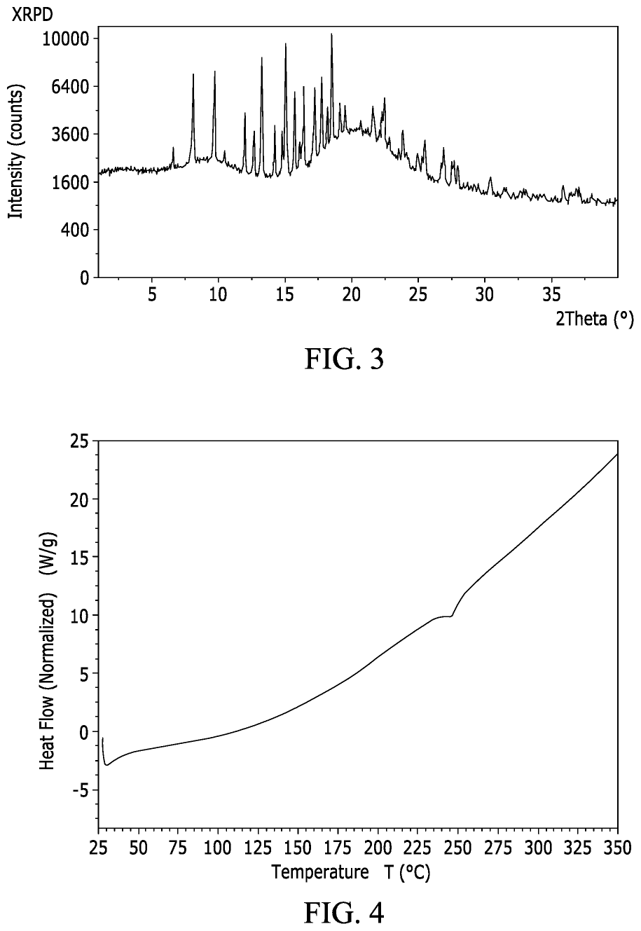

1-propanol solvate: The crystalline 1-propanol solvate can be characterized by an X-ray powder diffraction pattern, obtained as set forth in the Examples, having peaks at 13.3, 15.1, and 18.5±0.2° 2θ using Cu Kα radiation, optionally further characterized by additional peaks at 8.1, 9.7, 15.7, 16.4, 17.2, and 17.7±0.2° 2θ using Cu Kα radiation, and/or additional peaks at 12.0, 12.7, 14.2, 14.8, 17.1, 18.2, 19.1, 19.5, 20.7, 21.2, 21.6, 21.7, 22.1, 22.3, 22.4, 22.8, 23.5, 23.8, 23.9, and 25.5±0.2° 2θ using Cu Kα radiation. In some embodiments, the crystalline 1-propanol solvate has an X-ray powder diffraction pattern substantially as shown in FIG. 3, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

Differential scanning calorimetry (DSC) thermographs were obtained, as set forth in the Examples, for the crystalline 1-propanol solvate. The DSC curve indicates an endothermic transition at 234° C.±3° C. Thus, in some embodiments, the crystalline 1-propanol solvate can be characterized by a DSC thermograph having a transition endotherm with an onset of 231° C. to 237° C. For example, in some embodiments the crystalline 1-propanol solvate is characterized by DSC, as shown in FIG. 4.

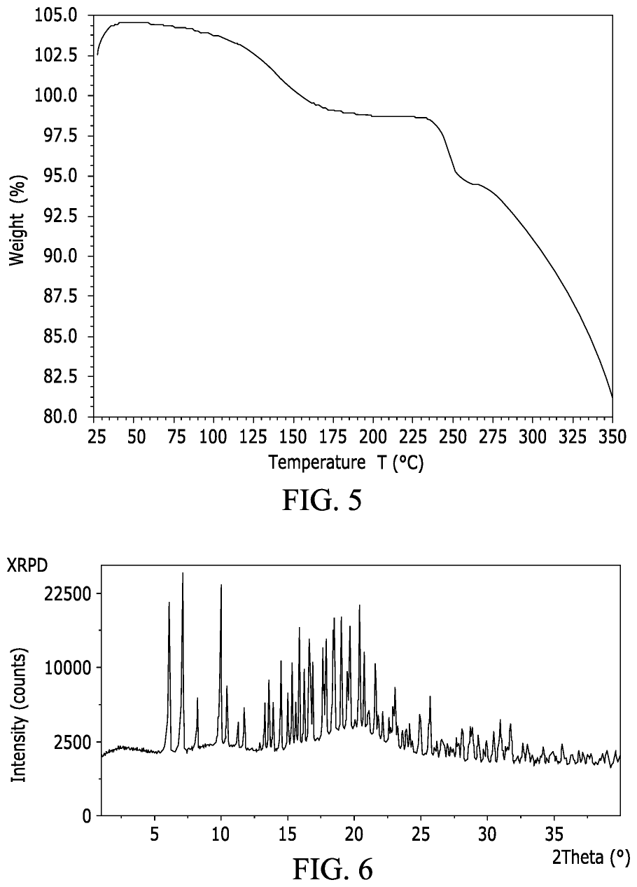

The crystalline 1-propanol solvate can be characterized by thermogravimetric analysis (TGA). Thus, the crystalline 1-propanol solvate can be characterized by a weight loss in a range of about 5.6% with an onset temperature of 38° C. to 190° C. In some embodiments, the crystalline 1-propanol solvate has a thermogravimetric analysis substantially as depicted in FIG. 5, wherein by “substantially” is meant that the reported TGA features can vary by ±5° C.

Isopropanol solvate form 1: The crystalline isoropanol solvate form 1 can be characterized by an X-ray powder diffraction pattern, obtained as set forth in the Examples, having peaks at 6.1, 7.1, and 10.0±0.2° 2θ using Cu Kα radiation, optionally further characterized by additional peaks at 18.5, 19.0, 19.7, and 20.4±0.2° 2θ using Cu Kα radiation, and/or additional peaks at 10.5, 13.6, 14.5, 15.0, 15.3, 15.9, 16.2, 16.6, 16.7, 16.9, 17.7, 17.9, 18.4, 19.5, 20.7, 21.6, 23.1, and 25.7±0.2° 2θ using Cu Kα radiation. In some embodiments, the crystalline isoropanol solvate form 1 has an X-ray powder diffraction pattern substantially as shown in FIG. 6, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

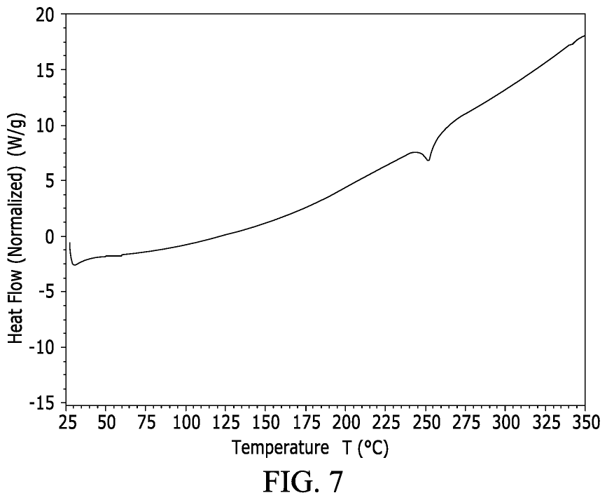

Differential scanning calorimetry (DSC) thermographs were obtained, as set forth in the Examples, for the crystalline isoropanol solvate form 1. The DSC curve indicates an endothermic transition at 247° C.±3° C. Thus, in some embodiments, the crystalline isopropanol solvate form 1 can be characterized by a DSC thermograph having a transition endotherm with an onset of 244° C. to 250° C. For example, in some embodiments the crystalline isoropanol solvate form 1 is characterized by DSC, as shown in FIG. 7.

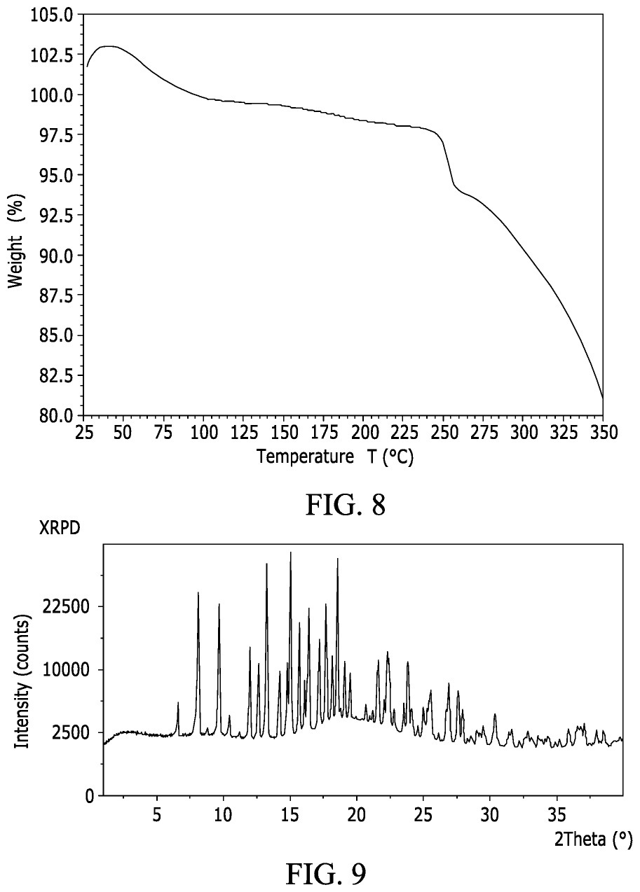

The crystalline isoropanol solvate form 1 can be characterized by thermogravimetric analysis (TGA). Thus, the crystalline isoropanol solvate form 1 can be characterized by a weight loss in a range of about 0.5% with an onset temperature of 39° C. to 120° C. In some embodiments, the crystalline isoropanol solvate form 1 has a thermogravimetric analysis substantially as depicted in FIG. 8, wherein by “substantially” is meant that the reported TGA features can vary by ±5° C.

Isopropanol solvate form 2: The crystalline isoropanol solvate form 2 can be characterized by an X-ray powder diffraction pattern, obtained as set forth in the Examples, having peaks at 13.3, 15.1, and 18.6±0.2° 2θ using Cu Kα radiation, optionally further characterized by additional peaks at 8.1, 9.7, 16.4, and 17.7±0.2° 2θ using Cu Kα radiation, and/or additional peaks at 12.0, 12.6, 14.2, 14.8, 15.7, 17.1, 17.2, 18.2, 19.1, 19.5, 21.5, 21.6, 22.3, 22.4, and 23.8±0.2° 2θ using Cu Kα radiation. In some embodiments, the crystalline isoropanol solvate form 2 has an X-ray powder diffraction pattern substantially as shown in FIG. 9, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

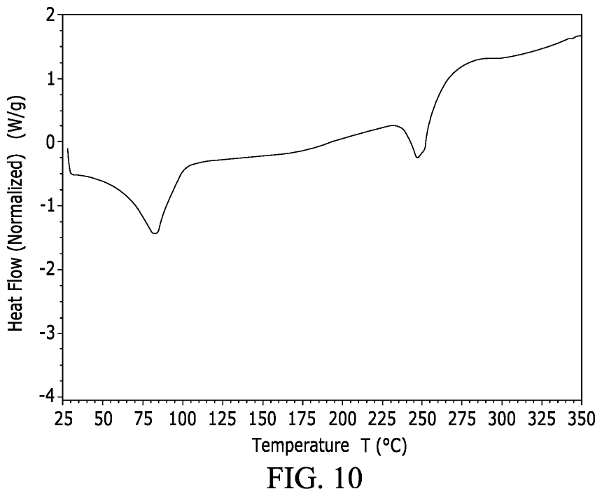

Differential scanning calorimetry (DSC) thermographs were obtained, as set forth in the Examples, for the crystalline isoropanol solvate form 1. The DSC curve indicates endothermic transitions at 83° C.±3° C. and 239° C.±3° C. Thus, in some embodiments, the crystalline isopropanol solvate form 2 can be characterized by a DSC thermograph having a transition endotherm with an onset of 80° C. to 86° C. and 236° C. to 242° C. For example, in some embodiments the crystalline isoropanol solvate form 2 is characterized by DSC, as shown in FIG. 10.

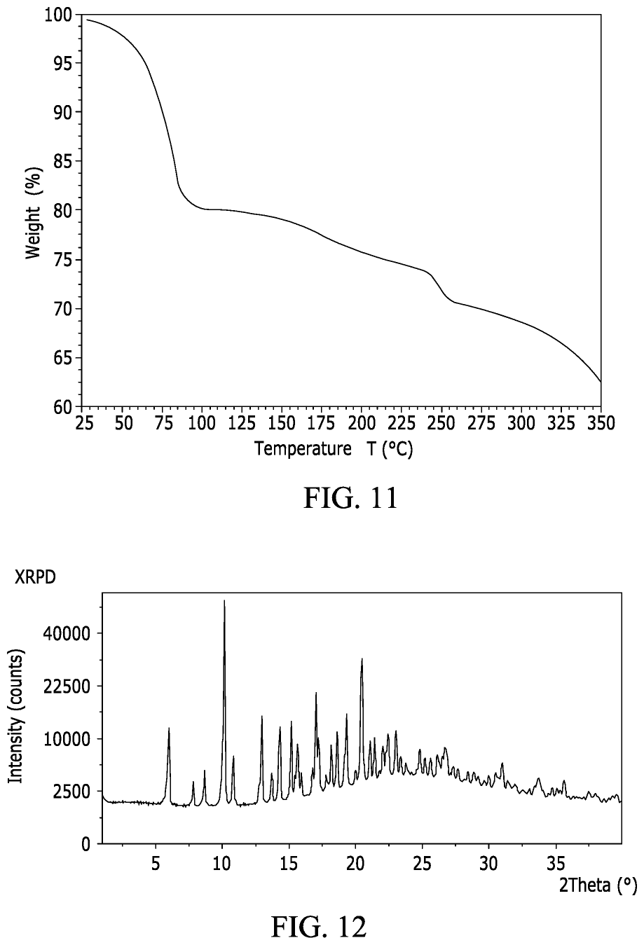

The crystalline isoropanol solvate form 2 can be characterized by thermogravimetric analysis (TGA). Thus, the crystalline isoropanol solvate form 2 can be characterized by a weight loss in a range of about 19.0% with an onset temperature of 37° C. to 111° C. In some embodiments, the crystalline isoropanol solvate form 2 has a thermogravimetric analysis substantially as depicted in FIG. 11, wherein by “substantially” is meant that the reported TGA features can vary by ±5° C.

Acetonitrile solvate: The crystalline acetonitrile solvate can be characterized by an X-ray powder diffraction pattern, obtained as set forth in the Examples, having peaks at 10.2, 17.0, and 20.5±0.2° 2θ using Cu Kα radiation, optionally further characterized by additional peaks at 6.0, 13.0, 14.3, 15.2, 18.6, and 23.0±0.2° 2θ using Cu Kα radiation, and/or additional peaks at 10.9, 15.6, 17.2, 18.2, 19.2, 21.0, 21.4, 22.1, 22.3, 22.5, 23.4, 24.8, 25.2, 25.6, 26.1, 26.5, 26.7, and 26.8±0.2° 2θ using Cu Kα radiation. In some embodiments, the crystalline acetonitrile solvate has an X-ray powder diffraction pattern substantially as shown in FIG. 12, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

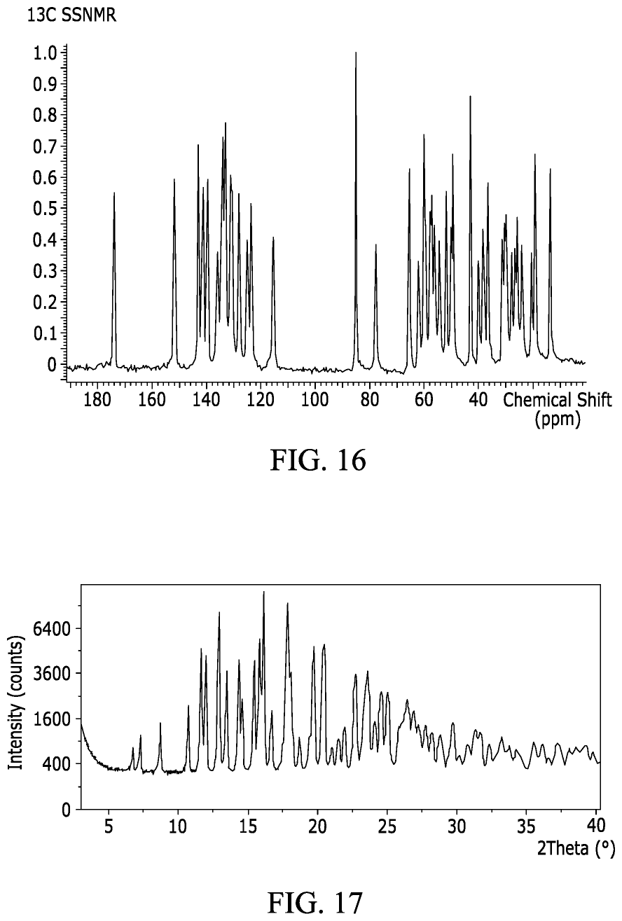

Acetic acid solvate: The crystalline acetic acid solvate can be characterized by solid state ¹³C NMR, obtained as set forth in the Examples, having peaks at 13.63, 19.22, 20.40, 24.22, 25.69, 26.57, 27.75, 29.81, 30.40, 31.28, 36.57, 38.34, 40.10, 43.04, 49.51, 50.10, 51.86, 54.51, 56.28, 57.16, 57.75, 60.10, 62.16, 65.39, 77.75, 85.10, 115.39, 123.63, 125.10, 128.04, 131.27, 133.04, 133.92, 135.98, 139.80, 141.27, 143.04, 151.86, and 173.92±0.5 ppm. In some embodiments, the crystalline acetic acid solvate has a solid state ¹³C NMR substantially as shown in FIG. 16, wherein by “substantially” is meant that the reported peaks can vary by ±0.5 ppm.

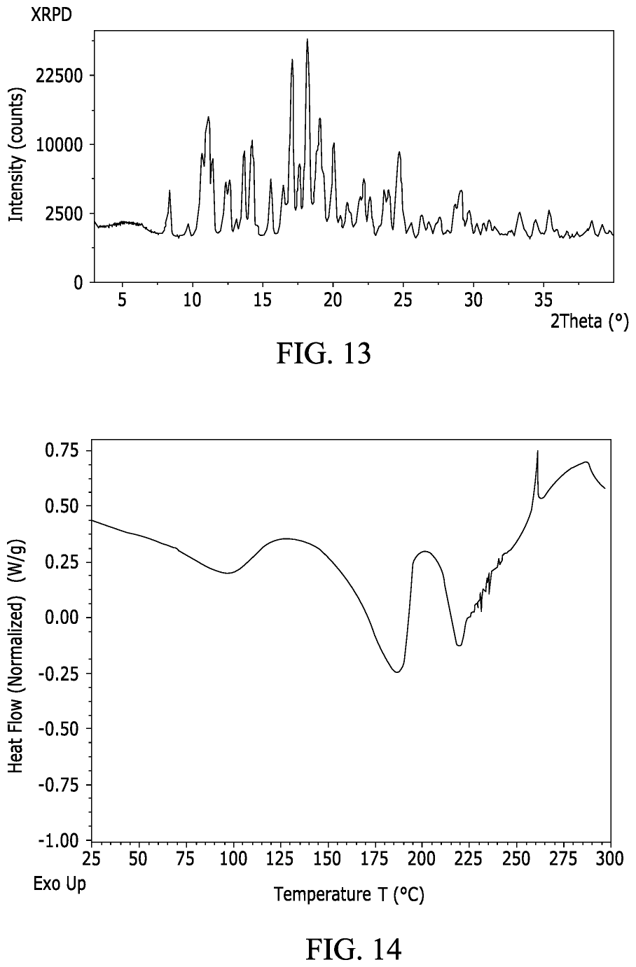

The crystalline acetic acid solvate can be characterized by an X-ray powder diffraction pattern, obtained as set forth in the Examples, having peaks at 11.1, 17.1, 18.2, and 19.1 0.2° 2θ using Cu Kα radiation, optionally further characterized by additional peaks at 10.7, 10.9, 11.5, 13.7, 14.3, 18.8, 20.1, and 24.8±0.2° 2θ using Cu Kα radiation, and/or additional peaks at 8.4, 12.4, 12.7, 15.6, 16.5, 17.6, 19.3, 22.2, 23.6, 24.0, 24.6, and 29.0±0.2° 2θ using Cu Kα radiation. In some embodiments, the crystalline acetic acid solvate has an X-ray powder diffraction pattern substantially as shown in FIG. 13, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

Differential scanning calorimetry (DSC) thermographs were obtained, as set forth in the Examples, for the crystalline acetic acid solvate. The DSC curve indicates an endothermic transition at 95° C.±3° C. and 155° C.±3° C. Thus, in some embodiments, hydrate form 2 can be characterized by a DSC thermograph having a transition endotherm with an onset of 92° C. to 98° C. and 152° C. to 158° C. For example, in some embodiments the crystalline acetic acid solvate is characterized by DSC, as shown in FIG. 14.

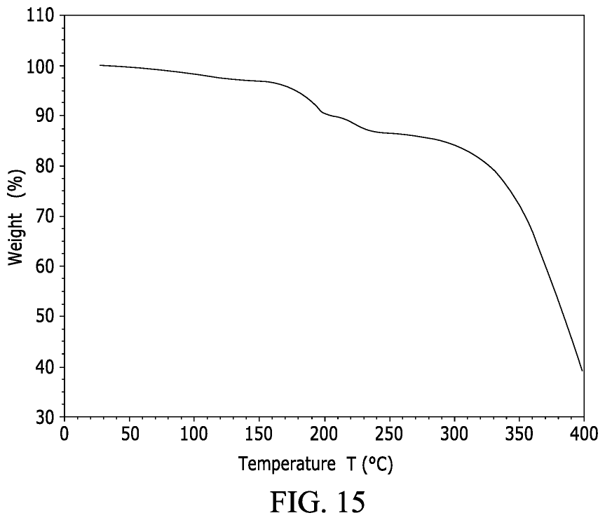

The crystalline acetic acid solvate can be characterized by thermogravimetric analysis (TGA). Thus, the crystalline acetic acid solvate can be characterized by a weight loss in a range of about 0% to about 3.1% to about 150° C., with additional weight loss in a range of about 0% to about 10.4% to about 250° C. In some embodiments, the crystalline acetic acid solvate has a thermogravimetric analysis substantially as depicted in FIG. 15, wherein by “substantially” is meant that the reported TGA features can vary by ±5° C.

Hydrochloride salt form 1: Crystalline hydrochloride salt form 1 can be characterized by an X-ray powder diffraction pattern, obtained as set forth in the Examples, having peaks at 12.9, 16.2, and 17.9±0.2° 2θ using Cu Kα radiation, optionally further characterized by additional peaks at 11.7, 12.0, 15.9, 19.8, and 20.5±0.2° 2θ using Cu Kα radiation, and/or additional peaks at 10.7, 13.5, 14.4, 14.6, 15.5, 18.1, 22.8, 23.7, 24.6, 25.1, and 26.5±0.2° 2θ using Cu Kα radiation. In some embodiments, the crystalline hydrochloride salt form 1 has an X-ray powder diffraction pattern substantially as shown in FIG. 17, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

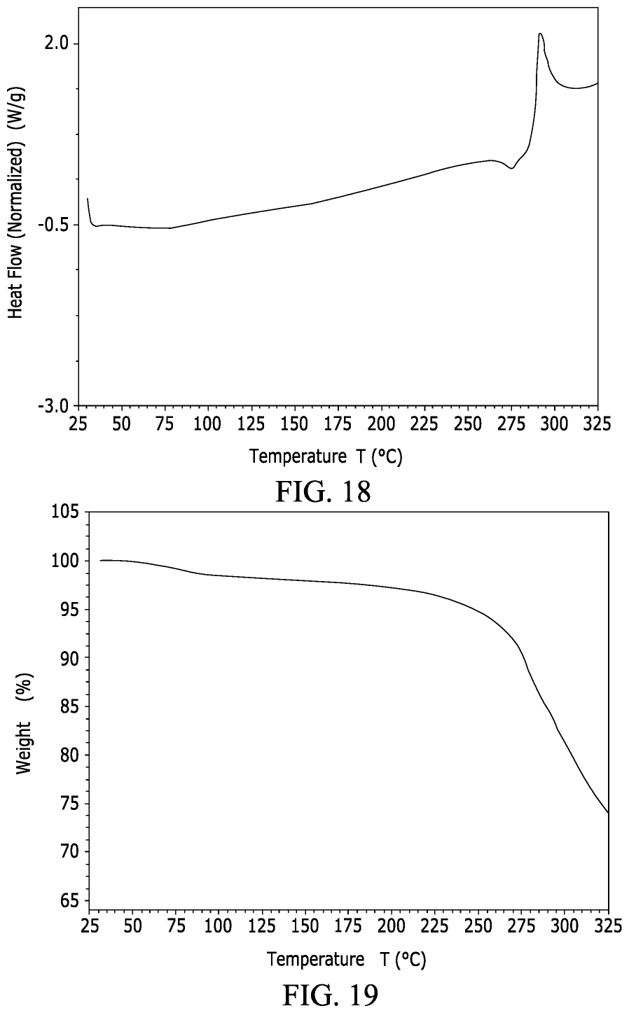

Differential scanning calorimetry (DSC) thermographs were obtained, as set forth in the Examples, for the crystalline hydrochloride salt form 1. The DSC curve indicates an endothermic transition at 267° C.±3° C. Thus, in some embodiments, the crystalline hydrochloride salt 1 can be characterized by a DSC thermograph having a transition endotherm with an onset of 264° C. to 270° C. For example, in some embodiments the crystalline hydrochloride salt 1 is characterized by DSC, as shown in FIG. 18.

The crystalline hydrochloride salt form 1 can be characterized by thermogravimetric analysis (TGA). Thus, the crystalline hydrochloride salt form 1 can be characterized by a weight loss in a range of about 0% to about 9.5% from 35° C. to 275° C., and weight loss in a range of about 0% to about 5.3% to about 250° C. In some embodiments, the crystalline hydrochloride salt form 1 has a thermogravimetric analysis substantially as depicted in FIG. 19, wherein by “substantially” is meant that the reported TGA features can vary by ±5° C.

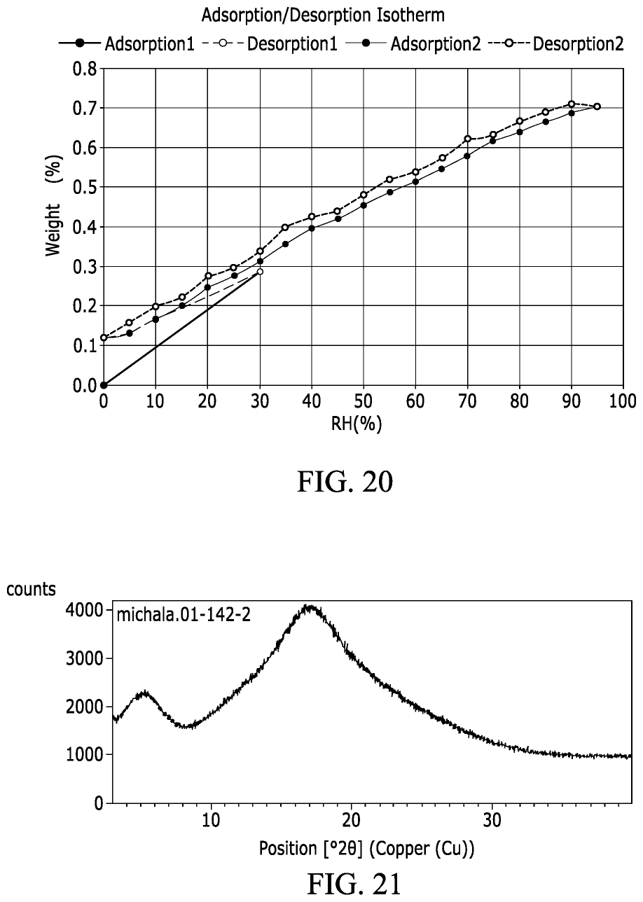

The crystalline hydrochloride salt form 1 can be characterized by a moisture sorption profile. For example, in some embodiments, the crystalline hydrochloride salt form 1 is characterized by the moisture sorption profile as shown in FIG. 20, showing a weight gain of 0.7% by 95% RH.

Sodium salt form 1: Amorphous sodium salt form 1 can be characterized by an X-ray powder diffraction pattern, obtained using Cu Kα radiation. In some embodiments, the amorphous sodium salt form 1 has an X-ray powder diffraction pattern substantially as shown in FIG. 21, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

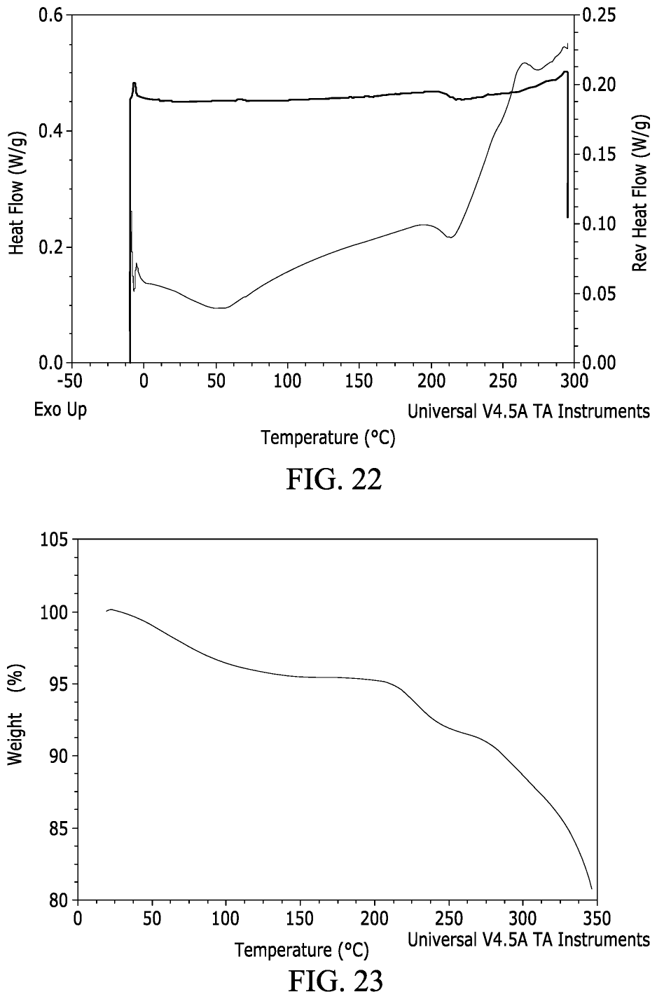

Differential scanning calorimetry (DSC) thermographs were obtained, as set forth in the Examples, for the amorphous sodium salt form 1. The DSC curve indicates an endothermic transition at 216° C.±3° C. Thus, in some embodiments, the amorphous sodium salt 1 can be characterized by a DSC thermograph having a transition endotherm with an onset of 213° C. to 219° C. For example, in some embodiments the amorphous sodium salt 1 is characterized by DSC, as shown in FIG. 22.

The amorphous sodium salt form 1 can be characterized by thermogravimetric analysis (TGA). Thus, the amorphous sodium salt form 1 can be characterized by a weight loss in a range of about 0% to about 4.9% to 210° C. In some embodiments, the amorphous sodium salt form 1 has a thermogravimetric analysis substantially as depicted in FIG. 23, wherein by “substantially” is meant that the reported TGA features can vary by ±5° C.

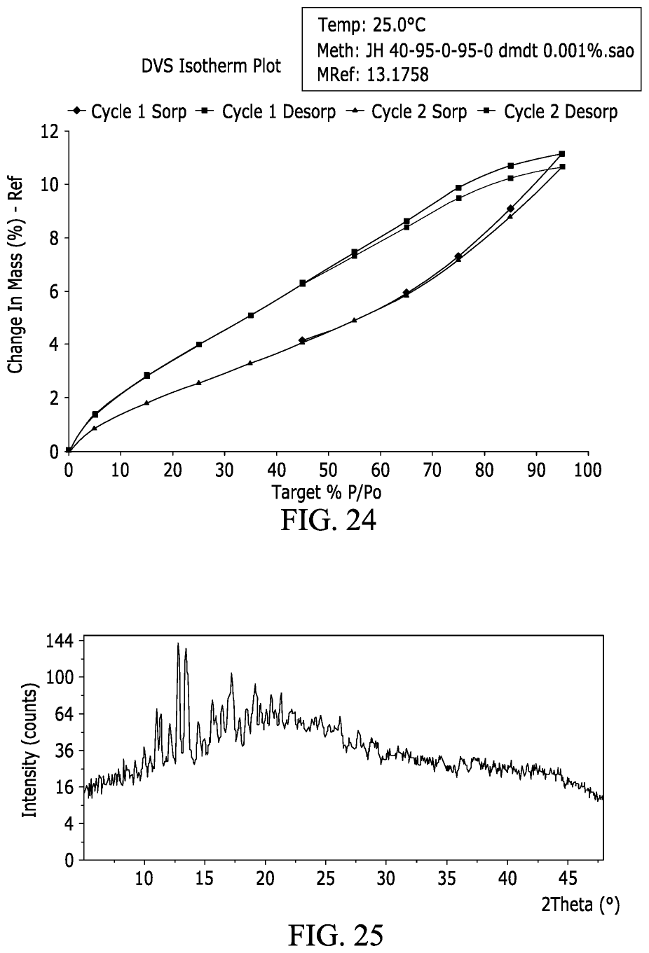

The amorphous sodium salt form 1 can be characterized by a moisture sorption profile. For example, in some embodiments, the amorphous sodium salt form 1 is characterized by the moisture sorption profile as shown in FIG. 24, showing a weight gain of 11.4% by 95% RH and no form change.

Potassium salt form 1: The crystalline potassium salt form 1 can be characterized by an X-ray powder diffraction pattern, obtained as set forth in the Examples, having peaks at 12.8, 13.4, and 17.2±0.2° 2θ using Cu Kα radiation, optionally further characterized by additional peaks at 11.0, 11.4, 14.5, 15.7, and 19.2±0.2° 2θ using Cu Kα radiation. In some embodiments, the crystalline potassium salt form 1 has an X-ray powder diffraction pattern substantially as shown in FIG. 25, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

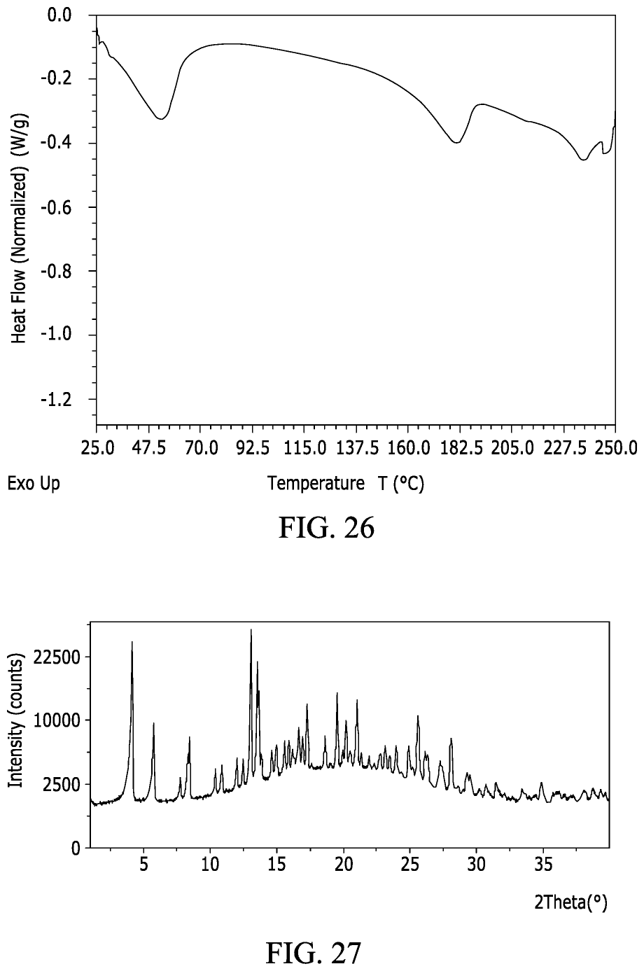

Differential scanning calorimetry (DSC) thermographs were obtained, as set forth in the Examples, for the crystalline potassium salt form 1. The DSC curve indicates an endothermic transition at 161° C.±3° C. and 227° C.±3° C. Thus, in some embodiments, the crystalline potassium salt 1 can be characterized by a DSC thermograph having a transition endotherm with an onset of 158° C. to 164° C. and 224° C. to 230° C. For example, in some embodiments the crystalline potassium salt 1 is characterized by DSC, as shown in FIG. 26.

Potassium salt form 2 (ethyl acetate solvate): The crystalline potassium salt form 2 (ethyl acetate solvate) can be characterized by an X-ray powder diffraction pattern, obtained as set forth in the Examples, having peaks at 2.7, 11.7, and 12.2±0.2° 2θ using Cu Kα radiation, optionally further characterized by additional peaks at 20.5, 20.9, 21.1, 21.6, and 22.9±0.2° 2θ using Cu Kα radiation, and/or additional peaks at 11.2, 15.1, 15.3, 15.4, 16.1, 16.3, 16.4, 16.6, 16.8, 16.9, 17.3, 17.5, 17.9, 18.5, 18.9, 19.2, 19.5, 19.721.7, 22.2, 22.5, 22.7, 23.3, 23.5, 23.9, and 24.4±0.2° 2θ using Cu Kα radiation. In some embodiments, the crystalline potassium salt form 2 (ethyl acetate solvate) has an X-ray powder diffraction pattern substantially as shown in FIG. 27, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

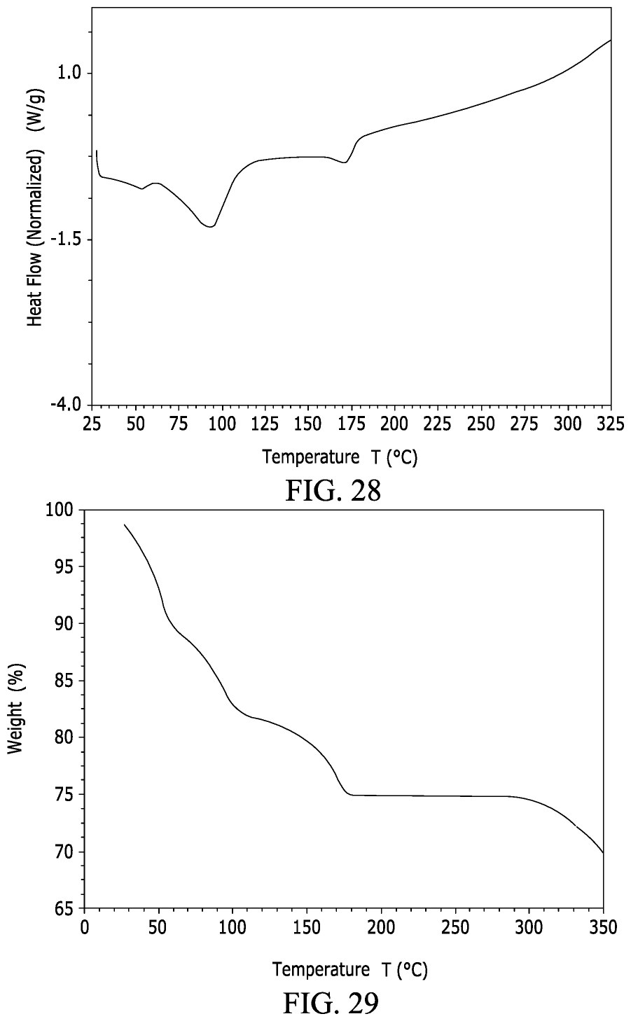

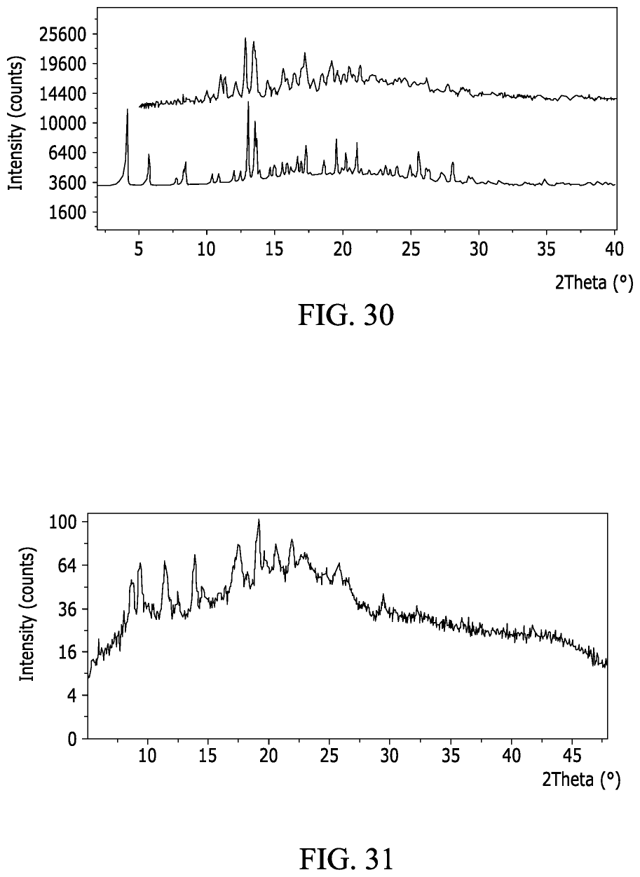

Differential scanning calorimetry (DSC) thermographs were obtained, as set forth in the Examples, for the crystalline potassium salt form 2 (ethyl acetate solvate). The DSC curve indicates an endothermic transition at 67° C.±3° C. and 149° C.±3° C. Thus, in some embodiments, the crystalline potassium salt 2 (ethyl acetate solvate) can be characterized by a DSC thermograph having a transition endotherm with an onset of 64° C. to 70° C. and 146° C. to 152° C. For example, in some embodiments the crystalline potassium salt 2 (ethyl acetate solvate) is characterized by DSC, as shown in FIG. 28.

The crystalline potassium salt form 2 (ethyl acetate solvate) can be characterized by thermogravimetric analysis (TGA). Thus, the crystalline potassium salt form 2 (ethyl acetate solvate) can be characterized by a weight loss in a range of about 0% to about 23.8% to 200° C. In some embodiments, the crystalline potassium salt form 2 (ethyl acetate solvate) has a thermogravimetric analysis substantially as depicted in FIG. 29, wherein by “substantially” is meant that the reported TGA features can vary by ±5° C.

Sulfate salt form 1: The crystalline sulfate salt form 1 can be characterized by an X-ray powder diffraction pattern, obtained as set forth in the Examples, having peaks at 9.3, 13.9, and 19.2±0.2° 2θ using Cu Kα radiation, optionally further characterized by additional peaks at 8.7, 11.5, 17.6, and 21.9±0.2° 2θ using Cu Kα radiation. In some embodiments, the crystalline sulfate salt form 1 has an X-ray powder diffraction pattern substantially as shown in FIG. 31, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

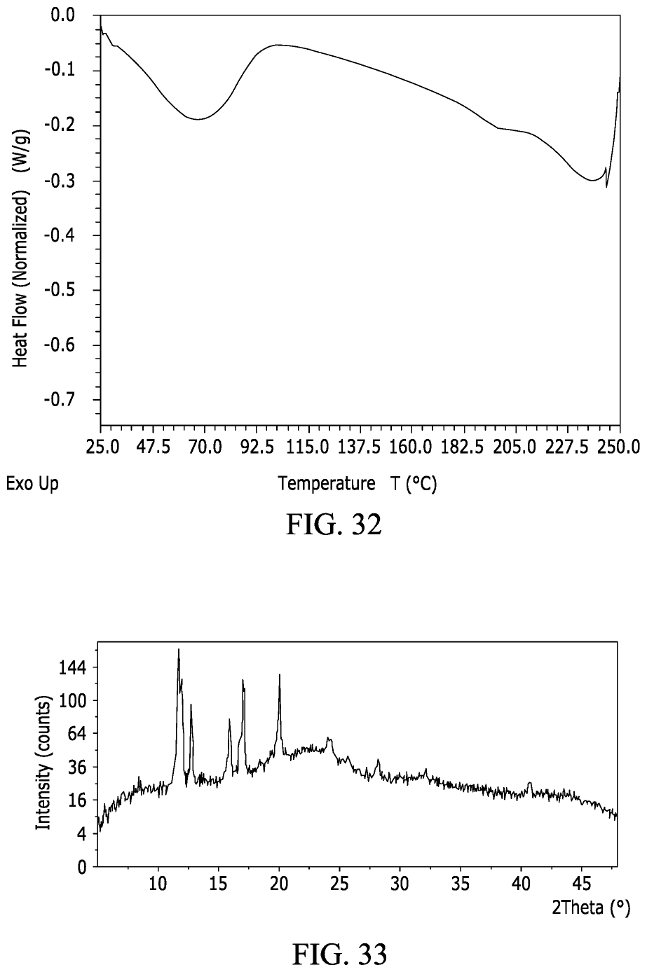

Differential scanning calorimetry (DSC) thermographs were obtained, as set forth in the Examples, for the crystalline sulfate salt form 1. The DSC curve indicates an endothermic transition at 191° C.±3° C. Thus, in some embodiments, the crystalline potassium salt 2 can be characterized by a DSC thermograph having a transition endotherm with an onset of 188° C. to 194° C. For example, in some embodiments the crystalline potassium salt 2 is characterized by DSC, as shown in FIG. 32.

Sulfate salt form 2: The crystalline sulfate salt form 2 can be characterized by an X-ray powder diffraction pattern, obtained as set forth in the Examples, having peaks at 11.7, 17.1, and 20.1±0.2° 2θ using Cu Kα radiation, optionally further characterized by additional peaks at 12.8, 15.9, and 24.1±0.2° 2θ using Cu Kα radiation. In some embodiments, the crystalline sulfate salt form 2 has an X-ray powder diffraction pattern substantially as shown in FIG. 33, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

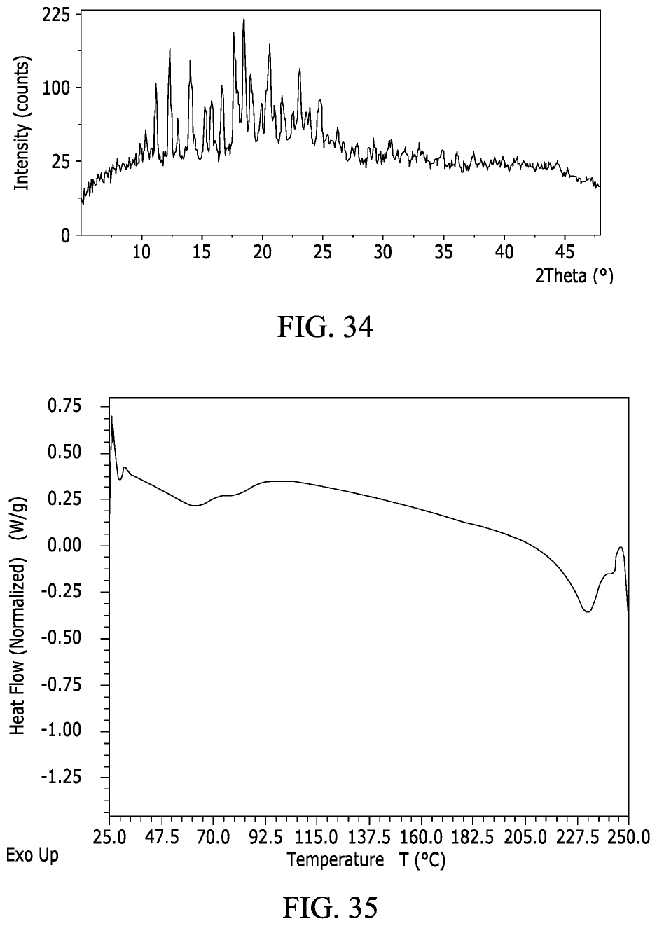

Sulfate salt form 3: The crystalline sulfate salt form 3 can be characterized by an X-ray powder diffraction pattern, obtained as set forth in the Examples, having peaks at 12.3, 17.7, 18.4, and 20.6±0.2° 2θ using Cu Kα radiation, optionally further characterized by additional peaks at 11.2, 14.0, 19.0, and 23.1±0.2° 2θ using Cu Kα radiation, and/or additional peaks at 13.0, 15.3, 15.8, 16.7, 19.0, 21.6, 23.9, and 24.8±0.2° 2θ using Cu Kα radiation. In some embodiments, the crystalline sulfate salt form 3 has an X-ray powder diffraction pattern substantially as shown in FIG. 34, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

Differential scanning calorimetry (DSC) thermographs were obtained, as set forth in the Examples, for the crystalline sulfate salt form 3. The DSC curve indicates an endothermic transition at 218° C.±3° C. Thus, in some embodiments, the crystalline potassium salt 3 can be characterized by a DSC thermograph having a transition endotherm with an onset of 215° C. to 221° C. For example, in some embodiments the crystalline potassium salt 3 is characterized by DSC, as shown in FIG. 35.

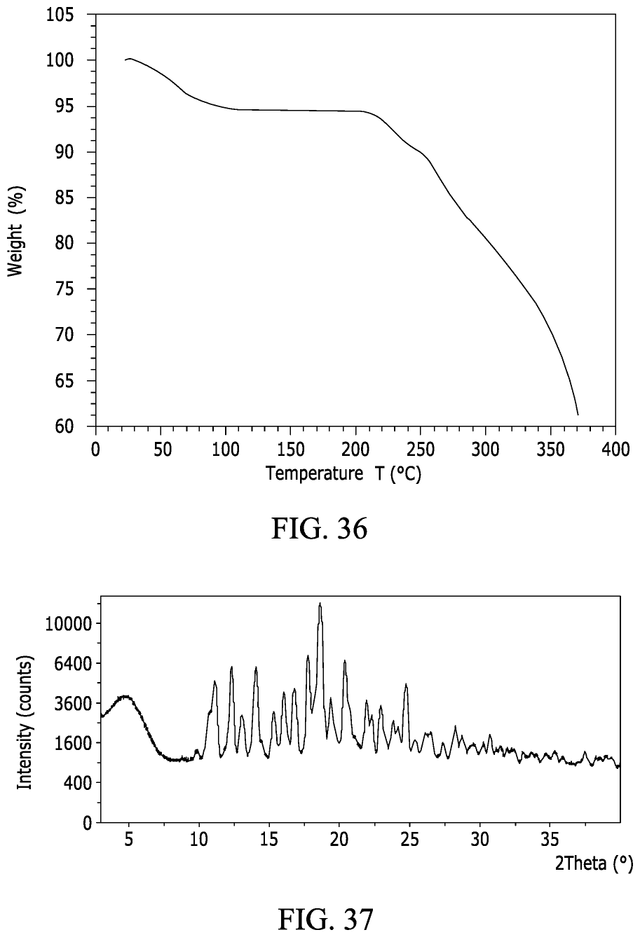

The crystalline sulfate salt form 3 can be characterized by thermogravimetric analysis (TGA). Thus, the crystalline sulfate salt form 3 can be characterized by a weight loss in a range of about 0% to about 5.6% to 150° C., with an additional weight loss in a range of about 0% to about 4.8% to 250° C. In some embodiments, the crystalline sulfate salt form 3 has a thermogravimetric analysis substantially as depicted in FIG. 36, wherein by “substantially” is meant that the reported TGA features can vary by ±5° C.

Phosphate salt form 1: The phosphate salt form 1 can be characterized by ¹³C NMR, obtained as set forth in the Examples, having peaks at 5.8, 15.0, 18.3, 21.2, 22.2, 23.6, 27.6, 27.6, 29.3, 31.7, 31.9, 35.7, 41.3, 43.6, 49.9, 51.7, 53.3, 53.7, 55.8, 57.7, 58.8, 58.9, 59.8, 61.0, 79.6, 80.9, 115.4, 117.3, 119.1, 126.0, 127.9, 128.7, 129.4, 129.5, 130.2, 139.2, 139.8, 139.9, 150.8, and 168.8±0.5 ppm.

Phosphate salt form 1: The crystalline phosphate salt form 1 can be characterized by an X-ray powder diffraction pattern, obtained as set forth in the Examples, having peaks at 17.7, 18.6, and 18.7±0.2° 2θ using Cu Kα radiation, optionally further characterized by additional peaks at 12.3, 14.0, and 20.3±0.2° 2θ using Cu Kα radiation, and/or additional peaks at 11.1, 11.2, 12.4, 16.0, 16.1, 16.7, 16.8, 19.3, 20.7, 21.9, 22.9, 23.0, 24.7, and 24.8±0.2° 2θ using Cu Kα radiation. In some embodiments, the crystalline phosphate salt form 1 has an X-ray powder diffraction pattern substantially as shown in FIG. 37, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

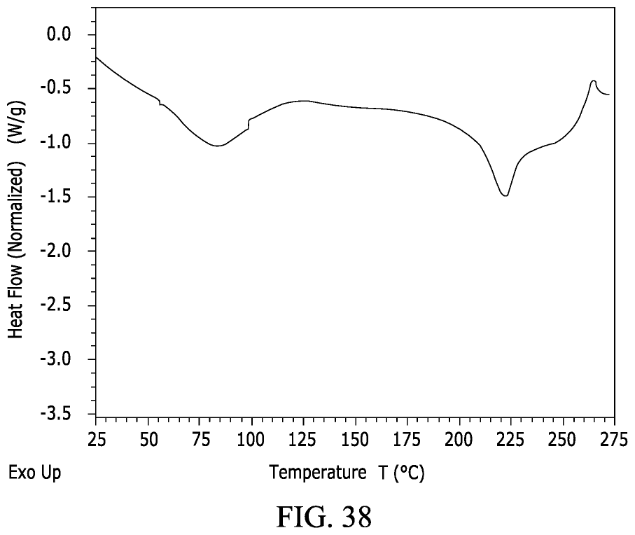

Differential scanning calorimetry (DSC) thermographs were obtained, as set forth in the Examples, for the crystalline phosphate salt form 1. The DSC curve indicates an endothermic transition at 210° C.±3° C. Thus, in some embodiments, the crystalline phosphate salt form 1 can be characterized by a DSC thermograph having a transition endotherm with an onset of 207° C. to 213° C. For example, in some embodiments the crystalline phosphate salt form 1 is characterized by DSC, as shown in FIG. 38.

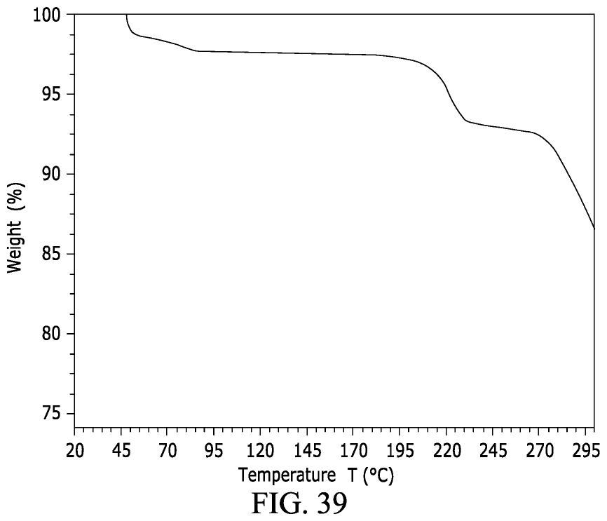

The crystalline phosphate salt form 1 can be characterized by thermogravimetric analysis (TGA). Thus, the crystalline phosphate salt form 1 can be characterized by a weight loss in a range of about 0% to about 2.3% to 100° C., with an additional weight loss in a range of about 0% to about 4.2% to 240° C. In some embodiments, the crystalline phosphate salt form 1 has a thermogravimetric analysis substantially as depicted in FIG. 39, wherein by “substantially” is meant that the reported TGA features can vary by ±5° C.

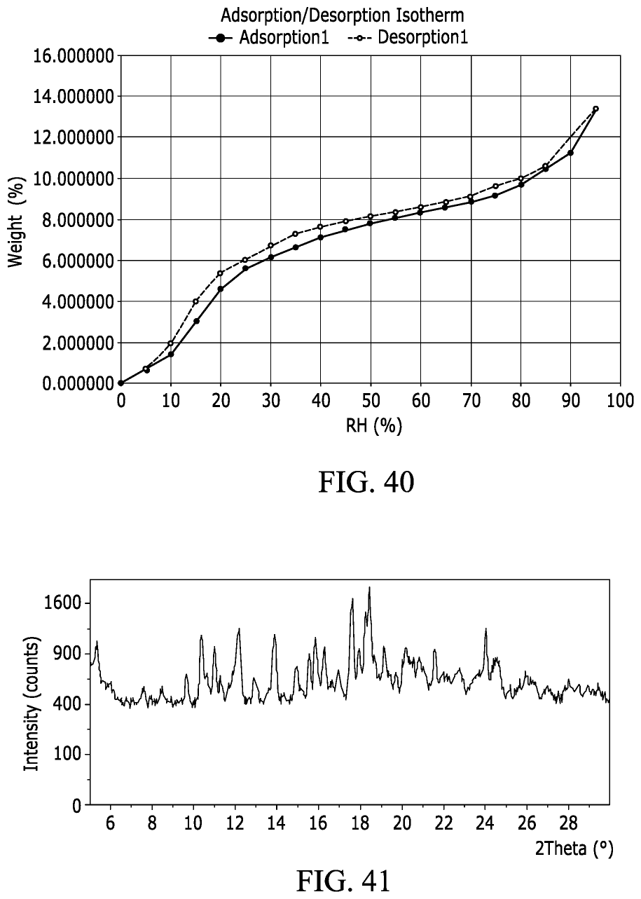

The crystalline phosphate salt form 1 can be characterized by a moisture sorption profile. For example, in some embodiments, the crystalline phosphate salt form 1 is characterized by the moisture sorption profile as shown in FIG. 40, showing a weight gain of 13% by 95% RH.

Fumarate salt form 1: The fumarate salt form 1 can be characterized by an X-ray powder diffraction pattern, obtained as set forth in the Examples, having peaks at 17.6, 18.2, and 18.4±0.2° 2θ using Cu Kα radiation, optionally further characterized by additional peaks at 5.3, 10.4, 12.2, 13.9, 15.8, and 24.0±0.2° 2θ using Cu Kα radiation, and/or additional peaks at 9.7, 11.0, 12.9, 14.9, 15.5, 16.3, 16.9, 17.9, 19.2, 20.2, 20.9, 21.6, 22.8, 24.7, and 26.1±0.2° 2θ using Cu Kα radiation. In some embodiments, the crystalline fumarate salt form 1 has an X-ray powder diffraction pattern substantially as shown in FIG. 41, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

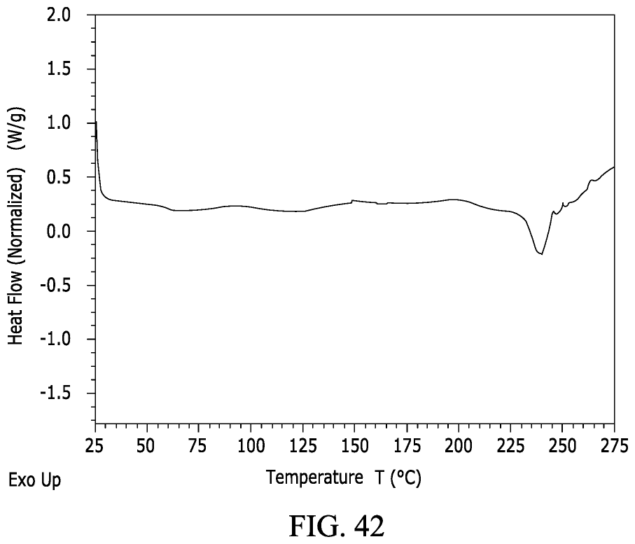

Differential scanning calorimetry (DSC) thermographs were obtained, as set forth in the Examples, for the crystalline fumarate salt form 1. The DSC curve indicates an endothermic transition at 232° C.±3° C. Thus, in some embodiments, the crystalline fumarate salt form 1 can be characterized by a DSC thermograph having a transition endotherm with an onset of 229° C. to 235° C. For example, in some embodiments the crystalline fumarate salt form 1 is characterized by DSC, as shown in FIG. 42.

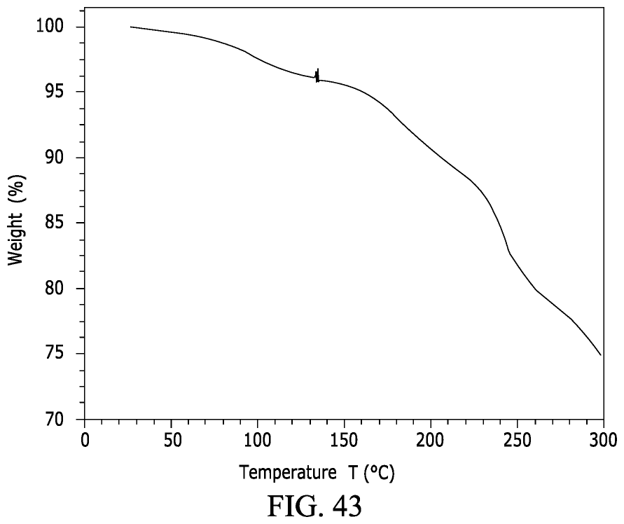

The crystalline fumarate salt form 1 can be characterized by thermogravimetric analysis (TGA). Thus, the crystalline fumarate salt form 1 can be characterized by a weight loss in a range of about 0% to about 21.3% to 271° C. In some embodiments, the crystalline fumarate salt form 1 has a thermogravimetric analysis substantially as depicted in FIG. 43, wherein by “substantially” is meant that the reported TGA features can vary by ±5° C.

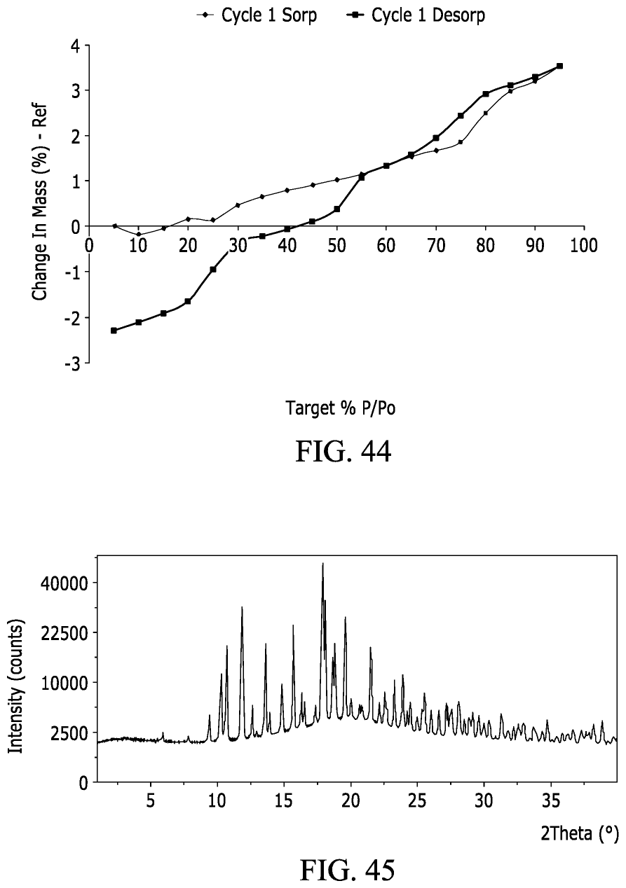

The crystalline fumarate salt form 1 can be characterized by a moisture sorption profile. For example, in some embodiments, the crystalline fumarate salt form 1 is characterized by the moisture sorption profile as shown in FIG. 44, showing a weight gain of 3.5% by 95% RH, and weight loss at 0% RH with form change.

Fumarate salt form 2 (acetone solvate): The fumarate salt form 2 (acetone solvate) can be characterized by an X-ray powder diffraction pattern, obtained as set forth in the Examples, having peaks at 11.9, 17.9, and 18.1±0.2° 2θ using Cu Kα radiation, optionally further characterized by additional peaks at 10.7, 13.6, 15.7, 18.6, 18.8, 19.6, and 21.5±0.2° 2θ using Cu Kα radiation, and/or additional peaks at 10.3, 14.9, 16.3, 16.5, 20.0, 22.2, 22.6, 13.3, 13.9, 24.5, 25.5, and 28.1±0.2° 2θ using Cu Kα radiation. In some embodiments, the crystalline fumarate salt form 2 (acetone solvate) has an X-ray powder diffraction pattern substantially as shown in FIG. 45, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

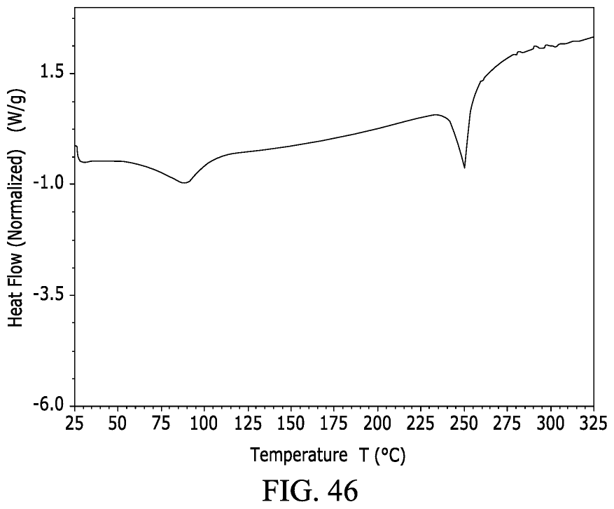

Differential scanning calorimetry (DSC) thermographs were obtained, as set forth in the Examples, for the crystalline fumarate salt form 2 (acetone solvate). The DSC curve indicates an endothermic transition at 243° C.±3° C. Thus, in some embodiments, the crystalline fumarate salt form 2 (acetone solvate) can be characterized by a DSC thermograph having a transition endotherm with an onset of 240° C. to 246° C. For example, in some embodiments the crystalline fumarate salt form 2 (acetone solvate) is characterized by DSC, as shown in FIG. 46.

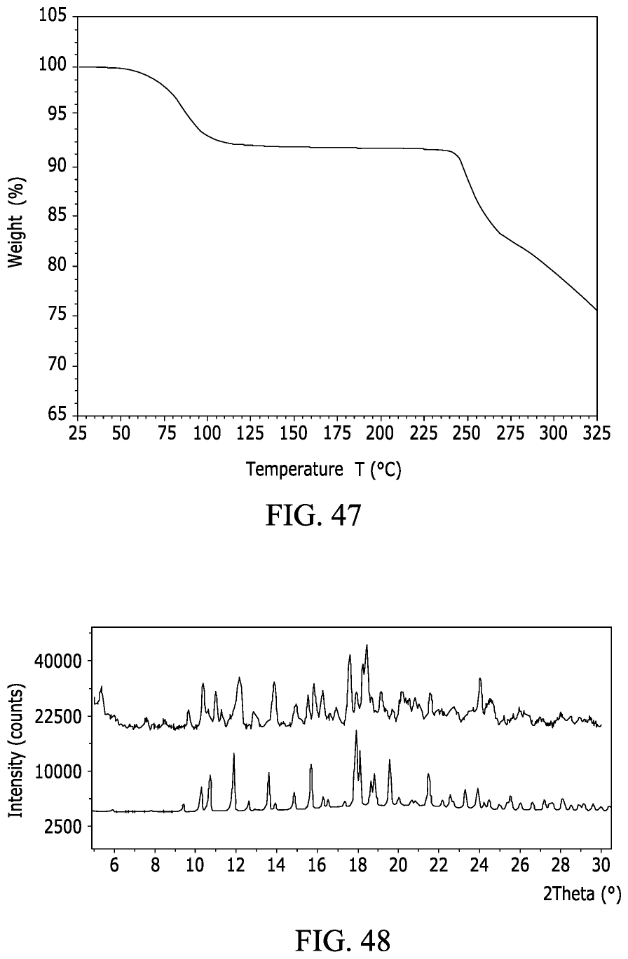

The crystalline fumarate salt form 2 (acetone solvate) can be characterized by thermogravimetric analysis (TGA). Thus, the crystalline fumarate salt form 2 (acetone solvate) can be characterized by a weight loss in a range of about 0% to about 8% to 150° C., with an additional weight loss in a range of about 0% to about 9.3% between 200° C. and 275° C. In some embodiments, the crystalline fumarate salt form 2 (acetone solvate) has a thermogravimetric analysis substantially as depicted in FIG. 47, wherein by “substantially” is meant that the reported TGA features can vary by ±5° C.

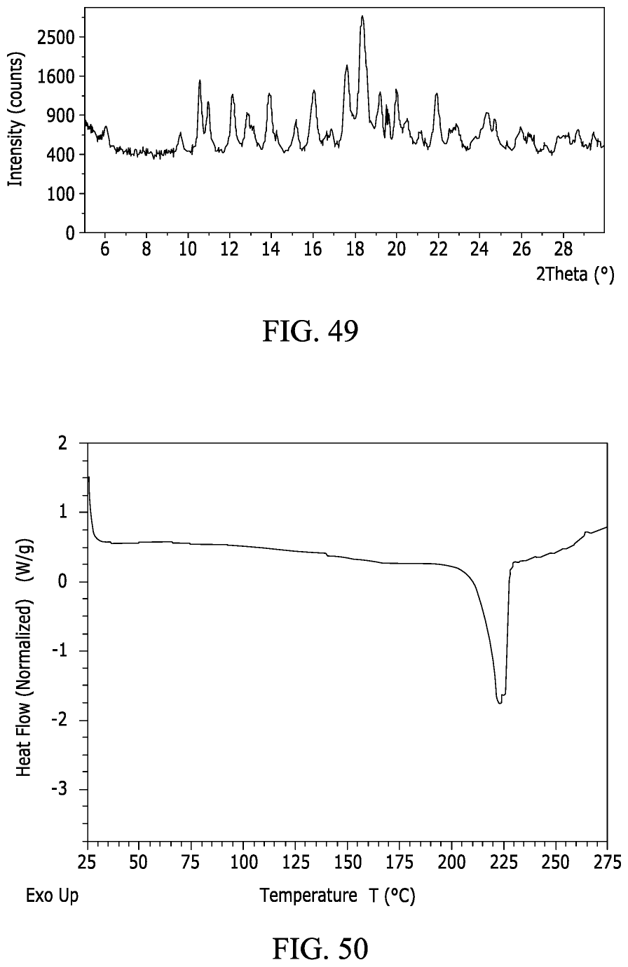

Citrate salt form 1: The citrate salt form 1 can be characterized by an X-ray powder diffraction pattern, obtained as set forth in the Examples, having peaks at 10.6, 17.6, and 18.3±0.2° 2θ using Cu Kα radiation, optionally further characterized by additional peaks at 12.1, 13.9, 16.0, 19.2, and 21.9±0.2° 2θ using Cu Kα radiation, and/or additional peaks at 6.1, 11.0, 12.8, 15.2, 16.9, 19.5, 20.0, 20.5, 21.1, 22.9, 24.4, 24.7, 25.9, and 28.7±0.2° 2θ using Cu Kα radiation. In some embodiments, the crystalline citrate salt form 1 has an X-ray powder diffraction pattern substantially as shown in FIG. 49, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

Differential scanning calorimetry (DSC) thermographs were obtained, as set forth in the Examples, for the crystalline citrate salt form 1. The DSC curve indicates an endothermic transition at 214° C.±3° C. Thus, in some embodiments, the crystalline citrate salt form 1 can be characterized by a DSC thermograph having a transition endotherm with an onset of 211° C. to 217° C. For example, in some embodiments the crystalline citrate salt form 1 is characterized by DSC, as shown in FIG. 50.

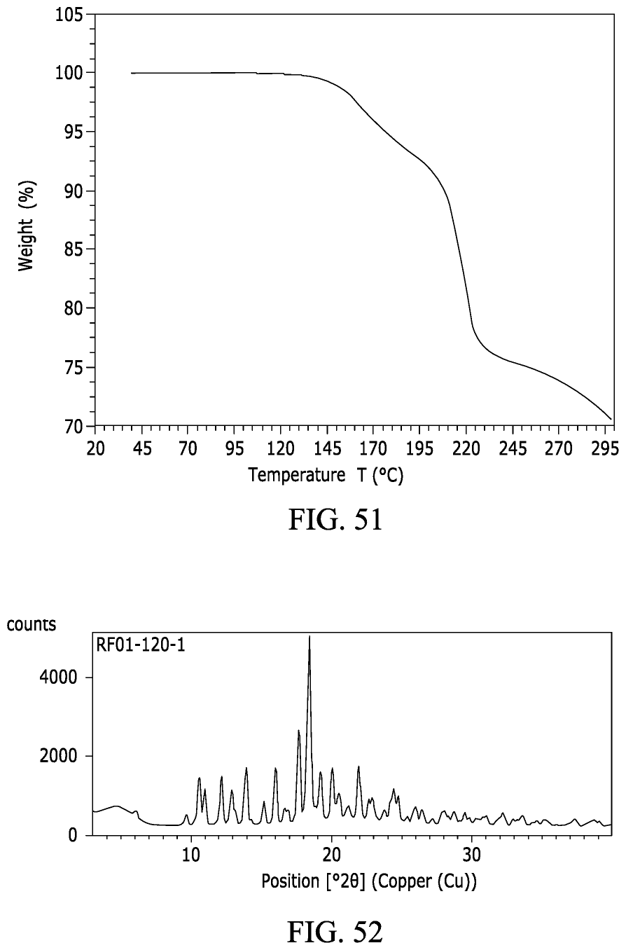

The crystalline citrate salt form 1 can be characterized by thermogravimetric analysis (TGA). Thus, the crystalline citrate salt form 1 can be characterized by a weight loss in a range of about 0% to about 7.1% to 190° C., with an additional weight loss in a range of about 0% to about 16.9% to 245° C. In some embodiments, the crystalline citrate salt form 1 has a thermogravimetric analysis substantially as depicted in FIG. 51, wherein by “substantially” is meant that the reported TGA features can vary by ±5° C.

Citrate salt form 2 (hydrate): The citrate salt form 2 (hydrate) can be characterized by an X-ray powder diffraction pattern, obtained as set forth in the Examples, having peaks at 17.7, 18.4, and 18.5±0.2° 2θ using Cu Kα radiation, optionally further characterized by additional peaks at 14.0, 16.0, 20.1, and 21.9±0.2° 2θ using Cu Kα radiation, and/or additional peaks at 10.7, 11.1, 12.2, 12.9, 15.2, 19.3, 20.6, 22.9, 24.4, and 24.8±0.2° 2θ using Cu Kα radiation. In some embodiments, the crystalline citrate salt form 2 (hydrate) has an X-ray powder diffraction pattern substantially as shown in FIG. 52, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

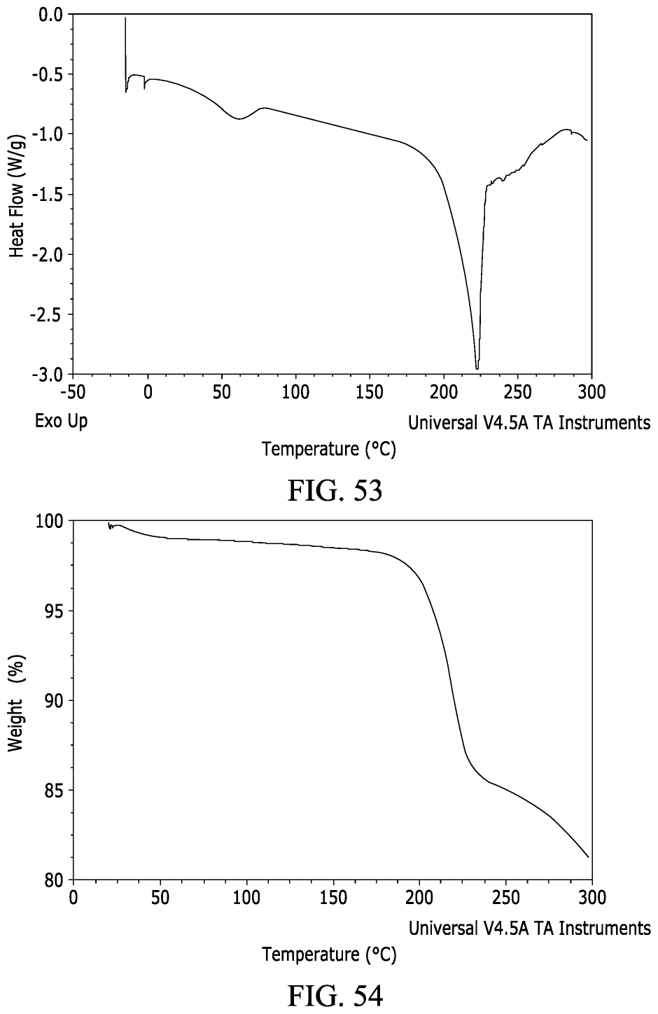

Differential scanning calorimetry (DSC) thermographs were obtained, as set forth in the Examples, for the crystalline citrate salt form 2 (hydrate). The DSC curve indicates an endothermic transition at 206° C.±3° C. Thus, in some embodiments, the crystalline citrate salt form 1 can be characterized by a DSC thermograph having a transition endotherm with an onset of 203° C. to 209° C. For example, in some embodiments the crystalline citrate salt form 2 (hydrate) is characterized by DSC, as shown in FIG. 53.

The crystalline citrate salt form 2 (hydrate) can be characterized by thermogravimetric analysis (TGA). Thus, the crystalline citrate salt form 2 (hydrate) can be characterized by a weight loss in a range of about 0% to about 1.6% to 178° C., with an additional weight loss in a range of about 0% to about 13.5% to 250° C. In some embodiments, the crystalline citrate salt form 2 (hydrate) has a thermogravimetric analysis substantially as depicted in FIG. 54, wherein by “substantially” is meant that the reported TGA features can vary by ±5° C.

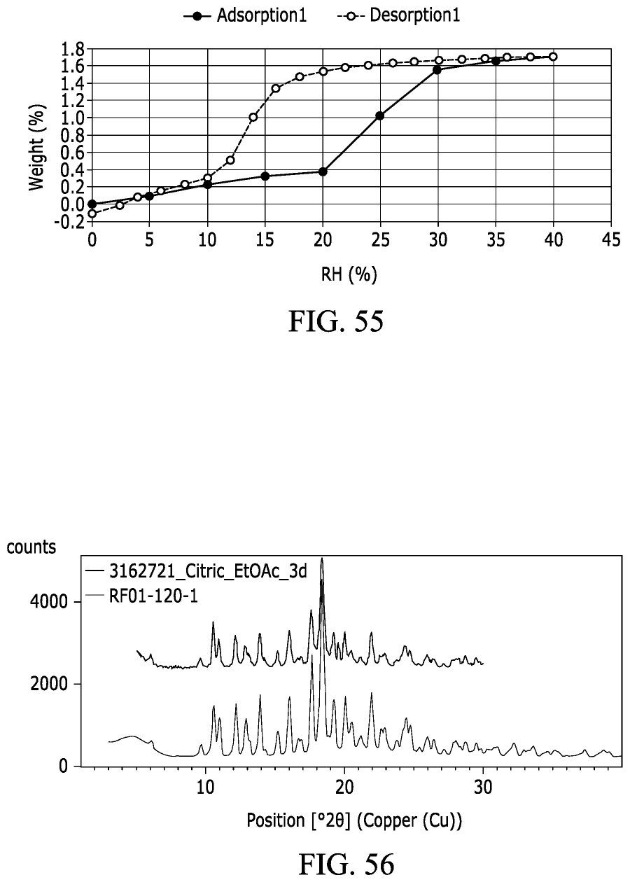

The crystalline citrate salt form 2 (hydrate) can be characterized by a moisture sorption profile. For example, in some embodiments, the crystalline citrate salt form 2 (hydrate) is characterized by the moisture sorption profile as shown in FIG. 55, showing a weight gain of 1.8% by 40% RH, and weight loss between 40% and 0% RH, indicating a monohydrate.

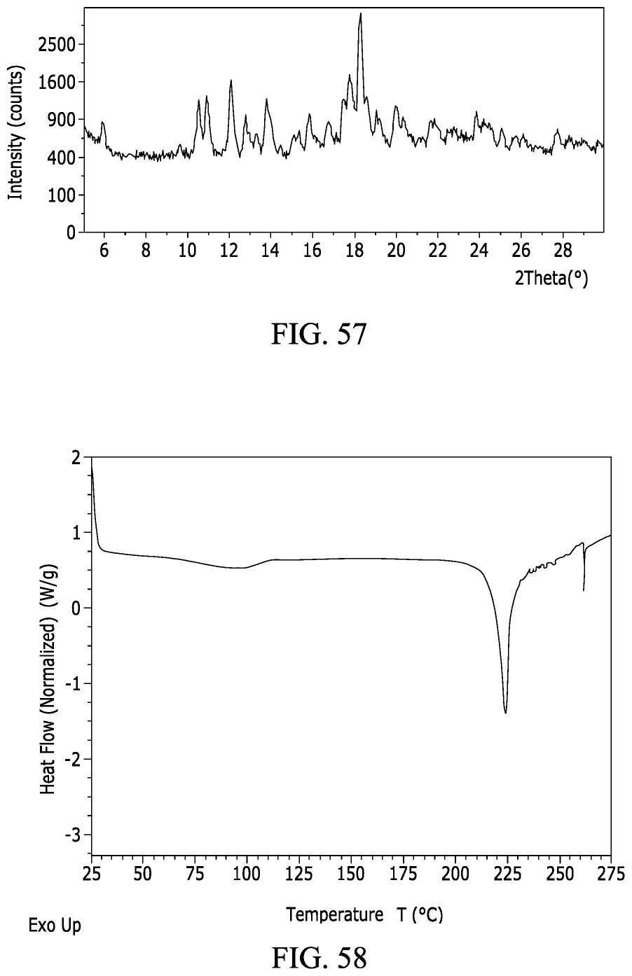

Lactate salt form 1: The lactate salt form 1 can be characterized by an X-ray powder diffraction pattern, obtained as set forth in the Examples, having peaks at 12.1, 17.8, and 18.3±0.2° 2θ using Cu Kα radiation, optionally further characterized by additional peaks at 10.5, 10.9, 13.8, 17.5, and 20.0±0.2° 2θ using Cu Kα radiation, and/or additional peaks at 5.9, 12.8, 15.9, 16.2, 19.1, 20.4, 21.7, 23.9, 24.6, and 25.1±0.2° 2θ using Cu Kα radiation. In some embodiments, the crystalline lactate salt form 1 has an X-ray powder diffraction pattern substantially as shown in FIG. 57, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

Differential scanning calorimetry (DSC) thermographs were obtained, as set forth in the Examples, for the crystalline lactate salt form 1. The DSC curve indicates an endothermic transition at 219° C.±3° C. Thus, in some embodiments, the crystalline lactate salt form 1 can be characterized by a DSC thermograph having a transition endotherm with an onset of 216° C. to 222° C. For example, in some embodiments the crystalline lactate salt form 1 is characterized by DSC, as shown in FIG. 58.

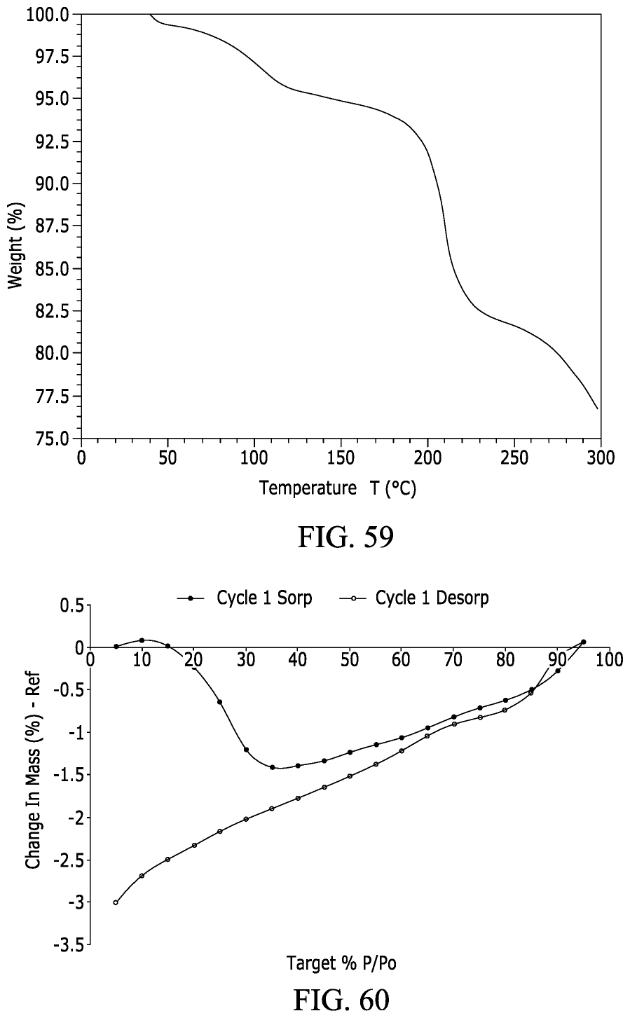

The crystalline lactate salt form 1 can be characterized by thermogravimetric analysis (TGA). Thus, the crystalline lactate salt form 1 can be characterized by a weight loss in a range of about 0% to about 4.7% to 150° C., with an additional weight loss in a range of about 0% to about 12.7% to 250° C. In some embodiments, the crystalline lactate salt form 1 has a thermogravimetric analysis substantially as depicted in FIG. 59, wherein by “substantially” is meant that the reported TGA features can vary by ±5° C.

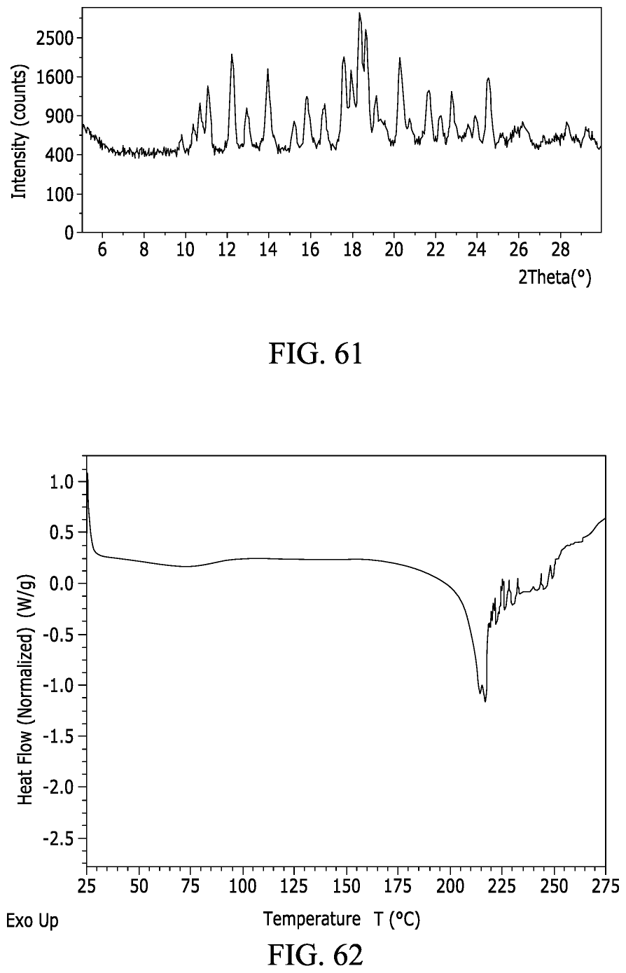

Succinate salt form 1: The succinate salt form 1 can be characterized by an X-ray powder diffraction pattern, obtained as set forth in the Examples, having peaks at 17.6, 18.4, and 18.7±0.2° 2θ using Cu Kα radiation, optionally further characterized by additional peaks at 12.2, 13.9, 18.0, 20.3, and 24.5±0.2° 2θ using Cu Kα radiation, and/or additional peaks at 5.2, 10.4, 10.7, 11.1, 12.9, 15.2, 15.8, 16.7, 19.1, 21.6, 22.2, 22.8, 23.9, 26.2, 28.3, and 29.2±0.2° 2θ using Cu Kα radiation. In some embodiments, the crystalline succinate salt form 1 has an X-ray powder diffraction pattern substantially as shown in FIG. 61, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

Differential scanning calorimetry (DSC) thermographs were obtained, as set forth in the Examples, for the crystalline succinate salt form 1. The DSC curve indicates an endothermic transition at 210° C.±3° C. Thus, in some embodiments, the crystalline succinate salt form 1 can be characterized by a DSC thermograph having a transition endotherm with an onset of 207° C. to 213° C. For example, in some embodiments the crystalline succinate salt form 1 is characterized by DSC, as shown in FIG. 62.

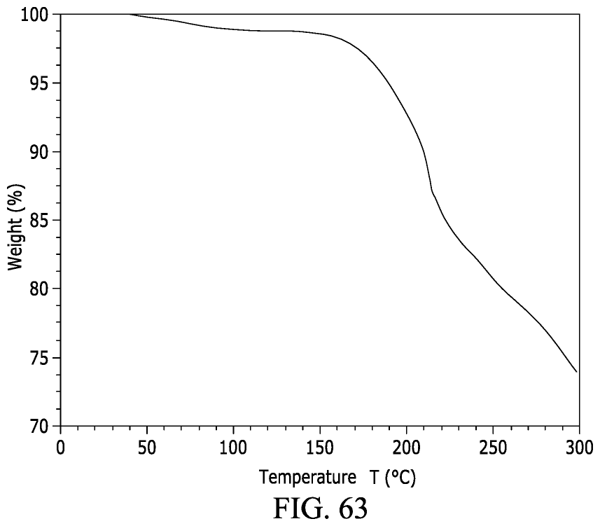

The crystalline succinate salt form 1 can be characterized by thermogravimetric analysis (TGA). Thus, the crystalline succinate salt form 1 can be characterized by a weight loss in a range of about 0% to about 1.1% to 115° C., with an additional weight loss in a range of about 0% to about 15.9% to 235° C. In some embodiments, the crystalline succinate salt form 1 has a thermogravimetric analysis substantially as depicted in FIG. 63, wherein by “substantially” is meant that the reported TGA features can vary by ±5° C.

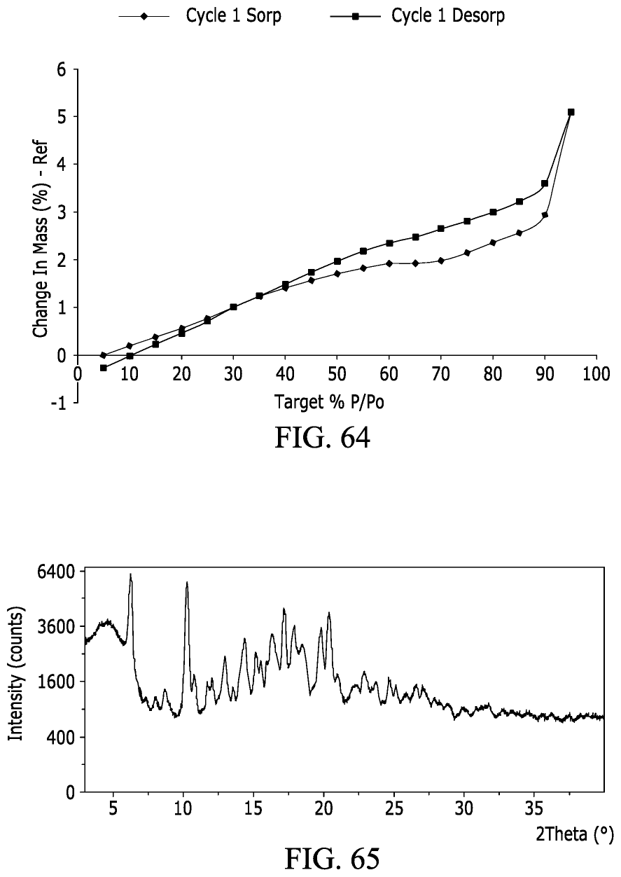

The crystalline succinate salt form 1 can be characterized by a moisture sorption profile. For example, in some embodiments, the crystalline succinate salt form 1 is characterized by the moisture sorption profile as shown in FIG. 64, showing a weight gain of 5.1% by 95% RH.

Ammonium salt form 1: The ammonium salt form 1 can be characterized by an X-ray powder diffraction pattern, obtained as set forth in the Examples, having peaks at 6.2, 10.3, and 17.2±0.2° 2θ using Cu Kα radiation, optionally further characterized by additional peaks at 4.0, 4.7, 17.3, 17.9, 19.8, and 20.3±0.2° 2θ using Cu Kα radiation, and/or additional peaks at 13.0, 14.4, 15.1, 15.5, 15.9, 16.2, 16.4, 17.7, 18.6, 19.7, and 22.8±0.2° 2θ using Cu Kα radiation. In some embodiments, the crystalline ammonium salt form 1 has an X-ray powder diffraction pattern substantially as shown in FIG. 65, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

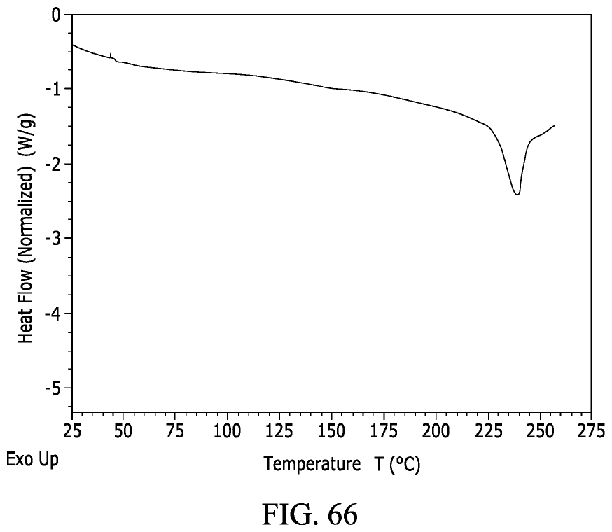

Differential scanning calorimetry (DSC) thermographs were obtained, as set forth in the Examples, for the crystalline ammonium salt form 1. The DSC curve indicates an endothermic transition at 227° C.±3° C. Thus, in some embodiments, the crystalline ammonium salt form 1 can be characterized by a DSC thermograph having a transition endotherm with an onset of 224° C. to 230° C. For example, in some embodiments the crystalline ammonium salt form 1 is characterized by DSC, as shown in FIG. 66.

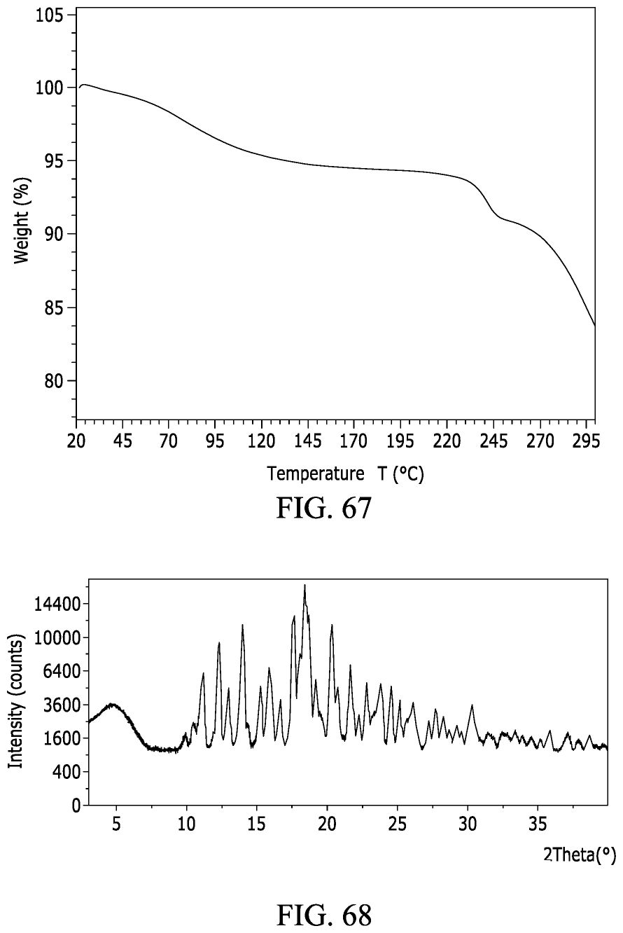

The crystalline ammonium salt form 1 can be characterized by thermogravimetric analysis (TGA). Thus, the crystalline ammonium salt form 1 can be characterized by a weight loss in a range of about 0% to about 5.7% to 170° C., with an additional weight loss in a range of about 0% to about 3.3% to 255° C. In some embodiments, the crystalline ammonium salt form 1 has a thermogravimetric analysis substantially as depicted in FIG. 67, wherein by “substantially” is meant that the reported TGA features can vary by ±5° C.

Besylate salt form 1 (hydrate): The besylate salt form 1 (hydrate) can be characterized by an X-ray powder diffraction pattern, obtained as set forth in the Examples, having peaks at 17.6, 18.4, and 18.7±0.2° 2θ using Cu Kα radiation, optionally further characterized by additional peaks at 14.0, 17.7, and 20.4±0.2° 2θ using Cu Kα radiation, and/or additional peaks at 11.2, 12.4, 13.8, 14.1, 15.9, 16.1, 18.0, 19.3, 20.8, 21.7, 22.9, 23.9, and 24.5±0.2° 2θ using Cu Kα radiation. In some embodiments, the crystalline besylate salt form 1 (hydrate) has an X-ray powder diffraction pattern substantially as shown in FIG. 68, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

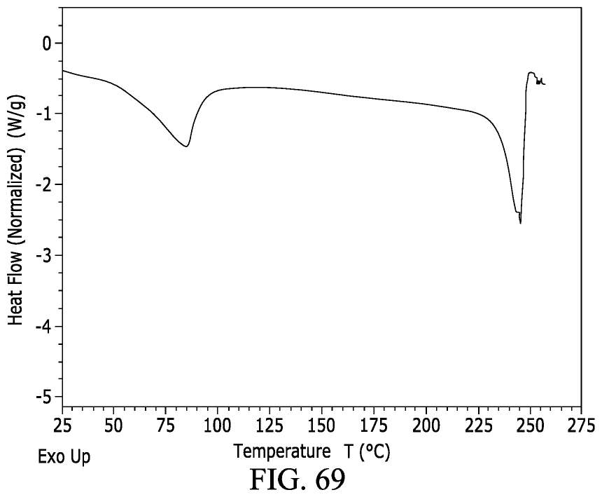

Differential scanning calorimetry (DSC) thermographs were obtained, as set forth in the Examples, for the crystalline besylate salt form 1 (hydrate). The DSC curve indicates endothermic transitions at 57° C.±3° C. and 234° C.±3° C. Thus, in some embodiments, the crystalline besylate salt form 1 (hydrate) can be characterized by a DSC thermograph having transition endotherms with an onset of 54° C. to 60° C. and 231° C. to 237° C. For example, in some embodiments the crystalline besylate salt form 1 (hydrate) is characterized by DSC, as shown in FIG. 69.

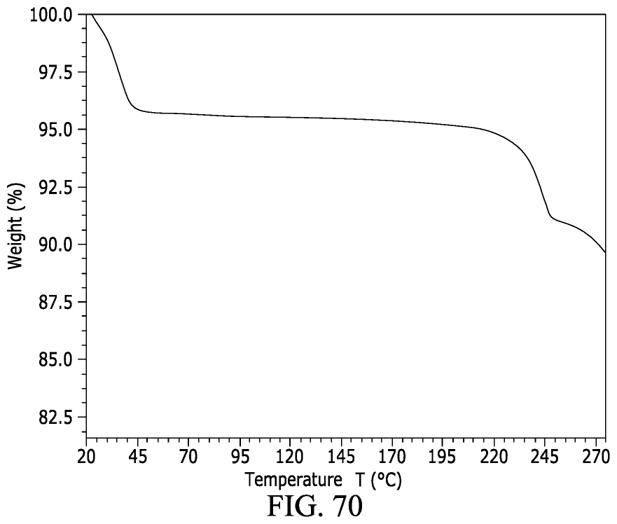

The crystalline besylate salt form 1 (hydrate) can be characterized by thermogravimetric analysis (TGA). Thus, the crystalline besylate salt form 1 (hydrate) can be characterized by a weight loss in a range of about 0% to about 4.1% to 75° C., with an additional weight loss in a range of about 0% to about 4.6% to 260° C. In some embodiments, the crystalline besylate salt form 1 (hydrate) has a thermogravimetric analysis substantially as depicted in FIG. 70, wherein by “substantially” is meant that the reported TGA features can vary by ±5° C.

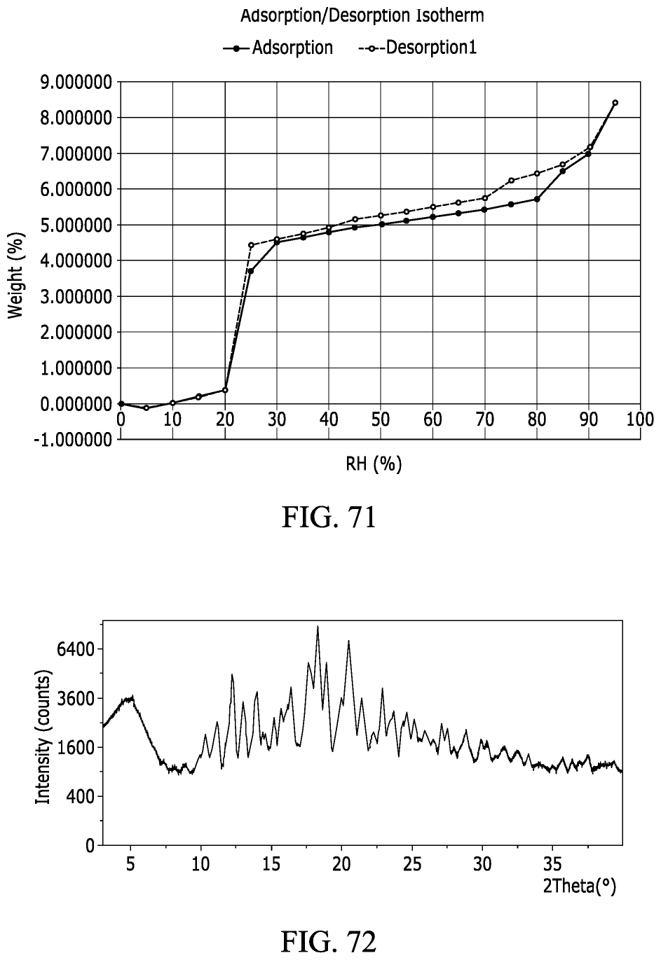

The crystalline besylate salt form 1 (hydrate) can be characterized by a moisture sorption profile. For example, in some embodiments, the crystalline besylate salt form 1 (hydrate) is characterized by the moisture sorption profile as shown in FIG. 71, showing a weight gain of 8.4% by 95% RH, with no form change.

Tosylate salt form 1: The tosylate salt form 1 can be characterized by an X-ray powder diffraction pattern, obtained as set forth in the Examples, having peaks at 18.2, 18.4, and 20.5±0.2° 2θ using Cu Kα radiation, optionally further characterized by additional peaks at 12.2, 12.3, 17.6, 18.9, and 19.1±0.2° 2θ using Cu Kα radiation, and/or additional peaks at 4.5, 5.2, 13.0, 13.8, 14.0, 15.2, 15.8, 16.2, 16.4, 19.8, 20.0, 21.4, 22.9, 23.4, 23.6, 23.8, 24.3, 24.6, 25.1, and 27.1±0.2° 2θ using Cu Kα radiation. In some embodiments, the crystalline tosylate salt form 1 has an X-ray powder diffraction pattern substantially as shown in FIG. 72, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

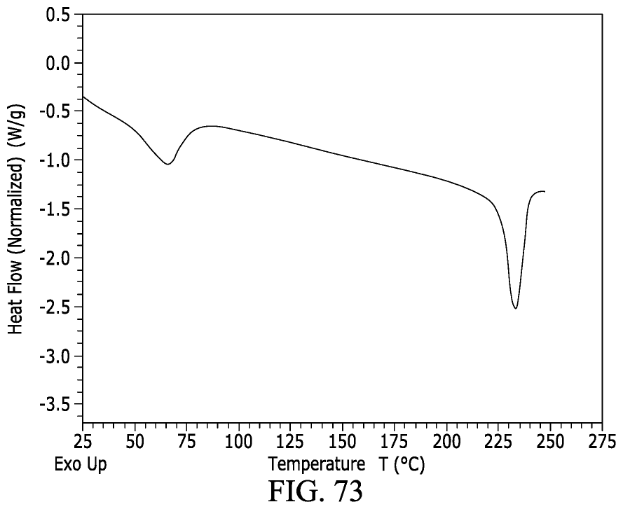

Differential scanning calorimetry (DSC) thermographs were obtained, as set forth in the Examples, for the crystalline tosylate salt form 1. The DSC curve indicates endothermic transitions at 40° C.±3° C. and 226° C.±3° C. Thus, in some embodiments, the crystalline tosylate salt form 1 can be characterized by a DSC thermograph having transition endotherms with an onset of 37° C. to 43° C. and 223° C. to 229° C. For example, in some embodiments the crystalline tosylate salt form 1 is characterized by DSC, as shown in FIG. 73.

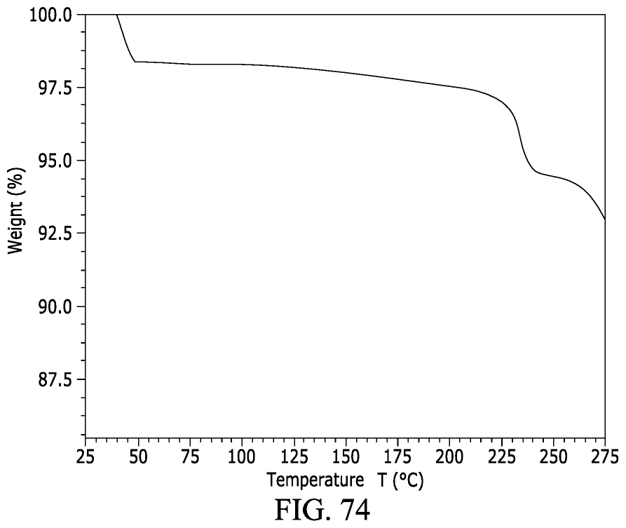

The crystalline tosylate salt form 1 can be characterized by thermogravimetric analysis (TGA). Thus, the crystalline tosylate salt form 1 can be characterized by a weight loss in a range of about 0% to about 1.6% to 75° C., with an additional weight loss in a range of about 0% to about 3.9% to 250° C. In some embodiments, the crystalline tosylate salt form 1 has a thermogravimetric analysis substantially as depicted in FIG. 74, wherein by “substantially” is meant that the reported TGA features can vary by ±5° C.

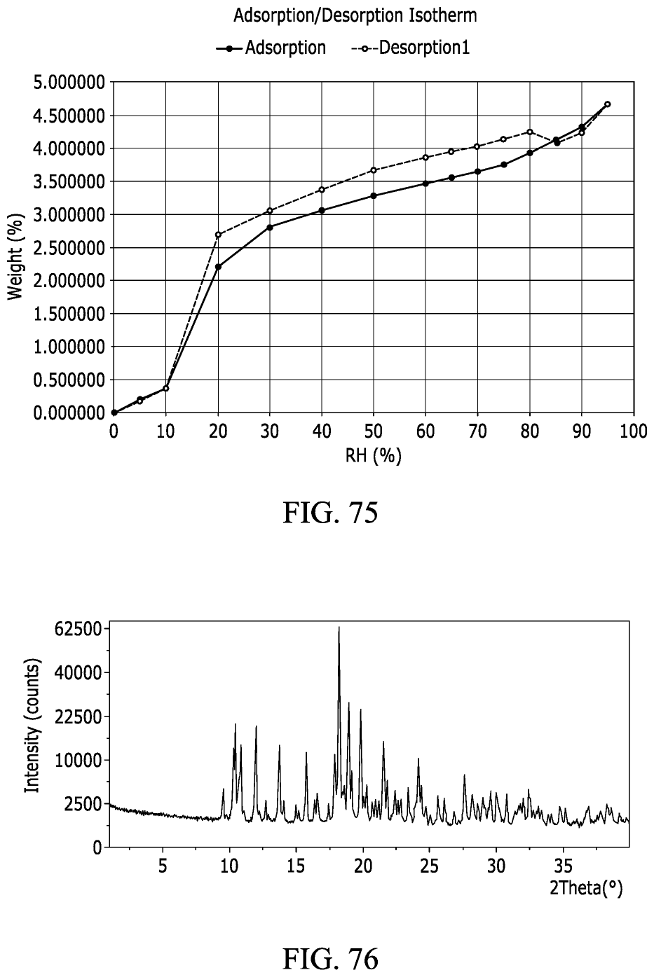

The crystalline tosylate salt form 1 can be characterized by a moisture sorption profile. For example, in some embodiments, the crystalline tosylate salt form 1 is characterized by the moisture sorption profile as shown in FIG. 75, showing a weight gain of 4.7% by 95% RH, with no form change.

Maleate salt form 1 (family of isostructural solvates): The maleate salt form 1 (family of isostructural solvates) can be characterized by an X-ray powder diffraction pattern, obtained as set forth in the Examples, having peaks at 18.2, 18.9, and 19.9±0.2° 2θ using Cu Kα radiation, optionally further characterized by additional peaks at 10.4, 10.9, 12.0, and 21.5±0.2° 2θ using Cu Kα radiation, and/or additional peaks at 10.3, 13.8, 15.8, 17.9, 19.2, and 24.2±0.2° 2θ using Cu Kα radiation. In some embodiments, the crystalline maleate salt form 1 (family of isostructural solvates) has an X-ray powder diffraction pattern substantially as shown in FIG. 76, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

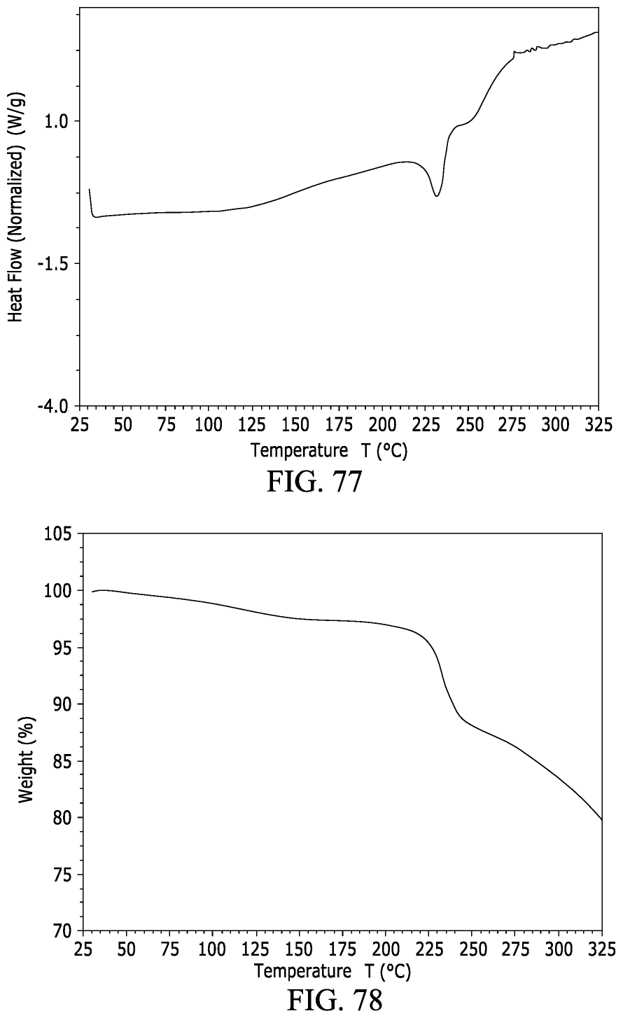

Differential scanning calorimetry (DSC) thermographs were obtained, as set forth in the Examples, for the crystalline maleate salt form 1 (family of isostructural solvates). The DSC curve indicates an endothermic transition at 222° C.±3° C. Thus, in some embodiments, the crystalline maleate salt form 1 (family of isostructural solvates) can be characterized by a DSC thermograph having a transition endotherm with an onset of 219° C. to 225° C. For example, in some embodiments the crystalline maleate salt form 1 (family of isostructural solvates) is characterized by DSC, as shown in FIG. 77.

The crystalline maleate salt form 1 (family of isostructural solvates) can be characterized by thermogravimetric analysis (TGA). Thus, the crystalline maleate salt form 1 (family of isostructural solvates) can be characterized by a weight loss in a range of about 0% to about 11.9% to 250° C. In some embodiments, the crystalline maleate salt form 1 (family of isostructural solvates) has a thermogravimetric analysis substantially as depicted in FIG. 78, wherein by “substantially” is meant that the reported TGA features can vary by ±5° C.

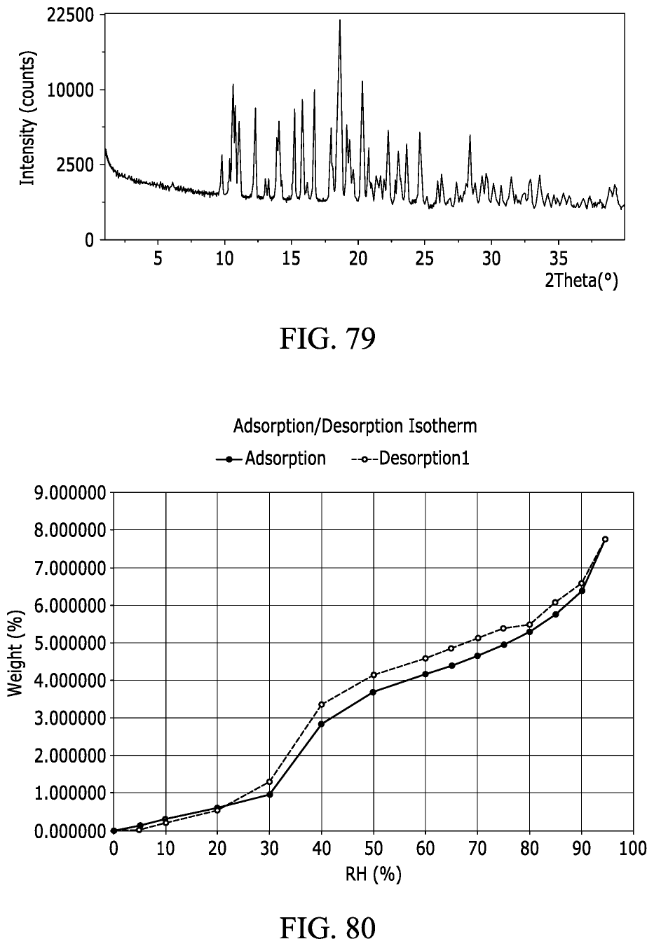

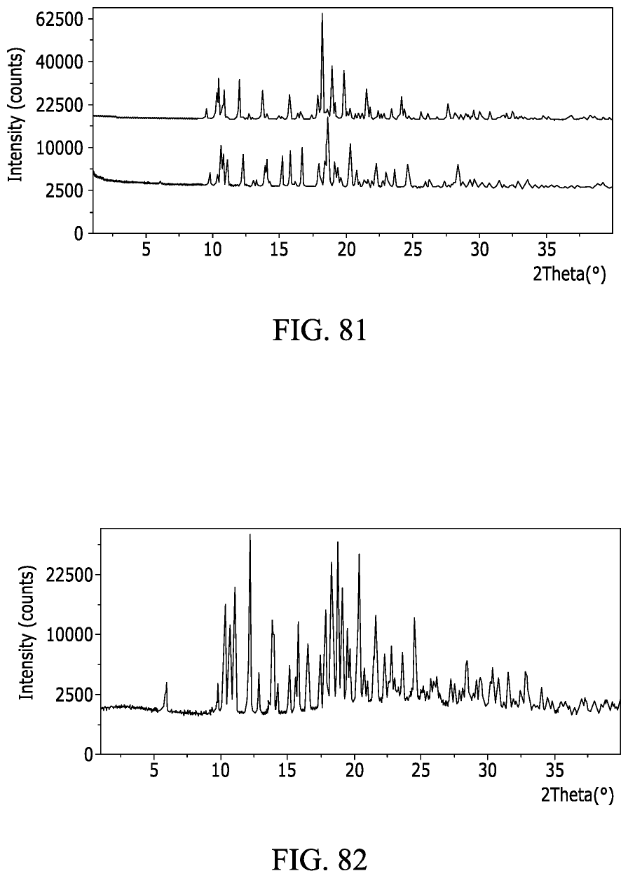

Maleate salt form 2: The maleate salt form 2 can be characterized by an X-ray powder diffraction pattern, obtained as set forth in the Examples, having peaks at 10.6, 18.6, and 20.3±0.2° 2θ using Cu Kα radiation, optionally further characterized by additional peaks at 10.8, 12.3, 15.2, 15.9, and 16.7±0.2° 2θ using Cu Kα radiation, and/or additional peaks at 9.8, 11.1, 13.9, 14.1, 18.0, 18.4, 19.2, 19.4, 20.8, 22.3, 23.0, 23.6, 24.6, and 28.4±0.2° 2θ using Cu Kα radiation. In some embodiments, the crystalline maleate salt form 2 has an X-ray powder diffraction pattern substantially as shown in FIG. 79, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

The crystalline maleate salt form 2 can be characterized by a moisture sorption profile. For example, in some embodiments, the crystalline maleate salt form 2 is characterized by the moisture sorption profile as shown in FIG. 80, showing a weight gain of 7.7% by 95% RH, with no form change.

Malonate salt form 1: The malonate salt form 1 can be characterized by an X-ray powder diffraction pattern, obtained as set forth in the Examples, having peaks at 12.2, 18.8, and 20.4±0.2° 2θ using Cu Kα radiation, optionally further characterized by additional peaks at 10.3, 11.1, 17.9, 18.3, and 19.1±0.2° 2θ using Cu Kα radiation, and/or additional peaks at 10.7, 13.9, 14.0, 15.8, 16.5, 18.4, 19.5, 19.7 21.6, 21.7, 22.8, and 24.5±0.2° 2θ using Cu Kα radiation. In some embodiments, the crystalline malonate salt form 1 has an X-ray powder diffraction pattern substantially as shown in FIG. 82, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

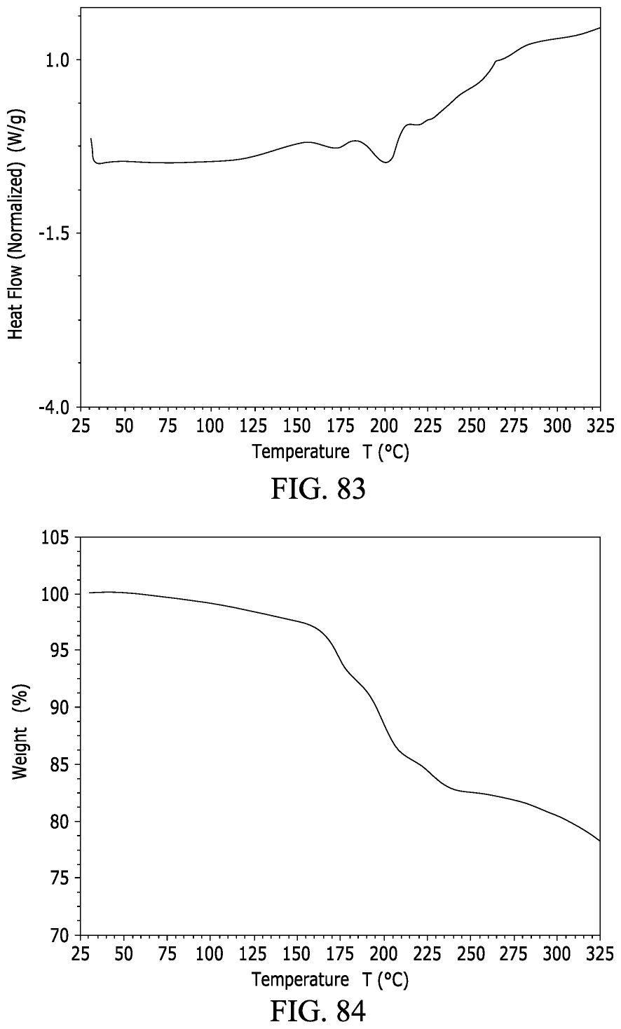

Differential scanning calorimetry (DSC) thermographs were obtained, as set forth in the Examples, for the crystalline malonate salt form 1. The DSC curve indicates endothermic transitions at 161° C.±3° C. and 187° C.±3° C. Thus, in some embodiments, the crystalline tosylate salt form 1 can be characterized by a DSC thermograph having transition endotherms with an onset of 158° C. to 164° C. and 184° C. to 190° C. For example, in some embodiments the crystalline malonate salt form 1 is characterized by DSC, as shown in FIG. 83.

The crystalline malonate salt form 1 can be characterized by thermogravimetric analysis (TGA). Thus, the crystalline malonate salt form 1 can be characterized by a weight loss in a range of about 0% to about 17.5% to 250° C. In some embodiments, the crystalline malonate salt form 1 has a thermogravimetric analysis substantially as depicted in FIG. 84, wherein by “substantially” is meant that the reported TGA features can vary by ±5° C.

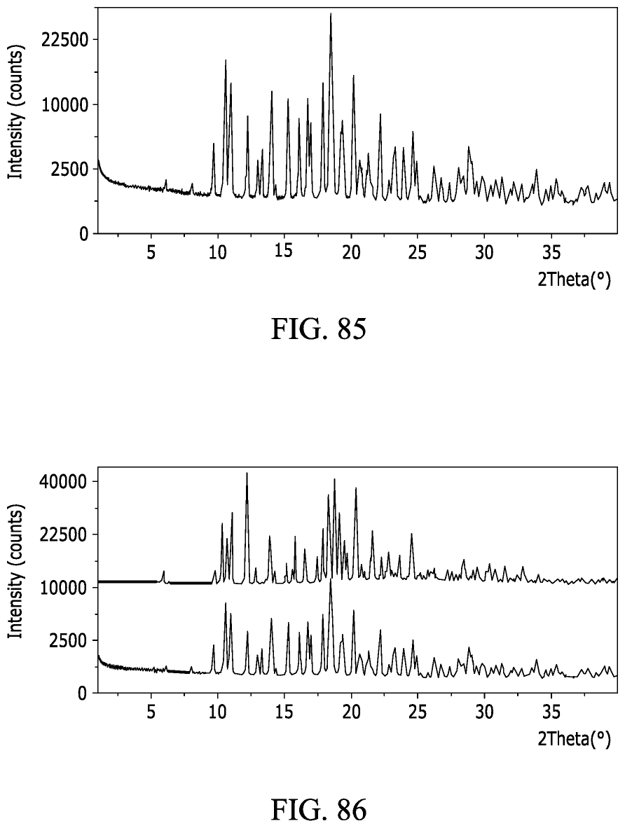

Malonate salt form 2: The malonate salt form 2 can be characterized by an X-ray powder diffraction pattern, obtained as set forth in the Examples, having peaks at 10.6, 18.5, and 20.2±0.2° 2θ using Cu Kα radiation, optionally further characterized by additional peaks at 11.0, 14.0, and 17.9±0.2° 2θ using Cu Kα radiation, and/or additional peaks at 11.1, 12.3, 15.3, 16.1, 16.8, 17.0, 18.6, 19.4, and 22.2±0.2° 2θ using Cu Kα radiation. In some embodiments, the crystalline malonate salt form 2 has an X-ray powder diffraction pattern substantially as shown in FIG. 85, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

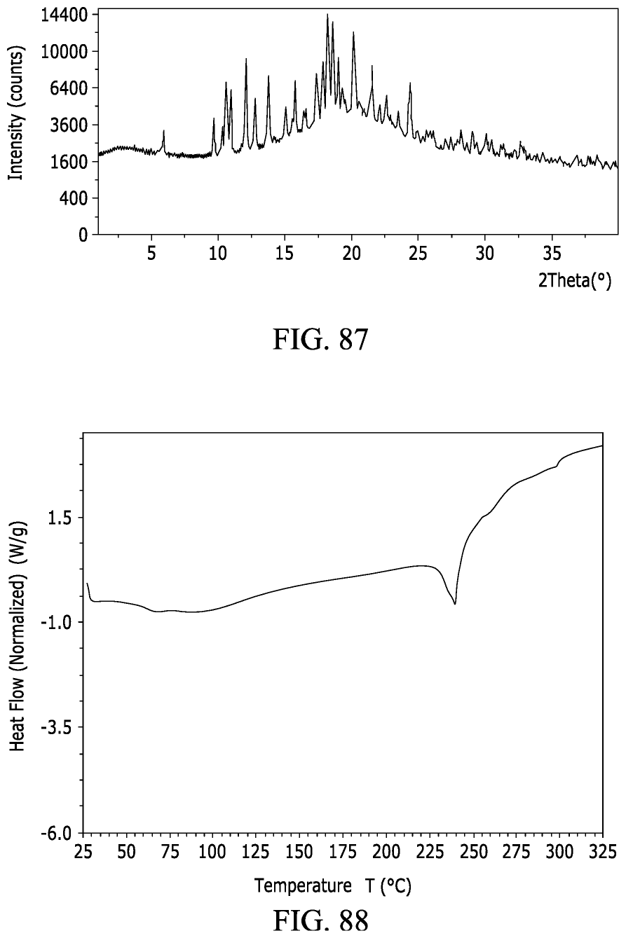

Tartrate salt form 1 (family of isostructural solvates): The tartrate salt form 1 (family of isostructural solvates) can be characterized by an X-ray powder diffraction pattern, obtained as set forth in the Examples, having peaks at 18.2, 18.6, and 20.2±0.2° 2θ using Cu Kα radiation, optionally further characterized by additional peaks at 12.1, 17.8, 19.0, and 21.5±0.2° 2θ using Cu Kα radiation, and/or additional peaks at 10.6, 11.0, 12.8, 13.8, 15.1, 15.8, 16.4, 16.6, 17.4, 19.3, 19.5, 20.6, 22.1, 22.6, 23.5, and 24.4±0.2° 2θ using Cu Kα radiation. In some embodiments, the crystalline tartrate salt form 1 (family of isostructural solvates) has an X-ray powder diffraction pattern substantially as shown in FIG. 87, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

Differential scanning calorimetry (DSC) thermographs were obtained, as set forth in the Examples, for the crystalline tartrate salt form 1 (family of isostructural solvates). The DSC curve indicates an endothermic transition at 227° C.±3° C. Thus, in some embodiments, the crystalline tartrate salt form 1 (family of isostructural solvates) can be characterized by a DSC thermograph having a transition endotherm with an onset of 224° C. to 230° C. For example, in some embodiments the crystalline tartrate salt form 1 (family of isostructural solvates) is characterized by DSC, as shown in FIG. 88.

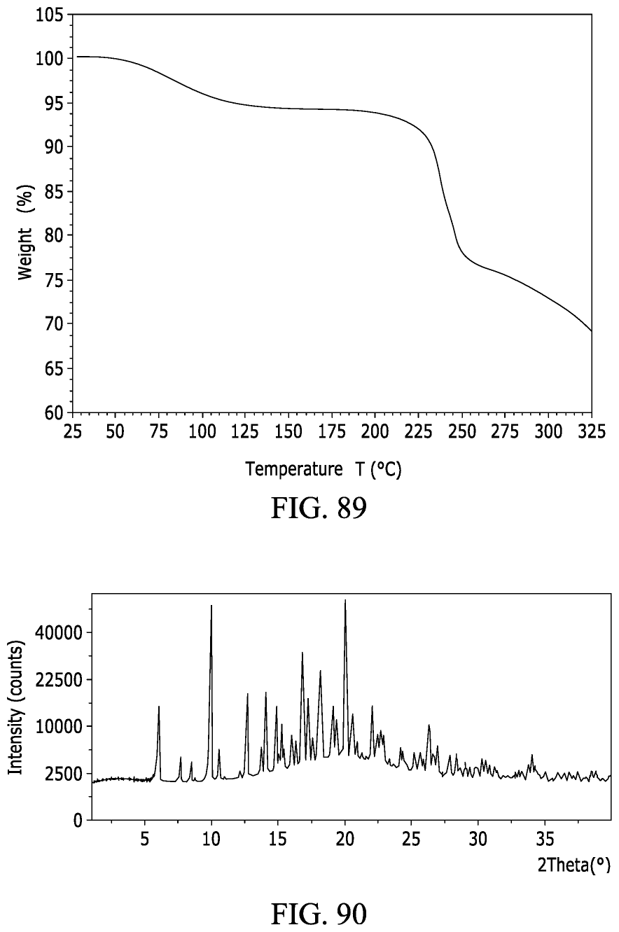

The crystalline tartrate salt form 1 (family of isostructural solvates) can be characterized by thermogravimetric analysis (TGA). Thus, the crystalline tartrate salt form 1 (family of isostructural solvates) can be characterized by a weight loss in a range of about 0% to about 23.0% to 255° C. In some embodiments, the crystalline tartrate salt form 1 (family of isostructural solvates) has a thermogravimetric analysis substantially as depicted in FIG. 89, wherein by “substantially” is meant that the reported TGA features can vary by ±5° C.

Tris(hydroxymethyl)aminomethane (tris) salt form 1 (acetone solvate): The crystalline tris(hydroxymethyl)aminomethane (tris) salt form 1 (acetone solvate) can be characterized by an X-ray powder diffraction pattern, obtained as set forth in the Examples, having peaks at 10.0, 16.8, and 20.0±0.2° 2θ using Cu Kα radiation, optionally further characterized by additional peaks at 12.7, 14.1, and 18.2±0.2° 2θ using Cu Kα radiation, and/or additional peaks at 6.1, 14.9, 15.3, 16.0, 17.3, 17.6, 18.0, 19.0, 19.1, 19.4, 20.6, 22.1, 22.5, 22.7, 22.9, 26.3, and 26.4±0.2° 2θ using Cu Kα radiation. In some embodiments, the crystalline tris(hydroxymethyl)aminomethane (tris) salt form 1 (acetone solvate) has an X-ray powder diffraction pattern substantially as shown in FIG. 90, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

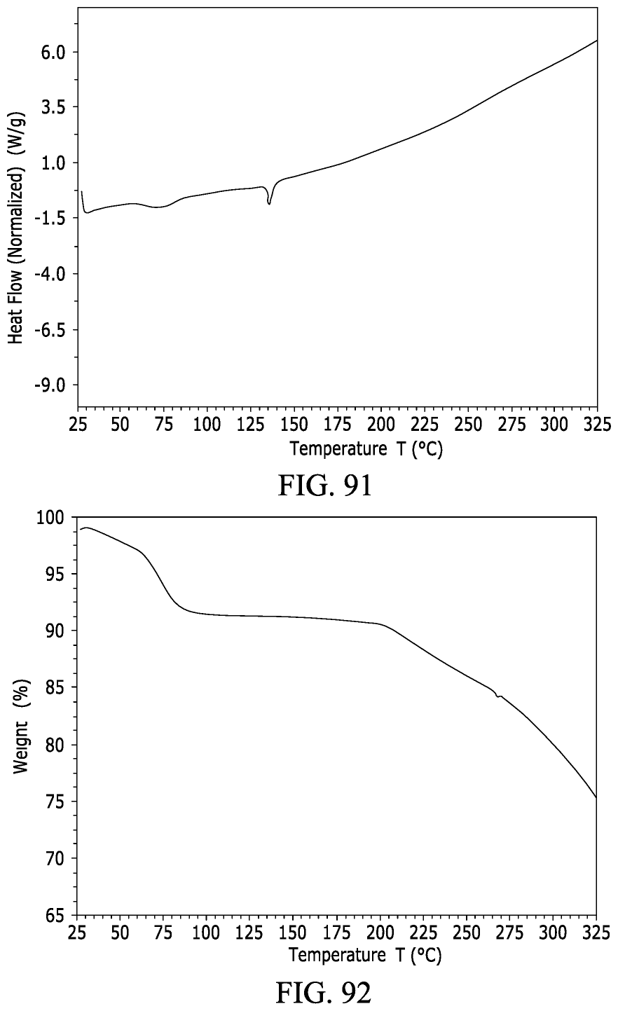

Differential scanning calorimetry (DSC) thermographs were obtained, as set forth in the Examples, for the crystalline tris(hydroxymethyl)aminomethane (tris) salt form 1 (acetone solvate). The DSC curve indicates endothermic transitions at 59° C.±3° C. and 134° C.±3° C. Thus, in some embodiments, the crystalline tris(hydroxymethyl)aminomethane (tris) salt 1 (acetone solvate) can be characterized by a DSC thermograph having a transition endotherm with an onset of 56° C. to 62° C. and 131° C. to 137° C. For example, in some embodiments the crystalline tris(hydroxymethyl)aminomethane (tris) salt 1 (acetone solvate) is characterized by DSC, as shown in FIG. 91.

The crystalline tris(hydroxymethyl)aminomethane (tris) salt form 1 (acetone solvate) can be characterized by thermogravimetric analysis (TGA). Thus, the crystalline tris(hydroxymethyl)aminomethane (tris) salt form 1 (acetone solvate) can be characterized by a weight loss in a range of about 0% to about 7.9% to 150° C. In some embodiments, the crystalline tris(hydroxymethyl)aminomethane (tris) salt form 1 (acetone solvate) has a thermogravimetric analysis substantially as depicted in FIG. 92, wherein by “substantially” is meant that the reported TGA features can vary by ±5° C.

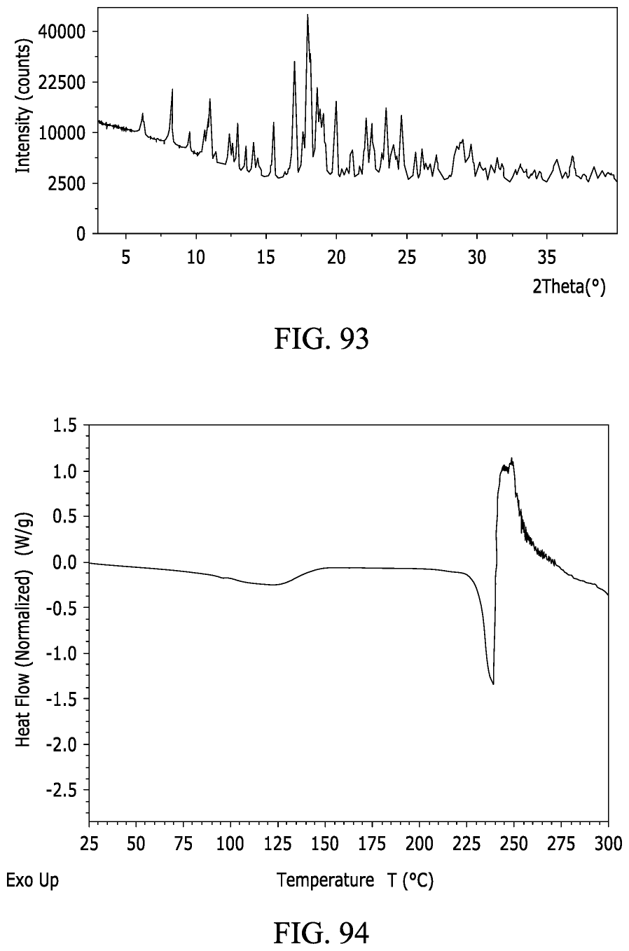

Iodide salt form 1: The iodide salt form 1 can be characterized by an X-ray powder diffraction pattern, obtained as set forth in the Examples, having peaks at 17.0, 18.0, and 18.1±0.2° 2θ using Cu Kα radiation, optionally further characterized by additional peaks at 8.3, 11.0, 18.6, 18.8, 19.1, 20.0, 22.1, 23.5, and 24.7±0.2° 2θ using Cu Kα radiation, and/or additional peaks at 6.2, 10.6, 10.8, 12.4, 13.0, 14.1, 15.5, 17.6, 22.5, 24.1, 28.6, 28.8, 29.0, and 29.5±0.2° 2θ using Cu Kα radiation. In some embodiments, the crystalline iodide salt form 1 has an X-ray powder diffraction pattern substantially as shown in FIG. 93, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

Differential scanning calorimetry (DSC) thermographs were obtained, as set forth in the Examples, for the crystalline iodide salt form 1. The DSC curve indicates an endothermic transition at 231° C.±3° C. Thus, in some embodiments, the crystalline iodide salt form 1 can be characterized by a DSC thermograph having a transition endotherm with an onset of 228° C. to 234° C. For example, in some embodiments the crystalline iodide salt form 1 is characterized by DSC, as shown in FIG. 94.

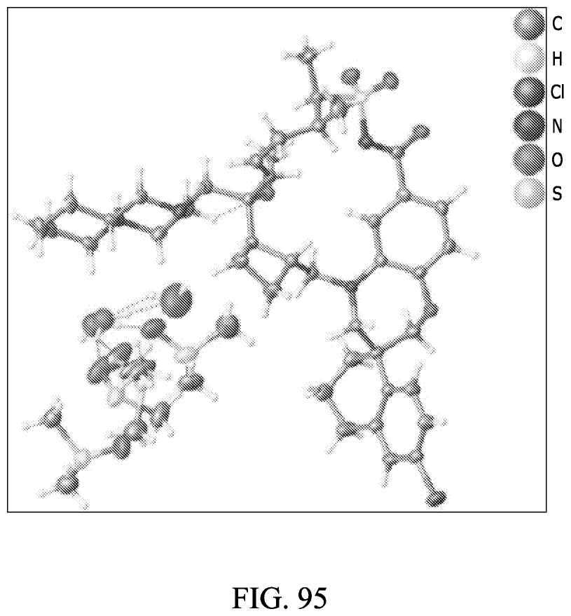

DMSO solvate: The DMSO solvate can be characterized by a single crystal structure substantially as shown in FIG. 95, or as set forth in the Examples.

### Pharmaceutical Formulations

Provided herein are pharmaceutical formulations comprising a salt or solvate of AMG 397 as disclosed herein and a pharmaceutically acceptable excipient.

In some embodiments, the pharmaceutical formulation is in the form of a tablet. In some embodiments, the pharmaceutical formulation is in the form of an immediate release tablet. Solid oral drug compositions (e.g., tablets) or preparations have various release profiles, such as an immediate release profile as referenced by FDA guidelines (“Dissolution Testing of Immediate Release Solid Oral Dosage Forms”, issued August 1997, Section IV-A). In the dissolution testing guideline for immediate release profiles, materials which dissolve at least 80% in the first 30 to 60 minutes in solution qualify as immediate release profiles.

Therefore, immediate release solid dosage forms permit the release of most or all of the active ingredient over a short period of time, such as 60 minutes or less, and make rapid absorption of the drug possible. In contrast, extended release solid oral dosage forms permit the release of the active ingredient over an extended period of time in an effort to maintain therapeutically effective plasma levels over similarly extended time intervals, improve dosing compliance, and/or to modify other pharmacokinetic properties of the active ingredient.

“Pharmaceutically acceptable excipient” refers to a broad range of ingredients that may be combined with a compound or salt of the present invention to prepare a pharmaceutical composition or formulation. Excipients are additives that are included in a formulation because they either impart or enhance the stability, delivery and manufacturability of a drug product, and are physiologically innocuous to the recipient thereof. Regardless of the reason for their inclusion, excipients are an integral component of a drug product and therefore need to be safe and well tolerated by patients. Given the teachings and guidance provided herein, those skilled in the art will readily be able to vary the amount or range of excipient without increasing viscosity to an undesirable level. Excipients may be chosen to achieve a desired bioavailability, desired stability, resistance to aggregation or degradation or precipitation, protection under conditions of freezing, lyophilization or high temperatures, or other properties. Typically, excipients include, but are not limited to, diluents, colorants, vehicles, anti-adherants, glidants, disintegrants, flavoring agents, coatings, binders, sweeteners, lubricants, sorbents, preservatives, and the like. Examples of suitable excipients are well known to the person skilled in the art of tablet formulation and may be found e.g. in Handbook of Pharmaceutical Excipients (eds. Rowe, Sheskey & Quinn), 6th edition 2009.

As used herein the term “excipients” is intended to refer to inter alia basifying agents, solubilizers, glidants, fillers, binders, lubricant, diluents, preservatives, surface active agents, dispersing agents and the like. The term also includes agents such as sweetening agents, flavoring agents, coloring agents and preserving agents. Such components will generally be present in admixture within the tablet.

Examples of solubilizers include, but are not limited to, ionic surfactants (including both ionic and non-ionic surfactants) such as sodium lauryl sulfate, cetyltrimethylammonium bromide, polysorbates (such as polysorbate 20 or 80), poloxamers (such as poloxamer 188 or 207), and macrogols.

Examples of lubricants, glidants and flow aids include, but are not limited to, magnesium stearate, calcium stearate, stearic acid, hydrogenated vegetable oil, glyceryl palmitostearate, glyceryl behenate, sodium stearyl fumarate, colloidal silicon dioxide, and talc. The amount of lubricant in a tablet can generally be between 0.1-5% by weight.

Examples of disintegrants include, but are not limited to, starches, celluloses, cross-linked PVP, sodium starch glycolate, croscarmellose sodium, etc.

Examples of fillers (also known as bulking agents or diluents) include, but are not limited to, starches, maltodextrins, polyols (such as lactose), and celluloses. Tablets provided herein may include lactose and/or microcrystalline cellulose. Lactose can be used in anhydrous or hydrated form (e.g. monohydrate), and is typically prepared by spray drying, fluid bed granulation, or roller drying.

Examples of binders include, but are not limited to, cross-linked PVP, HPMC, microcrystalline cellulose, sucrose, starches, etc.

In some embodiments, the pharmaceutically acceptable excipients can comprise one or more diluent, binder, or disintegrant. In embodiments, the pharmaceutically acceptable excipients can comprise a diluent comprising one or more of microcrystalline cellulose, starch, dicalcium phosphate, lactose, sorbitol, mannitol, sucrose, and methyl dextrins, a binder comprising one or more of povidone, hydroxypropyl methylcellulose, hydroxypropyl cellulose, and sodium carboxymethylcellulose, and a disintegrant comprising one or more of crospovidine, sodium starch glycolate, and croscarmellose sodium.

Tablets provided herein may be uncoated or coated (in which case they include a coating). Although uncoated tablets may be used, it is more usual to provide a coated tablet, in which case a conventional non-enteric coating may be used. Film coatings are known in the art and can be composed of hydrophilic polymer materials, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though the water soluble material included in the film coating of the present invention may include a single polymer material, it may also be formed using a mixture of more than one polymer. The coating may be white or colored e.g. gray. Suitable coatings include, but are not limited to, polymeric film coatings such as those comprising polyvinyl alcohol e.g. ‘Opadry® II’ (which includes part-hydrolysed PVA, titanium dioxide, macrogol 3350 and talc, with optional coloring such as iron oxide or indigo carmine or iron oxide yellow or FD&C yellow #6). The amount of coating will generally be between 2-4% of the core's weight, and in certain specific embodiments, 3%. Unless specifically stated otherwise, where the dosage form is coated, it is to be understood that a reference to % weight of the tablet means that of the total tablet, i.e. including the coating.

The pharmaceutical formulations disclosed herein can further comprise a surfactant. As used herein, the surfactant can be cationic, anionic, or non-ionic. In some embodiments, the pharmaceutical formulation can comprise a non-ionic surfactant. In some embodiments, the surfactant can comprise a polysorbate, a poloxamer, or a combination thereof. In some embodiments, the surfactant can comprise polysorbate 20, polysorbate 60, polysorbate 80, or a combination thereof.

### Methods of Treating a Subject

Further provided herein are methods of treating a subject suffering from cancer, comprising administering to the subject a therapeutically effective amount of a salt or solvate form of AMG 397 as disclosed herein, optionally as a pharmaceutical formulation as disclosed herein. In some embodiments, the cancer is multiple myeloma, non-Hodgkin's lymphoma, or acute myeloid leukemia.

### Preparation of Salt and Solvate Forms

The crystalline forms disclosed herein can be prepared by a variety of methods known to those of skill in the art. For example, the crystalline forms can be prepared from amorphous, crude, or another crystalline form of AMG 397. In some embodiments, AMG 397 is combined with a solvent to form a desired crystalline form, for example as discussed in the examples below. In some embodiments, AMG 397 is dissolved in a solvent, or is combined with a solvent to form a slurry. In some embodiments, AMG 397 is combined with a solvent and the solution or slurry thus formed is aged to form the crystalline forms. In some embodiments, the solution or slurry is heated prior to aging or crystal formation.

### OTHER EMBODIMENTS

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims.

### EXAMPLES

The following examples are provided for illustration and are not intended to limit the scope of the invention.

### Materials and Methods

Commercially available reagents are used as is without further purification unless specified.

The synthesis of the starting material (AMG 397) for the following methods is disclosed in U.S. Pat. No. 10,300,075. The crystalline forms disclosed herein may be characterized using conventional means, including physical constants and spectral data.

### X-Ray Powder Diffraction:

XRPD patterns were collected with a PANalytical X'Pert PRO MPD diffractometer or a PANalytical Empyrean diffractometer using an incident beam of Cu radiation produced using an Optix long, fine-focus source. An elliptically graded multilayer mirror was used to focus Cu Ká X-ray radiation through the specimen and onto the detector. Prior to the analysis, a silicon specimen (NIST SRM 640e) was analyzed to verify the observed position of the Si (111) peak is consistent with the NIST-certified position. A specimen of the sample was sandwiched between 3-im-thick films and analyzed in transmission geometry. A beam-stop, short antiscatter extension, and antiscatter knife edge, were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b or software v. 5.5.

Alternatively, X-ray powder diffraction (XRPD) data were obtained on a PANalytical X′Pert PRO X-ray diffraction system with RTMS detector. Samples were scanned at ambient temperature in a continuous mode from 5 to 45° (2θ) with step size of 0.0334° at 45 kV and 40 mA with CuKα radiation (1.541874 Å).

XRPD indexing was conducted with proprietary SSCI software, TRIADS™ disclosed in U.S. Pat. No. 8,576,985.

### Differential Scanning Calorimetry:

Differential scanning calorimetry (DSC) was performed using a Mettler-Toledo DSC3+differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, and the weight was accurately recorded. The pan lid was pierced by the instrument and then inserted into the DSC cell for analysis. A weighed aluminum pan configured as the sample pan was placed on the reference side of the cell.

Differential scanning calorimetry (DSC) analysis was conducted on a TA Instruments Discovery Series calorimeter at 10° C./min from 25 to 250-350° C. in an aluminum pan under dry nitrogen at 50 ml/min.

Thermal Analysis: Thermal gravimetric analysis (TGA) and TGA/DSC Combo analyses were performed using a Mettler-Toledo TGA/DSC3+analyzer. Temperature and enthalpy adjustments were performed using indium, tin, and zinc, and then verified with indium. The balance was verified with calcium oxalate. The sample was placed in an open aluminum pan. The pan was hermetically sealed, the lid pierced, then inserted into the TG furnace. A weighed aluminum pan configured as the sample pan was placed on the reference platform. The furnace was heated under nitrogen.

Thermal gravimetric analysis (TGA) was performed on a TA Instruments Discovery Series analyzer at 10° C./min from ambient temperature to 250-350° C. in a platinum pan under dry nitrogen at 25 ml/min.

Moisture Sorption: Moisture sorption data was collected using a VTI SGA 100 symmetrical vapor sorption analyzer. A sample size of approximately 5-10 mg was used in a platinum pan. Hygroscopicity was evaluated from 5 to 95% RH in increments of 5% RH. Data for adsorption and desorption cycles were collected. Equilibrium criteria were set at 0.001% weight change in 10 minutes with a maximum equilibration time of 180 minutes.

NMR: Solution proton NMR spectra were acquired by Spectral Data Services of Champaign, IL at 25° C. with a Varian UNITYINOVA-400 spectrometer. Samples were dissolved in DMSO-d6. In some cases, the solution NMR spectra were acquired at SSCI with an Agilent DD2-400 spectrometer using deuterated DMSO or methanol.

¹³C SSNMR data was collected on a Bruker DSX spectrometer operating at 600 MHZ (¹H). A 4 mm H/F/X spinning probe operating at a spinning frequency of 14 kHz was used for all experiments. CPMAS with TOSS program was used with a recycle delay of 10 s. A ¹H 90° pulse of 2.5 μs and ¹³C 180° pulse of 8 us were used. Decoupling was carried out using a spinal64 sequence. 4096 transients were acquired for signal averaging. The data was processed with Topspin 3.0 software.

### Example 1: AMG 397 Trifluoroethanol Solvate

Crystalline AMG 397 trifluoroethanol solvate was prepared by charging AMG 397 with a solution of L-arginine (1:1) in trifluoroethanol to form a solution. Upon stirring for 3 days at room temperature a suspension was formed. The isolated solids were then collected to provide AMG 397 trifluoroethanol solvate, which was characterized as shown in the below tables.

TABLE 1
XRPD Data Table

| Pos. | FWHM | d-spacing | Height | Rel. Int. |
|---|---|---|---|---|
| [°2θ] | [°2θ] | [Å] | [cts] | [%] |
| 1.81 | 0.67 | 48.9 | 314.58 | 1.4 |
| 2.65 | 0.4 | 33.39 | 637.34 | 2.84 |
| 3.18 | 0.4 | 27.75 | 723.24 | 3.22 |
| 5.56 | 0.05 | 15.91 | 3638.02 | 16.2 |
| 6.38 | 0.05 | 13.84 | 2760.59 | 12.29 |
| 6.69 | 0.07 | 13.22 | 5946.33 | 26.48 |
| 8 | 0.05 | 11.05 | 1472.7 | 6.56 |
| 8.38 | 0.05 | 10.56 | 1633.92 | 7.28 |
| 8.82 | 0.07 | 10.02 | 2119.88 | 9.44 |
| 9.27 | 0.12 | 9.54 | 711.07 | 3.17 |
| 10.06 | 0.05 | 8.79 | 3559.76 | 15.85 |
| 10.34 | 0.07 | 8.56 | 6337.17 | 28.22 |
| 11.14 | 0.07 | 7.95 | 3266.44 | 14.54 |
| 11.84 | 0.05 | 7.47 | 3676.36 | 16.37 |
| 12.18 | 0.05 | 7.27 | 1726.27 | 7.69 |
| 12.5 | 0.08 | 7.08 | 6036.56 | 26.88 |
| 13.04 | 0.07 | 6.79 | 2123.52 | 9.46 |
| 13.49 | 0.07 | 6.56 | 4906.37 | 21.85 |
| 13.78 | 0.07 | 6.43 | 5555.84 | 24.74 |
| 13.96 | 0.05 | 6.34 | 1556.44 | 6.93 |
| 14.24 | 0.08 | 6.22 | 2258.29 | 10.06 |
| 14.6 | 0.07 | 6.07 | 9328.18 | 41.54 |
| 14.79 | 0.07 | 5.99 | 2189 | 9.75 |
| 15.5 | 0.07 | 5.72 | 2293.05 | 10.21 |
| 15.88 | 0.07 | 5.58 | 4476.22 | 19.93 |
| 16.05 | 0.07 | 5.52 | 3778.79 | 16.83 |
| 16.29 | 0.05 | 5.44 | 2507.55 | 11.17 |
| 16.54 | 0.07 | 5.36 | 3751.51 | 16.7 |
| 16.8 | 0.08 | 5.28 | 3475.07 | 15.47 |
| 17.18 | 0.08 | 5.16 | 11012.31 | 49.04 |
| 17.53 | 0.08 | 5.06 | 12812.83 | 57.05 |
| 17.71 | 0.07 | 5.01 | 5097.81 | 22.7 |
| 17.84 | 0.05 | 4.97 | 4501.22 | 20.04 |
| 18.11 | 0.07 | 4.9 | 5709.03 | 25.42 |
| 18.37 | 0.08 | 4.83 | 7302.77 | 32.52 |
| 18.54 | 0.07 | 4.79 | 7122.14 | 31.71 |
| 18.75 | 0.07 | 4.73 | 8047.45 | 35.83 |
| 19.22 | 0.08 | 4.62 | 22457.86 | 100 |
| 19.43 | 0.08 | 4.57 | 22391.94 | 99.71 |
| 20.02 | 0.07 | 4.43 | 7117.75 | 31.69 |
| 20.18 | 0.07 | 4.4 | 7715.11 | 34.35 |
| 20.4 | 0.08 | 4.35 | 12129.16 | 54.01 |
| 20.99 | 0.08 | 4.23 | 9918.15 | 44.16 |
| 21.23 | 0.1 | 4.19 | 6974.13 | 31.05 |
| 21.51 | 0.15 | 4.13 | 8155.16 | 36.31 |
| 21.7 | 0.08 | 4.1 | 13241.77 | 58.96 |
| 21.9 | 0.05 | 4.06 | 4561.2 | 20.31 |
| 22.27 | 0.07 | 3.99 | 5212.17 | 23.21 |
| 22.43 | 0.08 | 3.96 | 5154.26 | 22.95 |
| 22.85 | 0.07 | 3.89 | 4875.17 | 21.71 |
| 23.1 | 0.08 | 3.85 | 4058.95 | 18.07 |
| 23.29 | 0.07 | 3.82 | 3111.41 | 13.85 |
| 23.58 | 0.07 | 3.77 | 2845.98 | 12.67 |
| 23.82 | 0.08 | 3.73 | 3408.69 | 15.18 |
| 23.94 | 0.04 | 3.72 | 3081.49 | 13.72 |
| 24.14 | 0.12 | 3.68 | 2517.43 | 11.21 |
| 24.63 | 0.29 | 3.61 | 1948.46 | 8.68 |
| 24.93 | 0.1 | 3.57 | 1599.7 | 7.12 |
| 25.23 | 0.12 | 3.53 | 2640.07 | 11.76 |
| 25.42 | 0.06 | 3.5 | 2123.93 | 9.46 |
| 25.6 | 0.08 | 3.48 | 1596.42 | 7.11 |
| 25.92 | 0.14 | 3.43 | 2930.59 | 13.05 |
| 26.25 | 0.08 | 3.39 | 1406.17 | 6.26 |
| 26.59 | 0.06 | 3.35 | 1682.31 | 7.49 |
| 26.93 | 0.12 | 3.31 | 1658.25 | 7.38 |
| 27.29 | 0.08 | 3.27 | 1130.31 | 5.03 |
| 27.59 | 0.14 | 3.23 | 1927.48 | 8.58 |
| 27.75 | 0.14 | 3.21 | 1367.87 | 6.09 |
| 28.1 | 0.08 | 3.17 | 1168.22 | 5.2 |
| 28.47 | 0.1 | 3.13 | 1739.8 | 7.75 |
| 28.74 | 0.1 | 3.1 | 944.66 | 4.21 |
| 29.43 | 0.16 | 3.03 | 1038.23 | 4.62 |
| 29.82 | 0.1 | 2.99 | 1415.07 | 6.3 |
| 30.27 | 0.12 | 2.95 | 1121.27 | 4.99 |
| 30.47 | 0.12 | 2.93 | 1126.89 | 5.02 |
| 30.7 | 0.06 | 2.91 | 990.11 | 4.41 |
| 30.91 | 0.08 | 2.89 | 1029.75 | 4.59 |
| 31.39 | 0.16 | 2.85 | 1135.28 | 5.06 |
| 31.91 | 0.08 | 2.8 | 1262.15 | 5.62 |
| 32.99 | 0.12 | 2.71 | 1320.48 | 5.88 |
| 33.45 | 0.2 | 2.68 | 1002.82 | 4.47 |
| 33.73 | 0.06 | 2.66 | 1128.55 | 5.03 |
| 34.09 | 0.08 | 2.63 | 1154.97 | 5.14 |
| 34.49 | 0.08 | 2.6 | 1112.8 | 4.96 |
| 35.01 | 0.1 | 2.56 | 1014.28 | 4.52 |
| 35.5 | 0.16 | 2.53 | 1150.89 | 5.12 |
| 35.78 | 0.12 | 2.51 | 1225.25 | 5.46 |
| 35.96 | 0.08 | 2.5 | 1340.9 | 5.97 |
| 36.22 | 0.08 | 2.48 | 1214.92 | 5.41 |
| 36.61 | 0.2 | 2.45 | 1038.43 | 4.62 |
| 37.34 | 0.16 | 2.41 | 1181.71 | 5.26 |
| 38.2 | 0.24 | 2.35 | 1070.15 | 4.77 |
| 38.95 | 0.24 | 2.31 | 1163.63 | 5.18 |
| 39.51 | 0.12 | 2.28 | 1049.66 | 4.67 |

### ¹H NMR Data

¹H NMR (400 MHZ, DMSO-d6) δ ppm 0.78-1.04 (m, 2H) 1.16 (br d, J=12.31 Hz, 2H) 1.29-1.51 (m, 2H) 1.51-1.71 (m, 3H) 1.71-1.82 (m, 1H) 1.85 (br s, 1H) 2.00 (br d, J=12.95 Hz, 1H) 2.12-2.18 (m, 1H) 2.19-2.29 (m, 1H) 2.29-2.47 (m, 2H) 2.53-2.85 (m, 1H) 2.63-2.85 (m, 1H) 2.86-3.01 (m, 1H) 3.12-3.30 (m, 2H) 3.45-3.62 (m, 1H) 3.77-4.07 (m, 7H) 5.31-5.51 (m, 1H) 5.59 (br s, 1H) 6.05 (t, J=6.66 Hz, 3H) 6.72-6.90 (m, 1H) 6.90-7.11 (m, 2H) 7.11-7.21 (m, 1H) 7.27 (br dd, J=8.50, 2.32 Hz, 1H) 7.67 (d, J=8.52 Hz, 1H).

TABLE 2
X-ray Single Structure Data

| Crystal system | Primitive Orthorhombic | |
|---|---|---|
| Space group | P2₁2₁2₁ (19) | |
| Extinction Symbol | P2₁2₁2₁ | |
| Unit cell dimensions | a = 14.491 Å | α = 90° |
| | b = 15.330 Å | β = 90° |
| | c = 31.814 Å | γ = 90° |

| | |
|---|---|
| Volume | 7067.4 Å³ |
| Chiral Contents? | Chiral |

### Example 2: AMG 397 Hexafluoroisopropanol Solvate

Crystalline AMG 397 hexafluoroisopropanol (HFIPA) solvate was formed by charging AMG 397 and L-Arginine (1:1) with hexafluoroisopropanol and stirring the slurry at room temperature for 2 days. Alternatively, the crystalline solvate was prepared by charging AMG 397 and L-Lysine (1:1) with hexafluoroisopropanol and stirring at 55° C. The isolated solids were AMG 397 hexafluoroisopropanol solvate, which was characterized as shown in the below tables.

TABLE 3
XRPD Data Table

| Pos. | FWHM | d-spacing | Height | Rel. Int. |
|---|---|---|---|---|
| [°2θ] | [°2θ] | [Å] | [cts] | [%] |
| 1.6 | 0.67 | 55.28 | 224.11 | 1.06 |
| 2.52 | 0.8 | 35.03 | 573.82 | 2.72 |
| 3.48 | 0.33 | 25.42 | 687.28 | 3.26 |
| 5.35 | 0.05 | 16.51 | 1410.39 | 6.69 |
| 6.13 | 0.05 | 14.42 | 5114.11 | 24.26 |
| 6.47 | 0.05 | 13.66 | 2065.84 | 9.8 |
| 7.69 | 0.07 | 11.5 | 3115.25 | 14.78 |
| 7.97 | 0.05 | 11.09 | 1775.3 | 8.42 |
| 8.51 | 0.07 | 10.39 | 17649.5 | 83.72 |
| 9.75 | 0.1 | 9.07 | 1743.6 | 8.27 |
| 9.98 | 0.05 | 8.87 | 1682.22 | 7.98 |
| 11.05 | 0.05 | 8.01 | 1876.56 | 8.9 |
| 11.37 | 0.08 | 7.78 | 21046.83 | 99.84 |
| 12.1 | 0.05 | 7.32 | 1151.72 | 5.46 |
| 12.28 | 0.07 | 7.21 | 2138.87 | 10.15 |
| 12.53 | 0.07 | 7.07 | 4183.31 | 19.84 |
| 12.82 | 0.05 | 6.91 | 12148.13 | 57.63 |
| 13.32 | 0.07 | 6.65 | 1681.72 | 7.98 |
| 13.45 | 0.05 | 6.58 | 2215.18 | 10.51 |
| 13.64 | 0.07 | 6.49 | 5029.8 | 23.86 |
| 14.1 | 0.05 | 6.28 | 1766.01 | 8.38 |
| 14.3 | 0.07 | 6.19 | 3279.81 | 15.56 |
| 14.68 | 0.05 | 6.04 | 2248.57 | 10.67 |
| 14.9 | 0.07 | 5.94 | 2689.63 | 12.76 |
| 15.34 | 0.07 | 5.78 | 5855.38 | 27.78 |
| 15.69 | 0.07 | 5.65 | 7285.96 | 34.56 |
| 15.98 | 0.05 | 5.55 | 3201.45 | 15.19 |
| 16.2 | 0.08 | 5.47 | 9816.99 | 46.57 |
| 16.42 | 0.05 | 5.4 | 9612.89 | 45.6 |
| 16.54 | 0.03 | 5.36 | 5089.65 | 24.14 |
| 16.82 | 0.07 | 5.27 | 16460.41 | 78.08 |
| 17.07 | 0.07 | 5.2 | 11252.58 | 53.38 |
| 17.43 | 0.07 | 5.09 | 7484.55 | 35.5 |
| 17.63 | 0.07 | 5.03 | 12314.9 | 58.42 |
| 17.83 | 0.05 | 4.97 | 6284.8 | 29.81 |
| 17.95 | 0.03 | 4.94 | 6817.32 | 32.34 |
| 18.09 | 0.08 | 4.9 | 7437.3 | 35.28 |
| 18.45 | 0.03 | 4.81 | 8185.79 | 38.83 |
| 18.6 | 0.07 | 4.77 | 21080.57 | 100 |
| 18.79 | 0.08 | 4.72 | 19326.55 | 91.68 |
| 19.39 | 0.1 | 4.58 | 6230.31 | 29.55 |
| 19.8 | 0.05 | 4.48 | 2755.84 | 13.07 |
| 20.04 | 0.07 | 4.43 | 2710.59 | 12.86 |
| 20.29 | 0.07 | 4.38 | 2980.78 | 14.14 |
| 20.6 | 0.07 | 4.31 | 7879.42 | 37.38 |
| 20.8 | 0.05 | 4.27 | 2143.73 | 10.17 |
| 21.12 | 0.1 | 4.21 | 11933.59 | 56.61 |
| 21.52 | 0.07 | 4.13 | 8545.84 | 40.54 |
| 21.65 | 0.05 | 4.1 | 4803.9 | 22.79 |
| 21.91 | 0.07 | 4.06 | 1425.12 | 6.76 |
| 22.21 | 0.05 | 4 | 4608.02 | 21.86 |
| 22.36 | 0.07 | 3.98 | 11154.36 | 52.91 |
| 22.84 | 0.05 | 3.89 | 2974.33 | 14.11 |
| 23.14 | 0.1 | 3.84 | 10600.73 | 50.29 |
| 23.61 | 0.07 | 3.77 | 3181.6 | 15.09 |
| 23.89 | 0.07 | 3.72 | 1447.14 | 6.86 |
| 24.39 | 0.07 | 3.65 | 2902.05 | 13.77 |
| 24.54 | 0.05 | 3.63 | 1923.96 | 9.13 |
| 24.71 | 0.05 | 3.6 | 1459.12 | 6.92 |
| 24.99 | 0.07 | 3.56 | 3092.14 | 14.67 |
| 25.2 | 0.05 | 3.53 | 2124.78 | 10.08 |
| 25.35 | 0.07 | 3.51 | 5747.92 | 27.27 |
| 25.79 | 0.07 | 3.45 | 898.38 | 4.26 |
| 26.12 | 0.05 | 3.41 | 752.84 | 3.57 |
| 26.49 | 0.05 | 3.36 | 1405.9 | 6.67 |
| 26.71 | 0.05 | 3.34 | 2345.29 | 11.13 |
| 27.09 | 0.08 | 3.29 | 976.35 | 4.63 |
| 27.37 | 0.07 | 3.26 | 1106.11 | 5.25 |
| 27.95 | 0.07 | 3.19 | 1000.01 | 4.74 |
| 28.2 | 0.05 | 3.16 | 856.25 | 4.06 |
| 28.37 | 0.08 | 3.15 | 2202.53 | 10.45 |
| 28.69 | 0.07 | 3.11 | 3131.49 | 14.85 |
| 29.01 | 0.1 | 3.08 | 4212.54 | 19.98 |
| 29.09 | 0.04 | 3.07 | 2819.28 | 13.37 |
| 29.38 | 0.1 | 3.04 | 954.88 | 4.53 |
| 29.71 | 0.06 | 3 | 2202.18 | 10.45 |
| 29.91 | 0.06 | 2.98 | 1549.28 | 7.35 |
| 30.17 | 0.06 | 2.96 | 1336.45 | 6.34 |
| 30.3 | 0.1 | 2.95 | 2009.15 | 9.53 |
| 30.46 | 0.04 | 2.93 | 1626.76 | 7.72 |
| 30.82 | 0.04 | 2.9 | 1079.5 | 5.12 |
| 30.91 | 0.04 | 2.89 | 1348.17 | 6.4 |
| 31.02 | 0.06 | 2.88 | 1802.27 | 8.55 |
| 31.7 | 0.06 | 2.82 | 1023.83 | 4.86 |
| 31.99 | 0.06 | 2.8 | 1301.32 | 6.17 |
| 32.27 | 0.08 | 2.77 | 982.26 | 4.66 |
| 32.97 | 0.1 | 2.71 | 1181.01 | 5.6 |
| 33.31 | 0.08 | 2.69 | 997.16 | 4.73 |
| 33.66 | 0.04 | 2.66 | 906.47 | 4.3 |
| 33.76 | 0.06 | 2.65 | 1037.91 | 4.92 |
| 34.21 | 0.06 | 2.62 | 1909.62 | 9.06 |
| 34.31 | 0.04 | 2.62 | 1557.86 | 7.39 |
| 34.54 | 0.06 | 2.59 | 1239.65 | 5.88 |
| 34.73 | 0.06 | 2.58 | 963.68 | 4.57 |
| 35.1 | 0.06 | 2.55 | 1128.37 | 5.35 |
| 35.38 | 0.06 | 2.53 | 867.06 | 4.11 |
| 35.94 | 0.06 | 2.5 | 1417.28 | 6.72 |
| 36.05 | 0.04 | 2.5 | 1096.75 | 5.2 |
| 36.51 | 0.08 | 2.46 | 826.15 | 3.92 |
| 36.72 | 0.06 | 2.45 | 1201.55 | 5.7 |
| 37.12 | 0.06 | 2.42 | 909.55 | 4.31 |
| 37.37 | 0.06 | 2.4 | 1518.92 | 7.21 |
| 37.58 | 0.06 | 2.39 | 1248.47 | 5.92 |
| 37.86 | 0.12 | 2.37 | 912.41 | 4.33 |
| 38.13 | 0.08 | 2.36 | 1024.46 | 4.86 |
| 38.3 | 0.06 | 2.35 | 1136.37 | 5.39 |
| 38.61 | 0.08 | 2.33 | 864.24 | 4.1 |
| 39.23 | 0.06 | 2.29 | 821.14 | 3.9 |
| 39.41 | 0.06 | 2.28 | 850.62 | 4.04 |
| 39.81 | 0.08 | 2.26 | 905.49 | 4.3 |

### ¹H NMR Data

¹H NMR (400 MHZ, DMSO-d6) δ ppm 0.86-0.97 (m, 3H) 1.10-1.19 (m, 3H) 1.22-1.45 (m, 4H) 1.47-1.71 (m, 6H) 1.75-1.91 (m, 3H) 1.94-2.11 (m, 3H) 2.13-2.44 (m, 6H) 2.53-2.85 (m, 1H) 2.53-2.85 (m, 4H) 2.87-3.00 (m, 1H) 2.94 (br dd, J=14.85, 10.36 Hz, 1H) 3.01-3.16 (m, 1H) 3.02-3.20 (m, 1H) 3.25-3.48 (m, 3H) 3.27-3.45 (m, 1H) 3.49-3.63 (m, 2H) 3.62-3.74 (m, 1H) 3.63-3.77 (m, 1H) 3.80-3.92 (m, 1H) 3.91-4.07 (m, 2H) 3.92-4.05 (m, 2H) 5.05-5.24 (m, 1H) 5.05-5.24 (m, 1H) 5.15 (dt, J=13.04, 6.30 Hz, 5H) 5.42-5.53 (m, 1H) 5.42-5.53 (m, 1H) 5.44-5.54 (m, 1H) 5.55-5.69 (m, 1H) 5.56-5.70 (m, 1H) 5.57-5.69 (m, 1H) 6.75-6.84 (m, 1H) 6.77-6.86 (m, 1H) 6.77-6.86 (m, 1H) 6.96-7.06 (m, 1H) 6.97-7.05 (m, 1H) 6.98-7.05 (m, 1H) 7.12-7.23 (m, 2H) 7.13-7.22 (m, 2H) 7.13-7.22 (m, 2H) 7.13-7.22 (m, 2H) 7.14-7.22 (m, 2H) 7.22-7.31 (m, 1H) 7.23-7.30 (m, 1H) 7.24-7.30 (m, 1H) 7.24-7.31 (m, 1H) 7.69 (s, 1H) 7.61-7.73 (m, 1H) 7.64-7.74 (m, 1H) 7.96-8.21 (m, 1H) 7.98-8.16 (m, 1H) 8.07 (br s, 5H).

TABLE 4
X-ray Single Structure Data

| Crystal system | Primitive Orthorhombic | |
|---|---|---|
| Space group | P2₁2₁2₁ (19) | |
| Extinction Symbol | P2₁2₁2₁ | |
| Unit cell dimensions | a = 14.983 Å | α = 90° |
| | b = 16.011 Å | β = 90° |
| | c = 32.950 Å | γ = 90° |

| | |
|---|---|
| Volume | 7904.4 Å³ |
| Chiral Contents? | Chiral |

### Example 3: AMG 397 1-Propanol Solvate

Crystalline AMG 397 1-propanol (1-PrOH) solvate was formed by charging AMG 397 with 1-propanol and stirring the slurry at 55° C. for 2 days. The isolated solids were AMG 397 1-propanol solvate, which was characterized as shown in the below tables.

TABLE 5
XRPD Data Table

| Pos. | FWHM | d-spacing | Height | Rel. Int. |
|---|---|---|---|---|
| [°2θ] | [°2θ] | [Å] | [cts] | [%] |
| 2.25 | 0.07 | 39.23 | 164.6 | 1.94 |
| 3.14 | 0.27 | 28.1 | 130.62 | 1.54 |
| 6.61 | 0.05 | 13.37 | 970.48 | 11.42 |
| 8.1 | 0.07 | 10.91 | 5379.63 | 63.31 |
| 9.71 | 0.07 | 9.11 | 5555.76 | 65.38 |
| 10.47 | 0.08 | 8.45 | 883.2 | 10.39 |
| 11.24 | 0.08 | 7.87 | 421.38 | 4.96 |
| 12 | 0.07 | 7.38 | 2897.8 | 34.1 |
| 12.67 | 0.07 | 6.99 | 1959.58 | 23.06 |
| 13.26 | 0.07 | 6.68 | 6502.96 | 76.53 |
| 14.23 | 0.07 | 6.22 | 2352.38 | 27.68 |
| 14.81 | 0.05 | 5.98 | 2064.01 | 24.29 |
| 15.06 | 0.07 | 5.88 | 7728.98 | 90.96 |
| 15.71 | 0.07 | 5.64 | 4343.65 | 51.12 |
| 16.09 | 0.07 | 5.51 | 1588.24 | 18.69 |
| 16.25 | 0.03 | 5.46 | 1498.12 | 17.63 |
| 16.4 | 0.07 | 5.4 | 4657.77 | 54.81 |
| 17.09 | 0.05 | 5.19 | 1919.48 | 22.59 |
| 17.23 | 0.08 | 5.15 | 4524.92 | 53.25 |
| 17.74 | 0.08 | 5 | 5466.63 | 64.33 |
| 18.17 | 0.07 | 4.88 | 3419.72 | 40.24 |
| 18.53 | 0.08 | 4.79 | 8497.41 | 100 |
| 19.12 | 0.07 | 4.64 | 3667.37 | 43.16 |
| 19.5 | 0.07 | 4.55 | 3502.43 | 41.22 |
| 20.66 | 0.08 | 4.3 | 2773.65 | 32.64 |
| 21.21 | 0.07 | 4.19 | 2412.99 | 28.4 |
| 21.57 | 0.07 | 4.12 | 3669.18 | 43.18 |
| 21.68 | 0.05 | 4.1 | 3014.31 | 35.47 |
| 22.08 | 0.07 | 4.03 | 2221.04 | 26.14 |
| 22.3 | 0.05 | 3.99 | 3297.98 | 38.81 |
| 22.44 | 0.08 | 3.96 | 4157.16 | 48.92 |
| 22.81 | 0.1 | 3.9 | 2012.89 | 23.69 |
| 23.53 | 0.08 | 3.78 | 1434.08 | 16.88 |
| 23.81 | 0.08 | 3.73 | 2353.26 | 27.69 |
| 23.89 | 0.04 | 3.73 | 2164.38 | 25.47 |
| 24.57 | 0.12 | 3.62 | 771.15 | 9.08 |
| 24.92 | 0.1 | 3.57 | 1307.54 | 15.39 |
| 25.28 | 0.08 | 3.52 | 1197.34 | 14.09 |
| 25.49 | 0.16 | 3.49 | 2007.7 | 23.63 |
| 26.09 | 0.12 | 3.41 | 416.88 | 4.91 |
| 26.69 | 0.06 | 3.34 | 867.59 | 10.21 |
| 26.89 | 0.08 | 3.31 | 1578.64 | 18.58 |
| 27.52 | 0.06 | 3.24 | 1053.8 | 12.4 |
| 27.67 | 0.08 | 3.22 | 1134.74 | 13.35 |
| 27.94 | 0.14 | 3.19 | 951.63 | 11.2 |
| 28.37 | 0.12 | 3.14 | 233.36 | 2.75 |
| 28.69 | 0.08 | 3.11 | 336.53 | 3.96 |
| 28.95 | 0.08 | 3.08 | 192.21 | 2.26 |
| 29.17 | 0.16 | 3.06 | 252.11 | 2.97 |
| 29.49 | 0.16 | 3.03 | 198.32 | 2.33 |
| 30.38 | 0.08 | 2.94 | 490.61 | 5.77 |
| 31.42 | 0.1 | 2.85 | 240.64 | 2.83 |
| 31.61 | 0.12 | 2.83 | 227.19 | 2.67 |
| 32.14 | 0.12 | 2.78 | 84.09 | 0.99 |
| 32.65 | 0.06 | 2.74 | 174.17 | 2.05 |
| 33.08 | 0.08 | 2.71 | 184.37 | 2.17 |
| 33.63 | 0.2 | 2.66 | 100.65 | 1.18 |
| 33.89 | 0.1 | 2.64 | 109.36 | 1.29 |
| 34.09 | 0.16 | 2.63 | 97.19 | 1.14 |
| 34.43 | 0.16 | 2.6 | 106.59 | 1.25 |
| 34.95 | 0.08 | 2.56 | 48.2 | 0.57 |
| 35.24 | 0.12 | 2.54 | 69.06 | 0.81 |
| 35.84 | 0.08 | 2.5 | 385.79 | 4.54 |
| 36.34 | 0.08 | 2.47 | 162.45 | 1.91 |
| 37.05 | 0.08 | 2.42 | 398.67 | 4.69 |
| 37.97 | 0.16 | 2.37 | 133.29 | 1.57 |
| 38.44 | 0.08 | 2.34 | 93.33 | 1.1 |
| 39.14 | 0.08 | 2.3 | 23.6 | 0.28 |

### ¹H NMR Data

¹H NMR (400 MHZ, DMSO-d6) δ ppm 0.79-0.89 (m, 1H) 0.92 (br d, J=6.41 Hz, 2H) 0.97-1.12 (m, 1H) 1.19 (br s, 2H) 1.30-1.50 (m, 3H) 1.57-1.71 (m, 2H) 1.71-1.92 (m, 3H) 2.01 (br d, J=13.46 Hz, 2H) 2.13-2.30 (m, 2H) 2.30-2.49 (m, 2H) 2.53-2.65 (m, 2H) 2.66-2.83 (m, 2H) 2.87-3.02 (m, 2H) 3.02-3.15 (m, 1H) 3.16-3.30 (m, 3H) 3.34-3.42 (m, 3H) 3.52-3.76 (m, 2H) 3.99 (br d, J=9.40 Hz, 3H) 4.35 (t, J=5.24 Hz, 1H) 5.35-5.51 (m, 1H) 5.54-5.70 (m, 1H) 6.74-6.88 (m, 1H) 7.00 (dd, J=8.12, 1.71 Hz, 1H) 7.18 (d, J=2.35 Hz, 1H) 7.28 (dd, J=8.55, 2.35 Hz, 1H) 7.68 (d, J=8.55 Hz, 1H).

TABLE 6
X-ray Single Structure Data

| | |
|---|---|
| Crystal system | Primitive Orthorhombic |
| Space group | P2₁2₁2₁ (19) |
| Extinction Symbol | P2₁2₁2₁ |

| | | |
|---|---|---|
| Unit cell dimensions | a = 12.440 Å | α = 90° |
| | b = 16.887 Å | β = 90° |
| | c = 21.771 Å | γ = 90° |

| | |
|---|---|
| Volume | 4573.6 Å³ |
| Chiral Contents? | Chiral |

### Example 4: AMG 397 Isopropanol Solvate Form 1

Crystalline AMG 397 Isopropanol (IPA) solvate form 1 was formed by charging AMG 397 and Ca(OAc)₂, Mg(OAc)₂ or NaOAc (1:1) with IPA and stirring the slurry for 3-6 days at room temperature. The isolated solids were AMG 397 isopropanol solvate form 1, which was characterized as shown in the below tables.

TABLE 7
XRPD Data Table

| Pos. [°2θ] | FWHM [°2θ] | d-spacing [Å] | Height [cts] | Rel. Int. [%] |
|---|---|---|---|---|
| 1.31 | 0.33 | 67.29 | 60.7 | 0.25 |
| 6.14 | 0.07 | 14.4 | 18135.23 | 75.4 |
| 7.14 | 0.07 | 12.38 | 24053.56 | 100 |
| 8.22 | 0.05 | 10.75 | 4411.81 | 18.34 |
| 9.79 | 0.03 | 9.03 | 2601.23 | 10.81 |
| 10 | 0.07 | 8.85 | 21961.42 | 91.3 |
| 10.46 | 0.07 | 8.46 | 5855.56 | 24.34 |
| 11.26 | 0.07 | 7.86 | 2185.64 | 9.09 |
| 11.74 | 0.05 | 7.54 | 3571.3 | 14.85 |
| 12.92 | 0.07 | 6.85 | 697.88 | 2.9 |
| 13.28 | 0.07 | 6.67 | 3962.94 | 16.48 |
| 13.62 | 0.07 | 6.5 | 6692.19 | 27.82 |
| 13.91 | 0.07 | 6.37 | 4082.8 | 16.97 |
| 14.5 | 0.08 | 6.11 | 9005.31 | 37.44 |
| 15.02 | 0.08 | 5.9 | 5204.99 | 21.64 |
| 15.34 | 0.07 | 5.78 | 8880.35 | 36.92 |
| 15.6 | 0.07 | 5.68 | 4250.81 | 17.67 |
| 15.89 | 0.08 | 5.58 | 14360.69 | 59.7 |
| 16.24 | 0.08 | 5.46 | 7990.2 | 33.22 |
| 16.61 | 0.07 | 5.34 | 12360.83 | 51.39 |
| 16.73 | 0.03 | 5.3 | 6450.92 | 26.82 |
| 16.9 | 0.07 | 5.25 | 8977.42 | 37.32 |
| 17.22 | 0.1 | 5.15 | 1133.49 | 4.71 |
| 17.65 | 0.08 | 5.02 | 11291.96 | 46.95 |
| 17.89 | 0.07 | 4.96 | 12369.64 | 51.43 |
| 18.39 | 0.05 | 4.82 | 13693.64 | 56.93 |
| 18.51 | 0.07 | 4.79 | 15134.01 | 62.92 |
| 19.03 | 0.08 | 4.66 | 16187.19 | 67.3 |
| 19.46 | 0.07 | 4.56 | 7864.59 | 32.7 |
| 19.67 | 0.12 | 4.51 | 14701.83 | 61.12 |
| 20.06 | 0.05 | 4.43 | 2523.58 | 10.49 |
| 20.4 | 0.08 | 4.35 | 18575.65 | 77.23 |
| 20.73 | 0.08 | 4.29 | 10611.38 | 44.12 |
| 21.01 | 0.07 | 4.23 | 2988.13 | 12.42 |
| 21.12 | 0.07 | 4.21 | 3462.16 | 14.39 |
| 21.58 | 0.08 | 4.12 | 8853.16 | 36.81 |
| 21.78 | 0.1 | 4.08 | 3057.81 | 12.71 |
| 22.13 | 0.08 | 4.02 | 3325.53 | 13.83 |
| 22.59 | 0.07 | 3.94 | 2835.6 | 11.79 |
| 22.73 | 0.03 | 3.91 | 1963.13 | 8.16 |
| 22.89 | 0.08 | 3.89 | 3695.94 | 15.37 |
| 23.07 | 0.08 | 3.86 | 5762.95 | 23.96 |
| 23.24 | 0.07 | 3.83 | 2118.18 | 8.81 |
| 23.61 | 0.07 | 3.77 | 1724.35 | 7.17 |
| 23.89 | 0.1 | 3.72 | 1920.47 | 7.98 |
| 24.14 | 0.08 | 3.69 | 2319.17 | 9.64 |
| 24.33 | 0.07 | 3.66 | 994.58 | 4.13 |
| 24.88 | 0.08 | 3.58 | 3075.16 | 12.78 |
| 24.96 | 0.06 | 3.57 | 2504.25 | 10.41 |
| 25.58 | 0.04 | 3.48 | 2134.04 | 8.87 |
| 25.7 | 0.12 | 3.46 | 5070.05 | 21.08 |
| 26.01 | 0.08 | 3.42 | 655.32 | 2.72 |
| 26.19 | 0.1 | 3.4 | 970.88 | 4.04 |
| 26.52 | 0.06 | 3.36 | 1154.32 | 4.8 |
| 26.76 | 0.06 | 3.33 | 802.11 | 3.33 |
| 26.99 | 0.12 | 3.3 | 871.19 | 3.62 |
| 27.21 | 0.08 | 3.27 | 627.62 | 2.61 |
| 27.42 | 0.12 | 3.25 | 547.13 | 2.27 |
| 27.65 | 0.08 | 3.22 | 1256.16 | 5.22 |
| 27.81 | 0.12 | 3.2 | 1011.6 | 4.21 |
| 28.1 | 0.12 | 3.17 | 2058.15 | 8.56 |
| 28.57 | 0.04 | 3.12 | 780.04 | 3.24 |
| 28.71 | 0.08 | 3.11 | 2115.93 | 8.8 |
| 28.87 | 0.08 | 3.09 | 2096.94 | 8.72 |
| 29.31 | 0.08 | 3.04 | 1464.27 | 6.09 |
| 29.71 | 0.12 | 3 | 573.42 | 2.38 |
| 29.9 | 0.1 | 2.99 | 1065.13 | 4.43 |
| 30.43 | 0.16 | 2.94 | 1748.37 | 7.27 |
| 30.78 | 0.06 | 2.9 | 869.12 | 3.61 |
| 30.95 | 0.08 | 2.89 | 2623.89 | 10.91 |
| 31.15 | 0.1 | 2.87 | 554.24 | 2.3 |
| 31.35 | 0.08 | 2.85 | 649.84 | 2.7 |
| 31.5 | 0.06 | 2.84 | 728.67 | 3.03 |
| 31.67 | 0.2 | 2.82 | 2229.63 | 9.27 |
| 32.29 | 0.06 | 2.77 | 192.37 | 0.8 |
| 32.65 | 0.12 | 2.74 | 948.86 | 3.94 |
| 32.95 | 0.06 | 2.72 | 845.38 | 3.51 |
| 33.05 | 0.04 | 2.71 | 873.49 | 3.63 |
| 33.35 | 0.06 | 2.68 | 281.64 | 1.17 |
| 33.73 | 0.08 | 2.66 | 50.7 | 0.21 |
| 34 | 0.06 | 2.63 | 218.06 | 0.91 |
| 34.17 | 0.12 | 2.62 | 738.97 | 3.07 |
| 34.44 | 0.06 | 2.6 | 131.79 | 0.55 |
| 34.7 | 0.08 | 2.58 | 277.87 | 1.16 |
| 34.92 | 0.06 | 2.57 | 504.56 | 2.1 |
| 35.17 | 0.08 | 2.55 | 131.59 | 0.55 |
| 35.58 | 0.08 | 2.52 | 976.16 | 4.06 |
| 35.93 | 0.1 | 2.5 | 115.27 | 0.48 |
| 36.23 | 0.12 | 2.48 | 195.87 | 0.81 |
| 36.4 | 0.08 | 2.47 | 380.83 | 1.58 |
| 36.84 | 0.08 | 2.44 | 490.05 | 2.04 |
| 37.14 | 0.08 | 2.42 | 427.92 | 1.78 |
| 37.27 | 0.04 | 2.42 | 187.85 | 0.78 |
| 37.5 | 0.06 | 2.4 | 288.8 | 1.2 |
| 37.79 | 0.12 | 2.38 | 372.72 | 1.55 |
| 38.43 | 0.08 | 2.34 | 119.46 | 0.5 |
| 38.61 | 0.08 | 2.33 | 458.86 | 1.91 |
| 38.86 | 0.06 | 2.32 | 406.91 | 1.69 |
| 39 | 0.08 | 2.31 | 627.55 | 2.61 |
| 39.53 | 0.14 | 2.28 | 374.12 | 1.56 |

### ¹H NMR Data

¹H NMR (400 MHZ, DMSO-d6) δ ppm 0.81-0.98 (m, 3H) 1.04 (d, J=6.18 Hz, 3H) 1.12-1.20 (m, 2H) 1.21-1.28 (m, 2H) 1.28-1.47 (m, 3H) 1.47-1.72 (m, 4H) 1.73-1.82 (m, 1H) 1.82-1.88 (m, 1H) 1.88-1.95 (m, 1H) 1.95-2.05 (m, 2H) 2.12-2.29 (m, 3H) 2.29-2.38 (m, 1H) 2.39-2.48 (m, 1H) 2.52-2.65 (m, 2H) 2.65-2.79 (m, 2H) 2.86-3.01 (m, 2H) 3.01-3.17 (m, 2H) 3.17-3.25 (m, 3H) 3.38-3.64 (m, 3H) 3.65-3.90 (m, 5H) 3.91-4.06 (m, 3H) 4.34 (d, J=4.26 Hz, 2H) 5.44 (br d, J=15.77 Hz, 2H) 5.61 (br d, J=16.62 Hz, 2H) 6.80 (br d, J=7.67 Hz, 2H) 6.90-7.09 (m, 2H) 7.09-7.21 (m, 2H) 7.27 (dd, J=8.52, 2.34 Hz, 1H) 7.67 (d, J=8.52 Hz, 1H).

TABLE 8
X-ray Single Structure Data

| | |
|---|---|
| Crystal system | Primitive Orthorhombic |
| Space group | P2₁2₁2₁ (19) |
| Extinction Symbol | P2₁2₁2₁ |
| Chiral Contents? | Chiral |

| | | |
|---|---|---|
| Unit cell dimensions | a = 11.575 Å | α = 90° |
| | b = 13.699 Å | β = 90° |
| | c = 28.757 Å | γ = 90° |

| | |
|---|---|
| Volume | 4559.7 Å³ |

### Example 5: AMG 397 Isopropanol Solvate Form 2

Crystalline AMG 397 Isopropanol (IPA) solvate form 2 was formed by charging AMG 397 and Mg(OAc)₂ (2:1) with IPA and stirring the slurry at 77° C. for 2 days. The isolated solids were AMG 397 isopropanol solvate form 2, which was characterized as shown in the below tables.

TABLE 9
XRPD Data Table

| Pos. [°2θ] | FWHM [°2θ] | d-spacing [Å] | Height [cts] | Rel. Int. [%] |
|---|---|---|---|---|
| 1.9 | 0.27 | 46.38 | 287.53 | 0.83 |
| 6.61 | 0.05 | 13.38 | 3113.13 | 8.99 |
| 7.15 | 0.05 | 12.37 | 270.38 | 0.78 |
| 8.12 | 0.07 | 10.89 | 24057.93 | 69.45 |
| 8.82 | 0.07 | 10.03 | 702.22 | 2.03 |
| 9.7 | 0.08 | 9.11 | 20832.21 | 60.14 |
| 10 | 0.08 | 8.84 | 294.61 | 0.85 |
| 10.45 | 0.07 | 8.47 | 1953.91 | 5.64 |
| 11.21 | 0.05 | 7.89 | 418.59 | 1.21 |
| 12 | 0.08 | 7.38 | 11647.03 | 33.62 |
| 12.64 | 0.08 | 7 | 8842.25 | 25.53 |
| 13.26 | 0.08 | 6.68 | 30992.96 | 89.47 |
| 14.23 | 0.08 | 6.23 | 7514.26 | 21.69 |
| 14.8 | 0.07 | 5.99 | 9216.93 | 26.61 |
| 15.05 | 0.07 | 5.89 | 34640.25 | 100 |
| 15.7 | 0.08 | 5.64 | 16956.05 | 48.95 |
| 16.09 | 0.07 | 5.51 | 6487.2 | 18.73 |
| 16.27 | 0.03 | 5.45 | 6065.75 | 17.51 |
| 16.41 | 0.07 | 5.4 | 19537.77 | 56.4 |
| 17.11 | 0.03 | 5.18 | 7343.41 | 21.2 |
| 17.23 | 0.08 | 5.15 | 13203.8 | 38.12 |
| 17.71 | 0.1 | 5.01 | 21536.55 | 62.17 |
| 18.15 | 0.08 | 4.89 | 10236.61 | 29.55 |
| 18.55 | 0.08 | 4.78 | 33536.85 | 96.81 |
| 18.79 | 0.07 | 4.72 | 2837.54 | 8.19 |
| 19.1 | 0.08 | 4.65 | 9321.2 | 26.91 |
| 19.5 | 0.08 | 4.55 | 7503.6 | 21.66 |
| 19.94 | 0.07 | 4.45 | 2036.71 | 5.88 |
| 20.67 | 0.08 | 4.3 | 3387.65 | 9.78 |
| 20.99 | 0.07 | 4.23 | 2104.02 | 6.07 |
| 21.19 | 0.08 | 4.19 | 2653.92 | 7.66 |
| 21.53 | 0.06 | 4.12 | 7206.26 | 20.8 |
| 21.64 | 0.1 | 4.11 | 9559.99 | 27.6 |
| 22.05 | 0.07 | 4.03 | 3999.79 | 11.55 |
| 22.31 | 0.1 | 3.99 | 11112.8 | 32.08 |
| 22.43 | 0.07 | 3.96 | 9976.34 | 28.8 |
| 22.78 | 0.08 | 3.9 | 2937.21 | 8.48 |
| 23.54 | 0.1 | 3.78 | 3573.14 | 10.31 |
| 23.81 | 0.12 | 3.74 | 9379.97 | 27.08 |
| 24.12 | 0.1 | 3.69 | 2909.26 | 8.4 |
| 24.57 | 0.08 | 3.62 | 1295.29 | 3.74 |
| 24.95 | 0.08 | 3.57 | 3167.54 | 9.14 |
| 25.27 | 0.06 | 3.52 | 2908.15 | 8.4 |
| 25.39 | 0.05 | 3.51 | 3819.84 | 11.03 |
| 25.57 | 0.12 | 3.48 | 5321.84 | 15.36 |
| 25.79 | 0.03 | 3.45 | 648.73 | 1.87 |
| 26.11 | 0.07 | 3.41 | 719.87 | 2.08 |
| 26.68 | 0.07 | 3.34 | 2578.63 | 7.44 |
| 26.89 | 0.12 | 3.32 | 6237.73 | 18.01 |
| 27.61 | 0.15 | 3.23 | 5262.6 | 15.19 |
| 27.91 | 0.12 | 3.2 | 2911.67 | 8.41 |
| 28.29 | 0.07 | 3.15 | 367.07 | 1.06 |
| 28.53 | 0.07 | 3.13 | 533.89 | 1.54 |
| 28.97 | 0.08 | 3.08 | 1010.49 | 2.92 |
| 29.26 | 0.1 | 3.05 | 712.75 | 2.06 |
| 29.45 | 0.07 | 3.03 | 1375.83 | 3.97 |
| 29.62 | 0.1 | 3.02 | 676.95 | 1.95 |
| 30.35 | 0.15 | 2.95 | 2521.98 | 7.28 |
| 30.62 | 0.07 | 2.92 | 360.73 | 1.04 |
| 31.4 | 0.05 | 2.85 | 963.49 | 2.78 |
| 31.61 | 0.08 | 2.83 | 1167.69 | 3.37 |
| 32.17 | 0.05 | 2.78 | 302.96 | 0.87 |
| 32.53 | 0.07 | 2.75 | 437.73 | 1.26 |
| 32.85 | 0.08 | 2.73 | 1019.78 | 2.94 |
| 33.1 | 0.08 | 2.71 | 546.18 | 1.58 |
| 33.58 | 0.07 | 2.67 | 693.39 | 2 |
| 33.91 | 0.05 | 2.64 | 378.66 | 1.09 |
| 34.13 | 0.07 | 2.63 | 428.96 | 1.24 |
| 34.31 | 0.1 | 2.61 | 538.26 | 1.55 |
| 34.45 | 0.06 | 2.61 | 507.95 | 1.47 |
| 34.86 | 0.08 | 2.57 | 138.73 | 0.4 |
| 35.19 | 0.08 | 2.55 | 405.96 | 1.17 |
| 35.86 | 0.1 | 2.5 | 1221.53 | 3.53 |
| 36.42 | 0.08 | 2.46 | 1012.23 | 2.92 |
| 36.57 | 0.08 | 2.46 | 1466.66 | 4.23 |
| 36.78 | 0.06 | 2.44 | 1453.84 | 4.2 |
| 36.89 | 0.06 | 2.43 | 1199.7 | 3.46 |
| 37.06 | 0.1 | 2.42 | 1734.48 | 5.01 |
| 37.43 | 0.12 | 2.4 | 167.09 | 0.48 |
| 37.66 | 0.08 | 2.39 | 188.66 | 0.54 |
| 37.99 | 0.12 | 2.37 | 1123.02 | 3.24 |
| 38.47 | 0.06 | 2.34 | 1030.75 | 2.98 |
| 38.58 | 0.06 | 2.33 | 838.88 | 2.42 |
| 39.34 | 0.12 | 2.29 | 350.8 | 1.01 |
| 39.78 | 0.14 | 2.26 | 629.82 | 1.82 |

### ¹H NMR Data

¹H NMR (400 MHZ, DMSO-d6) δ ppm 0.83-0.95 (m, 1H) 1.00-1.08 (m, 8H) 1.10-1.26 (m, 2H) 1.26-1.53 (m, 2H) 1.54-1.71 (m, 2H) 1.71-1.88 (m, 2H) 1.88-1.93 (m, 1H) 2.00 (br d, J=12.79 Hz, 1H) 2.10-2.28 (m, 2H) 2.28-2.36 (m, 1H) 2.37-2.46 (m, 1H) 2.52-2.86 (m, 3H) 2.87-2.99 (m, 1H) 2.99-3.12 (m, 1H) 3.16-3.28 (m, 2H) 3.49-3.61 (m, 1H) 3.69-3.82 (m, 2H) 3.82-3.91 (m, 1H) 3.98 (br d, J=10.23 Hz, 1H) 4.33 (d, J=4.05 Hz, 7H) 5.36-5.51 (m, 1H) 5.53-5.72 (m, 1H) 6.71-6.88 (m, 1H) 6.93-7.21 (m, 4H) 7.26 (br d, J=8.52 Hz, 3H) 7.67 (br d, J=8.74 Hz, 1H).

TABLE 10
X-ray Single Structure Data

| | |
|---|---|
| Crystal system | Primitive Orthorhombic |
| Space group | P2₁2₁2₁ (19) |
| Extinction Symbol | P2₁2₁2₁ |
| Chiral Contents? | Chiral |

| | | |
|---|---|---|
| Unit cell dimensions | a = 12.443 Å | α = 90° |
| | b = 16.920 Å | β = 90° |
| | c = 21.746 Å | γ = 90° |

| | |
|---|---|
| Volume | 4578.3 Å³ |

### Example 6: AMG 397 Acetonitrile Solvate

Crystalline AMG 397 Acetonitrile (MeCN) solvate was formed by charging AMG 397 with MeCN to form a suspension, then charging with a solution of L-lactic acid (1:1) in MeCN and stirring the suspension at room temperature for 6 days. The isolated solids were AMG 397 acetonitrile solvate, which was characterized as shown in the below tables.

TABLE 11
XRPD Data Table

| Pos. [°2θ] | FWHM [°2θ] | d-spacing [Å] | Height [cts] | Rel. Int. [%] |
|---|---|---|---|---|
| 6.03 | 0.07 | 14.65 | 11050.23 | 21.98 |
| 6.48 | 0.12 | 13.63 | 60.75 | 0.12 |
| 7.85 | 0.07 | 11.27 | 2249.72 | 4.47 |
| 8.69 | 0.07 | 10.18 | 3672.97 | 7.31 |
| 10.19 | 0.08 | 8.68 | 50273.99 | 100 |
| 10.85 | 0.08 | 8.16 | 5735.85 | 11.41 |
| 12.76 | 0.05 | 6.94 | 1697.48 | 3.38 |
| 12.98 | 0.1 | 6.82 | 13247.18 | 26.35 |
| 13.73 | 0.08 | 6.45 | 3097.69 | 6.16 |
| 14.33 | 0.08 | 6.18 | 11422.69 | 22.72 |
| 15.19 | 0.1 | 5.83 | 12199.46 | 24.27 |
| 15.45 | 0.07 | 5.73 | 2803.12 | 5.58 |
| 15.64 | 0.08 | 5.67 | 7812.1 | 15.54 |
| 15.95 | 0.08 | 5.56 | 3260.16 | 6.48 |
| 16.77 | 0.08 | 5.29 | 4029.97 | 8.02 |
| 17.04 | 0.1 | 5.2 | 19213.78 | 38.22 |
| 17.23 | 0.08 | 5.15 | 9141.12 | 18.18 |
| 17.77 | 0.08 | 4.99 | 3118.16 | 6.2 |
| 18.19 | 0.1 | 4.88 | 7467.56 | 14.85 |
| 18.62 | 0.1 | 4.77 | 10052.72 | 20 |
| 19.16 | 0.05 | 4.63 | 5076.94 | 10.1 |
| 19.33 | 0.1 | 4.59 | 13756.28 | 27.36 |
| 20 | 0.1 | 4.44 | 3507.89 | 6.98 |
| 20.45 | 0.1 | 4.34 | 29888.73 | 59.45 |
| 21.07 | 0.12 | 4.22 | 8447.51 | 16.8 |
| 21.43 | 0.1 | 4.15 | 8854.54 | 17.61 |
| 21.81 | 0.08 | 4.08 | 3533.23 | 7.03 |
| 22.05 | 0.12 | 4.03 | 7453.83 | 14.83 |
| 22.26 | 0.07 | 3.99 | 6127.45 | 12.19 |
| 22.45 | 0.12 | 3.96 | 9550.21 | 19 |
| 23.03 | 0.13 | 3.86 | 10407.73 | 20.7 |
| 23.39 | 0.1 | 3.8 | 5444.14 | 10.83 |
| 23.77 | 0.12 | 3.74 | 4261.18 | 8.48 |
| 24.19 | 0.1 | 3.68 | 3355.22 | 6.67 |
| 24.59 | 0.05 | 3.62 | 3695.54 | 7.35 |
| 24.83 | 0.1 | 3.59 | 6938.03 | 13.8 |
| 25.2 | 0.12 | 3.53 | 5349.01 | 10.64 |
| 25.62 | 0.12 | 3.48 | 5358.73 | 10.66 |
| 26.13 | 0.1 | 3.41 | 5772.44 | 11.48 |
| 26.3 | 0.08 | 3.39 | 4655.81 | 9.26 |
| 26.51 | 0.07 | 3.36 | 5591.28 | 11.12 |
| 26.7 | 0.08 | 3.34 | 7003.91 | 13.93 |
| 26.83 | 0.12 | 3.32 | 6435.49 | 12.8 |
| 27.12 | 0.07 | 3.29 | 3221.11 | 6.41 |
| 27.34 | 0.12 | 3.26 | 4072.42 | 8.1 |
| 27.68 | 0.12 | 3.22 | 3762.16 | 7.48 |
| 28.18 | 0.08 | 3.17 | 2444.99 | 4.86 |
| 28.46 | 0.12 | 3.14 | 3307.33 | 6.58 |
| 28.9 | 0.15 | 3.09 | 3174.76 | 6.31 |
| 29.23 | 0.13 | 3.06 | 2756.21 | 5.48 |
| 29.66 | 0.07 | 3.01 | 2417.79 | 4.81 |
| 30.02 | 0.12 | 2.98 | 2881.84 | 5.73 |
| 30.44 | 0.06 | 2.93 | 2887.7 | 5.74 |
| 30.51 | 0.07 | 2.93 | 3257.17 | 6.48 |
| 31.02 | 0.12 | 2.88 | 4584.61 | 9.12 |
| 31.41 | 0.12 | 2.85 | 2324.89 | 4.62 |
| 31.95 | 0.1 | 2.8 | 1834.35 | 3.65 |
| 32.51 | 0.07 | 2.75 | 1381.36 | 2.75 |
| 32.72 | 0.27 | 2.74 | 1109.21 | 2.21 |
| 33.07 | 0.07 | 2.71 | 1352.61 | 2.69 |
| 33.44 | 0.08 | 2.68 | 1702.24 | 3.39 |
| 33.67 | 0.08 | 2.66 | 2457.71 | 4.89 |
| 33.76 | 0.05 | 2.66 | 2505.63 | 4.98 |
| 34.09 | 0.05 | 2.63 | 1202.61 | 2.39 |
| 34.47 | 0.05 | 2.6 | 1054.18 | 2.1 |
| 34.8 | 0.13 | 2.58 | 1423.94 | 2.83 |
| 35.11 | 0.12 | 2.56 | 1489.86 | 2.96 |
| 35.35 | 0.08 | 2.54 | 1214.58 | 2.42 |
| 35.67 | 0.12 | 2.52 | 2298.17 | 4.57 |
| 36.05 | 0.1 | 2.49 | 668.93 | 1.33 |
| 36.35 | 0.1 | 2.47 | 719.99 | 1.43 |
| 36.6 | 0.1 | 2.46 | 685.42 | 1.36 |
| 36.84 | 0.1 | 2.44 | 647.53 | 1.29 |
| 37.14 | 0.12 | 2.42 | 671.63 | 1.34 |
| 37.58 | 0.08 | 2.39 | 982.37 | 1.95 |
| 37.93 | 0.08 | 2.37 | 899.28 | 1.79 |
| 38.22 | 0.13 | 2.36 | 662.31 | 1.32 |
| 38.64 | 0.13 | 2.33 | 464.24 | 0.92 |
| 39.01 | 0.08 | 2.31 | 525.22 | 1.04 |
| 39.29 | 0.13 | 2.29 | 728.43 | 1.45 |
| 39.57 | 0.17 | 2.28 | 872.06 | 1.73 |

### ¹H NMR Data

¹H NMR (400 MHZ, DMSO-d6) δ ppm 0.46 (s, 1H) 0.75 (br d, J=6.55 Hz, 1H) 0.91 (br d, J=6.65 Hz, 2H) 1.17 (br d, J=6.11 Hz, 2H) 1.27-1.46 (m, 2H) 1.47-1.72 (m, 3H) 1.74-1.90 (m, 2H) 1.98-2.11 (m, 6H) 2.18-2.40 (m, 3H) 2.42-2.48 (m, 1H) 2.52-2.74 (m, 2H) 2.76-2.83 (m, 1H) 2.93 (br dd, J=14.77, 10.47 Hz, 2H) 3.01-3.13 (m, 1H) 3.13-3.20 (m, 1H) 3.25-3.44 (m, 4H) 3.55 (br d, J=14.23 Hz, 1H) 3.71 (br s, 1H) 3.86 (br d, J=14.53 Hz, 1H) 3.91-4.07 (m, 1H) 4.14 (s, 1H) 5.44 (br d, J=16.04 Hz, 1H) 5.52-5.73 (m, 1H) 6.80 (br d, J=8.02 Hz, 1H) 7.00 (dd, J=8.07, 1.66 Hz, 1H) 7.17 (d, J=2.35 Hz, 1H) 7.27 (dd, J=8.49, 2.27 Hz, 1H) 7.67 (d, J=8.51 Hz, 1H) 8.62 (s, 1H).

TABLE 12
X-ray Single Structure Data

| | |
|---|---|
| Crystal system | Primitive Monoclinic |
| Space group | P2₁ (4) |
| Extinction Symbol | P 1 2₁ 1 |
| Chiral Contents? | Chiral |

| | | |
|---|---|---|
| Unit cell dimensions | a = 11.582 Å | α = 90° |
| | b = 13.640 Å | β = 103.81° |
| | c = 15.054 Å | γ = 90° |

| | |
|---|---|
| Volume | 2309.4 Å³ |

### Example 7: AMG 397 Acetic Acid Solvate

Crystalline AMG 397 Acetic Acid solvate was formed as follows. AMG 397 is combined with ethanol and aqueous sodium hydroxide. Aqueous acetic acid is added and the resulting slurry aged. The product is collected by filtration. The isolated solids were AMG 397 acetic acid solvate, which was characterized as shown in the below tables.

TABLE 13
XRPD Data Table

| Pos. [°2θ] | FWHM [°2θ] | d-spacing [Å] | Height [cts] | Rel. Int. [%] |
|---|---|---|---|---|
| 6.36 | 0.2 | 13.9 | 266.82 | 0.88 |
| 8.4 | 0.17 | 10.53 | 3214.39 | 10.64 |
| 9.69 | 0.13 | 9.13 | 655.34 | 2.17 |
| 10.66 | 0.15 | 8.3 | 7642.42 | 25.3 |
| 10.92 | 0.08 | 8.1 | 10337.93 | 34.23 |
| 11.14 | 0.15 | 7.95 | 12643.42 | 41.86 |
| 11.45 | 0.15 | 7.73 | 6684.57 | 22.13 |
| 12.37 | 0.13 | 7.16 | 4115.1 | 13.62 |
| 12.66 | 0.15 | 6.99 | 4070.95 | 13.48 |
| 13.13 | 0.15 | 6.75 | 962.66 | 3.19 |
| 13.72 | 0.18 | 6.45 | 7577.21 | 25.09 |
| 14.25 | 0.2 | 6.22 | 9427.68 | 31.21 |
| 14.65 | 0.12 | 6.05 | 537.18 | 1.78 |
| 15.59 | 0.18 | 5.68 | 4359.86 | 14.43 |
| 16.47 | 0.17 | 5.38 | 3847.03 | 12.74 |
| 17.11 | 0.2 | 5.18 | 24032.15 | 79.57 |
| 17.63 | 0.17 | 5.03 | 5909.43 | 19.57 |
| 18.16 | 0.27 | 4.89 | 30204.06 | 100 |
| 18.81 | 0.13 | 4.72 | 7778.47 | 25.75 |
| 19.08 | 0.15 | 4.65 | 12743.14 | 42.19 |
| 19.33 | 0.12 | 4.59 | 4846.44 | 16.05 |
| 20.07 | 0.17 | 4.42 | 9042.29 | 29.94 |
| 20.56 | 0.15 | 4.32 | 1214.12 | 4.02 |
| 21.04 | 0.2 | 4.22 | 2200.06 | 7.28 |
| 21.29 | 0.12 | 4.17 | 1276.75 | 4.23 |
| 21.93 | 0.07 | 4.05 | 2777.13 | 9.19 |
| 22.21 | 0.17 | 4 | 4507.63 | 14.92 |
| 22.67 | 0.17 | 3.92 | 2588.57 | 8.57 |
| 23.64 | 0.17 | 3.76 | 3405.83 | 11.28 |
| 24.01 | 0.17 | 3.71 | 3154.9 | 10.45 |
| 24.57 | 0.08 | 3.62 | 5590.03 | 18.51 |
| 24.79 | 0.18 | 3.59 | 7133.26 | 23.62 |
| 25.59 | 0.17 | 3.48 | 674.16 | 2.23 |
| 26.25 | 0.13 | 3.4 | 1162.68 | 3.85 |
| 26.82 | 0.2 | 3.32 | 870.9 | 2.88 |
| 27.31 | 0.1 | 3.27 | 668.15 | 2.21 |
| 27.66 | 0.15 | 3.23 | 980.67 | 3.25 |
| 28.19 | 0.13 | 3.17 | 296.25 | 0.98 |
| 28.7 | 0.2 | 3.11 | 2098.02 | 6.95 |
| 29.02 | 0.12 | 3.08 | 3251.02 | 10.76 |
| 29.19 | 0.13 | 3.06 | 2937.52 | 9.73 |
| 29.74 | 0.3 | 3 | 1494.04 | 4.95 |
| 30.22 | 0.2 | 2.96 | 665.04 | 2.2 |
| 30.72 | 0.17 | 2.91 | 723.41 | 2.4 |
| 31.16 | 0.18 | 2.87 | 809.8 | 2.68 |
| 31.51 | 0.2 | 2.84 | 517.49 | 1.71 |
| 32.74 | 0.13 | 2.74 | 310.07 | 1.03 |
| 33.3 | 0.23 | 2.69 | 1494.24 | 4.95 |
| 34.42 | 0.3 | 2.61 | 946.63 | 3.13 |
| 35.36 | 0.15 | 2.54 | 1549.81 | 5.13 |
| 35.98 | 0.17 | 2.5 | 364.42 | 1.21 |
| 36.67 | 0.17 | 2.45 | 269.09 | 0.89 |
| 37.4 | 0.2 | 2.4 | 161.32 | 0.53 |
| 38.42 | 0.23 | 2.34 | 792.12 | 2.62 |
| 39.12 | 0.12 | 2.3 | 546.56 | 1.81 |
| 39.72 | 0.17 | 2.27 | 267.27 | 0.88 |

TABLE 14
Solid State ¹³C NMR Data

| Peak | v(F1) [ppm] | Intensity [rel] |
|---|---|---|
| 1 | 13.63 | 0.63 |
| 2 | 19.22 | 0.67 |
| 3 | 20.4 | 0.36 |
| 4 | 24.22 | 0.38 |
| 5 | 25.69 | 0.47 |
| 6 | 26.57 | 0.37 |
| 7 | 27.75 | 0.36 |
| 8 | 29.81 | 0.48 |
| 9 | 30.4 | 0.45 |
| 10 | 31.28 | 0.4 |
| 11 | 36.57 | 0.58 |
| 12 | 38.34 | 0.43 |
| 13 | 40.1 | 0.33 |
| 14 | 43.04 | 0.86 |
| 15 | 49.51 | 0.68 |
| 16 | 50.1 | 0.44 |
| 17 | 51.86 | 0.56 |
| 18 | 54.51 | 0.4 |
| 19 | 56.28 | 0.44 |
| 20 | 57.16 | 0.54 |
| 21 | 57.75 | 0.49 |
| 22 | 60.1 | 0.74 |
| 23 | 62.16 | 0.33 |
| 24 | 65.39 | 0.63 |
| 25 | 77.75 | 0.38 |
| 26 | 85.1 | 1 |
| 27 | 115.39 | 0.41 |
| 28 | 123.63 | 0.52 |
| 29 | 125.1 | 0.4 |
| 30 | 128.04 | 0.55 |
| 31 | 131.27 | 0.61 |
| 32 | 133.04 | 0.78 |
| 33 | 133.92 | 0.72 |
| 34 | 135.98 | 0.36 |
| 35 | 139.8 | 0.6 |
| 36 | 141.27 | 0.57 |
| 37 | 143.04 | 0.7 |
| 38 | 151.86 | 0.59 |
| 39 | 173.92 | 0.55 |

### Example 8: AMG 397 Hydrochloride Salt Form 1

Crystalline AMG 397 hydrochloride salt form 1 was formed by 1:1 (mol/mol, API/acid) salt reaction with HCl in EtOH stirred at room temperature for 2 h followed by 75° C. for 1h. Alternatively, it was prepared by 1:1 (mol/mol, API/acid) salt reaction with HCl in dioxane stirred at room temperature for 6 days. The isolated solids were AMG 397 hydrochloride salt form 1, which was characterized as shown in the below tables.

TABLE 15
XRPD Data Table

| Pos. [°2θ] | FWHM [°2θ] | d-spacing [Å] | Height [cts] | Rel. Int. [%] |
|---|---|---|---|---|
| 6.75 | 0.06 | 13.1 | 459.42 | 5.59 |
| 7.3 | 0.06 | 12.12 | 765.59 | 9.32 |
| 8.74 | 0.09 | 10.11 | 1211.9 | 14.76 |
| 10.74 | 0.09 | 8.24 | 1824.21 | 22.21 |
| 11.68 | 0.11 | 7.58 | 4708.85 | 57.34 |
| 12 | 0.11 | 7.37 | 4222.42 | 51.42 |
| 12.94 | 0.11 | 6.84 | 7183.16 | 87.47 |
| 13.5 | 0.11 | 6.56 | 3305.7 | 40.26 |
| 14.36 | 0.11 | 6.17 | 4103.65 | 49.97 |
| 14.6 | 0.11 | 6.07 | 2038.64 | 24.83 |
| 15.49 | 0.13 | 5.72 | 3916.23 | 47.69 |
| 15.88 | 0.13 | 5.58 | 5293.12 | 64.46 |
| 16.17 | 0.15 | 5.48 | 8211.73 | 100 |
| 16.73 | 0.15 | 5.3 | 1520.88 | 18.52 |
| 17.47 | 0.11 | 5.08 | 562.99 | 6.86 |
| 17.88 | 0.17 | 4.96 | 7899.36 | 96.2 |
| 18.11 | 0.11 | 4.9 | 3178.36 | 38.71 |
| 18.68 | 0.17 | 4.75 | 729.41 | 8.88 |
| 19.44 | 0.15 | 4.57 | 918.38 | 11.18 |
| 19.78 | 0.19 | 4.49 | 4363.5 | 53.14 |
| 20.51 | 0.19 | 4.33 | 4369.75 | 53.21 |
| 21.11 | 0.09 | 4.21 | 378.9 | 4.61 |
| 21.56 | 0.13 | 4.12 | 679.86 | 8.28 |
| 21.98 | 0.22 | 4.04 | 1083.02 | 13.19 |
| 22.76 | 0.22 | 3.91 | 3028.49 | 36.88 |
| 23.67 | 0.21 | 3.76 | 2878.9 | 35.06 |
| 24.17 | 0.13 | 3.68 | 1119.53 | 13.63 |
| 24.59 | 0.26 | 3.62 | 2376.39 | 28.94 |
| 25.13 | 0.24 | 3.54 | 1803.66 | 21.96 |
| 25.79 | 0.22 | 3.45 | 854.65 | 10.41 |
| 26.45 | 0.09 | 3.37 | 2107.63 | 25.67 |
| 26.95 | 0.07 | 3.31 | 1585.65 | 19.31 |
| 27.24 | 0.22 | 3.27 | 1083.88 | 13.2 |
| 27.77 | 0.09 | 3.21 | 1099.95 | 13.39 |
| 28.28 | 0.13 | 3.16 | 856.31 | 10.43 |
| 28.81 | 0.3 | 3.1 | 797.38 | 9.71 |
| 29.63 | 0.15 | 3.02 | 1037.72 | 12.64 |
| 29.82 | 0.11 | 3 | 958.3 | 11.67 |
| 30.2 | 0.15 | 2.96 | 294.98 | 3.59 |
| 30.73 | 0.26 | 2.91 | 536.06 | 6.53 |
| 31.36 | 0.11 | 2.85 | 958.52 | 11.67 |
| 31.8 | 0.09 | 2.81 | 773.58 | 9.42 |
| 32.38 | 0.26 | 2.77 | 506.61 | 6.17 |
| 33.23 | 0.22 | 2.7 | 696.42 | 8.48 |
| 33.78 | 0.15 | 2.65 | 461.89 | 5.62 |
| 34.25 | 0.19 | 2.62 | 389.46 | 4.74 |
| 35.49 | 0.13 | 2.53 | 588.04 | 7.16 |
| 36.18 | 0.22 | 2.48 | 552.76 | 6.73 |
| 37.11 | 0.15 | 2.42 | 533.14 | 6.49 |
| 37.47 | 0.15 | 2.4 | 480.35 | 5.85 |
| 38.02 | 0.26 | 2.37 | 391.29 | 4.77 |
| 38.56 | 0.22 | 2.34 | 477.15 | 5.81 |
| 39.34 | 0.22 | 2.29 | 489.24 | 5.96 |
| 39.77 | 0.19 | 2.27 | 372.3 | 4.53 |

### ¹H NMR Data

¹H NMR (400 MHZ, DMSO-d6) δ ppm 0.96 (br d, J=6.18 Hz, 5H) 1.27-1.52 (m, 7H) 1.53-1.72 (m, 3H) 1.79 (br d, J=10.28 Hz, 4H) 1.82-1.91 (m, 2H) 1.92-2.15 (m, 4H) 2.15-2.27 (m, 1H) 2.27-2.37 (m, 2H) 2.37-2.47 (m, 2H) 2.52-2.63 (m, 2H) 2.64-2.87 (m, 3H) 2.88-3.10 (m, 3H) 3.10-3.30 (m, 6H) 3.57 (s, 10H) 3.79-4.07 (m, 9H) 5.34 (br d, J=16.09 Hz, 2H) 5.58 (br d, J=15.50 Hz, 3H) 6.66-6.95 (m, 4H) 7.01 (br dd, J=8.12, 1.57 Hz, 3H) 7.07-7.15 (m, 2H) 7.18 (d, J=2.18 Hz, 2H) 7.27 (dd, J=8.52, 2.18 Hz, 2H) 7.66 (d, J=8.58 Hz, 1H) 9.67 (br s, 3H) 11.87 (s, 2H).

TABLE 16
X-ray Single Structure Data

| Crystal system | Primitive Hexagonal | |
|---|---|---|
| Space group(s) | P6₁ (169), P6₁22 (178), P6₅ (170), P6₅22 (179) | |
| Extinction Symbol | P 6₁ - - | |
| Chiral Contents? | Chiral | |
| Unit cell dimensions | a = 15.189 Å | α = 90° |
| | b = 15.189 Å | β = 90° |
| | c = 31.844 Å | γ = 120° |

| | |
|---|---|
| Volume | 6362.0 Å³ |

### Example 9: AMG 397 Amorphous Sodium Salt Form 1

AMG 397 amorphous sodium salt form 1 was formed by extracting AMG 397 from 100 mg drug product tablet using Me-THF and solvent exchange to ethanol, then adding NaOH to form the sodium salt. The wet-cake was vacuum-dried under N₂ flow to yield the sodium salt.

### Example 10: AMG 397 Potassium Salt Form 1

Crystalline AMG 397 potassium salt form 1 was formed by 1:1 (mol/mol, API/base) salt reaction with KOMe in DMF/H₂O 1:1 stirred at 55° C. for 8 h. The isolated solids were AMG 397 potassium salt form 1, which was characterized as shown in the below tables.

TABLE 17
XRPD Data Table

| Pos. [°2θ] | FWHM [°2θ] | d-spacing [Å] | Height [cts] | Rel. Int. [%] |
|---|---|---|---|---|
| 11 | 0.15 | 8.05 | 34.87 | 33.4 |
| 11.35 | 0.15 | 7.8 | 33.11 | 31.71 |
| 12.81 | 0.15 | 6.91 | 104.42 | 100 |
| 13.42 | 0.3 | 6.6 | 96.93 | 92.83 |
| 14.46 | 0.3 | 6.12 | 16.83 | 16.12 |
| 15.69 | 0.39 | 5.65 | 15.35 | 14.7 |
| 17.18 | 0.3 | 5.16 | 46.34 | 44.38 |
| 19.23 | 0.59 | 4.61 | 15.19 | 14.54 |

### Example 11: AMG 397 Potassium Salt Form 2 (Ethyl Acetate Solvate)

Crystalline AMG 397 potassium salt form 2 (ethyl acetate solvate) was formed by 1:1 (mol/mol, API/base) salt reaction with KOH in EtOAc stirred at room temperature for 2 days. The isolated solids were AMG 397 potassium salt form 2 (ethyl acetate solvate), which was characterized as shown in the below tables.

TABLE 18
XRPD Data Table

| Pos. [°2θ] | FWHM [°2θ] | d-spacing [Å] | Height [cts] | Rel. Int. [%] |
|---|---|---|---|---|
| 2.73 | 0.07 | 32.37 | 13092.85 | 100 |
| 5.48 | 0.07 | 16.14 | 871.87 | 6.66 |
| 5.81 | 0.08 | 15.2 | 1377.88 | 10.52 |
| 6.76 | 0.07 | 13.08 | 1100.56 | 8.41 |
| 7.78 | 0.05 | 11.36 | 1378.31 | 10.53 |
| 8.47 | 0.07 | 10.44 | 1895.54 | 14.48 |
| 8.81 | 0.08 | 10.04 | 934.35 | 7.14 |
| 10.69 | 0.07 | 8.28 | 1894.4 | 14.47 |
| 10.97 | 0.07 | 8.06 | 2446.66 | 18.69 |
| 11.19 | 0.07 | 7.91 | 3187 | 24.34 |
| 11.37 | 0.05 | 7.78 | 1447.53 | 11.06 |
| 11.66 | 0.08 | 7.59 | 7869.27 | 60.1 |
| 12.2 | 0.08 | 7.25 | 6764.2 | 51.66 |
| 13.11 | 0.1 | 6.76 | 1103.15 | 8.43 |
| 13.65 | 0.33 | 6.49 | 1128.5 | 8.62 |
| 14.24 | 0.13 | 6.22 | 1506.36 | 11.51 |
| 15.07 | 0.07 | 5.88 | 3722.69 | 28.43 |
| 15.26 | 0.07 | 5.81 | 4800.53 | 36.67 |
| 15.44 | 0.08 | 5.74 | 3845.35 | 29.37 |
| 16.05 | 0.08 | 5.52 | 4383 | 33.48 |
| 16.28 | 0.05 | 5.44 | 3487.57 | 26.64 |
| 16.41 | 0.08 | 5.4 | 4138.5 | 31.61 |
| 16.61 | 0.07 | 5.34 | 4076.75 | 31.14 |
| 16.79 | 0.05 | 5.28 | 3710.31 | 28.34 |
| 16.94 | 0.07 | 5.23 | 4338.95 | 33.14 |
| 17.3 | 0.08 | 5.13 | 4384.67 | 33.49 |
| 17.53 | 0.1 | 5.06 | 3924.81 | 29.98 |
| 17.86 | 0.07 | 4.97 | 3686.99 | 28.16 |
| 18.48 | 0.13 | 4.8 | 4347.98 | 33.21 |
| 18.9 | 0.13 | 4.69 | 3652.49 | 27.9 |
| 19.17 | 0.13 | 4.63 | 3915.25 | 29.9 |
| 19.5 | 0.1 | 4.55 | 4143.13 | 31.64 |
| 19.74 | 0.08 | 4.5 | 4154.68 | 31.73 |
| 20.45 | 0.1 | 4.34 | 5638.63 | 43.07 |
| 20.86 | 0.1 | 4.26 | 5350.42 | 40.87 |
| 21.13 | 0.08 | 4.21 | 5248.56 | 40.09 |
| 21.58 | 0.05 | 4.12 | 5377.67 | 41.07 |
| 21.73 | 0.08 | 4.09 | 4644 | 35.47 |
| 22.22 | 0.08 | 4 | 4175.57 | 31.89 |
| 22.48 | 0.1 | 3.96 | 3683.91 | 28.14 |
| 22.69 | 0.07 | 3.92 | 3962.68 | 30.27 |
| 22.86 | 0.12 | 3.89 | 5271.89 | 40.27 |
| 23.26 | 0.1 | 3.82 | 3116.1 | 23.8 |
| 23.54 | 0.08 | 3.78 | 3292.75 | 25.15 |
| 23.9 | 0.17 | 3.72 | 3278.89 | 25.04 |
| 24.44 | 0.05 | 3.64 | 3323.95 | 25.39 |
| 24.81 | 0.1 | 3.59 | 2106.36 | 16.09 |
| 25.55 | 0.1 | 3.49 | 2137.1 | 16.32 |
| 25.84 | 0.1 | 3.45 | 1864.52 | 14.24 |
| 27.15 | 0.13 | 3.29 | 1667.33 | 12.73 |
| 27.32 | 0.13 | 3.26 | 1630.91 | 12.46 |
| 28.03 | 0.33 | 3.18 | 933.7 | 7.13 |
| 28.75 | 0.33 | 3.11 | 785.78 | 6 |
| 29.4 | 0.13 | 3.04 | 961.8 | 7.35 |
| 30.79 | 0.13 | 2.9 | 634.31 | 4.84 |
| 32.98 | 0.13 | 2.72 | 359.67 | 2.75 |
| 36.12 | 0.27 | 2.49 | 135.41 | 1.03 |

### ¹H NMR Data

¹H NMR (400 MHZ, DMSO-d6) δ ppm 0.79-0.96 (m, 3H) 0.96-1.13 (m, 3H) 1.13-1.25 (m, 2H) 1.29-1.50 (m, 3H) 1.50-1.59 (m, 2H) 1.64 (br d, J=14.70 Hz, 1H) 1.71-1.81 (m, 1H) 1.82-2.04 (m, 5H) 2.06-2.26 (m, 3H) 2.27-2.47 (m, 3H) 2.53-2.64 (m, 1H) 2.65-2.84 (m, 2H) 2.85-2.98 (m, 1H) 3.03 (br d, J=7.03 Hz, 1H) 3.13-3.28 (m, 3H) 3.47-3.56 (m, 1H) 3.80-4.06 (m, 3H) 5.14-5.59 (m, 3H) 5.67 (dt, J=16.84, 4.79 Hz, 2H) 6.71 (d, J=8.10 Hz, 1H) 6.98 (dd, J=8.10, 1.49 Hz, 1H) 7.08-7.19 (m, 1H) 7.19-7.34 (m, 2H) 7.69 (d, J=8.52 Hz, 1H).

TABLE 19
X-ray Single Structure Data

| | |
|---|---|
| Crystal system | Primitive Monoclinic |
| Space group(s) | P2₁ (4) |
| Extinction Symbol | P 1 2₁ 1 |
| Chiral Contents? | Chiral |

| | | |
|---|---|---|
| Unit cell dimensions | a = 17.075 Å | α = 90° |
| | b = 7.138 Å | β = 107.62° |
| | c = 22.322 Å | γ = 120° |

| | |
|---|---|
| Volume | 2593.1 Å³ |

### Example 12: AMG 397 Sulfate Salt Form 1

Crystalline AMG 397 Sulfate salt form 1 was formed by 1:1 (mol/mol, API/base) salt reaction with H₂SO₄ In EtOH/H₂O 9:1 stirred at 55° C. for 8 h. The isolated solids were AMG 397 sulfate salt form 1, which was characterized as shown in the below tables.

TABLE 20
XRPD Data Table

| Pos. [°2θ] | FWHM [°2θ] | d-spacing [Å] | Height [cts] | Rel. Int. [%] |
|---|---|---|---|---|
| 8.72 | 0.3 | 10.14 | 28.74 | 60.56 |
| 9.34 | 0.3 | 9.47 | 37.32 | 78.65 |
| 11.45 | 0.3 | 7.73 | 34.17 | 72.02 |
| 13.87 | 0.3 | 6.38 | 34.35 | 72.39 |
| 17.61 | 0.39 | 5.04 | 31.1 | 65.54 |
| 19.16 | 0.3 | 4.63 | 47.45 | 100 |
| 21.9 | 0.34 | 4.06 | 20.44 | 43.07 |

### Example 13: AMG 397 Sulfate Salt Form 2

Crystalline AMG 397 Sulfate salt form 2 was formed by 1:1 (mol/mol, API/base) salt reaction with H₂SO₄ in DMF/H₂O 1:1 stirred at 55° C. for 8 h. The isolated solids were AMG 397 sulfate salt form 2, which was characterized as shown in the below tables.

TABLE 21
XRPD Data Table

| Pos. [°2θ] | FWHM [°2θ] | d-spacing [Å] | Height [cts] | Rel. Int. [%] |
|---|---|---|---|---|
| 11.68 | 0.15 | 7.57 | 140.94 | 100 |
| 12.75 | 0.15 | 6.94 | 71.85 | 50.98 |
| 15.93 | 0.2 | 5.56 | 48.48 | 34.4 |
| 17.07 | 0.15 | 5.19 | 90.12 | 63.94 |
| 20.06 | 0.15 | 4.43 | 87.59 | 62.14 |
| 24.13 | 0.59 | 3.69 | 14.59 | 10.35 |

### Example 14: AMG 397 Sulfate Salt Form 3

Crystalline AMG 397 Sulfate salt form 3 was formed by 1:1 (mol/mol, API/acid) salt reaction with H₂SO₄ In EtOH stirred at 55° C. for 8 h. The isolated solids were AMG 397 sulfate salt form 3, which was characterized as shown in the below tables.

TABLE 22
XRPD Data Table

| Pos. [°2θ] | FWHM [°2θ] | d-spacing [Å] | Height [cts] | Rel. Int. [%] |
|---|---|---|---|---|
| 11.19 | 0.2 | 7.9 | 80.75 | 44.57 |
| 12.32 | 0.2 | 7.18 | 131.14 | 72.37 |
| 13.01 | 0.15 | 6.81 | 34.72 | 19.16 |
| 14.01 | 0.2 | 6.32 | 110.24 | 60.84 |
| 15.26 | 0.15 | 5.81 | 45.22 | 24.95 |
| 15.78 | 0.2 | 5.62 | 51.41 | 28.37 |
| 16.67 | 0.2 | 5.32 | 72.11 | 39.8 |
| 17.65 | 0.15 | 5.02 | 151.6 | 83.67 |
| 18.44 | 0.2 | 4.81 | 181.2 | 100 |
| 18.99 | 0.2 | 4.67 | 85.17 | 47 |
| 20.58 | 0.2 | 4.32 | 130.88 | 72.23 |
| 21.62 | 0.2 | 4.11 | 54.22 | 29.92 |
| 23.07 | 0.2 | 3.86 | 91 | 50.22 |
| 23.85 | 0.39 | 3.73 | 29.84 | 16.47 |
| 24.8 | 0.34 | 3.59 | 45.23 | 24.96 |

### Example 15: AMG 397 Phosphate Salt Form 1

Crystalline AMG 397 Phosphate salt form 1 was formed by 1:1 (mol/mol, API/base) salt reaction with H₃PO₄ in EtOH/THF 1:1 by evaporative cooling. The isolated solids were AMG 397 phosphate salt form 1, which was characterized as shown in the below tables.

TABLE 23
XRPD Data Table

| Pos. [°2θ] | FWHM [°2θ] | d-spacing [Å] | Height [cts] | Rel. Int. [%] |
|---|---|---|---|---|
| 5.09 | 0.44 | 17.37 | 1429.11 | 12.89 |
| 8.84 | 0.25 | 10 | 70.73 | 0.64 |
| 9.8 | 0.19 | 9.02 | 333.25 | 3.01 |
| 10.67 | 0.16 | 8.29 | 1969.3 | 17.76 |
| 11.09 | 0.09 | 7.98 | 4018.8 | 36.24 |
| 11.16 | 0.08 | 7.93 | 3795.38 | 34.23 |
| 12.26 | 0.2 | 7.22 | 4856.52 | 43.8 |
| 12.41 | 0.06 | 7.14 | 4080.44 | 36.8 |
| 12.99 | 0.13 | 6.82 | 1785.41 | 16.1 |
| 13.08 | 0.06 | 6.77 | 1976.8 | 17.83 |
| 14.03 | 0.17 | 6.31 | 5106.78 | 46.05 |
| 14.48 | 0.09 | 6.12 | 633.1 | 5.71 |
| 15.31 | 0.16 | 5.79 | 2151.38 | 19.4 |
| 15.97 | 0.13 | 5.55 | 3063.64 | 27.63 |
| 16.07 | 0.06 | 5.51 | 3209.51 | 28.94 |
| 16.69 | 0.14 | 5.31 | 3050.2 | 27.51 |
| 16.83 | 0.11 | 5.27 | 3499.65 | 31.56 |
| 17.7 | 0.27 | 5.01 | 5448.7 | 49.14 |
| 18.61 | 0.17 | 4.77 | 11088.6 | 100 |
| 18.72 | 0.08 | 4.74 | 9088.57 | 81.96 |
| 19.34 | 0.19 | 4.59 | 2890.37 | 26.07 |
| 20.3 | 0.24 | 4.37 | 5202.27 | 46.92 |
| 20.72 | 0.09 | 4.29 | 2407.23 | 21.71 |
| 21.92 | 0.06 | 4.05 | 2833.98 | 25.56 |
| 22.32 | 0.16 | 3.98 | 1902.75 | 17.16 |
| 22.92 | 0.21 | 3.88 | 2526.78 | 22.79 |
| 22.99 | 0.08 | 3.87 | 2275.66 | 20.52 |
| 23.82 | 0.27 | 3.73 | 1687.9 | 15.22 |
| 24.2 | 0.23 | 3.67 | 1321.19 | 11.91 |
| 24.71 | 0.21 | 3.6 | 3914.21 | 35.3 |
| 24.8 | 0.12 | 3.6 | 3531.82 | 31.85 |
| 25.27 | 0.19 | 3.52 | 774.11 | 6.98 |
| 25.45 | 0.15 | 3.5 | 757.13 | 6.83 |
| 26.05 | 0.27 | 3.42 | 1081.13 | 9.75 |
| 26.52 | 0.35 | 3.36 | 1138.49 | 10.27 |
| 27.33 | 0.31 | 3.26 | 709.04 | 6.39 |
| 28.23 | 0.1 | 3.16 | 1392.2 | 12.56 |
| 28.69 | 0.31 | 3.11 | 973.72 | 8.78 |
| 29.32 | 0.23 | 3.04 | 606.89 | 5.47 |
| 29.67 | 0.15 | 3.01 | 621.4 | 5.6 |
| 30.23 | 0.23 | 2.95 | 687.18 | 6.2 |
| 30.7 | 0.13 | 2.91 | 1054.83 | 9.51 |
| 31.45 | 0.23 | 2.84 | 608.61 | 5.49 |
| 31.95 | 0.15 | 2.8 | 525.8 | 4.74 |
| 32.42 | 0.27 | 2.76 | 471.18 | 4.25 |
| 33.02 | 0.19 | 2.71 | 376.16 | 3.39 |
| 33.62 | 0.19 | 2.66 | 390.03 | 3.52 |
| 34.08 | 0.23 | 2.63 | 268.2 | 2.42 |
| 34.69 | 0.38 | 2.58 | 335.11 | 3.02 |
| 35.29 | 0.31 | 2.54 | 414.03 | 3.73 |
| 35.92 | 0.19 | 2.5 | 257.95 | 2.33 |
| 37.46 | 0.23 | 2.4 | 456.58 | 4.12 |
| 38.24 | 0.19 | 2.35 | 208.6 | 1.88 |
| 39 | 0.27 | 2.31 | 307.07 | 2.77 |
| 39.37 | 0.27 | 2.29 | 255.77 | 2.31 |

TABLE 24
¹³C NMR Data

| Peak | v(F1) [ppm] |
|---|---|
| 1 | 168.8 |
| 2 | 150.8 |
| 3 | 139.9 |
| 4 | 139.8 |
| 5 | 139.2 |
| 6 | 130.6 |
| 7 | 130.2 |
| 8 | 129.5 |
| 9 | 129.4 |
| 10 | 128.7 |
| 11 | 127.9 |
| 12 | 126 |
| 13 | 119.1 |
| 14 | 117.3 |
| 15 | 115.4 |
| 16 | 80.9 |
| 17 | 79.6 |
| 18 | 61 |
| 19 | 59.8 |
| 20 | 58.9 |
| 21 | 58.8 |
| 22 | 57.7 |
| 23 | 55.8 |
| 24 | 53.7 |
| 25 | 53.3 |
| 26 | 51.7 |
| 27 | 49.9 |
| 28 | 43.6 |
| 29 | 41.3 |
| 30 | 35.7 |
| 31 | 31.9 |
| 32 | 31.7 |
| 33 | 29.3 |
| 34 | 27.6 |
| 35 | 27.6 |
| 36 | 26.1 |
| 37 | 23.6 |
| 38 | 22.2 |
| 39 | 21.2 |
| 40 | 18.3 |
| 41 | 15 |
| 42 | 5.8 |

TABLE 25
¹H NMR Data

| Peak | δ/ppm | multiplicity |
|---|---|---|
| 1 | 7.667 | d, J = 8.6 Hz, 1H |
| 2 | 7.246 | dd, J = 8.6, 2.1 Hz, 1H |
| 3 | 7.171 | d, J = 1.6 Hz, 1H |
| 4 | 7.158 | d, J = 2.1 Hz, 1H |
| 5 | 7.014 | dd, J = 8.1, 1.6 Hz, 1H |
| 6 | 6.84 | d, J = 8.1 Hz, 1H |
| 7 | 5.624 | dt, J = 16.1, 5.4 Hz, 1H |
| 8 | 5.414 | br d, J = 16.1 Hz, 1H |
| 9 | 4.033 | d, J = 12.2 Hz, 1H |
| 10 | 3.988 | d, J = 12.2 Hz, 1H |
| 11 | 3.869 | br d, J = 14.7 Hz, 1H |
| 12 | 3.836 | br q, J = 7.2 Hz, 1H |
| 13 | 3.579 | d, J = 14.1 Hz, 1H |
| 14 | 3.257 | s, 3H |
| 15 | 3.208 | d, J = 14.1 Hz, 1H |
| 16 | 3.039 | br d, J = 11.5 Hz, 1H |
| 17 | 3.007 | br d, J = 11.0 Hz, 1H |
| 18 | 2.956 | dd, J = 14.7, 10.8 Hz, 1H |
| 19 | 2.911 | br d, J = 11.5 Hz, 1H |
| 20 | 2.795 | dt, J = 17.0, 4.2 Hz, 1H |
| 21 | 2.717 | m, 1H |
| 22 | 2.66 | br d, J = 11.4 Hz, 1H |
| 23 | 2.587 | m, 1H |
| 24 | 2.582 | m, 1H |
| 25 | 2.527 | m, 1H |
| 26 | 2.472 | m, 1H |
| 27 | 2.468 | br d, J = 14.4 Hz, 1H |
| 28 | 2.439 | m, 1H |
| 29 | 2.396 | br t, J = 11.5 Hz, 1H |
| 30 | 2.291 | br d, J = 14.4 Hz, 1H |
| 31 | 2.2 | m, 1H |
| 32 | 2.178 | m, 1H |
| 33 | 2.158 | m, 1H |
| 34 | 2.043 | m, 1H |
| 35 | 1.998 | dt, J = 14.0, 4.0 Hz, 1H |
| 36 | 1.856 | m, 2H |
| 37 | 1.783 | m, 1H |
| 38 | 1.702 | m, 1H |
| 39 | 1.672 | m, 1H |
| 40 | 1.656 | m, 1H |
| 41 | 1.621 | m, 1H |
| 42 | 1.596 | m, 1H |
| 43 | 1.58 | m, 1H |
| 44 | 1.453 | m, 1H |
| 45 | 1.398 | m, 1H |
| 46 | 1.351 | m, 1H |
| 47 | 1.275 | m, 1H |
| 48 | 1.244 | d, J = 7.2 Hz, 3H |
| 49 | 0.95 | d, J = 6.6 Hz, 3H |

TABLE 26
¹⁵N NMR Data

| Peak | δ/ppm |
|---|---|
| 1 | 64.5 |
| 2 | 58.4 |
| 3 | 37.1 |

### Example 16: AMG 397 Fumarate Salt Form 1

Crystalline AMG 397 fumarate salt form 1 was formed by 1:2 (mol/mol, API/acid) salt reaction with fumaric acid in EtOAc mixed at room temperature for 3 days. The isolated solids were AMG 397 fumarate salt form 1, which was characterized as shown in the below tables.

TABLE 27
XRPD Data Table

| Pos. [°2θ] | FWHM [°2θ] | d-spacing [Å] | Height [cts] | Rel. Int. [%] |
|---|---|---|---|---|
| 5.33 | 0.13 | 16.57 | 629.59 | 43.46 |
| 7.57 | 0.19 | 11.68 | 117.6 | 8.12 |
| 8.46 | 0.19 | 10.45 | 99.36 | 6.86 |
| 9.65 | 0.1 | 9.16 | 262.44 | 18.12 |
| 10.36 | 0.13 | 8.54 | 716.45 | 49.46 |
| 10.99 | 0.13 | 8.05 | 550.5 | 38 |
| 12.21 | 0.23 | 7.25 | 745.41 | 51.46 |
| 12.85 | 0.13 | 6.89 | 229.82 | 15.87 |
| 13.89 | 0.16 | 6.37 | 693.54 | 47.88 |
| 14.94 | 0.16 | 5.93 | 332.36 | 22.94 |
| 15.54 | 0.13 | 5.7 | 476.72 | 32.91 |
| 15.82 | 0.13 | 5.6 | 635.27 | 43.86 |
| 16.27 | 0.13 | 5.45 | 546.51 | 37.73 |
| 16.93 | 0.13 | 5.24 | 275.76 | 19.04 |
| 17.6 | 0.13 | 5.04 | 1202.62 | 83.02 |
| 17.93 | 0.1 | 4.95 | 539.55 | 37.25 |
| 18.23 | 0.1 | 4.87 | 1008.19 | 69.6 |
| 18.44 | 0.13 | 4.81 | 1448.54 | 100 |
| 19.15 | 0.13 | 4.64 | 544.84 | 37.61 |
| 20.19 | 0.1 | 4.4 | 540.97 | 37.35 |
| 20.86 | 0.19 | 4.26 | 391.82 | 27.05 |
| 21.57 | 0.13 | 4.12 | 500.5 | 34.55 |
| 22.81 | 0.32 | 3.9 | 233.13 | 16.09 |
| 24.03 | 0.13 | 3.7 | 764.98 | 52.81 |
| 24.67 | 0.26 | 3.61 | 310.33 | 21.42 |
| 26.14 | 0.52 | 3.41 | 146.87 | 10.14 |
| 26.95 | 0.26 | 3.31 | 85.57 | 5.91 |
| 28.04 | 0.26 | 3.18 | 139.3 | 9.62 |

### ¹H NMR Data

¹H NMR (400 MHZ, DMSO-d6) δ ppm 0.91 (br d, J=6.65 Hz, 3H) 1.06-1.21 (m, 3H) 1.21-1.48 (m, 3H) 1.49-1.71 (m, 4H) 1.74-1.88 (m, 2H) 1.95-2.13 (m, 2H) 2.13-2.39 (m, 3H) 2.39-2.48 (m, 1H) 2.52-2.79 (m, 3H) 2.80-2.99 (m, 2H) 3.01-3.17 (m, 1H) 3.17-3.25 (m, 2H) 3.47-3.65 (m, 2H) 3.66-3.79 (m, 1H) 3.86 (br d, J=14.23 Hz, 1H) 3.91-4.10 (m, 2H) 5.32-5.52 (m, 1H) 5.61 (br d, J=16.24 Hz, 1H) 6.51-6.71 (m, 2H) 6.81 (br d, J=7.97 Hz, 1H) 6.91-7.12 (m, 1H) 7.12-7.21 (m, 1H) 7.27 (dd, J=8.53, 2.27 Hz, 1H) 7.67 (d, J=8.56 Hz, 1H).

### Example 17: AMG 397 Fumarate Salt Form 2 (Acetone Solvate)

AMG 397 fumarate salt form 2 (acetone solvate) was formed by 1:1 (mol/mol, API/acid) salt reaction with fumaric acid in Acetone mixed at room temperature for 3 days. The isolated solids were AMG 397 fumarate salt form 2 (acetone solvate), which was characterized as shown in the below tables.

TABLE 28
XRPD Data Table

| Pos. [°2θ] | FWHM [°2θ] | d-spacing [Å] | Height [cts] | Rel. Int. [%] |
|---|---|---|---|---|
| 2.06 | 0.94 | 42.82 | 106.51 | 0.23 |
| 5.91 | 0.05 | 14.96 | 677.57 | 1.47 |
| 7.83 | 0.05 | 11.3 | 523.07 | 1.14 |
| 9.41 | 0.07 | 9.4 | 2826.89 | 6.15 |
| 10.3 | 0.08 | 8.59 | 10033.81 | 21.83 |
| 10.72 | 0.08 | 8.25 | 16641.23 | 36.21 |
| 11.89 | 0.07 | 7.44 | 28754.35 | 62.56 |
| 12.62 | 0.07 | 7.02 | 4284.34 | 9.32 |
| 12.92 | 0.07 | 6.85 | 913.28 | 1.99 |
| 13.63 | 0.08 | 6.5 | 17303.95 | 37.65 |
| 13.94 | 0.07 | 6.35 | 3294.65 | 7.17 |
| 14.86 | 0.07 | 5.96 | 8271.9 | 18 |
| 15.7 | 0.08 | 5.64 | 23378.71 | 50.87 |
| 16.29 | 0.07 | 5.44 | 6249.48 | 13.6 |
| 16.53 | 0.07 | 5.36 | 4958.01 | 10.79 |
| 17.36 | 0.08 | 5.11 | 4219.75 | 9.18 |
| 17.92 | 0.07 | 4.95 | 45961.55 | 100 |
| 18.1 | 0.08 | 4.9 | 31023.22 | 67.5 |
| 18.64 | 0.08 | 4.76 | 13962.89 | 30.38 |
| 18.82 | 0.08 | 4.72 | 17647.04 | 38.4 |
| 19.58 | 0.1 | 4.53 | 25561.01 | 55.61 |
| 20.03 | 0.13 | 4.43 | 5294.54 | 11.52 |
| 20.67 | 0.1 | 4.3 | 4284.1 | 9.32 |
| 20.82 | 0.05 | 4.27 | 4196.65 | 9.13 |
| 21.46 | 0.13 | 4.14 | 16271.64 | 35.4 |
| 22.16 | 0.1 | 4.01 | 4599.29 | 10.01 |
| 22.55 | 0.08 | 3.94 | 6555.1 | 14.26 |
| 22.72 | 0.07 | 3.91 | 3862.24 | 8.4 |
| 23.28 | 0.1 | 3.82 | 8748.82 | 19.04 |
| 23.91 | 0.1 | 3.72 | 9835.41 | 21.4 |
| 24.23 | 0.08 | 3.67 | 3446.75 | 7.5 |
| 24.47 | 0.1 | 3.64 | 4857.08 | 10.57 |
| 24.98 | 0.12 | 3.56 | 2768.18 | 6.02 |
| 25.35 | 0.07 | 3.51 | 3425.88 | 7.45 |
| 25.51 | 0.1 | 3.49 | 6367.64 | 13.85 |
| 26.01 | 0.13 | 3.43 | 3311.24 | 7.2 |
| 26.6 | 0.1 | 3.35 | 3597.5 | 7.83 |
| 27.18 | 0.12 | 3.28 | 4579.81 | 9.96 |
| 27.43 | 0.05 | 3.25 | 3179.51 | 6.92 |
| 27.58 | 0.1 | 3.23 | 3699.02 | 8.05 |
| 28.07 | 0.15 | 3.18 | 4631.6 | 10.08 |
| 28.49 | 0.1 | 3.13 | 2321.93 | 5.05 |
| 28.87 | 0.12 | 3.09 | 2547.04 | 5.54 |
| 29.13 | 0.1 | 3.07 | 3148.25 | 6.85 |
| 29.6 | 0.12 | 3.02 | 2820.44 | 6.14 |
| 29.99 | 0.1 | 2.98 | 1855.8 | 4.04 |
| 30.36 | 0.12 | 2.94 | 2188.38 | 4.76 |
| 30.87 | 0.1 | 2.9 | 443.89 | 0.97 |
| 31.28 | 0.17 | 2.86 | 2994.89 | 6.52 |
| 31.81 | 0.17 | 2.81 | 1041.95 | 2.27 |
| 32.2 | 0.07 | 2.78 | 1480.53 | 3.22 |
| 32.57 | 0.07 | 2.75 | 1803.07 | 3.92 |
| 32.9 | 0.1 | 2.72 | 1785.94 | 3.89 |
| 32.99 | 0.08 | 2.72 | 1954.18 | 4.25 |
| 33.45 | 0.13 | 2.68 | 356.78 | 0.78 |
| 33.7 | 0.07 | 2.66 | 1465.55 | 3.19 |
| 33.89 | 0.08 | 2.64 | 1001.54 | 2.18 |
| 34.35 | 0.07 | 2.61 | 928.08 | 2.02 |
| 34.76 | 0.1 | 2.58 | 2242.09 | 4.88 |
| 35.13 | 0.1 | 2.55 | 184.79 | 0.4 |
| 35.44 | 0.17 | 2.53 | 496.07 | 1.08 |
| 35.85 | 0.08 | 2.5 | 731.68 | 1.59 |
| 36.27 | 0.2 | 2.48 | 706.95 | 1.54 |
| 36.65 | 0.1 | 2.45 | 1099.48 | 2.39 |
| 37.29 | 0.07 | 2.41 | 1031.07 | 2.24 |
| 37.63 | 0.05 | 2.39 | 958.92 | 2.09 |
| 37.89 | 0.1 | 2.37 | 791.68 | 1.72 |
| 38.23 | 0.17 | 2.35 | 1658.55 | 3.61 |
| 38.85 | 0.12 | 2.32 | 1893.59 | 4.12 |
| 39.31 | 0.1 | 2.29 | 96.05 | 0.21 |
| 39.76 | 0.2 | 2.27 | 448.61 | 0.98 |

### ¹H NMR Data

¹H NMR (400 MHZ, DMSO-d6) δ ppm 0.92 (d, J=6.70 Hz, 3H) 1.15-1.44 (m, 5H) 1.49-1.71 (m, 4H) 1.71-1.95 (m, 3H) 1.96-2.18 (m, 5H) 2.18-2.34 (m, 2H) 2.39-2.48 (m, 2H) 2.53-2.78 (m, 3H) 2.87-3.14 (m, 3H) 3.14-3.22 (m, 1H) 3.56 (br d, J=13.99 Hz, 4H) 3.65-3.78 (m, 3H) 3.86 (br d, J=14.33 Hz, 4H) 3.91-4.18 (m, 7H) 5.33-5.51 (m, 3H) 5.61 (dt, J=16.24, 5.11 Hz, 3H) 6.61 (s, 3H) 6.81 (d, J=8.12 Hz, 3H) 6.91-7.12 (m, 3H) 7.12-7.21 (m, 3H) 7.27 (dd, J=8.49, 2.27 Hz, 3H) 7.67 (d, J=8.51 Hz, 1H).

TABLE 29
X-ray Single Structure Data

| Crystal system | Primitive Orthorhombic | | |
|---|---|---|---|
| Space group(s) | P2₁2₁2₁ (19) | | |
| Extinction Symbol | P 2₁ 2₁ 2₁ | | |
| Chiral Contents? | Chiral | | |
| Unit cell dimensions | a = 9.891 | Å | α = 90° |
| | b = 17.226 | Å | β = 90° |
| | c = 29.805 | Å | γ = 90° |

| | |
|---|---|
| Volume | 5078.2 A³ |

### Example 18: AMG 397 Citrate Salt Form 1

AMG 397 citrate salt form 1 was formed by 1:1 (mol/mol, API/acid) salt reaction with citric acid in EtOAc mixed at room temperature for 3 days. The isolated solids were AMG 397 citrate salt form 1, which was characterized as shown in the below tables.

TABLE 30
XRPD Data Table

| Pos. [°2θ] | FWHM [°2θ] | d-spacing [Å] | Height [cts] | Rel. Int. [%] |
|---|---|---|---|---|
| 6.05 | 0.19 | 14.6 | 284.85 | 10.91 |
| 9.62 | 0.16 | 9.19 | 250.84 | 9.61 |
| 10.55 | 0.13 | 8.39 | 1155.75 | 44.26 |
| 10.96 | 0.13 | 8.07 | 719.15 | 27.54 |
| 12.12 | 0.16 | 7.3 | 844.35 | 32.34 |
| 12.82 | 0.13 | 6.91 | 557.46 | 21.35 |
| 13.89 | 0.19 | 6.38 | 851.02 | 32.59 |
| 15.18 | 0.13 | 5.84 | 423.38 | 16.21 |
| 16.04 | 0.23 | 5.53 | 929.71 | 35.6 |
| 16.87 | 0.19 | 5.25 | 286.51 | 10.97 |
| 17.6 | 0.16 | 5.04 | 1457.36 | 55.81 |
| 18.31 | 0.26 | 4.84 | 2611.21 | 100 |
| 19.23 | 0.16 | 4.62 | 864.31 | 33.1 |
| 19.54 | 0.16 | 4.54 | 630.96 | 24.16 |
| 19.99 | 0.16 | 4.44 | 899.64 | 34.45 |
| 20.49 | 0.19 | 4.33 | 431.78 | 16.54 |
| 21.13 | 0.19 | 4.2 | 273.31 | 10.47 |
| 21.92 | 0.16 | 4.05 | 885.19 | 33.9 |
| 22.91 | 0.26 | 3.88 | 360.41 | 13.8 |
| 24.39 | 0.19 | 3.65 | 539.03 | 20.64 |
| 24.72 | 0.13 | 3.6 | 439.36 | 16.83 |
| 25.93 | 0.26 | 3.44 | 306.53 | 11.74 |
| 26.44 | 0.26 | 3.37 | 209.56 | 8.03 |
| 27.95 | 0.39 | 3.19 | 203.95 | 7.81 |
| 28.68 | 0.16 | 3.11 | 292.57 | 11.2 |
| 29.44 | 0.13 | 3.03 | 245.73 | 9.41 |

### ¹H NMR Data

¹H NMR (400 MHZ, DMSO-d6) δ ppm 0.92-0.99 (m, 2H) 1.22-1.31 (m, 2H) 1.32-1.51 (m, 3H) 1.55-1.68 (m, 2H) 1.70-1.89 (m, 4H) 2.02-2.11 (m, 12H) 2.15-2.36 (m, 2H) 2.37-2.47 (m, 1H) 2.55-2.64 (m, 4H) 2.65-2.73 (m, 3H) 2.75-2.87 (m, 2H) 2.89-3.03 (m, 2H) 3.08-3.22 (m, 3H) 3.29-3.45 (m, 5H) 3.53-3.63 (m, 1H) 3.80-3.92 (m, 2H) 3.94-4.11 (m, 2H) 5.31-5.42 (m, 1H) 5.52-5.68 (m, 1H) 6.83-6.91 (m, 1H) 6.97-7.04 (m, 1H) 7.09-7.15 (m, 1H) 7.15-7.21 (m, 1H) 7.22-7.32 (m, 1H) 7.66 (d, J=8.56 Hz, 1H).

TABLE 31
X-ray Single Structure Data

| Crystal system | Primitive Orthorhombic | | |
|---|---|---|---|
| Space group(s) | P2₁2₁2₁ (19) | | |
| Extinction Symbol | P 2₁ 2₁ 2₁ | | |
| Chiral Contents? | Chiral | | |
| Unit cell dimensions | a = 9.718 | Å | α = 90° |
| | b = 16.919 | Å | β = 90° |
| | c = 29.622 | Å | γ = 90° |

| | |
|---|---|
| Volume | 4870.4 Å³ |

### Example 19: AMG 397 Citrate Salt Form 2 (Hydrate)

AMG 397 citrate salt form 2 (hydrate) was formed by 1:1 (mol/mol, API/acid) salt reaction with citric acid in EtOAc mixed at room temperature for 3 days. The isolated solids were AMG 397 citrate salt form 2 (hydrate), which was characterized as shown in the below tables.

TABLE 32
XRPD Data Table

| Pos. [°2θ] | FWHM [°2θ] | d-spacing [Å] | Height [cts] | Rel. Int. [%] |
|---|---|---|---|---|
| 6.2041 | 0.1728 | 14.23458 | 63.95 | 2.01 |
| 9.7246 | 0.192 | 9.08787 | 161.7 | 5.09 |
| 10.655 | 0.288 | 8.29627 | 715.1 | 22.49 |
| 11.0677 | 0.288 | 7.98786 | 466.25 | 14.67 |
| 12.2267 | 0.2688 | 7.23315 | 789.33 | 24.83 |
| 12.8961 | 0.288 | 6.85914 | 576.26 | 18.13 |
| 13.2201 | 0.1728 | 6.69177 | 165.44 | 5.2 |
| 13.9802 | 0.3264 | 6.32958 | 873.68 | 27.48 |
| 14.3933 | 0.1344 | 6.14885 | 37.1 | 1.17 |
| 15.2349 | 0.0768 | 5.81102 | 378.03 | 11.89 |
| 15.9859 | 0.2304 | 5.53967 | 917.31 | 28.85 |
| 16.6298 | 0.2304 | 5.32661 | 208.88 | 6.57 |
| 17.0047 | 0.1536 | 5.21002 | 81.65 | 2.57 |
| 17.6721 | 0.0864 | 5.01471 | 1586.16 | 49.89 |
| 18.4097 | 0.0864 | 4.81542 | 3179.11 | 100 |
| 18.506 | 0.096 | 4.79059 | 2472.87 | 77.79 |
| 19.2843 | 0.3072 | 4.59896 | 719.32 | 22.63 |
| 20.0622 | 0.1152 | 4.42235 | 864.23 | 27.18 |
| 20.5827 | 0.3072 | 4.31169 | 364.34 | 11.46 |
| 21.225 | 0.192 | 4.18263 | 173.14 | 5.45 |
| 21.9391 | 0.288 | 4.04809 | 909.55 | 28.61 |
| 22.6049 | 0.2304 | 3.93033 | 297.13 | 9.35 |
| 22.9149 | 0.192 | 3.87786 | 349.98 | 11.01 |
| 23.8201 | 0.384 | 3.7325 | 136.26 | 4.29 |
| 24.1049 | 0.192 | 3.68905 | 234.8 | 7.39 |
| 24.4296 | 0.1728 | 3.64075 | 536.28 | 16.87 |
| 24.7811 | 0.2112 | 3.58989 | 385.2 | 12.12 |
| 25.0497 | 0.0576 | 3.55201 | 13.93 | 0.44 |
| 25.4315 | 0.0768 | 3.49954 | 51.81 | 1.63 |
| 25.7373 | 0.192 | 3.45865 | 90.11 | 2.83 |
| 25.9854 | 0.2688 | 3.42619 | 243.07 | 7.65 |
| 26.3936 | 0.1344 | 3.37411 | 222.73 | 7.01 |
| 27.192 | 0.2304 | 3.27683 | 84.15 | 2.65 |
| 27.8268 | 0.2688 | 3.2035 | 176.37 | 5.55 |
| 28.0443 | 0.1536 | 3.17916 | 195.79 | 6.16 |
| 28.3424 | 0.1152 | 3.14639 | 93.27 | 2.93 |
| 28.785 | 0.1536 | 3.09901 | 147.68 | 4.65 |
| 29.1593 | 0.0768 | 3.06007 | 15.7 | 0.49 |
| 29.4553 | 0.1344 | 3.03 | 159.97 | 5.03 |
| 29.8742 | 0.096 | 2.98845 | 39.8 | 1.25 |

### Example 20: AMG 397 Lactate Salt Form 1

AMG 397 lactate salt form 1 was formed by 1:1 (mol/mol, API/acid) salt reaction with lactic acid in EtOAc mixed at room temperature for 3 days. The isolated solids were AMG 397 lactate salt form 1, which was characterized as shown in the below tables.

TABLE 33
XRPD Data Table

| Pos. [°2θ] | FWHM [°2θ] | d-spacing [Å] | Height [cts] | Rel. Int. [%] |
|---|---|---|---|---|
| 5.94 | 0.13 | 14.87 | 427.58 | 14.96 |
| 9.61 | 0.19 | 9.2 | 128.92 | 4.51 |
| 10.53 | 0.13 | 8.4 | 772.91 | 27.04 |
| 10.9 | 0.16 | 8.11 | 879.81 | 30.78 |
| 12.07 | 0.16 | 7.33 | 1157.72 | 40.5 |
| 12.77 | 0.13 | 6.93 | 517.29 | 18.1 |
| 13.29 | 0.16 | 6.66 | 262.42 | 9.18 |
| 13.77 | 0.13 | 6.43 | 785.15 | 27.47 |
| 15.06 | 0.19 | 5.88 | 214.33 | 7.5 |
| 15.87 | 0.23 | 5.59 | 530.13 | 18.55 |
| 16.82 | 0.26 | 5.27 | 398.89 | 13.96 |
| 17.45 | 0.13 | 5.08 | 772.13 | 27.01 |
| 17.75 | 0.19 | 5 | 1206.75 | 42.22 |
| 18.27 | 0.16 | 4.86 | 2858.39 | 100 |
| 19.14 | 0.32 | 4.64 | 420.5 | 14.71 |
| 19.97 | 0.19 | 4.45 | 677.33 | 23.7 |
| 20.36 | 0.1 | 4.36 | 492.66 | 17.24 |
| 21.67 | 0.26 | 4.1 | 418.26 | 14.63 |
| 22.7 | 0.39 | 3.92 | 282.2 | 9.87 |
| 23.85 | 0.13 | 3.73 | 529.61 | 18.53 |
| 24.55 | 0.19 | 3.63 | 312.73 | 10.94 |
| 25.08 | 0.19 | 3.55 | 289.13 | 10.12 |
| 25.69 | 0.19 | 3.47 | 172.65 | 6.04 |
| 27.78 | 0.26 | 3.21 | 226.68 | 7.93 |

### ¹H NMR Data

¹H NMR (500 MHZ, CHLOROFORM-d) δ ppm 0.76-0.99 (m, 3H) 1.06 (d, J=6.23 Hz, 4H) 1.18-1.34 (m, 3H) 1.34-1.55 (m, 8H) 1.55-1.61 (m, 1H) 1.64-1.71 (m, 2H) 1.75-1.89 (m, 3H) 1.89-2.01 (m, 3H) 2.01-2.24 (m, 6H) 2.32 (br d, J=14.27 Hz, 1H) 2.39-2.51 (m, 1H) 2.51-2.65 (m, 3H) 2.65-2.83 (m, 6H) 2.92 (br dd, J=14.92, 9.47 Hz, 2H) 3.04-3.19 (m, 1H) 3.19-3.33 (m, 2H) 3.33-3.38 (m, 2H) 3.41 (br d, J=10.12 Hz, 1H) 3.51 (br d, J=9.34 Hz, 1H) 3.73 (br d, J=14.27 Hz, 1H) 4.00-4.19 (m, 5H) 5.34 (br d, J=16.09 Hz, 1H) 5.61-5.74 (m, 1H) 6.84-7.01 (m, 2H) 7.01-7.15 (m, 1H) 7.15-7.26 (m, 1H) 7.29-7.53 (m, 8H) 7.72 (d, J=8.56 Hz, 1H).

### Example 21: AMG 397 Succinate Salt Form 1

AMG 397 succinate salt form 1 was formed by 1:1 (mol/mol, API/acid) salt reaction with lactic acid in EtOAc mixed at room temperature for 3 days. The isolated solids were AMG 397 succinate salt form 1, which was characterized as shown in the below tables.

TABLE 34
XRPD Data Table

| Pos. [°2θ] | FWHM [°2θ] | d-spacing [Å] | Height [cts] | Rel. Int. [%] |
|---|---|---|---|---|
| 5.16 | 0.52 | 17.13 | 275.47 | 10.17 |
| 9.78 | 0.13 | 9.04 | 161.66 | 5.97 |
| 10.36 | 0.13 | 8.54 | 319.23 | 11.78 |
| 10.67 | 0.13 | 8.29 | 627.23 | 23.15 |
| 11.08 | 0.19 | 7.99 | 888.87 | 32.81 |
| 12.22 | 0.16 | 7.24 | 1558.38 | 57.52 |
| 12.91 | 0.19 | 6.86 | 548.81 | 20.26 |
| 13.93 | 0.16 | 6.36 | 1263.02 | 46.62 |
| 15.23 | 0.23 | 5.82 | 368.69 | 13.61 |
| 15.8 | 0.19 | 5.61 | 767.52 | 28.33 |
| 16.66 | 0.19 | 5.32 | 617.05 | 22.78 |
| 17.59 | 0.16 | 5.04 | 1591.74 | 58.76 |
| 17.95 | 0.13 | 4.94 | 1296.36 | 47.85 |
| 18.36 | 0.16 | 4.83 | 2709.06 | 100 |
| 18.67 | 0.16 | 4.75 | 2187.28 | 80.74 |
| 19.13 | 0.16 | 4.64 | 734.28 | 27.1 |
| 20.27 | 0.23 | 4.38 | 1489.98 | 55 |
| 21.64 | 0.23 | 4.11 | 892.93 | 32.96 |
| 22.23 | 0.16 | 4 | 468.9 | 17.31 |
| 22.78 | 0.16 | 3.9 | 878.51 | 32.43 |
| 23.91 | 0.16 | 3.72 | 478.54 | 17.66 |
| 24.53 | 0.19 | 3.63 | 1146.47 | 42.32 |
| 25.19 | 0.19 | 3.54 | 217.75 | 8.04 |
| 26.21 | 0.32 | 3.4 | 362.69 | 13.39 |
| 28.34 | 0.19 | 3.15 | 358.42 | 13.23 |
| 29.21 | 0.23 | 3.06 | 300.23 | 11.08 |

### ¹H NMR Data

¹H NMR (500 MHZ, CHLOROFORM-d) δ ppm 0.77-0.99 (m, 2H) 0.99-1.15 (m, 3H) 1.15-1.33 (m, 2H) 1.33-1.44 (m, 2H) 1.45-1.67 (m, 6H) 1.68-1.78 (m, 1H) 1.83 (br d, J=13.75 Hz, 2H) 1.89-2.00 (m, 2H) 2.01-2.17 (m, 2H) 2.22 (br dd, J=15.05, 6.23 Hz, 1H) 2.28-2.49 (m, 1H) 2.49-2.65 (m, 3H) 2.70-2.85 (m, 3H) 2.92 (br dd, J=14.92, 9.73 Hz, 1H) 3.05-3.19 (m, 1H) 3.19-3.31 (m, 1H) 3.36 (s, 2H) 3.49 (br d, J=11.42 Hz, 1H) 3.73 (br d, J=14.53 Hz, 1H) 4.00-4.20 (m, 3H) 5.33 (br d, J=16.87 Hz, 1H) 5.51-5.74 (m, 1H) 6.87-6.99 (m, 1H) 7.01-7.15 (m, 1H) 7.20 (dd, J=8.56, 2.34 Hz, 1H) 7.48 (s, 1H) 7.72 (d, J=8.56 Hz, 1H).

### Example 22: AMG 397 Besylate Salt Form 1 (Hydrate)

AMG 397 besylate salt form 1 (hydrate) was formed by 1:1 (mol/mol, API/base) salt reaction with BSA in EtOH heat cycled to 60° C. for 1 h. The isolated solids were AMG 397 besylate salt form 1 (hydrate), which was characterized as shown in the below tables.

TABLE 35
XRPD Data Table

| Pos. [°2θ] | FWHM [°2θ] | d-spacing [Å] | Height [cts] | Rel. Int. [%] |
|---|---|---|---|---|
| 4.83 | 0.5 | 18.28 | 1357.06 | 8.76 |
| 6 | 0.38 | 14.74 | 746.45 | 4.82 |
| 9.95 | 0.14 | 8.89 | 640.41 | 4.14 |
| 10.48 | 0.09 | 8.44 | 1249.17 | 8.07 |
| 11.18 | 0.04 | 7.91 | 5022.25 | 32.43 |
| 12.4 | 0.13 | 7.14 | 6273.02 | 40.51 |
| 13.07 | 0.2 | 6.77 | 2971.85 | 19.19 |
| 13.84 | 0.06 | 6.4 | 6337.39 | 40.93 |
| 14.03 | 0.12 | 6.31 | 9882.62 | 63.82 |
| 14.09 | 0.06 | 6.3 | 6842.3 | 44.19 |
| 14.42 | 0.13 | 6.14 | 1045.16 | 6.75 |
| 15.33 | 0.09 | 5.78 | 3306.49 | 21.35 |
| 15.86 | 0.29 | 5.58 | 5433.49 | 35.09 |
| 16.08 | 0.15 | 5.51 | 3304.91 | 21.34 |
| 16.65 | 0.19 | 5.32 | 2723.06 | 17.59 |
| 17.65 | 0.06 | 5.02 | 11063.37 | 71.45 |
| 17.7 | 0.05 | 5.02 | 9292.64 | 60.01 |
| 18.03 | 0.25 | 4.92 | 6596.59 | 42.6 |
| 18.4 | 0.07 | 4.82 | 15484.5 | 100 |
| 18.7 | 0.06 | 4.74 | 11533.3 | 74.48 |
| 19.26 | 0.35 | 4.6 | 3940.98 | 25.45 |
| 19.69 | 0.1 | 4.5 | 1534.82 | 9.91 |
| 20.38 | 0.08 | 4.35 | 9905.31 | 63.97 |
| 20.75 | 0.19 | 4.28 | 3753.39 | 24.24 |
| 21.71 | 0.4 | 4.09 | 5563.87 | 35.93 |
| 22.22 | 0.1 | 4 | 1671.85 | 10.8 |
| 22.32 | 0.12 | 3.98 | 1551.72 | 10.02 |
| 22.86 | 0.36 | 3.89 | 3941.98 | 25.46 |
| 23.29 | 0.15 | 3.82 | 1830.53 | 11.82 |
| 23.92 | 0.21 | 3.72 | 3423.92 | 22.11 |
| 24.53 | 0.07 | 3.63 | 3744.67 | 24.18 |
| 25.15 | 0.33 | 3.54 | 2600.22 | 16.79 |
| 25.61 | 0.19 | 3.48 | 1470.62 | 9.5 |
| 26.17 | 0.21 | 3.4 | 2445.78 | 15.8 |
| 27.21 | 0.27 | 3.27 | 1284.25 | 8.29 |
| 27.78 | 0.27 | 3.21 | 1855.45 | 11.98 |
| 28.19 | 0.27 | 3.16 | 1379.11 | 8.91 |
| 28.67 | 0.15 | 3.11 | 826.27 | 5.34 |
| 29.19 | 0.23 | 3.06 | 1056.61 | 6.82 |
| 29.57 | 0.15 | 3.02 | 710.92 | 4.59 |
| 30.45 | 0.19 | 2.93 | 1897.74 | 12.26 |
| 31.43 | 0.27 | 2.84 | 752.64 | 4.86 |
| 31.84 | 0.23 | 2.81 | 511.39 | 3.3 |
| 32.44 | 0.35 | 2.76 | 740.06 | 4.78 |
| 32.85 | 0.23 | 2.72 | 657.54 | 4.25 |
| 33.42 | 0.27 | 2.68 | 801.31 | 5.17 |
| 33.93 | 0.17 | 2.64 | 604.53 | 3.9 |
| 34.56 | 0.38 | 2.59 | 534.06 | 3.45 |
| 35.14 | 0.31 | 2.55 | 389.16 | 2.51 |
| 35.81 | 0.38 | 2.51 | 791.06 | 5.11 |
| 37.21 | 0.38 | 2.41 | 645.26 | 4.17 |
| 37.91 | 0.23 | 2.37 | 404.08 | 2.61 |
| 38.64 | 0.31 | 2.33 | 548.57 | 3.54 |

### Example 23: AMG 397 Tosylate Salt Form 1

AMG 397 tosylate salt form 1 was formed by 1:1 (mol/mol, API/base) salt reaction with TSA in EtOH heat cycled to 60° C. for 1h. The isolated solids were AMG 397 tosylate salt form 1, which was characterized as shown in the below tables.

TABLE 36
XRPD Data Table

| Pos. [°2θ] | FWHM [°2θ] | d-spacing [Å] | Height [cts] | Rel. Int. [%] |
|---|---|---|---|---|
| 4.45 | 0.88 | 19.85 | 1447.96 | 22.33 |
| 5.15 | 0.16 | 17.17 | 1840.46 | 28.39 |
| 7.4 | 0.25 | 11.95 | 25.58 | 0.39 |
| 8.85 | 0.31 | 9.99 | 90.16 | 1.39 |
| 9.8 | 0.13 | 9.02 | 341.6 | 5.27 |
| 10.3 | 0.13 | 8.58 | 962.85 | 14.85 |
| 11.23 | 0.11 | 7.88 | 1288.89 | 19.88 |
| 11.7 | 0.09 | 7.56 | 691.04 | 10.66 |
| 12.18 | 0.13 | 7.27 | 3584.08 | 55.28 |
| 12.31 | 0.17 | 7.19 | 3409.4 | 52.59 |
| 12.95 | 0.14 | 6.83 | 2228.32 | 34.37 |
| 13.84 | 0.09 | 6.4 | 2491.74 | 38.43 |
| 14.01 | 0.16 | 6.32 | 2717.93 | 41.92 |
| 14.38 | 0.09 | 6.16 | 1086.63 | 16.76 |
| 14.61 | 0.09 | 6.06 | 1040.38 | 16.05 |
| 15.22 | 0.16 | 5.82 | 1680.05 | 25.91 |
| 15.75 | 0.09 | 5.63 | 1985.59 | 30.63 |
| 16.19 | 0.5 | 5.48 | 2248.24 | 34.68 |
| 16.36 | 0.13 | 5.42 | 3008.41 | 46.4 |
| 17.59 | 0.06 | 5.04 | 4262.04 | 65.74 |
| 18.17 | 0.13 | 4.88 | 5692.13 | 87.79 |
| 18.36 | 0.2 | 4.83 | 6483.5 | 100 |
| 18.9 | 0.14 | 4.7 | 4392.15 | 67.74 |
| 19.05 | 0.16 | 4.66 | 3403.46 | 52.49 |
| 19.79 | 0.13 | 4.49 | 2074.34 | 31.99 |
| 19.95 | 0.11 | 4.45 | 2570.16 | 39.64 |
| 20.48 | 0.13 | 4.34 | 5860.77 | 90.4 |
| 21.42 | 0.2 | 4.15 | 2502.64 | 38.6 |
| 22.22 | 0.28 | 4 | 1147.59 | 17.7 |
| 22.86 | 0.11 | 3.89 | 3003.09 | 46.32 |
| 23.44 | 0.19 | 3.8 | 1614.24 | 24.9 |
| 23.64 | 0.11 | 3.76 | 1934.67 | 29.84 |
| 23.81 | 0.11 | 3.74 | 1550.86 | 23.92 |
| 24.31 | 0.22 | 3.66 | 1438.2 | 22.18 |
| 24.64 | 0.19 | 3.61 | 1896.56 | 29.25 |
| 25.1 | 0.25 | 3.55 | 1583.36 | 24.42 |
| 25.89 | 0.19 | 3.44 | 1178.8 | 18.18 |
| 26.64 | 0.25 | 3.35 | 798.77 | 12.32 |
| 27.1 | 0.09 | 3.29 | 1430.5 | 22.06 |
| 27.53 | 0.22 | 3.24 | 1202.14 | 18.54 |
| 27.98 | 0.16 | 3.19 | 590.8 | 9.11 |
| 28.88 | 0.19 | 3.09 | 1133.54 | 17.48 |
| 29.9 | 0.11 | 2.99 | 853.62 | 13.17 |
| 30.36 | 0.16 | 2.94 | 783.25 | 12.08 |
| 31.46 | 0.5 | 2.84 | 627.32 | 9.68 |
| 32.49 | 0.19 | 2.76 | 527.16 | 8.13 |
| 33.23 | 0.22 | 2.7 | 361.84 | 5.58 |
| 34.08 | 0.38 | 2.63 | 145.91 | 2.25 |
| 34.95 | 0.19 | 2.57 | 83.56 | 1.29 |
| 35.72 | 0.22 | 2.51 | 307.27 | 4.74 |
| 36.38 | 0.22 | 2.47 | 210.95 | 3.25 |
| 36.88 | 0.19 | 2.44 | 201.41 | 3.11 |
| 37.55 | 0.25 | 2.4 | 356.35 | 5.5 |
| 38.51 | 0.5 | 2.34 | 136.41 | 2.1 |
| 39.39 | 0.31 | 2.29 | 203.08 | 3.13 |

### Example 24: AMG 397 Maleate Salt Form 1 (Family of Isostructural Solvates)

AMG 397 maleate salt form 1 (family of isostructural solvates) was formed by 1:1 (mol/mol, API/acid) salt reaction with maleic acid in acetone mixed at RT for 1 day. Family of isostructural solvates from acetone, MeCN, DCM, DMF/ACN, DMF/EtOH, and THF. The isolated solids were AMG 397 maleate salt form 1 (family of isostructural solvates), which was characterized as shown in the below tables.

TABLE 37
XRPD Data Table

| Pos. [°2θ] | FWHM [°2θ] | d-spacing [Å] | Height [cts] | Rel. Int. [%] |
|---|---|---|---|---|
| 9.55 | 0.07 | 9.26 | 3331.71 | 5.48 |
| 10.3 | 0.05 | 8.59 | 11492.74 | 18.91 |
| 10.44 | 0.07 | 8.47 | 18076.67 | 29.74 |
| 10.85 | 0.07 | 8.15 | 12455.23 | 20.49 |
| 11.09 | 0.1 | 7.98 | 591.17 | 0.97 |
| 12.02 | 0.08 | 7.37 | 17841.06 | 29.35 |
| 12.27 | 0.08 | 7.21 | 677.48 | 1.11 |
| 12.72 | 0.07 | 6.96 | 1682.3 | 2.77 |
| 12.95 | 0.07 | 6.84 | 351.74 | 0.58 |
| 13.75 | 0.08 | 6.44 | 12096.12 | 19.9 |
| 14.07 | 0.07 | 6.3 | 1715.56 | 2.82 |
| 14.99 | 0.07 | 5.91 | 1317.56 | 2.17 |
| 15.21 | 0.12 | 5.82 | 649.36 | 1.07 |
| 15.77 | 0.08 | 5.62 | 10585.86 | 17.42 |
| 16.4 | 0.07 | 5.41 | 1860.46 | 3.06 |
| 16.59 | 0.07 | 5.34 | 2770.25 | 4.56 |
| 17.43 | 0.08 | 5.09 | 1386.75 | 2.28 |
| 17.88 | 0.07 | 4.96 | 10091.3 | 16.6 |
| 18.21 | 0.08 | 4.87 | 60778.28 | 100 |
| 18.45 | 0.05 | 4.81 | 3032.63 | 4.99 |
| 18.61 | 0.1 | 4.77 | 3746.34 | 6.16 |
| 18.94 | 0.08 | 4.69 | 25805.84 | 42.46 |
| 19.17 | 0.08 | 4.63 | 6307.55 | 10.38 |
| 19.86 | 0.08 | 4.47 | 23552.9 | 38.75 |
| 20.09 | 0.07 | 4.42 | 2263.19 | 3.72 |
| 20.3 | 0.08 | 4.37 | 4093.9 | 6.74 |
| 20.7 | 0.08 | 4.29 | 1751.12 | 2.88 |
| 20.94 | 0.1 | 4.24 | 2001.12 | 3.29 |
| 21.18 | 0.08 | 4.2 | 1908.56 | 3.14 |
| 21.54 | 0.1 | 4.13 | 13328.61 | 21.93 |
| 21.8 | 0.1 | 4.08 | 4730.75 | 7.78 |
| 22.4 | 0.08 | 3.97 | 3218.02 | 5.29 |
| 22.62 | 0.07 | 3.93 | 1745.01 | 2.87 |
| 22.83 | 0.1 | 3.9 | 2084.5 | 3.43 |
| 23.42 | 0.1 | 3.8 | 3596.72 | 5.92 |
| 23.86 | 0.05 | 3.73 | 1080.04 | 1.78 |
| 23.98 | 0.05 | 3.71 | 1950.67 | 3.21 |
| 24.16 | 0.1 | 3.68 | 9166.45 | 15.08 |
| 24.36 | 0.07 | 3.65 | 3920.46 | 6.45 |
| 24.73 | 0.1 | 3.6 | 1107.13 | 1.82 |
| 25.05 | 0.07 | 3.55 | 481.57 | 0.79 |
| 25.63 | 0.1 | 3.48 | 2541.04 | 4.18 |
| 26.12 | 0.1 | 3.41 | 2069.54 | 3.41 |
| 26.84 | 0.12 | 3.32 | 802.39 | 1.32 |
| 27.19 | 0.1 | 3.28 | 100.38 | 0.17 |
| 27.62 | 0.08 | 3.23 | 5752.71 | 9.47 |
| 28.19 | 0.08 | 3.17 | 2572.4 | 4.23 |
| 28.6 | 0.08 | 3.12 | 1570.37 | 2.58 |
| 28.99 | 0.07 | 3.08 | 2169.17 | 3.57 |
| 29.15 | 0.07 | 3.06 | 1482 | 2.44 |
| 29.39 | 0.05 | 3.04 | 1129.86 | 1.86 |
| 29.56 | 0.08 | 3.02 | 3214.16 | 5.29 |
| 30.01 | 0.1 | 2.98 | 2694.99 | 4.43 |
| 30.23 | 0.05 | 2.96 | 1205.11 | 1.98 |
| 30.77 | 0.08 | 2.91 | 2824.77 | 4.65 |
| 31.41 | 0.07 | 2.85 | 833.61 | 1.37 |
| 31.67 | 0.06 | 2.82 | 1489.73 | 2.45 |
| 31.82 | 0.05 | 2.81 | 1646.57 | 2.71 |
| 32.02 | 0.08 | 2.79 | 2197.01 | 3.61 |
| 32.45 | 0.07 | 2.76 | 3357.1 | 5.52 |
| 32.76 | 0.07 | 2.73 | 920.13 | 1.51 |
| 32.99 | 0.08 | 2.71 | 867.96 | 1.43 |
| 33.15 | 0.12 | 2.7 | 1185.9 | 1.95 |
| 33.37 | 0.08 | 2.69 | 925.54 | 1.52 |
| 33.87 | 0.1 | 2.65 | 470.88 | 0.77 |
| 34.13 | 0.07 | 2.63 | 559.42 | 0.92 |
| 34.76 | 0.13 | 2.58 | 1350.34 | 2.22 |
| 35.16 | 0.08 | 2.55 | 1126.59 | 1.85 |
| 36.03 | 0.13 | 2.49 | 184.59 | 0.3 |
| 36.73 | 0.08 | 2.44 | 953 | 1.57 |
| 36.92 | 0.1 | 2.43 | 1415.13 | 2.33 |
| 37.56 | 0.07 | 2.39 | 581.07 | 0.96 |
| 37.79 | 0.08 | 2.38 | 821.66 | 1.35 |
| 38.31 | 0.05 | 2.35 | 1525.63 | 2.51 |
| 38.63 | 0.07 | 2.33 | 1147.22 | 1.89 |
| 39.23 | 0.07 | 2.3 | 723.71 | 1.19 |
| 39.74 | 0.1 | 2.27 | 421.61 | 0.69 |

### ¹H NMR Data

¹H NMR (400 MHZ, DMSO-d6) δ ppm 0.96 (br d, J=6.02 Hz, 2H) 1.22-1.54 (m, 4H) 1.54-1.71 (m, 2H) 1.71-1.88 (m, 3H) 1.91-2.13 (m, 2H) 2.15-2.37 (m, 2H) 2.37-2.48 (m, 1H) 2.52-2.65 (m, 1H) 2.65-2.86 (m, 2H) 2.88-3.12 (m, 2H) 3.12-3.28 (m, 4H) 3.58 (br d, J=13.96 Hz, 3H) 3.81-4.08 (m, 7H) 5.23-5.46 (m, 3H) 5.58 (br d, J=15.77 Hz, 2H) 6.02 (s, 2H) 6.76-6.95 (m, 2H) 6.95-7.08 (m, 2H) 7.08-7.15 (m, 1H) 7.18 (d, J=2.24 Hz, 1H) 7.27 (dd, J=8.52, 2.29 Hz, 2H) 7.66 (d, J=8.52 Hz, 1H) 8.97 (s, 2H) 11.88 (br s, 1H).

TABLE 38
X-ray Single Structure Data

| Crystal system | Primitive Orthorhombic | | |
|---|---|---|---|
| Space group(s) | P2₁2₁2₁ (19) | | |
| Extinction Symbol | P 2₁ 2₁ 2₁ | | |
| Chiral Contents? | Chiral | | |
| Unit cell dimensions | a = 9.631 | Å | α = 90° |
| | b = 17.210 | Å | β = 90° |
| | c = 29.703 | Å | γ = 90° |

| | |
|---|---|
| Volume | 4923.1 Å³ |

### Example 25: AMG 397 Maleate Salt Form 2

AMG 397 maleate salt form 2 was formed by 1:1 (mol/mol, API/acid) salt reaction with maleic acid in EtOH heat cycled to 60° C. for 1h. Alternatively, maleate form 2 was prepared by stressing Maleate Form 1 at 40° C./75% relative humidity for 10 days The isolated solids were AMG 397 maleate salt form 2, which was characterized as shown in the below tables.

TABLE 39
XRPD Data Table

| Pos. [°2θ] | FWHM [°2θ] | d-spacing [Å] | Height [cts] | Rel. Int. [%] |
|---|---|---|---|---|
| 6.1 | 0.1 | 14.48 | 186.71 | 0.91 |
| 9.78 | 0.08 | 9.05 | 2179.78 | 10.6 |
| 10.34 | 0.07 | 8.55 | 1881.51 | 9.15 |
| 10.63 | 0.07 | 8.32 | 9587.78 | 46.6 |
| 10.79 | 0.05 | 8.2 | 7002.02 | 34.04 |
| 11.09 | 0.13 | 7.98 | 5192.93 | 25.24 |
| 12.28 | 0.1 | 7.21 | 6620.09 | 32.18 |
| 13.06 | 0.08 | 6.78 | 730.17 | 3.55 |
| 13.31 | 0.08 | 6.65 | 695.73 | 3.38 |
| 13.92 | 0.07 | 6.36 | 3688.61 | 17.93 |
| 14.07 | 0.08 | 6.29 | 5278.09 | 25.66 |
| 14.26 | 0.05 | 6.21 | 675.21 | 3.28 |
| 15.22 | 0.1 | 5.82 | 6679.87 | 32.47 |
| 15.85 | 0.08 | 5.59 | 7835.58 | 38.09 |
| 16.19 | 0.07 | 5.47 | 715.17 | 3.48 |
| 16.72 | 0.1 | 5.3 | 9118.39 | 44.32 |
| 17.96 | 0.08 | 4.94 | 4744 | 23.06 |
| 18.4 | 0.07 | 4.82 | 5174.24 | 25.15 |
| 18.64 | 0.15 | 4.76 | 20572.58 | 100 |
| 19.16 | 0.08 | 4.63 | 4932.13 | 23.97 |
| 19.36 | 0.1 | 4.58 | 3661.76 | 17.8 |
| 19.63 | 0.1 | 4.52 | 1407.89 | 6.84 |
| 20.33 | 0.15 | 4.37 | 10361.75 | 50.37 |
| 20.79 | 0.1 | 4.27 | 2969.64 | 14.43 |
| 21.01 | 0.08 | 4.23 | 758.45 | 3.69 |
| 21.34 | 0.12 | 4.16 | 1078.63 | 5.24 |
| 21.68 | 0.08 | 4.1 | 1127.59 | 5.48 |
| 21.95 | 0.1 | 4.05 | 884.54 | 4.3 |
| 22.25 | 0.13 | 4 | 4552.79 | 22.13 |
| 22.76 | 0.1 | 3.91 | 919.9 | 4.47 |
| 23.01 | 0.1 | 3.87 | 2795.11 | 13.59 |
| 23.2 | 0.07 | 3.83 | 1018.29 | 4.95 |
| 23.63 | 0.12 | 3.76 | 3385.1 | 16.45 |
| 24.62 | 0.13 | 3.62 | 4284.79 | 20.83 |
| 25.18 | 0.12 | 3.54 | 225.28 | 1.1 |
| 25.97 | 0.13 | 3.43 | 890.34 | 4.33 |
| 26.24 | 0.12 | 3.4 | 1183.34 | 5.75 |
| 26.9 | 0.1 | 3.31 | 192.21 | 0.93 |
| 27.35 | 0.12 | 3.26 | 845.88 | 4.11 |
| 27.71 | 0.12 | 3.22 | 288.3 | 1.4 |
| 28.41 | 0.12 | 3.14 | 4229.41 | 20.56 |
| 28.76 | 0.15 | 3.1 | 823.82 | 4 |
| 29.27 | 0.12 | 3.05 | 1040.88 | 5.06 |
| 29.62 | 0.13 | 3.02 | 1327.33 | 6.45 |
| 30.2 | 0.08 | 2.96 | 774.54 | 3.76 |
| 30.73 | 0.12 | 2.91 | 662.57 | 3.22 |
| 31.51 | 0.07 | 2.84 | 1070.83 | 5.21 |
| 31.84 | 0.1 | 2.81 | 332.52 | 1.62 |
| 32.49 | 0.27 | 2.76 | 401.99 | 1.95 |
| 32.85 | 0.22 | 2.73 | 969.05 | 4.71 |
| 33.6 | 0.17 | 2.67 | 1196.04 | 5.81 |
| 34.16 | 0.2 | 2.63 | 352.93 | 1.72 |
| 34.67 | 0.13 | 2.59 | 298.39 | 1.45 |
| 34.96 | 0.13 | 2.57 | 163.7 | 0.8 |
| 35.4 | 0.17 | 2.54 | 370.17 | 1.8 |
| 35.86 | 0.17 | 2.5 | 289.4 | 1.41 |
| 36.45 | 0.13 | 2.47 | 44.18 | 0.21 |
| 36.91 | 0.12 | 2.44 | 232 | 1.13 |
| 37.33 | 0.2 | 2.41 | 263.58 | 1.28 |
| 38.17 | 0.13 | 2.36 | 156.75 | 0.76 |
| 38.89 | 0.13 | 2.32 | 667.43 | 3.24 |
| 39.28 | 0.12 | 2.29 | 839.77 | 4.08 |

### ¹H NMR Data

¹H NMR (400 MHZ, DMSO-d6) δ ppm 0.96 (br d, J=5.97 Hz, 3H) 1.23-1.52 (m, 4H) 1.54-1.70 (m, 2H) 1.70-1.90 (m, 3H) 1.91-2.13 (m, 2H) 2.14-2.37 (m, 2H) 2.37-2.47 (m, 1H) 2.53-2.63 (m, 1H) 2.64-2.86 (m, 2H) 2.87-3.07 (m, 2H) 3.07-3.27 (m, 4H) 3.58 (br d, J=14.07 Hz, 1H) 3.73-3.95 (m, 2H) 3.95-4.24 (m, 2H) 5.35 (br d, J=15.77 Hz, 1H) 5.58 (br d, J=15.98 Hz, 1H) 6.02 (s, 1H) 6.79-6.95 (m, 1H) 7.01 (dd, J=8.10, 1.70 Hz, 1H) 7.07-7.15 (m, 1H) 7.18 (d, J=2.13 Hz, 1H) 7.27 (dd, J=8.52, 2.34 Hz, 1H) 7.66 (d, J=8.52 Hz, 1H) 8.97 (br s, 1H) 11.87 (br s, 2H).

TABLE 40
X-ray Single Structure Data

| Crystal system | Primitive Orthorhombic | | |
|---|---|---|---|
| Space group(s) | P2₁2₁2₁ (19) | | |
| Extinction Symbol | P 2₁ 2₁ 2₁ | | |
| Chiral Contents? | Chiral | | |
| Unit cell dimensions | a = 9.514 | Å | α = 900 |
| | b = 17.077 | Å | β = 90° |
| | c = 28.864 | Å | γ = 90° |

| | |
|---|---|
| Volume | 4689.4 Å³ |

### Example 26: AMG 397 Malonate Salt Form 1

AMG 397 malonate salt form 1 was formed by 1:1 (mol/mol, API/acid) salt reaction with malonic acid in MeCN mixed at room temperature for 1 day. The isolated solids were AMG 397 malonate salt form 1, which was characterized as shown in the below tables.

TABLE 41
XRPD Data Table

| Pos. [°2θ] | FWHM [°2θ] | d-spacing [Å] | Height [cts] | Rel. Int. [%] |
|---|---|---|---|---|
| 5.94 | 0.07 | 14.89 | 2219.16 | 7.07 |
| 9.8 | 0.07 | 9.03 | 2214.32 | 7.05 |
| 10.34 | 0.07 | 8.56 | 14035.5 | 44.69 |
| 10.68 | 0.12 | 8.28 | 10474.76 | 33.36 |
| 11.07 | 0.08 | 8 | 17909.01 | 57.03 |
| 12.2 | 0.08 | 7.25 | 31403.38 | 100 |
| 12.86 | 0.07 | 6.89 | 3218.38 | 10.25 |
| 13.6 | 0.05 | 6.51 | 854.07 | 2.72 |
| 13.86 | 0.07 | 6.39 | 11327.76 | 36.07 |
| 13.97 | 0.05 | 6.34 | 8119.35 | 25.86 |
| 14.26 | 0.07 | 6.21 | 2293.69 | 7.3 |
| 15.15 | 0.08 | 5.85 | 4124.14 | 13.13 |
| 15.6 | 0.07 | 5.68 | 2909.93 | 9.27 |
| 15.82 | 0.08 | 5.6 | 10784.36 | 34.34 |
| 16.52 | 0.07 | 5.37 | 7097.36 | 22.6 |
| 16.63 | 0.05 | 5.33 | 4236.69 | 13.49 |
| 17.46 | 0.1 | 5.08 | 5596.24 | 17.82 |
| 17.89 | 0.08 | 4.96 | 13150.95 | 41.88 |
| 18.28 | 0.08 | 4.85 | 24044.09 | 76.57 |
| 18.43 | 0.05 | 4.81 | 7484.46 | 23.83 |
| 18.77 | 0.1 | 4.73 | 30113.9 | 95.89 |
| 19.1 | 0.1 | 4.65 | 17714.2 | 56.41 |
| 19.47 | 0.1 | 4.56 | 9755.13 | 31.06 |
| 19.69 | 0.1 | 4.51 | 6429.98 | 20.48 |
| 20.36 | 0.1 | 4.36 | 26408.23 | 84.09 |
| 20.76 | 0.08 | 4.28 | 3990.63 | 12.71 |
| 20.98 | 0.08 | 4.24 | 2382.84 | 7.59 |
| 21.45 | 0.05 | 4.14 | 5313.23 | 16.92 |
| 21.59 | 0.08 | 4.11 | 12268.19 | 39.07 |
| 21.68 | 0.07 | 4.1 | 9721.74 | 30.96 |
| 22.28 | 0.1 | 3.99 | 5738.95 | 18.27 |
| 22.58 | 0.05 | 3.94 | 2550.46 | 8.12 |
| 22.8 | 0.1 | 3.9 | 6915.96 | 22.02 |
| 23.01 | 0.07 | 3.86 | 2718.58 | 8.66 |
| 23.3 | 0.07 | 3.82 | 1923.22 | 6.12 |
| 23.62 | 0.12 | 3.77 | 5880.19 | 18.72 |
| 24.2 | 0.07 | 3.68 | 1431.62 | 4.56 |
| 24.5 | 0.13 | 3.63 | 11557.53 | 36.8 |
| 25 | 0.07 | 3.56 | 1780.85 | 5.67 |
| 25.17 | 0.08 | 3.54 | 2040.8 | 6.5 |
| 25.44 | 0.07 | 3.5 | 1416.64 | 4.51 |
| 25.73 | 0.08 | 3.46 | 2639.03 | 8.4 |
| 25.93 | 0.07 | 3.44 | 2391 | 7.6 |
| 26.2 | 0.08 | 3.4 | 2975.97 | 9.48 |
| 26.42 | 0.08 | 3.37 | 1312.42 | 4.18 |
| 27.2 | 0.12 | 3.28 | 2438.31 | 7.76 |
| 27.54 | 0.1 | 3.24 | 2151.67 | 6.85 |
| 27.87 | 0.1 | 3.2 | 1572.37 | 5.01 |
| 28.12 | 0.1 | 3.17 | 1861.31 | 5.93 |
| 28.36 | 0.1 | 3.14 | 4025.3 | 12.82 |
| 28.46 | 0.12 | 3.14 | 4925.85 | 15.69 |
| 29.14 | 0.12 | 3.06 | 2583.12 | 8.23 |
| 29.41 | 0.12 | 3.03 | 2635.07 | 8.39 |
| 29.51 | 0.07 | 3.03 | 3016.41 | 9.61 |
| 30.14 | 0.1 | 2.97 | 2265.87 | 7.22 |
| 30.37 | 0.13 | 2.94 | 3870.58 | 12.33 |
| 30.78 | 0.13 | 2.91 | 2728.57 | 8.69 |
| 31.22 | 0.1 | 2.86 | 666.52 | 2.12 |
| 31.52 | 0.12 | 2.84 | 3392.77 | 10.8 |
| 31.93 | 0.1 | 2.8 | 928.48 | 2.96 |
| 32.43 | 0.13 | 2.76 | 1484.41 | 4.73 |
| 32.84 | 0.1 | 2.72 | 3256.48 | 10.37 |
| 32.93 | 0.06 | 2.72 | 3290.36 | 10.48 |
| 33.46 | 0.12 | 2.68 | 560.9 | 1.79 |
| 34.02 | 0.16 | 2.63 | 1754.14 | 5.59 |
| 34.46 | 0.14 | 2.6 | 846.19 | 2.69 |
| 34.8 | 0.06 | 2.58 | 681.02 | 2.17 |
| 35.5 | 0.12 | 2.53 | 761.17 | 2.42 |
| 35.87 | 0.14 | 2.5 | 810.1 | 2.58 |
| 36.15 | 0.12 | 2.48 | 356.1 | 1.13 |
| 36.52 | 0.12 | 2.46 | 294.67 | 0.94 |
| 37.03 | 0.1 | 2.43 | 913.19 | 2.91 |
| 37.28 | 0.08 | 2.41 | 943.49 | 3 |
| 38.03 | 0.29 | 2.36 | 816.92 | 2.6 |
| 38.51 | 0.16 | 2.34 | 635.78 | 2.02 |
| 38.94 | 0.33 | 2.31 | 812.21 | 2.59 |
| 39.32 | 0.24 | 2.29 | 628.93 | 2 |

### ¹H NMR Data

¹H NMR (400 MHZ, DMSO-d6) δ ppm 0.94 (br d, J=6.13 Hz, 2H) 1.20-1.30 (m, 3H) 1.30-1.51 (m, 3H) 1.52-1.58 (m, 1H) 1.59-1.87 (m, 6H) 1.92-2.13 (m, 3H) 2.19 (br d, J=12.95 Hz, 2H) 2.25-2.36 (m, 1H) 2.36-2.48 (m, 2H) 2.54-2.66 (m, 1H) 2.66-2.87 (m, 4H) 2.87-3.04 (m, 3H) 3.05-3.23 (m, 4H) 3.49-3.71 (m, 4H) 3.72-3.92 (m, 4H) 3.92-4.13 (m, 4H) 5.24-5.49 (m, 2H) 5.59 (br d, J=15.93 Hz, 2H) 6.79-6.94 (m, 2H) 6.94-7.10 (m, 2H) 7.10-7.22 (m, 2H) 7.27 (dd, J=8.50, 2.26 Hz, 2H) 7.66 (d, J=8.52 Hz, 1H).

TABLE 42
X-ray Single Structure Data

| Crystal system | Primitive Orthorhombic | | |
|---|---|---|---|
| Space group(s) | P2₁2₁2₁ (19) | | |
| Extinction Symbol | P 2₁ 2₁ 2₁ | | |
| Chiral Contents? | Chiral | | |
| Unit cell dimensions | a = 9.454 | Å | α = 90° |
| | b = 17.101 | Å | β = 90° |
| | c = 30.078 | Å | γ = 90° |

| | |
|---|---|
| Volume | 4862.7 Å³ |

### Example 27: AMG 397 Malonate Salt Form 2

AMG 397 malonate salt form 2 was formed by stressing Malonate Form 1 at 40° C./75% relative humidity for 10 days. The isolated solids were AMG 397 malonate salt form 2, which was characterized as shown in the below tables.

TABLE 43
XRPD Data Table

| Pos. [°2θ] | FWHM [°2θ] | d-spacing [Å] | Height [cts] | Rel. Int. [%] |
|---|---|---|---|---|
| 6.1 | 0.05 | 14.49 | 537.78 | 1.93 |
| 8.06 | 0.07 | 10.97 | 446.68 | 1.6 |
| 9.7 | 0.07 | 9.12 | 3837.09 | 13.76 |
| 10.6 | 0.1 | 8.35 | 16570.99 | 59.41 |
| 10.96 | 0.08 | 8.07 | 12146.79 | 43.55 |
| 11.06 | 0.05 | 8 | 9635.07 | 34.55 |
| 12.25 | 0.08 | 7.23 | 7239.79 | 25.96 |
| 13.01 | 0.07 | 6.81 | 2198.65 | 7.88 |
| 13.33 | 0.08 | 6.64 | 3321.23 | 11.91 |
| 14.03 | 0.1 | 6.31 | 11185.85 | 40.11 |
| 14.33 | 0.07 | 6.18 | 509.36 | 1.83 |
| 15.29 | 0.08 | 5.79 | 9823.05 | 35.22 |
| 16.12 | 0.1 | 5.5 | 7145.05 | 25.62 |
| 16.76 | 0.1 | 5.29 | 10095.15 | 36.2 |
| 16.98 | 0.08 | 5.22 | 6613.11 | 23.71 |
| 17.9 | 0.1 | 4.95 | 12718.57 | 45.6 |
| 18.46 | 0.12 | 4.81 | 27890.91 | 100 |
| 18.63 | 0.07 | 4.76 | 11125.84 | 39.89 |
| 19.2 | 0.08 | 4.62 | 5077.68 | 18.21 |
| 19.35 | 0.08 | 4.59 | 6677.32 | 23.94 |
| 20.18 | 0.12 | 4.4 | 13955.76 | 50.04 |
| 20.67 | 0.1 | 4.3 | 2317.47 | 8.31 |
| 20.81 | 0.1 | 4.27 | 1798.48 | 6.45 |
| 21.15 | 0.08 | 4.2 | 1545.77 | 5.54 |
| 21.31 | 0.12 | 4.17 | 2947.6 | 10.57 |
| 22.18 | 0.13 | 4.01 | 7634.46 | 27.37 |
| 22.85 | 0.12 | 3.89 | 954.63 | 3.42 |
| 23.19 | 0.1 | 3.84 | 2874.46 | 10.31 |
| 23.33 | 0.1 | 3.81 | 3565.53 | 12.78 |
| 23.92 | 0.12 | 3.72 | 3557.32 | 12.75 |
| 24.64 | 0.15 | 3.61 | 5307.56 | 19.03 |
| 24.91 | 0.12 | 3.57 | 2238.37 | 8.03 |
| 25.78 | 0.12 | 3.46 | 188.68 | 0.68 |
| 26.21 | 0.13 | 3.4 | 1890.53 | 6.78 |
| 26.72 | 0.15 | 3.34 | 1065.95 | 3.82 |
| 27.38 | 0.13 | 3.26 | 699.36 | 2.51 |
| 28.05 | 0.13 | 3.18 | 1861.32 | 6.67 |
| 28.43 | 0.12 | 3.14 | 1196.79 | 4.29 |
| 28.82 | 0.12 | 3.1 | 3717.27 | 13.33 |
| 29.05 | 0.13 | 3.07 | 2466.15 | 8.84 |
| 29.43 | 0.07 | 3.03 | 881.38 | 3.16 |
| 29.82 | 0.27 | 3 | 1276.72 | 4.58 |
| 30.03 | 0.13 | 2.98 | 882.03 | 3.16 |
| 30.51 | 0.12 | 2.93 | 672.57 | 2.41 |
| 30.85 | 0.13 | 2.9 | 995.66 | 3.57 |
| 31.27 | 0.08 | 2.86 | 1127.97 | 4.04 |
| 31.96 | 0.07 | 2.8 | 495.37 | 1.78 |
| 32.2 | 0.12 | 2.78 | 848.01 | 3.04 |
| 32.81 | 0.1 | 2.73 | 736.56 | 2.64 |
| 33.56 | 0.13 | 2.67 | 660.76 | 2.37 |
| 33.9 | 0.17 | 2.64 | 1751.73 | 6.28 |
| 34.64 | 0.12 | 2.59 | 441.43 | 1.58 |
| 34.94 | 0.12 | 2.57 | 650.39 | 2.33 |
| 35.41 | 0.08 | 2.53 | 986.18 | 3.54 |
| 35.82 | 0.17 | 2.51 | 377.12 | 1.35 |
| 37.35 | 0.27 | 2.41 | 553.7 | 1.99 |
| 37.8 | 0.23 | 2.38 | 620.19 | 2.22 |
| 38.37 | 0.13 | 2.35 | 273.54 | 0.98 |
| 39 | 0.2 | 2.31 | 876.68 | 3.14 |
| 39.36 | 0.13 | 2.29 | 849.63 | 3.05 |

### ¹H NMR Data

¹H NMR (400 MHZ, DMSO-d6) δ ppm 0.94 (br d, J=5.97 Hz, 2H) 1.20-1.30 (m, 2H) 1.30-1.51 (m, 2H) 1.59-1.87 (m, 5H) 1.92-2.12 (m, 2H) 2.19 (br d, J=13.00 Hz, 2H) 2.25-2.48 (m, 2H) 2.54-2.65 (m, 1H) 2.66-2.86 (m, 4H) 2.87-3.04 (m, 3H) 3.05-3.23 (m, 4H) 3.57 (br d, J=14.49 Hz, 7H) 3.86 (br d, J=14.49 Hz, 5H) 3.92-4.21 (m, 5H) 5.23-5.49 (m, 3H) 5.59 (br d, J=15.77 Hz, 3H) 6.86 (br d, J=8.10 Hz, 2H) 7.00 (dd, J=8.10, 1.70 Hz, 2H) 7.08-7.22 (m, 3H) 7.27 (dd, J=8.52, 2.34 Hz, 2H) 7.66 (d, J=8.52 Hz, 1H).

TABLE 44
X-ray Single Structure Data

| Crystal system | Primitive Orthorhombic | | |
|---|---|---|---|
| Space group(s) | P2₁2₁2₁ (19) | | |
| Extinction Symbol | P 2₁ 2₁ 2₁ | | |
| Chiral Contents? | Chiral | | |
| Unit cell dimensions | a = 9.601 | Å | α = 90° |
| | b = 16.782 | Å | β = 90° |
| | c = 28.910 | Å | γ = 90° |

| | |
|---|---|
| Volume | 4658.1 Å³ |

### Example 28: AMG 397 Tartrate Salt Form 1 (Family of Isostructural Solvates)

AMG 397 tartrates salt form 1 (family of isostructural solvates) was formed by 1:1 (mol/mol, API/acid) salt reaction with tartaric acid in acetone mixed at room temperature for 3 days. Family of isostructural solvates from acetone, MeCN, DCM, EtOH, MeOH and water. The isolated solids were AMG 397 tartrate salt form 1 which was characterized as shown in the below tables.

TABLE 45
XRPD Data Table

| Pos. [°2θ] | FWHM [°2θ] | d-spacing [Å] | Height [cts] | Rel. Int. [%] |
|---|---|---|---|---|
| 2.34 | 0.94 | 37.8 | 161.24 | 1.29 |
| 5.91 | 0.05 | 14.94 | 1282.55 | 10.27 |
| 9.7 | 0.07 | 9.12 | 2132.33 | 17.07 |
| 10.3 | 0.05 | 8.59 | 1572.43 | 12.59 |
| 10.59 | 0.07 | 8.35 | 4900.35 | 39.23 |
| 10.97 | 0.1 | 8.07 | 4363.84 | 34.93 |
| 12.11 | 0.08 | 7.31 | 7138.04 | 57.14 |
| 12.77 | 0.08 | 6.93 | 3662.09 | 29.31 |
| 13.77 | 0.08 | 6.43 | 5660.07 | 45.31 |
| 15.07 | 0.08 | 5.88 | 2986.02 | 23.9 |
| 15.43 | 0.54 | 5.74 | 1610.33 | 12.89 |
| 15.76 | 0.07 | 5.62 | 5259.72 | 42.1 |
| 16.43 | 0.07 | 5.4 | 2641.17 | 21.14 |
| 16.57 | 0.05 | 5.35 | 2880.12 | 23.05 |
| 17.36 | 0.08 | 5.11 | 5916.95 | 47.36 |
| 17.83 | 0.08 | 4.98 | 7014.21 | 56.15 |
| 18.19 | 0.1 | 4.88 | 12492.75 | 100 |
| 18.56 | 0.08 | 4.78 | 11577.92 | 92.68 |
| 19.01 | 0.08 | 4.67 | 7542.45 | 60.37 |
| 19.28 | 0.08 | 4.6 | 4711.48 | 37.71 |
| 19.49 | 0.05 | 4.56 | 3738.7 | 29.93 |
| 20.16 | 0.1 | 4.41 | 10288.3 | 82.35 |
| 20.56 | 0.13 | 4.32 | 3604.57 | 28.85 |
| 21.53 | 0.1 | 4.13 | 6753.62 | 54.06 |
| 22.08 | 0.1 | 4.03 | 3338.14 | 26.72 |
| 22.61 | 0.1 | 3.93 | 3977.43 | 31.84 |
| 22.9 | 0.07 | 3.88 | 2492.68 | 19.95 |
| 23.49 | 0.12 | 3.79 | 2782.99 | 22.28 |
| 24.39 | 0.12 | 3.65 | 5223.11 | 41.81 |
| 24.93 | 0.2 | 3.57 | 1589.73 | 12.73 |
| 25.35 | 0.1 | 3.51 | 1254.41 | 10.04 |
| 25.65 | 0.1 | 3.47 | 1562.69 | 12.51 |
| 26.13 | 0.08 | 3.41 | 1622.83 | 12.99 |
| 27.01 | 0.12 | 3.3 | 1044.78 | 8.36 |
| 27.43 | 0.08 | 3.25 | 1241.8 | 9.94 |
| 28.15 | 0.08 | 3.17 | 1547.23 | 12.39 |
| 28.23 | 0.05 | 3.16 | 1689.27 | 13.52 |
| 28.65 | 0.13 | 3.12 | 884.49 | 7.08 |
| 29.02 | 0.08 | 3.08 | 1551.32 | 12.42 |
| 29.43 | 0.13 | 3.04 | 883.7 | 7.07 |
| 30.08 | 0.12 | 2.97 | 1375.55 | 11.01 |
| 30.48 | 0.13 | 2.93 | 989.96 | 7.92 |
| 31.23 | 0.1 | 2.86 | 758.2 | 6.07 |
| 32.28 | 0.2 | 2.77 | 567.15 | 4.54 |
| 32.66 | 0.05 | 2.74 | 1017.68 | 8.15 |
| 32.97 | 0.1 | 2.72 | 616.1 | 4.93 |
| 33.92 | 0.1 | 2.64 | 454.82 | 3.64 |
| 34.33 | 0.13 | 2.61 | 523.01 | 4.19 |
| 35.58 | 0.13 | 2.52 | 325.63 | 2.61 |
| 36.88 | 0.13 | 2.44 | 386.9 | 3.1 |
| 37.73 | 0.2 | 2.38 | 314.96 | 2.52 |
| 38.45 | 0.33 | 2.34 | 274.94 | 2.2 |
| 39.11 | 0.2 | 2.3 | 101.18 | 0.81 |

### ¹H NMR Data

¹H NMR (400 MHZ, DMSO-d6) δ ppm 0.93 (br d, J=6.55 Hz, 3H) 1.21 (br d, J=6.70 Hz, 6H) 1.63 (br s, 4H) 1.74-1.89 (m, 2H) 1.90-2.04 (m, 1H) 2.09 (s, 9H) 2.12-2.25 (m, 2H) 2.28-2.48 (m, 3H) 2.54-2.62 (m, 1H) 2.63-2.79 (m, 2H) 2.87-3.00 (m, 2H) 3.02-3.22 (m, 3H) 3.49-3.62 (m, 1H) 3.72-3.91 (m, 2H) 3.99 (br d, J=9.39 Hz, 5H) 4.19 (s, 6H) 5.34-5.46 (m, 1H) 5.54-5.66 (m, 1H) 6.79-6.87 (m, 1H) 6.99 (br d, J=1.71 Hz, 2H) 7.07-7.31 (m, 6H) 7.67 (d, J=8.56 Hz, 1H).

TABLE 46
X-ray Single Structure Data

| Crystal system | Primitive Orthorhombic | | |
|---|---|---|---|
| Space group(s) | P2₁2₁2₁ (19) | | |
| Extinction Symbol | P 2₁ 2₁ 2₁ | | |
| Chiral Contents? | Chiral | | |
| Unit cell dimensions | a = 9.550 | Å | α = 90° |
| | b = 17.152 | Å | β = 90° |
| | c = 30.221 | Å | γ = 90° |

| | |
|---|---|
| Volume | 4950.1 Å³ |

### Example 29: AMG 397 Tris(Hydroxymethyl)Aminomethane (Tris) Salt Form 1 (Acetone Solvate)

AMG 397 tris(hydroxymethyl)aminomethane (tris) salt form 1 (acetone solvate) was formed by 1:1 (mol/mol, API/base) salt reaction with tromethamine in acetone mixed at room temperature for 8 days. The isolated solids were AMG 397 tris(hydroxymethyl)aminomethane (tris) salt form 1 (acetone solvate) which was characterized as shown in the below tables.

TABLE 47
XRPD Data Table

| Pos. [°2θ] | FWHM [°2θ] | d-spacing [Å] | Height [cts] | Rel. Int. [%] |
|---|---|---|---|---|
| 6.07 | 0.05 | 14.57 | 12731.79 | 24.25 |
| 7.7 | 0.05 | 11.48 | 2751.72 | 5.24 |
| 8.47 | 0.05 | 10.43 | 1919.33 | 3.66 |
| 8.77 | 0.07 | 10.08 | 380.99 | 0.73 |
| 9.98 | 0.07 | 8.86 | 49525.67 | 94.34 |
| 10.59 | 0.07 | 8.36 | 3854.87 | 7.34 |
| 10.98 | 0.08 | 8.06 | 486.51 | 0.93 |
| 12.17 | 0.05 | 7.27 | 767.61 | 1.46 |
| 12.69 | 0.07 | 6.98 | 15984.66 | 30.45 |
| 13.73 | 0.07 | 6.45 | 4199.86 | 8 |
| 14.08 | 0.08 | 6.29 | 16643.7 | 31.7 |
| 14.86 | 0.07 | 5.96 | 12681.02 | 24.16 |
| 15.05 | 0.05 | 5.89 | 3186.81 | 6.07 |
| 15.28 | 0.07 | 5.8 | 8414.08 | 16.03 |
| 15.45 | 0.07 | 5.74 | 3904.33 | 7.44 |
| 16.04 | 0.07 | 5.53 | 6381.69 | 12.16 |
| 16.35 | 0.08 | 5.42 | 5013.62 | 9.55 |
| 16.82 | 0.08 | 5.27 | 29996.99 | 57.14 |
| 17.26 | 0.08 | 5.14 | 14865.68 | 28.32 |
| 17.61 | 0.07 | 5.04 | 6015.33 | 11.46 |
| 18.03 | 0.05 | 4.92 | 9597.33 | 18.28 |
| 18.17 | 0.08 | 4.88 | 23150.43 | 44.1 |
| 19.01 | 0.05 | 4.67 | 8296.28 | 15.8 |
| 19.14 | 0.07 | 4.64 | 13037.82 | 24.84 |
| 19.38 | 0.1 | 4.58 | 9739.38 | 18.55 |
| 19.74 | 0.05 | 4.5 | 4300.58 | 8.19 |
| 20.04 | 0.1 | 4.43 | 52495.81 | 100 |
| 20.61 | 0.08 | 4.31 | 11094.07 | 21.13 |
| 20.91 | 0.08 | 4.25 | 5219.75 | 9.94 |
| 21.28 | 0.05 | 4.18 | 3263.27 | 6.22 |
| 22.08 | 0.15 | 4.03 | 12685.76 | 24.17 |
| 22.45 | 0.07 | 3.96 | 6577.66 | 12.53 |
| 22.69 | 0.08 | 3.92 | 7276.25 | 13.86 |
| 22.93 | 0.1 | 3.88 | 6346.73 | 12.09 |
| 23.35 | 0.07 | 3.81 | 2423.74 | 4.62 |
| 24.16 | 0.08 | 3.68 | 4316.72 | 8.22 |
| 24.34 | 0.08 | 3.66 | 3573.79 | 6.81 |
| 24.66 | 0.17 | 3.61 | 1902.08 | 3.62 |
| 25.2 | 0.08 | 3.53 | 3530.27 | 6.72 |
| 25.52 | 0.05 | 3.49 | 2554.45 | 4.87 |
| 25.66 | 0.08 | 3.47 | 3385.53 | 6.45 |
| 25.87 | 0.08 | 3.44 | 2508.23 | 4.78 |
| 26.27 | 0.1 | 3.39 | 7924.32 | 15.1 |
| 26.37 | 0.06 | 3.39 | 7092.61 | 13.51 |
| 26.59 | 0.08 | 3.35 | 3226.8 | 6.15 |
| 26.72 | 0.08 | 3.33 | 2722.97 | 5.19 |
| 26.94 | 0.16 | 3.31 | 4487.71 | 8.55 |
| 27.88 | 0.12 | 3.2 | 3042 | 5.79 |
| 28.37 | 0.1 | 3.14 | 3176.69 | 6.05 |
| 28.65 | 0.08 | 3.11 | 1431.49 | 2.73 |
| 29.03 | 0.12 | 3.07 | 1942.04 | 3.7 |
| 29.35 | 0.12 | 3.04 | 1382.02 | 2.63 |
| 29.8 | 0.16 | 3 | 1498.32 | 2.85 |
| 29.99 | 0.12 | 2.98 | 1266.35 | 2.41 |
| 30.29 | 0.12 | 2.95 | 2394.64 | 4.56 |
| 30.56 | 0.16 | 2.92 | 2262.84 | 4.31 |
| 30.84 | 0.12 | 2.9 | 1562.24 | 2.98 |
| 31.2 | 0.12 | 2.86 | 1476.86 | 2.81 |
| 31.44 | 0.12 | 2.84 | 1097.05 | 2.09 |
| 31.9 | 0.12 | 2.8 | 520.32 | 0.99 |
| 32.21 | 0.24 | 2.78 | 500.78 | 0.95 |
| 33.05 | 0.12 | 2.71 | 966.53 | 1.84 |
| 33.3 | 0.24 | 2.69 | 907.64 | 1.73 |
| 33.76 | 0.1 | 2.65 | 1728.06 | 3.29 |
| 34.02 | 0.14 | 2.63 | 2909.4 | 5.54 |
| 34.26 | 0.06 | 2.62 | 1530.61 | 2.92 |
| 35.04 | 0.18 | 2.56 | 924.63 | 1.76 |
| 35.96 | 0.24 | 2.5 | 861.19 | 1.64 |
| 36.49 | 0.14 | 2.46 | 801.09 | 1.53 |
| 36.83 | 0.1 | 2.44 | 867.76 | 1.65 |
| 37.16 | 0.12 | 2.42 | 551.08 | 1.05 |
| 37.52 | 0.18 | 2.4 | 838.35 | 1.6 |
| 38.12 | 0.12 | 2.36 | 376.41 | 0.72 |
| 38.46 | 0.14 | 2.34 | 830.24 | 1.58 |
| 38.83 | 0.08 | 2.32 | 1035.39 | 1.97 |
| 39.35 | 0.12 | 2.29 | 283.57 | 0.54 |

### ¹H NMR Data

¹H NMR (400 MHZ, DMSO-d6) δ ppm 0.79-0.97 (m, 5H) 0.97-1.16 (m, 5H) 1.16-1.40 (m, 5H) 1.57 (br s, 6H) 1.71-1.79 (m, 1H) 1.81-2.04 (m, 6H) 2.09 (s, 10H) 2.12-2.28 (m, 3H) 2.39 (br d, J=14.38 Hz, 2H) 2.54-2.63 (m, 2H) 2.64-2.95 (m, 4H) 2.97-3.09 (m, 1H) 3.15-3.30 (m, 4H) 3.34-3.48 (m, 13H) 3.53 (br d, J=13.55 Hz, 9H) 3.94 (br d, J=10.56 Hz, 10H) 5.06 (br s, 10H) 5.43-5.56 (m, 1H) 5.59-5.73 (m, 1H) 6.62-6.85 (m, 5H) 6.85-7.08 (m, 6H) 7.08-7.19 (m, 4H) 7.19-7.39 (m, 7H) 7.69 (d, J=8.56 Hz, 1H).

TABLE 48
X-ray Single Structure Data

| Crystal system | Primitive Monoclinic | | |
|---|---|---|---|
| Space group(s) | P2₁ (4) | | |
| Extinction Symbol | P 1 2₁ 1 | | |
| Chiral Contents? | Chiral | | |
| Unit cell dimensions | a = 11.877 | Å | α = 90° |
| | b = 13.941 | Å | β = 105.09° |
| | c = 15.065 | Å | γ = 90° |

| | |
|---|---|
| Volume | 2408.6 Å³ |

### Example 30: AMG 397 Iodide Salt Form 1

AMG 397 iodide salt form 1 was formed by 1:1 (mol/mol, API/acid) salt reaction with hydriodic acid in EtOH at room temperature by precipitation. The isolated solids were AMG 397 iodide salt form 1 which was characterized as shown in the below tables.

TABLE 49
XRPD Data Table

| Pos. [°2θ] | FWHM [°2θ] | d-spacing [Å] | Height [cts] | Rel. Int. [%] |
|---|---|---|---|---|
| 6.22 | 0.07 | 14.22 | 4447.25 | 10.23 |
| 6.33 | 0.05 | 13.97 | 2657.96 | 6.11 |
| 8.29 | 0.08 | 10.66 | 11959.1 | 27.5 |
| 9.57 | 0.07 | 9.24 | 3228.27 | 7.42 |
| 10.59 | 0.07 | 8.35 | 4670.78 | 10.74 |
| 10.8 | 0.07 | 8.19 | 7284.08 | 16.75 |
| 10.98 | 0.08 | 8.06 | 12419.75 | 28.56 |
| 11.41 | 0.08 | 7.75 | 1360.58 | 3.13 |
| 12.4 | 0.08 | 7.14 | 5106.84 | 11.74 |
| 12.62 | 0.08 | 7.02 | 3711.81 | 8.54 |
| 12.98 | 0.1 | 6.82 | 7737.14 | 17.79 |
| 13.56 | 0.08 | 6.53 | 3450.3 | 7.93 |
| 14.1 | 0.08 | 6.28 | 4437.83 | 10.2 |
| 14.4 | 0.08 | 6.15 | 2144.03 | 4.93 |
| 14.58 | 0.07 | 6.08 | 934.23 | 2.15 |
| 15.54 | 0.1 | 5.7 | 8443.48 | 19.42 |
| 17.02 | 0.12 | 5.21 | 25184.28 | 57.91 |
| 17.62 | 0.08 | 5.03 | 6813.67 | 15.67 |
| 17.97 | 0.08 | 4.94 | 43488.38 | 100 |
| 18.14 | 0.07 | 4.89 | 26516.14 | 60.97 |
| 18.63 | 0.08 | 4.76 | 17728.5 | 40.77 |
| 18.81 | 0.07 | 4.72 | 11877.94 | 27.31 |
| 19.07 | 0.1 | 4.65 | 10975.89 | 25.24 |
| 19.95 | 0.12 | 4.45 | 13703.06 | 31.51 |
| 20.4 | 0.08 | 4.35 | 830.71 | 1.91 |
| 20.73 | 0.12 | 4.28 | 1058.56 | 2.43 |
| 21.03 | 0.08 | 4.23 | 3140.8 | 7.22 |
| 21.13 | 0.1 | 4.2 | 3342.86 | 7.69 |
| 21.65 | 0.08 | 4.11 | 1242.79 | 2.86 |
| 22.13 | 0.18 | 4.02 | 9143.3 | 21.02 |
| 22.45 | 0.1 | 3.96 | 8560.76 | 19.69 |
| 22.69 | 0.1 | 3.92 | 3269.41 | 7.52 |
| 23.2 | 0.1 | 3.83 | 3193.85 | 7.34 |
| 23.51 | 0.12 | 3.78 | 12298.61 | 28.28 |
| 23.87 | 0.1 | 3.73 | 2937.48 | 6.75 |
| 24.05 | 0.08 | 3.7 | 4489.26 | 10.32 |
| 24.26 | 0.07 | 3.67 | 2579.49 | 5.93 |
| 24.65 | 0.13 | 3.61 | 10204.98 | 23.47 |
| 25.65 | 0.13 | 3.47 | 3246.89 | 7.47 |
| 26.08 | 0.12 | 3.42 | 3662.55 | 8.42 |
| 26.38 | 0.08 | 3.38 | 1688.09 | 3.88 |
| 26.67 | 0.08 | 3.34 | 1598.47 | 3.68 |
| 27.12 | 0.17 | 3.29 | 3043.17 | 7 |
| 28.34 | 0.1 | 3.15 | 2623.16 | 6.03 |
| 28.57 | 0.07 | 3.12 | 4543.77 | 10.45 |
| 28.78 | 0.05 | 3.1 | 5329.56 | 12.26 |
| 28.99 | 0.08 | 3.08 | 5674.28 | 13.05 |
| 29.53 | 0.13 | 3.03 | 4821.13 | 11.09 |
| 30.19 | 0.1 | 2.96 | 2015.19 | 4.63 |
| 30.58 | 0.1 | 2.92 | 1274.3 | 2.93 |
| 31 | 0.13 | 2.88 | 1660.45 | 3.82 |
| 31.4 | 0.12 | 2.85 | 2674.89 | 6.15 |
| 31.76 | 0.08 | 2.82 | 1885.98 | 4.34 |
| 32.7 | 0.13 | 2.74 | 1013.19 | 2.33 |
| 33.07 | 0.2 | 2.71 | 1543.3 | 3.55 |
| 33.49 | 0.08 | 2.68 | 859.01 | 1.98 |
| 34.04 | 0.13 | 2.63 | 988.63 | 2.27 |
| 34.41 | 0.12 | 2.61 | 830.75 | 1.91 |
| 35.48 | 0.1 | 2.53 | 1709.92 | 3.93 |
| 35.7 | 0.07 | 2.51 | 2500.38 | 5.75 |
| 36.28 | 0.12 | 2.48 | 756.03 | 1.74 |
| 36.78 | 0.15 | 2.44 | 3076.03 | 7.07 |
| 37.72 | 0.27 | 2.39 | 438.67 | 1.01 |
| 38.3 | 0.08 | 2.35 | 1477.03 | 3.4 |
| 38.88 | 0.13 | 2.32 | 1029.02 | 2.37 |
| 39.26 | 0.17 | 2.29 | 714.6 | 1.64 |
| 39.68 | 0.2 | 2.27 | 582.56 | 1.34 |

### Example 31: AMG 397 DMSO Solvate

AMG 397 DMSO solvate was formed by charging AMG 397 with hot DMSO to form a solution then allowing to cool to room temperature. The isolated solids were AMG 397 DMSO solvate which was characterized as shown in the below table.

TABLE 50
X-ray Single Structure Data

| Wavelength | 0.710730 | Å | |
|---|---|---|---|
| Crystal system | Orthorhombic | | |
| Space group(s) | P2₁2₁2₁ | | |
| Chiral Contents? | Chiral | | |
| Unit cell dimensions | a = 14.8422(10) | Å | α = 90° |
| | b = 17.8516(12) | Å | β = 90° |
| | c = 20.1556(13) | Å | γ = 90° |

| Volume | 5329.6(6) | Å³ |
|---|---|---|
| Z | 4 | |
| Density (calculated) | 1.291 | g/cm³ |

| | |
|---|---|
| Absolute structure parameter | −0.006(17) |

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

The use of the terms “a,” “an,” “the,” and similar referents in the context of the disclosure herein (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure herein and is not a limitation on the scope of the disclosure herein unless otherwise indicated. No language in the specification should be construed as indicating any non-claimed element as essential to the practice of the disclosure herein.

All patents, publications and references cited herein are hereby fully incorporated by reference. In case of conflict between the present disclosure and incorporated patents, publications and references, the present disclosure should control.

### EMBODIMENTS

The foregoing disclosure may be better understood through the following embodiments.

Embodiment 1. A crystalline form of AMG 397 as a trifluoroethanol solvate, characterized by XRPD pattern peaks at 17.5, 19.2, 19.4, and 21.7±0.2° 2θ using Cu Kα radiation.

Embodiment 2. The crystalline form of embodiment 1, further characterized by XRPD pattern peaks at 14.6, 17.2, 18.4, 18.5, 18.8, 20.0, 20.2, 20.4, 21.0, 21.2, and 21.5±0.2° 2θ using Cu Kα radiation.

Embodiment 3. The crystalline form of embodiment 2, further characterized by XRPD pattern peaks at 6.7, 10.3, 12.5, 13.5, 13.8, 17.7, 17.8, 18.1, 21.9, 22.3, 22.4, and 22.9±0.2° 2θ using Cu Kα radiation.

Embodiment 4. The crystalline form of any one of embodiments 1 to 3, having an XRPD pattern substantially as shown in FIG. 1.

Embodiment 5. A crystalline form of AMG 397 as a hexafluoroisopropanol solvate, characterized by XRPD pattern peaks at 11.4, 18.6, and 18.8±0.2° 2θ using Cu Kα radiation.

Embodiment 6. The crystalline form of embodiment 5, further characterized by XRPD pattern peaks at 8.5, 12.8, 17.1, 17.6, 21.1, 22.4, and 23.1±0.2° 2θ using Cu Kα radiation.

Embodiment 7. The crystalline form of embodiment 6, further characterized by XRPD pattern peaks at 6.1, 13.6, 15.3, 15.7, 16.2, 16.4, 16.5, 17.4, 17.8, 18.0, 18.1, 19.4, 20.6, 21.5, 21.7, 22.2, and 25.4±0.2° 2θ using Cu Kα radiation.

Embodiment 8. The crystalline form of any one of embodiments 5 to 7, having an XRPD pattern substantially as shown in FIG. 2.

Embodiment 9. A crystalline form of AMG 397 as a 1-propanol solvate, characterized by XRPD pattern peaks at 13.3, 15.1, and 18.5±0.2° 2θ using Cu Kα radiation.

Embodiment 10. The crystalline form of embodiment 9, further characterized by XRPD pattern peaks at 8.1, 9.7, 15.7, 16.4, 17.2, and 17.7±0.2° 2θ using Cu Kα radiation.

Embodiment 11. The crystalline form of embodiment 10, further characterized by XRPD pattern peaks at 12.0, 12.7, 14.2, 14.8, 17.1, 18.2, 19.1, 19.5, 20.7, 21.2, 21.6, 21.7, 22.1, 22.3, 22.4, 22.8, 23.5, 23.8, 23.9, and 25.5±0.2° 2θ using Cu Kα radiation.

Embodiment 12. The crystalline form of any one of embodiments 9 to 11, having an XRPD pattern substantially as shown in FIG. 3.

Embodiment 13. The crystalline form of any one of embodiments 9 to 12, having an endothermic transition at 231° C. to 237° C., as measured by differential scanning calorimetry.

Embodiment 14. The crystalline form of embodiment 13, wherein the endothermic transition is at 234° C.±3° C.

Embodiment 15. The crystalline form of any one of embodiments 9 to 14, having a thermogravimetric analysis (“TGA”) substantially as shown in FIG. 5.

Embodiment 16. A crystalline form of AMG 397 as an isopropanol solvate, characterized by XRPD pattern peaks at 6.1, 7.1, and 10.0±0.2° 2θ using Cu Kα radiation.

Embodiment 17. The crystalline form of embodiment 16, further characterized by XRPD pattern peaks at 18.5, 19.0, 19.7, and 20.4±0.2° 2θ using Cu Kα radiation.

Embodiment 18. The crystalline form of embodiment 17, further characterized by XRPD pattern peaks at 10.5, 13.6, 14.5, 15.0, 15.3, 15.9, 16.2, 16.6, 16.7, 16.9, 17.7, 17.9, 18.4, 19.5, 20.7, 21.6, 23.1, and 25.7±0.2° 2θ using Cu Kα radiation.

Embodiment 19. The crystalline form of any one of embodiments 16 to 18, having an XRPD pattern substantially as shown in FIG. 6.

Embodiment 20. The crystalline form of any one of embodiments 16 to 19, having an endothermic transition at 244° C. to 250° C., as measured by differential scanning calorimetry.

Embodiment 21. The crystalline form of embodiment 20, wherein the endothermic transition is at 247° C.±3° C.

Embodiment 22. The crystalline form of any one of embodiments 16 to 21, having a thermogravimetric analysis (“TGA”) substantially as shown in FIG. 8.

Embodiment 23. A crystalline form of AMG 397 as an isopropanol solvate, characterized by XRPD pattern peaks at 13.3, 15.1, and 18.6±0.2° 2θ using Cu Kα radiation.

Embodiment 24. The crystalline form of embodiment 23, further characterized by XRPD pattern peaks at 8.1, 9.7, 16.4, and 17.7±0.2° 2θ using Cu Kα radiation.

Embodiment 25. The crystalline form of embodiment 24, further characterized by XRPD pattern peaks at 12.0, 12.6, 14.2, 14.8, 15.7, 17.1, 17.2, 18.2, 19.1, 19.5, 21.5, 21.6, 22.3, 22.4, and 23.8±0.2° 2θ using Cu Kα radiation.

Embodiment 26. The crystalline form of any one of embodiments 23 to 25, having an XRPD pattern substantially as shown in FIG. 9.

Embodiment 27. The crystalline form of any one of embodiments 23 to 26, having an endothermic transition at 80° C. to 86° C. and 236° C. to 242° C., as measured by differential scanning calorimetry.

Embodiment 28. The crystalline form of embodiment 27, wherein the endothermic transitions are at 83° C.±3° C. and 239° C.±3° C.

Embodiment 29. The crystalline form of any one of embodiments 23 to 28, having a thermogravimetric analysis (“TGA”) substantially as shown in FIG. 11.

Embodiment 30. A crystalline form of AMG 397 as an acetonitrile solvate, characterized by XRPD pattern peaks at 10.2, 17.0, and 20.5±0.2° 2θ using Cu Kα radiation.

Embodiment 31. The crystalline form of embodiment 30, further characterized by XRPD pattern peaks at 6.0, 13.0, 14.3, 15.2, 18.6, and 23.0±0.2° 2θ using Cu Kα radiation.

Embodiment 32. The crystalline form of embodiment 31, further characterized by XRPD pattern peaks at 10.9, 15.6, 17.2, 18.2, 19.2, 21.0, 21.4, 22.1, 22.3, 22.5, 23.4, 24.8, 25.2, 25.6, 26.1, 26.5, 26.7, and 26.8±0.2° 2θ using Cu Kα radiation.

Embodiment 33. The crystalline form of any one of embodiments 30 to 32, having an XRPD pattern substantially as shown in FIG. 12.

Embodiment 34. A crystalline form of AMG 397 as an acetic acid solvate, characterized by solid state ¹³C NMR peaks at 13.63, 19.22, 20.40, 24.22, 25.69, 26.57, 27.75, 29.81, 30.40, 31.28, 36.57, 38.34, 40.10, 43.04, 49.51, 50.10, 51.86, 54.51, 56.28, 57.16, 57.75, 60.10, 62.16, 65.39, 77.75, 85.10, 115.39, 123.63, 125.10, 128.04, 131.27, 133.04, 133.92, 135.98, 139.80, 141.27, 143.04, 151.86, and 173.92±0.5 ppm.

Embodiment 35. The crystalline form of embodiment 34, further characterized by XRPD pattern peaks at 11.1, 17.1, 18.2, and 19.1±0.2° 2θ using Cu Kα radiation.

Embodiment 36. The crystalline form of embodiment 35, further characterized by XRPD pattern peaks at 10.7, 10.9, 11.5, 13.7, 14.3, 18.8, 20.1, and 24.8±0.2° 2θ using Cu Kα radiation.

Embodiment 37. The crystalline form of embodiment 36, further characterized by XRPD pattern peaks at 8.4, 12.4, 12.7, 15.6, 16.5, 17.6, 19.3, 22.2, 23.6, 24.0, 24.6, and 29.0±0.2° 2θ using Cu Kα radiation.

Embodiment 38. The crystalline form of any one of embodiments 34 to 37, having an XRPD pattern substantially as shown in FIG. 13.

Embodiment 39. The crystalline form of any one of embodiments 34 to 38, having an endothermic transition at 92° C. to 98° C. and 152° C. to 158° C., as measured by differential scanning calorimetry.

Embodiment 40. The crystalline form of embodiment 39, wherein the endothermic transitions are at 95° C.±3° C. and 155° C.±3° C.

Embodiment 41. The crystalline form of any one of embodiments 34 to 40, having a thermogravimetric analysis (“TGA”) substantially as shown in FIG. 15.

Embodiment 42. A crystalline form of AMG 397 as a hydrochloride salt, characterized by XRPD pattern peaks at 12.9, 16.2, and 17.9±0.2° 2θ using Cu Kα radiation.

Embodiment 43. The crystalline form of embodiment 42, further characterized by XRPD pattern peaks at 11.7, 12.0, 15.9, 19.8, and 20.5±0.2° 2θ using Cu Kα radiation.

Embodiment 44. The crystalline form of embodiment 43, further characterized by XRPD pattern peaks at 10.7, 13.5, 14.4, 14.6, 15.5, 18.1, 22.8, 23.7, 24.6, 25.1, and 26.5±0.2° 2θ using Cu Kα radiation.

Embodiment 45. The crystalline form of any one of embodiments 42 to 44, having an XRPD pattern substantially as shown in FIG. 17.

Embodiment 46. The crystalline form of any one of embodiments 42 to 45, having an endothermic transition at 264° C. to 270° C., as measured by differential scanning calorimetry.

Embodiment 47. The crystalline form of embodiment 46, wherein the endothermic transition is at 267° C.±3° C.

Embodiment 48. The crystalline form of any one of embodiments 42 to 47, having a thermogravimetric analysis (“TGA”) substantially as shown in FIG. 19.

Embodiment 49. An amorphous form of AMG 397 as a sodium salt, having an XRPD pattern substantially as shown in FIG. 21.

Embodiment 50. The amorphous form of embodiment 49, having an endothermic transition at 213° C. to 219° C., as measured by differential scanning calorimetry.

Embodiment 51. The amorphous form of embodiment 50, wherein the endothermic transition is at 216° C.±3° C.

Embodiment 52. The amorphous form of any one of embodiments 49 to 51, having a thermogravimetric analysis (“TGA”) substantially as shown in FIG. 23.

Embodiment 53. A crystalline form of AMG 397 as a potassium salt, characterized by XRPD pattern peaks at 12.8, 13.4, and 17.2±0.2° 2θ using Cu Kα radiation.

Embodiment 54. The crystalline form of embodiment 53, further characterized by XRPD pattern peaks at 11.0, 11.4, 14.5, 15.7, and 19.2±0.2° 2θ using Cu Kα radiation.

Embodiment 55. The crystalline form of embodiment 53 or 54, having an XRPD pattern substantially as shown in FIG. 25.

Embodiment 56. The crystalline form of any one of embodiments 53 to 55, having endothermic transitions at 158° C. to 164° C. and 224° C. to 230° C., as measured by differential scanning calorimetry.

Embodiment 57. The crystalline form of embodiment 56, wherein the endothermic transitions are at 161° C.±3° C. and 227° C.±3° C.

Embodiment 58. A crystalline form of AMG 397 as a potassium salt (ethyl acetate solvate), characterized by XRPD pattern peaks at 2.7, 11.7, and 12.2±0.2° 2θ using Cu Kα radiation.

Embodiment 59. The crystalline form of embodiment 58, further characterized by XRPD pattern peaks at 20.5, 20.9, 21.1, 21.6, and 22.9±0.2° 2θ using Cu Kα radiation.

Embodiment 60. The crystalline form of embodiment 59, further characterized by XRPD pattern peaks at 11.2, 15.1, 15.3, 15.4, 16.1, 16.3, 16.4, 16.6, 16.8, 16.9, 17.3, 17.5, 17.9, 18.5, 18.9, 19.2, 19.5, 19.721.7, 22.2, 22.5, 22.7, 23.3, 23.5, 23.9, and 24.4±0.2° 2θ using Cu Kα radiation.

Embodiment 61. The crystalline form of any one of embodiments 58 to 60, having an XRPD pattern substantially as shown in FIG. 27.

Embodiment 62. The crystalline form of any one of embodiments 58 to 61, having endothermic transitions at 64° C. to 70° C. and 146° C. to 152° C., as measured by differential scanning calorimetry.

Embodiment 63. The crystalline form of embodiment 62, wherein the endothermic transitions are at 67° C.±3° C. and 149° C.±3° C.

Embodiment 64. The crystalline form of any one of embodiments 58 to 63, having a thermogravimetric analysis (“TGA”) substantially as shown in FIG. 29.

Embodiment 65. A crystalline form of AMG 397 as a sulfate salt, characterized by XRPD pattern peaks at 9.3, 13.9, and 19.2±0.2° 2θ using Cu Kα radiation.

Embodiment 66. The crystalline form of embodiment 65, further characterized by XRPD pattern peaks at 8.7, 11.5, 17.6, and 21.9±0.2° 2θ using Cu Kα radiation.

Embodiment 67. The crystalline form of embodiment 65 or 66, having an XRPD pattern substantially as shown in FIG. 31.

Embodiment 68. The crystalline form of any one of embodiments 65 to 67, having an endothermic transition at 188° C. to 194° C., as measured by differential scanning calorimetry.

Embodiment 69. The crystalline form of embodiment 68, wherein the endothermic transition is at 191° C.±3° C.

Embodiment 70. A crystalline form of AMG 397 as a sulfate salt, characterized by XRPD pattern peaks at 11.7, 17.1, and 20.1±0.2° 2θ using Cu Kα radiation.

Embodiment 71. The crystalline form of embodiment 70, further characterized by XRPD pattern peaks at 12.8, 15.9, and 24.1±0.2° 2θ using Cu Kα radiation.

Embodiment 72. The crystalline form of embodiment 70 or 71, having an XRPD pattern substantially as shown in FIG. 33.

Embodiment 73. A crystalline form of AMG 397 as a sulfate salt, characterized by XRPD pattern peaks at 12.3, 17.7, 18.4, and 20.6±0.2° 2θ using Cu Kα radiation.

Embodiment 74. The crystalline form of embodiment 73, further characterized by XRPD pattern peaks at 11.2, 14.0, 19.0, and 23.1±0.2° 2θ using Cu Kα radiation.

Embodiment 75. The crystalline form of embodiment 74, further characterized by XRPD pattern peaks at 13.0, 15.3, 15.8, 16.7, 19.0, 21.6, 13.9, and 24.8±0.2° 2θ using Cu Kα radiation.

Embodiment 76. The crystalline form of any one of embodiments 73 to 75, having an XRPD pattern substantially as shown in FIG. 34.

Embodiment 77. The crystalline form of any one of embodiments 73 to 76, having an endothermic transition at 215° C. to 221° C., as measured by differential scanning calorimetry.

Embodiment 78. The crystalline form of embodiment 77, wherein the endothermic transition is at 218° C.±3° C.

Embodiment 79. The crystalline form of any one of embodiments 73 to 78, having a thermogravimetric analysis (“TGA”) substantially as shown in FIG. 36.

Embodiment 80. A crystalline form of AMG 397 as a phosphate salt, characterized by ¹³C NMR peaks at 5.8, 15.0, 18.3, 21.2, 22.2, 23.6, 27.6, 27.6, 29.3, 31.7, 31.9, 35.7, 41.3, 43.6, 49.9, 51.7, 53.3, 53.7, 55.8, 57.7, 58.8, 58.9, 59.8, 61.0, 79.6, 80.9, 115.4, 117.3, 119.1, 126.0, 127.9, 128.7, 129.4, 129.5, 130.2, 139.2, 139.8, 139.9, 150.8, and 168.8±0.5 ppm.

Embodiment 81. The crystalline form of embodiment 80, further characterized by XRPD pattern peaks at 17.7, 18.6, and 18.7±0.2° 2θ using Cu Kα radiation.

Embodiment 82. The crystalline form of embodiment 81, further characterized by XRPD pattern peaks at 12.3, 14.0, and 20.3±0.2° 2θ using Cu Kα radiation.

Embodiment 83. The crystalline form of embodiment 82, further characterized by XRPD pattern peaks at 11.1, 11.2, 12.4, 16.0, 16.1, 16.7, 16.8, 19.3, 20.7, 21.9, 22.9, 23.0, 24.7, and 24.8±0.2° 2θ using Cu Kα radiation.

Embodiment 84. The crystalline form of any one of embodiments 80 to 83, having an XRPD pattern substantially as shown in FIG. 37.

Embodiment 85. The crystalline form of any one of embodiments 80 to 84, having an endothermic transition at 207° C. to 213° C., as measured by differential scanning calorimetry.

Embodiment 86. The crystalline form of embodiment 85, wherein the endothermic transition is at 210° C.±3° C.

Embodiment 87. The crystalline form of any one of embodiments 80 to 86, having a thermogravimetric analysis (“TGA”) substantially as shown in FIG. 39.

Embodiment 88. A crystalline form of AMG 397 as a fumarate salt acetone solvate, characterized by XRPD pattern peaks at 17.6, 18.2, and 18.4±0.2° 2θ using Cu Kα radiation.

Embodiment 89. The crystalline form of embodiment 88, further characterized by XRPD pattern peaks at 5.3, 10.4, 12.2, 13.9, 15.8, and 24.0±0.2° 2θ using Cu Kα radiation.

Embodiment 90. The crystalline form of embodiment 89, further characterized by XRPD pattern peaks at 9.7, 11.0, 12.9, 14.9, 15.5, 16.3, 16.9, 17.9, 19.2, 20.2, 20.9, 21.6, 22.8, 24.7, and 26.1±0.2° 2θ using Cu Kα radiation.

Embodiment 91. The crystalline form of any one of embodiments 88 to 90, having an XRPD pattern substantially as shown in FIG. 41.

Embodiment 92. The crystalline form of any one of embodiments 88 to 91, having an endothermic transition at 229° C. to 235° C., as measured by differential scanning calorimetry.

Embodiment 93. The crystalline form of embodiment 92, wherein the endothermic transition is at 232° C.±3° C.

Embodiment 94. The crystalline form of any one of embodiments 88 to 93, having a thermogravimetric analysis (“TGA”) substantially as shown in FIG. 43.

Embodiment 95. A crystalline form of AMG 397 as a fumarate salt, characterized by XRPD pattern peaks at 11.9, 17.9, and 18.1±0.2° 2θ using Cu Kα radiation.

Embodiment 96. The crystalline form of embodiment 95, further characterized by XRPD pattern peaks at 10.7, 13.6, 15.7, 18.6, 18.8, 19.6, and 21.5±0.2° 2θ using Cu Kα radiation.

Embodiment 97. The crystalline form of embodiment 96, further characterized by XRPD pattern peaks at 10.3, 14.9, 16.3, 16.5, 20.0, 22.2, 22.6, 13.3, 13.9, 24.5, 25.5, and 28.1±0.2° 2θ using Cu Kα radiation.

Embodiment 98. The crystalline form of any one of embodiments 95 to 97, having an XRPD pattern substantially as shown in FIG. 45.

Embodiment 99. The crystalline form of any one of embodiments 95 to 98, having an endothermic transition at 240° C. to 246° C., as measured by differential scanning calorimetry.

Embodiment 100. The crystalline form of embodiment 99, wherein the endothermic transition is at 243° C.±3° C.

Embodiment 101. The crystalline form of any one of embodiments 95 to 100, having a thermogravimetric analysis (“TGA”) substantially as shown in FIG. 47.

Embodiment 102. A crystalline form of AMG 397 as a citrate salt, characterized by XRPD pattern peaks at 10.6, 17.6, and 18.3±0.2° 2θ using Cu Kα radiation.

Embodiment 103. The crystalline form of embodiment 102, further characterized by XRPD pattern peaks at 12.1, 13.9, 16.0, 19.2, and 21.9±0.2° 2θ using Cu Kα radiation.

Embodiment 104. The crystalline form of embodiment 103, further characterized by XRPD pattern peaks at 6.1, 11.0, 12.8, 15.2, 16.9, 19.5, 20.0, 20.5, 21.1, 22.9, 24.4, 24.7, 25.9, and 28.7±0.2° 2θ using Cu Kα radiation.

Embodiment 105. The crystalline form of any one of embodiments 102 to 104, having an XRPD pattern substantially as shown in FIG. 49.

Embodiment 106. The crystalline form of any one of embodiments 102 to 105, having an endothermic transition at 211° C. to 217° C., as measured by differential scanning calorimetry.

Embodiment 107. The crystalline form of embodiment 106, wherein the endothermic transition is at 214° C.±3° C.

Embodiment 108. The crystalline form of any one of embodiments 102 to 107, having a thermogravimetric analysis (“TGA”) substantially as shown in FIG. 51.

Embodiment 109. A crystalline form of AMG 397 as a citrate salt, characterized by XRPD pattern peaks at 17.7, 18.4, and 18.5±0.2° 2θ using Cu Kα radiation.

Embodiment 110. The crystalline form of embodiment 109, further characterized by XRPD pattern peaks at 14.0, 16.0, 20.1, and 21.9±0.2° 2θ using Cu Kα radiation.

Embodiment 111. The crystalline form of embodiment 110, further characterized by XRPD pattern peaks at 10.7, 11.1, 12.2, 12.9, 15.2, 19.3, 20.6, 22.9, 24.4, and 24.8±0.2° 2θ using Cu Kα radiation.

Embodiment 112. The crystalline form of any one of embodiments 109 to 111, having an XRPD pattern substantially as shown in FIG. 52.

Embodiment 113. The crystalline form of any one of embodiments 109 to 112, having an endothermic transition at 203° C. to 209° C., as measured by differential scanning calorimetry.

Embodiment 114. The crystalline form of embodiment 113, wherein the endothermic transition is at 206° C.±3° C.

Embodiment 115. The crystalline form of any one of embodiments 109 to 114, having a thermogravimetric analysis (“TGA”) substantially as shown in FIG. 54.

Embodiment 116. A crystalline form of AMG 397 as a lactate salt, characterized by XRPD pattern peaks at 12.1, 17.8, and 18.3±0.2° 2θ using Cu Kα radiation.

Embodiment 117. The crystalline form of embodiment 116, further characterized by XRPD pattern peaks at 10.5, 10.9, 13.8, 17.5, and 20.0±0.2° 2θ using Cu Kα radiation.

Embodiment 118. The crystalline form of embodiment 117, further characterized by XRPD pattern peaks at 5.9, 12.8, 15.9, 16.2, 19.1, 20.4, 21.7, 23.9, 24.6, and 25.1±0.2° 2θ using Cu Kα radiation.

Embodiment 119. The crystalline form of any one of embodiments 116 to 118, having an XRPD pattern substantially as shown in FIG. 57.

Embodiment 120. The crystalline form of any one of embodiments 116 to 119, having an endothermic transition at 216° C. to 222° C., as measured by differential scanning calorimetry.

Embodiment 121. The crystalline form of embodiment 120, wherein the endothermic transition is at 219° C.±3° C.

Embodiment 122. The crystalline form of any one of embodiments 116 to 121, having a thermogravimetric analysis (“TGA”) substantially as shown in FIG. 59.

Embodiment 123. A crystalline form of AMG 397 as a succinate salt, characterized by XRPD pattern peaks at 17.6, 18.4, and 18.7±0.2° 2θ using Cu Kα radiation.

Embodiment 124. The crystalline form of embodiment 123, further characterized by XRPD pattern peaks at 12.2, 13.9, 18.0, 20.3, and 24.5±0.2° 2θ using Cu Kα radiation.

Embodiment 125. The crystalline form of embodiment 124, further characterized by XRPD pattern peaks at 5.2, 10.4, 10.7, 11.1, 12.9, 15.2, 15.8, 16.7, 19.1, 21.6, 22.2, 22.8, 23.9, 26.2, 28.3, and 29.2±0.2° 2θ using Cu Kα radiation.

Embodiment 126. The crystalline form of any one of embodiments 123 to 125, having an XRPD pattern substantially as shown in FIG. 61.

Embodiment 127. The crystalline form of any one of embodiments 123 to 126, having an endothermic transition at 207° C. to 213° C., as measured by differential scanning calorimetry.

Embodiment 128. The crystalline form of embodiment 127, wherein the endothermic transition is at 210° C.±3° C.

Embodiment 129. The crystalline form of any one of embodiments 123 to 128, having a thermogravimetric analysis (“TGA”) substantially as shown in FIG. 63.

Embodiment 130. A crystalline form of AMG 397 as an ammonium salt, characterized by XRPD pattern peaks at 6.2, 10.3, and 17.2±0.2±0.2° 2θ using Cu Kα radiation.

Embodiment 131. The crystalline form of embodiment 130, further characterized by XRPD pattern peaks at 4.0, 4.7, 17.3, 17.9, 19.8, and 20.3±0.2° 2θ using Cu Kα radiation.

Embodiment 132. The crystalline form of embodiment 131, further characterized by XRPD pattern peaks at 13.0, 14.4, 15.1, 15.5, 15.9, 16.2, 16.4, 17.7, 18.6, 19.7, and 22.8±0.2° 2θ using Cu Kα radiation.

Embodiment 133. The crystalline form of any one of embodiments 130 to 132, having an XRPD pattern substantially as shown in FIG. 65.

Embodiment 134. The crystalline form of any one of embodiments 130 to 133, having an endothermic transition at 224° C. to 230° C., as measured by differential scanning calorimetry.

Embodiment 135. The crystalline form of embodiment 134, wherein the endothermic transition is at 227° C.±3° C.

Embodiment 136. The crystalline form of any one of embodiments 130 to 135, having a thermogravimetric analysis (“TGA”) substantially as shown in FIG. 67.

Embodiment 137. A crystalline form AMG 397 as a besylate salt, characterized by XRPD pattern peaks at 17.6, 18.4, and 18.7±0.2° 2θ using Cu Kα radiation.

Embodiment 138. The crystalline form of embodiment 137, further characterized by XRPD pattern peaks at 14.0, 17.7, and 20.4±0.2° 2θ using Cu Kα radiation.

Embodiment 139. The crystalline form of embodiment 138, further characterized by XRPD pattern peaks at 11.2, 12.4, 13.8, 14.1, 15.9, 16.1, 18.0, 19.3, 20.8, 21.7, 22.9, 23.9, and 24.5±0.2° 2θ using Cu Kα radiation.

Embodiment 140. The crystalline form of any one of embodiments 137 to 139, having an XRPD pattern substantially as shown in FIG. 68.

Embodiment 141. The crystalline form of any one of embodiments 137 to 140, having endothermic transitions 54° C. to 60° C. and 231° C. to 237° C., as measured by differential scanning calorimetry.

Embodiment 142. The crystalline form of embodiment 141, wherein the endothermic transitions are at 57° C.±3° C. and 234° C.±3° C.

Embodiment 143. The crystalline form of any one of embodiments 137 to 142, having a thermogravimetric analysis (“TGA”) substantially as shown in FIG. 70.

Embodiment 144. A crystalline form of AMG 397 as a tosylate salt, characterized by XRPD pattern peaks at 18.2, 18.4, and 20.5±0.2° 2θ using Cu Kα radiation.

Embodiment 145. The crystalline form of embodiment 144, further characterized by XRPD pattern peaks at 12.2, 12.3, 17.6, 18.9, and 19.1±0.2° 2θ using Cu Kα radiation.

Embodiment 146. The crystalline form of embodiment 145, further characterized by XRPD pattern peaks at 4.5, 5.2, 13.0, 13.8, 14.0, 15.2, 15.8, 16.2, 16.4, 19.8, 20.0, 21.4, 22.9, 23.4, 23.6, 23.8, 24.3, 24.6, 25.1, and 27.1±0.2° 2θ using Cu Kα radiation.

Embodiment 147. The crystalline form of any one of embodiments 144 to 146, having an XRPD pattern substantially as shown in FIG. 72.

Embodiment 148. The crystalline form of any one of embodiments 144 to 147, having endothermic transitions 37° C. to 43° C. and 223° C. to 229° C., as measured by differential scanning calorimetry.

Embodiment 149. The crystalline form of embodiment 148, wherein the endothermic transitions are at 40° C.±3° C. and 226° C.±3° C.

Embodiment 150. The crystalline form of any one of embodiments 144 to 149, having a thermogravimetric analysis (“TGA”) substantially as shown in FIG. 74.

Embodiment 151. A crystalline form of AMG 397 as a maleate salt, characterized by XRPD pattern peaks at 18.2, 18.9, and 19.9±0.2° 2θ using Cu Kα radiation.

Embodiment 152. The crystalline form of embodiment 151, further characterized by XRPD pattern peaks at 10.4, 10.9, 12.0, and 21.5±0.2° 2θ using Cu Kα radiation.

Embodiment 153. The crystalline form of embodiment 152, further characterized by XRPD pattern peaks at 10.3, 13.8, 15.8, 17.9, 19.2, and 24.2±0.2° 2θ 0.2° 2θ using Cu Kα radiation.

Embodiment 154. The crystalline form of any one of embodiments 151 to 153, having an XRPD pattern substantially as shown in FIG. 76.

Embodiment 155. The crystalline form of any one of embodiments 151 to 154, having an endothermic transition at 219° C. to 225° C., as measured by differential scanning calorimetry.

Embodiment 156. The crystalline form of embodiment 155, wherein the endothermic transition is at 222° C.±3° C.

Embodiment 157. The crystalline form of any one of embodiments 151 to 156, having a thermogravimetric analysis (“TGA”) substantially as shown in FIG. 78.

Embodiment 158. A crystalline form of AMG 397 as a maleate salt, characterized by XRPD pattern peaks at 10.6, 18.6, and 20.3±0.2° 2θ using Cu Kα radiation.

Embodiment 159. The crystalline form of embodiment 158, further characterized by XRPD pattern peaks at 10.8, 12.3, 15.2, 15.9, and 16.7±0.2° 2θ using Cu Kα radiation.

Embodiment 160. The crystalline form of embodiment 159, further characterized by XRPD pattern peaks at 9.8, 11.1, 13.9, 14.1, 18.0, 18.4, 19.2, 19.4, 20.8, 22.3, 23.0, 23.6, 24.6, and 28.4±0.2° 2θ using Cu Kα radiation.

Embodiment 161. The crystalline form of any one of embodiments 158 to 160, having an XRPD pattern substantially as shown in FIG. 79.

Embodiment 162. A crystalline form of AMG 397 as a malonate salt, characterized by XRPD pattern peaks at 12.2, 18.8, and 20.4±0.2° 2θ using Cu Kα radiation.

Embodiment 163. The crystalline form of embodiment 162, further characterized by XRPD pattern peaks at 10.3, 11.1, 17.9, 18.3, and 19.1±0.2° 2θ using Cu Kα radiation.

Embodiment 164 The crystalline form of embodiment 163, further characterized by XRPD pattern peaks at 10.7, 13.9, 14.0, 15.8, 16.5, 18.4, 19.5, 19.7 21.6, 21.7, 22.8, and 24.5±0.2° 2θ using Cu Kα radiation.

Embodiment 165. The crystalline form of any one of embodiments 162 to 164, having an XRPD pattern substantially as shown in FIG. 82.

Embodiment 166. The crystalline form of any one of embodiments 162 to 165, having endothermic transitions 158° C. to 164° C. and 184° C. to 190° C., as measured by differential scanning calorimetry.

Embodiment 167. The crystalline form of embodiment 166, wherein the endothermic transitions are at 161° C.±3° C. and 187° C.±3° C.

Embodiment 168. The crystalline form of any one of embodiments 162 to 167, having a thermogravimetric analysis (“TGA”) substantially as shown in FIG. 84.

Embodiment 169. A crystalline form of AMG 397 as a malonate salt, characterized by XRPD pattern peaks at 10.6, 18.5, and 20.2±0.2° 2θ using Cu Kα radiation.

Embodiment 170. The crystalline form of embodiment 169, further characterized by XRPD pattern peaks at 11.0, 14.0, and 17.9±0.2° 2θ using Cu Kα radiation.

Embodiment 171. The crystalline form of embodiment 170, further characterized by XRPD pattern peaks at 11.1, 12.3, 15.3, 16.1, 16.8, 17.0, 18.6, 19.4, and 22.2±0.2° 2θ using Cu Kα radiation.

Embodiment 172. The crystalline form of any one of embodiments 169 to 171, having an XRPD pattern substantially as shown in FIG. 85.

Embodiment 173. A crystalline form of AMG 397 as a tartrate salt, characterized by XRPD pattern peaks at 18.2, 18.6, and 20.2±0.2° 2θ using Cu Kα radiation.

Embodiment 174. The crystalline form of embodiment 173, further characterized by XRPD pattern peaks at 12.1, 17.8, 19.0, and 21.5±0.2° 2θ using Cu Kα radiation.

Embodiment 175. The crystalline form of embodiment 174, further characterized by XRPD pattern peaks at 10.6, 11.0, 12.8, 13.8, 15.1, 15.8, 16.4, 16.6, 17.4, 19.3, 19.5, 20.6, 22.1, 22.6, 23.5, and 24.4+0.2° 2θ using Cu Kα radiation.

Embodiment 176. The crystalline form of any one of embodiments 173 to 175, having an XRPD pattern substantially as shown in FIG. 87.

Embodiment 177. The crystalline form of any one of embodiments 173 to 176, having an endothermic transition at 224° C. to 230° C., as measured by differential scanning calorimetry.

Embodiment 178. The crystalline form of embodiment 177, wherein the endothermic transition is at 227° C.±3° C.

Embodiment 179. The crystalline form of any one of embodiments 173 to 178, having a thermogravimetric analysis (“TGA”) substantially as shown in FIG. 89.

Embodiment 180. A crystalline form of AMG 397 as a tris(hydroxymethyl)aminomethane salt acetone solvate, characterized by XRPD pattern peaks at 10.0, 16.8, and 20.0±0.2° 2θ using Cu Kα radiation.

Embodiment 181. The crystalline form of embodiment 180, further characterized by XRPD pattern peaks at 12.7, 14.1, and 18.2±0.2° 2θ using Cu Kα radiation.

Embodiment 182. The crystalline form of embodiment 181, further characterized by XRPD pattern peaks at 6.1, 14.9, 15.3, 16.0, 17.3, 17.6, 18.0, 19.0, 19.1, 19.4, 20.6, 22.1, 22.5, 22.7, 22.9, 26.3, and 26.4±0.2° 2θ using Cu Kα radiation.

Embodiment 183. The crystalline form of any one of embodiments 180 to 182, having an XRPD pattern substantially as shown in FIG. 90.

Embodiment 184. The crystalline form of any one of embodiments 180 to 183, having endothermic transitions at 56° C. to 62° C. and 131° C. to 137° C., as measured by differential scanning calorimetry.

Embodiment 185. The crystalline form of embodiment 184, wherein the endothermic transitions are at 59° C.±3° C. and 134° C.±3° C.

Embodiment 186. The crystalline form of any one of embodiments 180 to 185, having a thermogravimetric analysis (“TGA”) substantially as shown in FIG. 92.

Embodiment 187. A crystalline form of AMG 397 as an iodide salt, characterized by XRPD pattern peaks at 17.0, 18.0, and 18.1±0.2° 2θ using Cu Kα radiation.

Embodiment 188. The crystalline form of embodiment 187, further characterized by XRPD pattern peaks at 8.3, 11.0, 18.6, 18.8, 19.1, 20.0, 22.1, 23.5, and 24.7±0.2° 2θ using Cu Kα radiation.

Embodiment 189. The crystalline form of embodiment 188 further characterized by XRPD pattern peaks at 6.2, 10.6, 10.8, 12.4, 13.0, 14.1, 15.5, 17.6, 22.5, 24.1, 28.6, 28.8, 29.0, and 29.5±0.2° 2θ using Cu Kα radiation.

Embodiment 190. The crystalline form of any one of embodiments 187 to 189, having an XRPD pattern substantially as shown in FIG. 93.

Embodiment 191. The crystalline form of any one of embodiments 187 to 190, having an endothermic transition at 228° C. to 234° C., as measured by differential scanning calorimetry.

Embodiment 192. The crystalline form of embodiment 191, wherein the endothermic transition is at 231° C.±3° C.

Embodiment 193. A pharmaceutical formulation comprising the crystalline form of any one of embodiments 1 to 192 and a pharmaceutically acceptable excipient.

Embodiment 194. A method of treating a subject suffering from cancer, comprising administering to the subject a therapeutically effective amount of the crystalline form of any one of embodiments 1 to 192 or the pharmaceutical formulation of embodiment 193.

Embodiment 195. The method of embodiment 194, wherein the cancer is multiple myeloma, non-Hodgkin's lymphoma, or acute myeloid leukemia.

What is claimed is:

1. A crystalline form of AMG 397 as a trifluoroethanol solvate, characterized by XRPD pattern peaks at 17.5, 19.2, 19.4, and 21.7±0.2° 2θ using Cu Kα radiation.

2. The crystalline form of claim 1, further characterized by XRPD pattern peaks at 14.6, 17.2, 18.4, 18.5, 18.8, 20.0, 20.2, 20.4, 21.0, 21.2, and 21.5±0.2° 2θ using Cu Kα radiation.

3. The crystalline form of claim 2, further characterized by XRPD pattern peaks at 6.7, 10.3, 12.5, 13.5, 13.8, 17.7, 17.8, 18.1, 21.9, 22.3, 22.4, and 22.9±0.2° 2θ using Cu Kα radiation.

4. The crystalline form of any one of claims 1 to 3, having an XRPD pattern substantially as shown in FIG. 1.

5. A crystalline form of AMG 397 as a hexafluoroisopropanol solvate, characterized by XRPD pattern peaks at 11.4, 18.6, and 18.8±0.2° 2θ using Cu Kα radiation.

6. The crystalline form of claim 5, further characterized by XRPD pattern peaks at 8.5, 12.8, 17.1, 17.6, 21.1, 22.4, and 23.1±0.2° 2θ using Cu Kα radiation.

7. The crystalline form of claim 6, further characterized by XRPD pattern peaks at 6.1, 13.6, 15.3, 15.7, 16.2, 16.4, 16.5, 17.4, 17.8, 18.0, 18.1, 19.4, 20.6, 21.5, 21.7, 22.2, and 25.4±0.2° 2θ using Cu Kα radiation.

8. The crystalline form of any one of claims 5 to 7, having an XRPD pattern substantially as shown in FIG. 2.

9. A crystalline form of AMG 397 as a 1-propanol solvate, characterized by XRPD pattern peaks at 13.3, 15.1, and 18.5±0.2° 2θ using Cu Kα radiation.

10. The crystalline form of claim 9, further characterized by XRPD pattern peaks at 8.1, 9.7, 15.7, 16.4, 17.2, and 17.7±0.2° 2θ using Cu Kα radiation.

11. The crystalline form of claim 10, further characterized by XRPD pattern peaks at 12.0, 12.7, 14.2, 14.8, 17.1, 18.2, 19.1, 19.5, 20.7, 21.2, 21.6, 21.7, 22.1, 22.3, 22.4, 22.8, 23.5, 23.8, 23.9, and 25.5±0.2° 2θ using Cu Kα radiation.

12. The crystalline form of any one of claims 9 to 11, having an XRPD pattern substantially as shown in FIG. 3.

13. The crystalline form of any one of claims 9 to 12, having an endothermic transition at 231° C. to 237° C., as measured by differential scanning calorimetry.

14. The crystalline form of claim 13, wherein the endothermic transition is at 234° C.±3° C.

15. The crystalline form of any one of claims 9 to 14, having a thermogravimetric analysis (“TGA”) substantially as shown in FIG. 5.

16. A crystalline form of AMG 397 as an isopropanol solvate, characterized by XRPD pattern peaks at 6.1, 7.1, and 10.0±0.2° 2θ using Cu Kα radiation.

17. The crystalline form of claim 16, further characterized by XRPD pattern peaks at 18.5, 19.0, 19.7, and 20.4±0.2° 2θ using Cu Kα radiation.

18. The crystalline form of claim 17, further characterized by XRPD pattern peaks at 10.5, 13.6, 14.5, 15.0, 15.3, 15.9, 16.2, 16.6, 16.7, 16.9, 17.7, 17.9, 18.4, 19.5, 20.7, 21.6, 23.1, and 25.7±0.2° 2θ using Cu Kα radiation.

19. The crystalline form of any one of claims 16 to 18, having an XRPD pattern substantially as shown in FIG. 6.

20. The crystalline form of any one of claims 16 to 19, having an endothermic transition at 244° C. to 250° C., as measured by differential scanning calorimetry.

21. The crystalline form of claim 20, wherein the endothermic transition is at 247° C.±3° C.

22. The crystalline form of any one of claims 16 to 21, having a thermogravimetric analysis (“TGA”) substantially as shown in FIG. 8.

23. A crystalline form of AMG 397 as an isopropanol solvate, characterized by XRPD pattern peaks at 13.3, 15.1, and 18.6±0.2° 2θ using Cu Kα radiation.

24. The crystalline form of claim 23, further characterized by XRPD pattern peaks at 8.1, 9.7, 16.4, and 17.7±0.2° 2θ using Cu Kα radiation.

25. The crystalline form of claim 24, further characterized by XRPD pattern peaks at 12.0, 12.6, 14.2, 14.8, 15.7, 17.1, 17.2, 18.2, 19.1, 19.5, 21.5, 21.6, 22.3, 22.4, and 23.8±0.2° 2θ using Cu Kα radiation.

26. The crystalline form of any one of claims 23 to 25, having an XRPD pattern substantially as shown in FIG. 9.

27. The crystalline form of any one of claims 23 to 26, having an endothermic transition at 80° C. to 86° C. and 236° C. to 242° C., as measured by differential scanning calorimetry.

28. The crystalline form of claim 27, wherein the endothermic transitions are at 83° C.±3° C. and 239° C.±3° C.

29. The crystalline form of any one of claims 23 to 28, having a thermogravimetric analysis (“TGA”) substantially as shown in FIG. 11.

30. A crystalline form of AMG 397 as an acetonitrile solvate, characterized by XRPD pattern peaks at 10.2, 17.0, and 20.5±0.2° 2θ using Cu Kα radiation.

31. The crystalline form of claim 30, further characterized by XRPD pattern peaks at 6.0, 13.0, 14.3, 15.2, 18.6, and 23.0±0.2° 2θ using Cu Kα radiation.

32. The crystalline form of claim 31, further characterized by XRPD pattern peaks at 10.9, 15.6, 17.2, 18.2, 19.2, 21.0, 21.4, 22.1, 22.3, 22.5, 23.4, 24.8, 25.2, 25.6, 26.1, 26.5, 26.7, and 26.8±0.2° 2θ using Cu Kα radiation.

33. The crystalline form of any one of claims 30 to 32, having an XRPD pattern substantially as shown in FIG. 12.

34. A crystalline form of AMG 397 as an acetic acid solvate, characterized by solid state ¹³C NMR peaks at 13.63, 19.22, 20.40, 24.22, 25.69, 26.57, 27.75, 29.81, 30.40, 31.28, 36.57, 38.34, 40.10, 43.04, 49.51, 50.10, 51.86, 54.51, 56.28, 57.16, 57.75, 60.10, 62.16, 65.39, 77.75, 85.10, 115.39, 123.63, 125.10, 128.04, 131.27, 133.04, 133.92, 135.98, 139.80, 141.27, 143.04, 151.86, and 173.92±0.5 ppm.

35. The crystalline form of claim 34, further characterized by XRPD pattern peaks at 11.1, 17.1, 18.2, and 19.1±0.2° 2θ using Cu Kα radiation.

36. The crystalline form of claim 35, further characterized by XRPD pattern peaks at 10.7, 10.9, 11.5, 13.7, 14.3, 18.8, 20.1, and 24.8±0.2° 2θ using Cu Kα radiation.

37. The crystalline form of claim 36, further characterized by XRPD pattern peaks at 8.4, 12.4, 12.7, 15.6, 16.5, 17.6, 19.3, 22.2, 23.6, 24.0, 24.6, and 29.0±0.2° 2θ using Cu Kα radiation.

38. The crystalline form of any one of claims 34 to 37, having an XRPD pattern substantially as shown in FIG. 13.

39. The crystalline form of any one of claims 34 to 38, having an endothermic transition at 92° C. to 98° C. and 152° C. to 158° C., as measured by differential scanning calorimetry.

40. The crystalline form of claim 39, wherein the endothermic transitions are at 95° C.±3° C. and 155° C.±3° C.

41. The crystalline form of any one of claims 34 to 40, having a thermogravimetric analysis (“TGA”) substantially as shown in FIG. 15.

42. A crystalline form of AMG 397 as a hydrochloride salt, characterized by XRPD pattern peaks at 12.9, 16.2, and 17.9±0.2° 2θ using Cu Kα radiation.

43. The crystalline form of claim 42, further characterized by XRPD pattern peaks at 11.7, 12.0, 15.9, 19.8, and 20.5±0.2° 2θ using Cu Kα radiation.

44. The crystalline form of claim 43, further characterized by XRPD pattern peaks at 10.7, 13.5, 14.4, 14.6, 15.5, 18.1, 22.8, 23.7, 24.6, 25.1, and 26.5±0.2° 2θ using Cu Kα radiation.

45. The crystalline form of any one of claims 42 to 44, having an XRPD pattern substantially as shown in FIG. 17.

46. The crystalline form of any one of claims 42 to 45, having an endothermic transition at 264° C. to 270° C., as measured by differential scanning calorimetry.

47. The crystalline form of claim 46, wherein the endothermic transition is at 267° C.±3° C.

48. The crystalline form of any one of claims 42 to 47, having a thermogravimetric analysis (“TGA”) substantially as shown in FIG. 19.

49. An amorphous form of AMG 397 as a sodium salt, having an XRPD pattern substantially as shown in FIG. 21.

50. The amorphous form of claim 49, having an endothermic transition at 213° C. to 219° C., as measured by differential scanning calorimetry.

51. The amorphous form of claim 50, wherein the endothermic transition is at 216° C.±3° C.

52. The amorphous form of any one of claims 49 to 51, having a thermogravimetric analysis (“TGA”) substantially as shown in FIG. 23.

53. A crystalline form of AMG 397 as a potassium salt, characterized by XRPD pattern peaks at 12.8, 13.4, and 17.2±0.2° 2θ using Cu Kα radiation.

54. The crystalline form of claim 53, further characterized by XRPD pattern peaks at 11.0, 11.4, 14.5, 15.7, and 19.2±0.2° 2θ using Cu Kα radiation.

55. The crystalline form of claim 53 or 54, having an XRPD pattern substantially as shown in FIG. 25.

56. The crystalline form of any one of claims 53 to 55, having endothermic transitions at 158° C. to 164° C. and 224° C. to 230° C., as measured by differential scanning calorimetry.

57. The crystalline form of claim 56, wherein the endothermic transitions are at 161° C.±3° C. and 227° C.±3° C.

58. A crystalline form of AMG 397 as a potassium salt (ethyl acetate solvate), characterized by XRPD pattern peaks at 2.7, 11.7, and 12.2±0.2° 2θ using Cu Kα radiation.

59. The crystalline form of claim 58, further characterized by XRPD pattern peaks at 20.5, 20.9, 21.1, 21.6, and 22.9±0.2° 2θ using Cu Kα radiation.

60. The crystalline form of claim 59, further characterized by XRPD pattern peaks at 11.2, 15.1, 15.3, 15.4, 16.1, 16.3, 16.4, 16.6, 16.8, 16.9, 17.3, 17.5, 17.9, 18.5, 18.9, 19.2, 19.5, 19.721.7, 22.2, 22.5, 22.7, 23.3, 23.5, 23.9, and 24.4±0.2° 2θ using Cu Kα radiation.

61. The crystalline form of any one of claims 58 to 60, having an XRPD pattern substantially as shown in FIG. 27.

62. The crystalline form of any one of claims 58 to 61, having endothermic transitions at 64° C. to 70° C. and 146° C. to 152° C., as measured by differential scanning calorimetry.

63. The crystalline form of claim 62, wherein the endothermic transitions are at 67° C.±3° C. and 149° C.±3° C.

64. The crystalline form of any one of claims 58 to 63, having a thermogravimetric analysis (“TGA”) substantially as shown in FIG. 29.

65. A crystalline form of AMG 397 as a sulfate salt, characterized by XRPD pattern peaks at 9.3, 13.9, and 19.2±0.2° 2θ using Cu Kα radiation.

66. The crystalline form of claim 65, further characterized by XRPD pattern peaks at 8.7, 11.5, 17.6, and 21.9±0.2° 2θ using Cu Kα radiation.

67. The crystalline form of claim 65 or 66, having an XRPD pattern substantially as shown in FIG. 31.

68. The crystalline form of any one of claims 65 to 67, having an endothermic transition at 188° C. to 194° C., as measured by differential scanning calorimetry.

69. The crystalline form of claim 68, wherein the endothermic transition is at 191° C.±3° C.

70. A crystalline form of AMG 397 as a sulfate salt, characterized by XRPD pattern peaks at 11.7, 17.1, and 20.1±0.2° 2θ using Cu Kα radiation.

71. The crystalline form of claim 70, further characterized by XRPD pattern peaks at 12.8, 15.9, and 24.1±0.2° 2θ using Cu Kα radiation.

72. The crystalline form of claim 70 or 71, having an XRPD pattern substantially as shown in FIG. 33.

73. A crystalline form of AMG 397 as a sulfate salt, characterized by XRPD pattern peaks at 12.3, 17.7, 18.4, and 20.6±0.2° 2θ using Cu Kα radiation.

74. The crystalline form of claim 73, further characterized by XRPD pattern peaks at 11.2, 14.0, 19.0, and 23.1±0.2° 2θ using Cu Kα radiation.

75. The crystalline form of claim 74, further characterized by XRPD pattern peaks at 13.0, 15.3, 15.8, 16.7, 19.0, 21.6, 13.9, and 24.8±0.2° 2θ using Cu Kα radiation.

76. The crystalline form of any one of claims 73 to 75, having an XRPD pattern substantially as shown in FIG. 34.

77. The crystalline form of any one of claims 73 to 76, having an endothermic transition at 215° C. to 221° C., as measured by differential scanning calorimetry.

78. The crystalline form of claim 77, wherein the endothermic transition is at 218° C.±3° C.

79. The crystalline form of any one of claims 73 to 78, having a thermogravimetric analysis (“TGA”) substantially as shown in FIG. 36.

80. A crystalline form of AMG 397 as a phosphate salt, characterized by ¹³C NMR peaks at 5.8, 15.0, 18.3, 21.2, 22.2, 23.6, 27.6, 27.6, 29.3, 31.7, 31.9, 35.7, 41.3, 43.6, 49.9, 51.7, 53.3, 53.7, 55.8, 57.7, 58.8, 58.9, 59.8, 61.0, 79.6, 80.9, 115.4, 117.3, 119.1, 126.0, 127.9, 128.7, 129.4, 129.5, 130.2, 139.2, 139.8, 139.9, 150.8, and 168.8±0.5 ppm.

81. The crystalline form of claim 80, further characterized by XRPD pattern peaks at 17.7, 18.6, and 18.7±0.2° 2θ using Cu Kα radiation.

82. The crystalline form of claim 81, further characterized by XRPD pattern peaks at 12.3, 14.0, and 20.3±0.2° 2θ using Cu Kα radiation.

83. The crystalline form of claim 82, further characterized by XRPD pattern peaks at 11.1, 11.2, 12.4, 16.0, 16.1, 16.7, 16.8, 19.3, 20.7, 21.9, 22.9, 23.0, 24.7, and 24.8±0.2° 2θ using Cu Kα radiation.

84. The crystalline form of any one of claims 80 to 83, having an XRPD pattern substantially as shown in FIG. 37.

85. The crystalline form of any one of claims 80 to 84, having an endothermic transition at 207° C. to 213° C., as measured by differential scanning calorimetry.

86. The crystalline form of claim 85, wherein the endothermic transition is at 210° C.±3° C.

87. The crystalline form of any one of claims 80 to 86, having a thermogravimetric analysis (“TGA”) substantially as shown in FIG. 39.

88. A crystalline form of AMG 397 as a fumarate salt acetone solvate, characterized by XRPD pattern peaks at 17.6, 18.2, and 18.4±0.2° 2θ using Cu Kα radiation.

89. The crystalline form of claim 88, further characterized by XRPD pattern peaks at 5.3, 10.4, 12.2, 13.9, 15.8, and 24.0±0.2° 2θ using Cu Kα radiation.

90. The crystalline form of claim 89, further characterized by XRPD pattern peaks at 9.7, 11.0, 12.9, 14.9, 15.5, 16.3, 16.9, 17.9, 19.2, 20.2, 20.9, 21.6, 22.8, 24.7, and 26.1±0.2° 2θ using Cu Kα radiation.

91. The crystalline form of any one of claims 88 to 90, having an XRPD pattern substantially as shown in FIG. 41.

92. The crystalline form of any one of claims 88 to 91, having an endothermic transition at 229° C. to 235° C., as measured by differential scanning calorimetry.

93. The crystalline form of claim 92, wherein the endothermic transition is at 232° C.±3° C.

94. The crystalline form of any one of claims 88 to 93, having a thermogravimetric analysis (“TGA”) substantially as shown in FIG. 43.

95. A crystalline form of AMG 397 as a fumarate salt, characterized by XRPD pattern peaks at 11.9, 17.9, and 18.1±0.2° 2θ using Cu Kα radiation.

96. The crystalline form of claim 95, further characterized by XRPD pattern peaks at 10.7, 13.6, 15.7, 18.6, 18.8, 19.6, and 21.5±0.2° 2θ using Cu Kα radiation.

97. The crystalline form of claim 96, further characterized by XRPD pattern peaks at 10.3, 14.9, 16.3, 16.5, 20.0, 22.2, 22.6, 13.3, 13.9, 24.5, 25.5, and 28.1±0.2° 2θ using Cu Kα radiation.

98. The crystalline form of any one of claims 95 to 97, having an XRPD pattern substantially as shown in FIG. 45.

99. The crystalline form of any one of claims 95 to 98, having an endothermic transition at 240° C. to 246° C., as measured by differential scanning calorimetry.

100. The crystalline form of claim 99, wherein the endothermic transition is at 243° C.±3° C.

101. The crystalline form of any one of claims 95 to 100, having a thermogravimetric analysis (“TGA”) substantially as shown in FIG. 47.

102. A crystalline form of AMG 397 as a citrate salt, characterized by XRPD pattern peaks at 10.6, 17.6, and 18.3±0.2° 2θ using Cu Kα radiation.

103. The crystalline form of claim 102, further characterized by XRPD pattern peaks at 12.1, 13.9, 16.0, 19.2, and 21.9±0.2° 2θ using Cu Kα radiation.

104. The crystalline form of claim 103, further characterized by XRPD pattern peaks at 6.1, 11.0, 12.8, 15.2, 16.9, 19.5, 20.0, 20.5, 21.1, 22.9, 24.4, 24.7, 25.9, and 28.7±0.2° 2θ using Cu Kα radiation.

105. The crystalline form of any one of claims 102 to 104, having an XRPD pattern substantially as shown in FIG. 49.

106. The crystalline form of any one of claims 102 to 105, having an endothermic transition at 211° C. to 217° C., as measured by differential scanning calorimetry.

107. The crystalline form of claim 106, wherein the endothermic transition is at 214° C.±3° C.

108. The crystalline form of any one of claims 102 to 107, having a thermogravimetric analysis (“TGA”) substantially as shown in FIG. 51.

109. A crystalline form of AMG 397 as a citrate salt, characterized by XRPD pattern peaks at 17.7, 18.4, and 18.5±0.2° 2θ using Cu Kα radiation.

110. The crystalline form of claim 109, further characterized by XRPD pattern peaks at 14.0, 16.0, 20.1, and 21.9±0.2° 2θ using Cu Kα radiation.

111. The crystalline form of claim 110, further characterized by XRPD pattern peaks at 10.7, 11.1, 12.2, 12.9, 15.2, 19.3, 20.6, 22.9, 24.4, and 24.8±0.2° 2θ using Cu Kα radiation.

112. The crystalline form of any one of claims 109 to 111, having an XRPD pattern substantially as shown in FIG. 52.

113. The crystalline form of any one of claims 109 to 112, having an endothermic transition at 203° C. to 209° C., as measured by differential scanning calorimetry.

114. The crystalline form of claim 113, wherein the endothermic transition is at 206° C.±3° C.

115. The crystalline form of any one of claims 109 to 114, having a thermogravimetric analysis (“TGA”) substantially as shown in FIG. 54.

116. A crystalline form of AMG 397 as a lactate salt, characterized by XRPD pattern peaks at 12.1, 17.8, and 18.3±0.2° 2θ using Cu Kα radiation.

117. The crystalline form of claim 116, further characterized by XRPD pattern peaks at 10.5, 10.9, 13.8, 17.5, and 20.0±0.2° 2θ using Cu Kα radiation.

118. The crystalline form of claim 117, further characterized by XRPD pattern peaks at 5.9, 12.8, 15.9, 16.2, 19.1, 20.4, 21.7, 23.9, 24.6, and 25.1±0.2° 2θ using Cu Kα radiation.

119. The crystalline form of any one of claims 116 to 118, having an XRPD pattern substantially as shown in FIG. 57.

120. The crystalline form of any one of claims 116 to 119, having an endothermic transition at 216° C. to 222° C., as measured by differential scanning calorimetry.

121. The crystalline form of claim 120, wherein the endothermic transition is at 219° C.±3° C.

122. The crystalline form of any one of claims 116 to 121, having a thermogravimetric analysis (“TGA”) substantially as shown in FIG. 59.

123. A crystalline form of AMG 397 as a succinate salt, characterized by XRPD pattern peaks at 17.6, 18.4, and 18.7±0.2° 2θ using Cu Kα radiation.

124. The crystalline form of claim 123, further characterized by XRPD pattern peaks at 12.2, 13.9, 18.0, 20.3, and 24.5±0.2° 2θ using Cu Kα radiation.

125. The crystalline form of claim 124, further characterized by XRPD pattern peaks at 5.2, 10.4, 10.7, 11.1, 12.9, 15.2, 15.8, 16.7, 19.1, 21.6, 22.2, 22.8, 23.9, 26.2, 28.3, and 29.2±0.2° 2θ using Cu Kα radiation.

126. The crystalline form of any one of claims 123 to 125, having an XRPD pattern substantially as shown in FIG. 61.

127. The crystalline form of any one of claims 123 to 126, having an endothermic transition at 207° C. to 213° C., as measured by differential scanning calorimetry.

128. The crystalline form of claim 127, wherein the endothermic transition is at 210° C.±3° C.

129. The crystalline form of any one of claims 123 to 128, having a thermogravimetric analysis (“TGA”) substantially as shown in FIG. 63.

130. A crystalline form of AMG 397 as an ammonium salt, characterized by XRPD pattern peaks at 6.2, 10.3, and 17.2±0.2° 2θ using Cu Kα radiation.

131. The crystalline form of claim 130, further characterized by XRPD pattern peaks at 4.0, 4.7, 17.3, 17.9, 19.8, and 20.3±0.2° 2θ using Cu Kα radiation.

132. The crystalline form of claim 131, further characterized by XRPD pattern peaks at 13.0, 14.4, 15.1, 15.5, 15.9, 16.2, 16.4, 17.7, 18.6, 19.7, and 22.8±0.2° 2θ using Cu Kα radiation.

133. The crystalline form of any one of claims 130 to 132, having an XRPD pattern substantially as shown in FIG. 65.

134. The crystalline form of any one of claims 130 to 133, having an endothermic transition at 224° C. to 230° C., as measured by differential scanning calorimetry.

135. The crystalline form of claim 134, wherein the endothermic transition is at 227° C.±3° C.

136. The crystalline form of any one of claims 130 to 135, having a thermogravimetric analysis (“TGA”) substantially as shown in FIG. 67.

137. A crystalline form AMG 397 as a besylate salt, characterized by XRPD pattern peaks at 17.6, 18.4, and 18.7±0.2° 2θ using Cu Kα radiation.

138. The crystalline form of claim 137, further characterized by XRPD pattern peaks at 14.0, 17.7, and 20.4±0.2° 2θ using Cu Kα radiation.

139. The crystalline form of claim 138, further characterized by XRPD pattern peaks at 11.2, 12.4, 13.8, 14.1, 15.9, 16.1, 18.0, 19.3, 20.8, 21.7, 22.9, 23.9, and 24.5±0.2° 2θ using Cu Kα radiation.

140. The crystalline form of any one of claims 137 to 139, having an XRPD pattern substantially as shown in FIG. 68.

141. The crystalline form of any one of claims 137 to 140, having endothermic transitions 54° C. to 60° C. and 231° C. to 237° C., as measured by differential scanning calorimetry.

142. The crystalline form of claim 141, wherein the endothermic transitions are at 57° C.±3° C. and 234° C.±3° C.

143. The crystalline form of any one of claims 137 to 142, having a thermogravimetric analysis (“TGA”) substantially as shown in FIG. 70.

144. A crystalline form of AMG 397 as a tosylate salt, characterized by XRPD pattern peaks at 18.2, 18.4, and 20.5±0.2° 2θ using Cu Kα radiation.

145. The crystalline form of claim 144, further characterized by XRPD pattern peaks at 12.2, 12.3, 17.6, 18.9, and 19.1±0.2° 2θ using Cu Kα radiation.

146. The crystalline form of claim 145, further characterized by XRPD pattern peaks at 4.5, 5.2, 13.0, 13.8, 14.0, 15.2, 15.8, 16.2, 16.4, 19.8, 20.0, 21.4, 22.9, 23.4, 23.6, 23.8, 24.3, 24.6, 25.1, and 27.1±0.2° 2θ using Cu Kα radiation.

147. The crystalline form of any one of claims 144 to 146, having an XRPD pattern substantially as shown in FIG. 72.

148. The crystalline form of any one of claims 144 to 147, having endothermic transitions 37° C. to 43° C. and 223° C. to 229° C., as measured by differential scanning calorimetry.

149. The crystalline form of claim 148, wherein the endothermic transitions are at 40° C.±3° C. and 226° C.±3° C.

150. The crystalline form of any one of claims 144 to 149, having a thermogravimetric analysis (“TGA”) substantially as shown in FIG. 74.

151. A crystalline form of AMG 397 as a maleate salt, characterized by XRPD pattern peaks at 18.2, 18.9, and 19.9±0.2° 2θ using Cu Kα radiation.

152. The crystalline form of claim 151, further characterized by XRPD pattern peaks at 10.4, 10.9, 12.0, and 21.5±0.2° 2θ using Cu Kα radiation.

153. The crystalline form of claim 152, further characterized by XRPD pattern peaks at 10.3, 13.8, 15.8, 17.9, 19.2, and 24.2±0.2° 2θ using Cu Kα radiation.

154. The crystalline form of any one of claims 151 to 153, having an XRPD pattern substantially as shown in FIG. 76.

155. The crystalline form of any one of claims 151 to 154, having an endothermic transition at 219° C. to 225° C., as measured by differential scanning calorimetry.

156. The crystalline form of claim 155, wherein the endothermic transition is at 222° C.±3° C.

157. The crystalline form of any one of claims 151 to 156, having a thermogravimetric analysis (“TGA”) substantially as shown in FIG. 78.

158. A crystalline form of AMG 397 as a maleate salt, characterized by XRPD pattern peaks at 10.6, 18.6, and 20.3±0.2° 2θ using Cu Kα radiation.

159. The crystalline form of claim 158, further characterized by XRPD pattern peaks at 10.8, 12.3, 15.2, 15.9, and 16.7±0.2° 2θ using Cu Kα radiation.

160. The crystalline form of claim 159, further characterized by XRPD pattern peaks at 9.8, 11.1, 13.9, 14.1, 18.0, 18.4, 19.2, 19.4, 20.8, 22.3, 23.0, 23.6, 24.6, and 28.4±0.2° 2θ using Cu Kα radiation.

161. The crystalline form of any one of claims 158 to 160, having an XRPD pattern substantially as shown in FIG. 79.

162. A crystalline form of AMG 397 as a malonate salt, characterized by XRPD pattern peaks at 12.2, 18.8, and 20.4±0.2° 2θ using Cu Kα radiation.

163. The crystalline form of claim 162, further characterized by XRPD pattern peaks at 10.3, 11.1, 17.9, 18.3, and 19.1±0.2° 2θ using Cu Kα radiation.

164. The crystalline form of claim 163, further characterized by XRPD pattern peaks at 10.7, 13.9, 14.0, 15.8, 16.5, 18.4, 19.5, 19.7 21.6, 21.7, 22.8, and 24.5±0.2° 2θ using Cu Kα radiation.

165. The crystalline form of any one of claims 162 to 164, having an XRPD pattern substantially as shown in FIG. 82.

166. The crystalline form of any one of claims 162 to 165, having endothermic transitions 158° C. to 164° C. and 184° C. to 190° C., as measured by differential scanning calorimetry.

167. The crystalline form of claim 166, wherein the endothermic transitions are at 161° C.±3° C. and 187° C.±3° C.

168. The crystalline form of any one of claims 162 to 167, having a thermogravimetric analysis (“TGA”) substantially as shown in FIG. 84.

169. A crystalline form of AMG 397 as a malonate salt, characterized by XRPD pattern peaks at 10.6, 18.5, and 20.2±0.2° 2θ using Cu Kα radiation.

170. The crystalline form of claim 169, further characterized by XRPD pattern peaks at 11.0, 14.0, and 17.9±0.2° 2θ using Cu Kα radiation.

171. The crystalline form of claim 170, further characterized by XRPD pattern peaks at 11.1, 12.3, 15.3, 16.1, 16.8, 17.0, 18.6, 19.4, and 22.2±0.2° 2θ using Cu Kα radiation.

172. The crystalline form of any one of claims 169 to 171, having an XRPD pattern substantially as shown in FIG. 85.

173. A crystalline form of AMG 397 as a tartrate salt, characterized by XRPD pattern peaks at 18.2, 18.6, and 20.2±0.2° 2θ using Cu Kα radiation.

174. The crystalline form of claim 173, further characterized by XRPD pattern peaks at 12.1, 17.8, 19.0, and 21.5±0.2° 2θ using Cu Kα radiation.

175. The crystalline form of claim 174, further characterized by XRPD pattern peaks at 10.6, 11.0, 12.8, 13.8, 15.1, 15.8, 16.4, 16.6, 17.4, 19.3, 19.5, 20.6, 22.1, 22.6, 23.5, and 24.4±0.2° 2θ using Cu Kα radiation.

176. The crystalline form of any one of claims 173 to 175, having an XRPD pattern substantially as shown in FIG. 87.

177. The crystalline form of any one of claims 173 to 176, having an endothermic transition at 224° C. to 230° C., as measured by differential scanning calorimetry.

178. The crystalline form of claim 177, wherein the endothermic transition is at 227° C.±3° C.

179. The crystalline form of any one of claims 173 to 178, having a thermogravimetric analysis (“TGA”) substantially as shown in FIG. 89.

180. A crystalline form of AMG 397 as a tris(hydroxymethyl)aminomethane salt acetone solvate, characterized by XRPD pattern peaks at 10.0, 16.8, and 20.0±0.2° 2θ using Cu Kα radiation.

181. The crystalline form of claim 180, further characterized by XRPD pattern peaks at 12.7, 14.1, and 18.2±0.2° 2θ using Cu Kα radiation.

182. The crystalline form of claim 181, further characterized by XRPD pattern peaks at 6.1, 14.9, 15.3, 16.0, 17.3, 17.6, 18.0, 19.0, 19.1, 19.4, 20.6, 22.1, 22.5, 22.7, 22.9, 26.3, and 26.4±0.2° 2θ using Cu Kα radiation.

183. The crystalline form of any one of claims 180 to 182, having an XRPD pattern substantially as shown in FIG. 90.

184. The crystalline form of any one of claims 180 to 183, having endothermic transitions at 56° C. to 62° C. and 131° C. to 137° C., as measured by differential scanning calorimetry.

185. The crystalline form of claim 184, wherein the endothermic transitions are at 59° C.±3° C. and 134° C.±3° C.

186. The crystalline form of any one of claims 180 to 185, having a thermogravimetric analysis (“TGA”) substantially as shown in FIG. 92.

187. A crystalline form of AMG 397 as an iodide salt, characterized by XRPD pattern peaks at 17.0, 18.0, and 18.1±0.2° 2θ using Cu Kα radiation.

188. The crystalline form of claim 187, further characterized by XRPD pattern peaks at 8.3, 11.0, 18.6, 18.8, 19.1, 20.0, 22.1, 23.5, and 24.7±0.2° 2θ using Cu Kα radiation.

189. The crystalline form of claim 188 further characterized by XRPD pattern peaks at 6.2, 10.6, 10.8, 12.4, 13.0, 14.1, 15.5, 17.6, 22.5, 24.1, 28.6, 28.8, 29.0, and 29.5±0.2° 2θ using Cu Kα radiation.

190. The crystalline form of any one of claims 187 to 189, having an XRPD pattern substantially as shown in FIG. 93.

191. The crystalline form of any one of claims 187 to 190, having an endothermic transition at 228° C. to 234° C., as measured by differential scanning calorimetry.

192. The crystalline form of claim 191, wherein the endothermic transition is at 231° C.±3° C.

193. A pharmaceutical formulation comprising the crystalline form of any one of claims 1 to 192 and a pharmaceutically acceptable excipient.

194. A method of treating a subject suffering from cancer, comprising administering to the subject a therapeutically effective amount of the crystalline form of any one of claims 1 to 192 or the pharmaceutical formulation of claim 193.

195. The method of claim 194, wherein the cancer is multiple myeloma, non-Hodgkin's lymphoma, or acute myeloid leukemia.
